(19) United States
(12) Patent Application Publication
(10) Pub. No.: US 2015/0011528 A1
(43) Pub. Date: Jan. 8, 2015

(54) PHENYLIMIDE-CONTAINING BENZOTHIAZOLE DERIVATIVE OF ITS SALT AND PHARMACEUTICAL COMPOSITION COMPRISING THE SAME

(71) Applicant: YUHAN CORPORATION, Seoul (KR)

(72) Inventors: Youn Hur, Gyeonggi-do (KR); Dong-Hyun Kim, Gyeonggi-do (KR); Eun-Kyung Kim, Gyeonggi-do (KR); Jin-Hwi Park, Gyeonggi-do (KR); Jae-Eun Joo, Gyeonggi-do (KR); Ho-Woong Kang, Gyeonggi-do (KR); Se-Woong Oh, Gyeonggi-do (KR); Dong-Kyun Kim, Gyeonggi-do (KR); Kyoung-Kyu Ahn, Gyeonggi-do (KR)

(73) Assignee: YUHAN CORPORATION, Seoul (KR)

(21) Appl. No.: 14/346,506

(22) PCT Filed: Sep. 21, 2012
(86) PCT No.: PCT/KR2012/007621

§ 371 Date: Mar. 21, 2014

(30) Foreign Application Priority Data
Sep. 23, 2011 (KR) 10-2011-0096128

(51) Int. Cl. C07D 277/82; C07D 417/12; C07D 277/60; C07D 513/04

(52) U.S. Cl. CPC C07D 277/82; C07D 513/04; C07D 417/12; C07D 277/60
USPC 514/210.18; 514/233.8; 514/275; 514/321; 514/366; 514/367; 544/135; 544/331; 546/198; 548/151; 548/163

(57) ABSTRACT

Provided is a phenylimide-containing benzothiazole derivative or its pharmaceutically acceptable salt, a process for the preparation thereof, and a pharmaceutical composition comprising the same. The phenylimide-containing benzothiazole derivative or its pharmaceutically acceptable salt can selectively inhibit the protein-protein interaction between KRS and a laminin receptor (LR), thereby inhibiting migration of cancer cells. Therefore, the phenylimide-containing benzothiazole derivative or its pharmaceutically acceptable salt may be usefully applied for preventing or treating the diseases associated with cancer cell metastasis.

### TECHNICAL FIELD

The present invention relates to a phenylimide-containing benzothiazole derivative or its pharmaceutically acceptable salt, a process for the preparation thereof, and a pharmaceutical composition comprising the same.

### BACKGROUND ART

In general, normal cells control their division and growth precisely in the body. However, when cells lose their regulatory function or divide and grow uncontrollably, they are abnormally over-proliferated, thereby forming malignant tumors. Cancer cells may also spread to other sites in the body. That is, cancer cells growing in a primary cancer may invade neighboring tissues directly, or be metastasized to more distant parts of the body along with the blood vessel or lymphatic vessel. Since cancer cells can freely pass through both the lymphatic system and the venous system, they are broadly metastasized by their vascular spread. Blood-borne cancer cells pass through vascular endothelial cells by aggregation and invasion, thereby entering into the blood stream.

Meanwhile, 67 kDa laminin receptor (LR) is a non-integrin type receptor embedded in plasma membrane and associated with cancer invasion and metastasis (Nelson, J. et al. The 67 kDa laminin receptor: structure, function and role in disease. Biosci. Rep. 28, 33-48 (2008)). LR is often observed at high level in a various cancers (Nelson, J. et al. The 67 kDa laminin receptor: structure, function and role in disease. Biosci. Rep. 28, 33-48 (2008); Menard, S., Castronovo, V., Tagliabue, E. & Sobel, M. E. New insights into the metastasis-associated 67 kD laminin receptor. J. Cell. Biochem. 67, 155-165 (1997)). It has been reported that the laminin signals mediated by LR induce cancer progress and metastasis, through various signaling pathways associated with a G protein, FAK, MAPK, a phosphatase, a phospholipase D, etc (Cancer Letter, 2005).

Recently, it was found that Lysyl-tRNA-synthetases (KRS), one of the aminoacyltRNA synthetases (ARSs), bind to laminin receptor (LR) so as to stabilize the laminin receptor (LR). That is, it was reported that KRS facilitates cell migration and cancer metastasis by stabilizing the laminin receptor (LR) and that KRS over-expression leads to increase in metastasis (WO 2011/056021).

Therefore, it is expected that a material inhibiting or blocking the interaction between KRS and LR can inhibit or block cancer metastasis, thereby usefully applying to prevention and treatment of cancer.

### DISCLOSURE OF INVENTION

### Technical Problem

The present inventors found that a phenylimide-containing benzothiazole derivative or its pharmaceutically acceptable salt selectively inhibit the protein-protein interaction between KRS and LR, thereby inhibiting migration of cancer cells, and therefore can be usefully applied for preventing or treating the diseases associated with cancer cell metastasis.

Therefore, the present invention provides said phenylimide-containing benzothiazole derivative or its pharmaceutically acceptable salt, a process for the preparation thereof, and a pharmaceutical composition comprising the same.

### Solution to Problem

According to an aspect of the present invention, there is provided a phenylimide-containing benzothiazole derivative or its pharmaceutically acceptable salt.

According to another aspect of the present invention, there is provided a process for preparing the phenylimide-containing benzothiazole derivative or its pharmaceutically acceptable salt.

According to still another aspect of the present invention, there is provided a pharmaceutical composition for preventing or treating a disease associated with cancer cell metastasis.

### Advantageous Effects of Invention

The compound of the present invention, i.e., the phenylimide-containing benzothiazole derivative or its pharmaceutically acceptable salt, can selectively inhibit the protein-protein interaction between KRS and LR, without affecting the KRS's irate function (i.e., protein synthesis function of the KRS), thereby inhibiting migration of cancer cells. Therefore, the phenylimide-containing benzothiazole derivative or its pharmaceutically acceptable salt may be usefully applied for preventing or treating the diseases associated with cancer cell metastasis.

### BEST MODE FOR CARRYING OUT THE INVENTION

As used herein, the term “alkyl” refers to a straight or branched aliphatic hydrocarbon radical. For example, C₁-C₆ alkyl means a straight or branched aliphatic hydrocarbon having 1 to 6 carbon atoms, such as methyl, ethyl, propyl, n-butyl, n-pentyl, n-hexyl, isopropyl, isobutyl, sec-butyl, tert-butyl, neopentyl, and isopentyl.

The term “alkoxy or alkyloxy” refers to a radical formed by substituting the hydrogen atom of a hydroxyl group with an alkyl. For example, C₁-C₆ alkoxy includes methoxy, ethoxy, propoxy, n-butoxy, n-pentyloxy, isopropoxy, sec-butoxy, tert-butoxy, neopentyloxy, and isopentyloxy.

The present invention provides a compound of Formula 1 or its pharmaceutically acceptable salt:

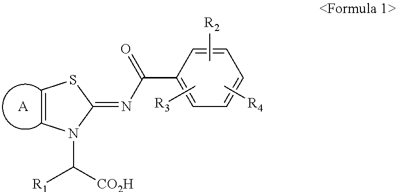

wherein,

R₁ is hydrogen; a C₁˜C₆ alkyl group optionally substituted with hydroxy; a C₂˜C₆ alkenyl group; a halogen group; a trifluoromethyl group; a phenyl group; or a benzyl group,

R₂, R₃, and R₄ are, independently each other, hydrogen; a C₁˜C₆ alkyl group optionally substituted with one or more substituents selected from the group consisting of C₁˜C₃ alkoxy and halogen; a C₁˜C₃ alkoxy group optionally one or more substituted with halogen; a halogen group; a nitro group; a cyano group; a phenoxy group; a benzyloxy group; an amino group; an amino group mono- or di-substituted with C₁˜C₆ alkyl; a phenyl group; or a hydroxycarbonyl group, or

the Ring A is a benzene ring of the following formula A, a cyclohexene ring of the following formula B, or a cycloheptene ring of the following formula C,

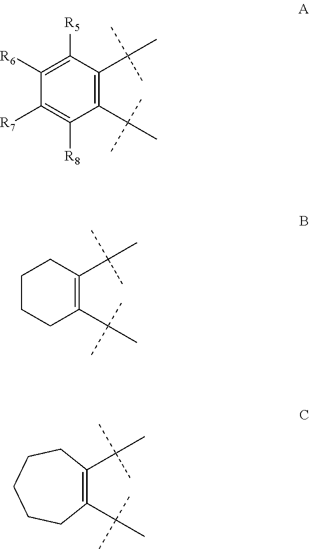

R₅, R₆, R₇, and R₈ are, independently each other, hydrogen; a halogen group; a trifluoromethyl group; a trifluoromethoxy group; a hydroxy group; a hydroxymethyl group; a C₁˜C₆ alkyl group; a C₁˜C₆ alkoxy group optionally substituted with morpholine; C₃˜C₆ cycloalkyloxy group; a phenyl group optionally substituted with halogen; a phenoxy group; a benzyloxy group; a nitro group; a thiophenyl group; an amino group; a dimethylamino group; a R₉—NH group; a R₁₀—C(═O)—NH group; a R₁₀—SO₂—NH group; a R₁₁-carbonyl group; or a R₁₂—NH-carbonyl group, or

R₆ and R₇ may be cyclized each other to form a tricyclic ring of the following formula D,

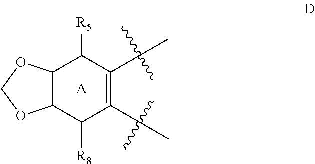

R₉ is C₁˜C₃ alkyl; phenyl optionally substituted with halogen; or pyrimidyl,

R₁₀ is C₁˜C₅ alkyl; C₃˜C₆ cycloalkyl; phenyl; phenylamino; or benzyl (the phenyl group, the phenylamino group, and the benzyl group may be optionally substituted with halogen or trifluoromethyl, respectively),

R₁₁ is hydroxy; C₁˜C₆ alkyl; azetidinyl; pyrrolidinyl; piperidinyl; or morpholinyl, and

R₁₂ is C₁˜C₆ alkyl; phenyl; or phenyl-C₁˜C₃ alkyl.

In a first embodiment of the present invention, the compound or its salt may be a compound or its pharmaceutically acceptable salt of claim 1, wherein the ring A is the benzene ring of the formula A, and R₅, R₆, R₇, and R₈ are not hydrogen at the same time.

In said embodiment, R₁ is hydrogen; or a C₁˜C₆ alkyl group,

R₂, R₃, and R₄ are, independently each other, hydrogen; a C₁˜C₆ alkyl group; a C₁˜C₃ alkoxy group optionally substituted with halogen; a trifluoromethyl group; or a halogen group, and

R₅, R₆, R₇, and R₈ are, independently each other, hydrogen; a halogen group; a trifluoromethyl group; a trifluoromethoxy group; a C₁˜C₆ alkyl group; a C₁˜C₆ alkoxy group; or a nitro group, or

R₆ and R₇ may be cyclized each other to form a tricyclic ring of the following formula D.

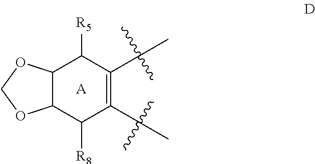

Examples of preferable compounds or its salts in said embodiment includes:
2-{5,6-dimethyl-2-[(4-methylbenzoyl)imino]benzo[d]thiazol-3(2H)-yl}butanoic acid;
2-{2-[(4-methylbenzoyl)imino]-6-(trifluoromethoxy)benzo[d]thiazol-3(2H)-yl}butanoic acid;
2-{5-fluoro-6-methyl-2-[(4-methylbenzoyl)imino]benzo[d]thiazol-3(2H)-yl}butanoic acid;
2-{2-[(4-methylbenzoyl)imino]-6-nitrobenzo[d]thiazol-3(2H)-yl}butanoic acid;
2-{2-[(3-chlorobenzoyl)imino]-6-methoxybenzo[d]thiazol-3(2H)-yl}propionic acid;
2-{2-[(3-chlorobenzoyl)imino]-6-ethoxybenzo[d]thiazol-3(2H)-yl}propionic acid;
2-{2-[(3-chlorobenzoyl)imino]-6-ethoxybenzo[d]thiazol-3(2H)-yl}butanoic acid;
2-{2-[(3-chlorobenzoyl)imino]-6-methylbenzo[d]thiazol-3(2H)-yl}butanoic acid;
2-[5-fluoro-2-(4-methylbenzoylimino)benzo[d]thiazol-3(2H)-yl]butanoic acid;
2-[7-fluoro-2-(4-methylbenzoylimino)benzo[d]thiazol-3(2H)-yl]butanoic acid;
2-[2-(4-methylbenzoylimino)-7-(trifluoromethyl)benzo[d]thiazol-3(2H)-yl]propionic acid;
2-[5-methyl-2-(4-methylbenzoylimino)benzo[d]thiazol-3(2H)-yl]butanoic acid;
2-[7-methyl-2-(4-methylbenzoylimino)benzo[d]thiazol-3(2H)-yl]propionic acid;
2-[5-chloro-2-(4-methylbenzoylimino)benzo[d]thiazol-3(2H)-yl]butanoic acid;
2-[7-chloro-2-(4-methylbenzoylimino)benzo[d]thiazol-3(2H)-yl]propionic acid;
2-[6-fluoro-2-(4-methylbenzoylimino)benzo[d]thiazol-3(2H)-yl]butanoic acid;
2-[2-(4-methylbenzoylimino)-6-(trifluoromethyl)benzo[d]thiazol-3(2H)-yl]butanoic acid;
2-(2-((3-chlorobenzoyl)imino)-6-(trifluoromethyl)benzo[d]thiazol-3(2H)-yl)propionic acid;
2-(2-((3-chlorobenzoyl)imino)-6-(trifluoromethyl)benzo[d]thiazol-3(2H)-yl)butanoic acid;
2-(2-((3-chlorobenzoyl)imino)-6-(trifluoromethoxy)benzo[d]thiazol-3(2H)-yl)butano is acid;
2-(6-methoxy-2-((3-(trifluoromethyl)benzoyl)imino)benzo[d]thiazol-3(2H)-yl)propionic acid;
2-(6-ethoxy-2-((3-(trifluoromethyl)benzoyl)imino)benzo[d]thiazol-3(2H)-yl)propionic acid;
2-(6-methyl-2-((3-(trifluoromethyl)benzoyl)imino)benzo[d]thiazol-3(2H)-yl)propionic acid;
2-(2-((3-chlorobenzoyl)imino)-5,6-dimethylbenzo[d]thiazol-3(2H)-yl)propionic acid;
2-(6-((3-chlorobenzoyl)imino)-[1,3]dioxolo[4′,5′:4,5]benzo[1,2-d]thiazol-7(6H)-yl)butanoic acid;
2-(2-((4-methylbenzoyl)imino)-6-(trifluoromethyl)benzo[d]thiazol-3(2H)-yl)acetic acid;
2-(2-(3-chlorobenzoylimino)-7-(trifluoromethyl)benzo[d]thiazol-3(2H)-yl)propionic acid;
2-(2-(3-chlorobenzoylimino)-4-methoxybenzo[d]thiazol-3(2H)-yl)propionic acid;
2-(4-methoxy-2-(3-(trifluoromethyl)benzoylimino)benzo[d]thiazol-3(2H)-yl)propionic acid;
2-(2-((3-chlorobenzoyl)imino)-5,6-difluorobenzo[d]thiazol-3(2H)-yl)propionic acid;
2-(2-((3-chlorobenzoyl)imino)-5,6-difluorobenzo[d]thiazol-3(2H)-yl)butanoic acid;
2-(5,6-difluoro-2-((3-(trifluoromethyl)benzoyl)imino)benzo[d]thiazol-3(2H)-yl)butanoic acid;
2-(7-chloro-2-((3-(trifluoromethyl)benzoyl)imino)benzo[d]thiazol-3(2H)-yl)propionic acid;
2-(7-chloro-2-((3-chlorobenzoyl)imino)benzo[d]thiazol-3(2H)-yl)propionic acid;
2-(7-chloro-2-((3-chlorobenzoyl)imino)benzo[d]thiazol-3(2H)-yl)butanoic acid;
2-(5-chloro-2-(3-chlorobenzoylimino)benzo[d]thiazol-3(2H)-yl)acetic acid;
2-(7-methyl-2-(3-(trifluoromethyl)benzoylimino)benzo[d]thiazol-3(2H)-yl)propionic acid;
2-(2-((3-chlorobenzoyl)imino)-6,7-difluorobenzo[d]thiazol-3(2H)-yl)butanoic acid;
2-(7-fluoro-2-((3-(trifluoromethyl)benzoyl)imino)benzo[d]thiazol-3(2H)-yl)propionic acid;
2-(2-((3-chlorobenzoyl)imino)-7-fluorobenzo[d]thiazol-3(2H)-yl)acetic acid;
2-(7-fluoro-2-((3-(trifluoromethyl)benzoyl)imino)benzo[d]thiazol-3(2H)-yl)acetic acid;
2-(7-chloro-2-((3-chlorobenzoyl)imino)benzo[d]thiazol-3(2H)-yl)acetic acid;
2-(5-chloro-2-((3-chlorobenzoyl)imino)benzo[d]thiazol-3(2H)-yl)butanoic acid;
2-(2-((3-chlorobenzoyl)imino)-7-fluorobenzo[d]thiazol-3(2H)-yl)propionic acid;
2-(5,6,7-trifluoro-2-((3-(trifluoromethyl)benzoyl)imino)benzo[d]thiazol-3(2H)-yl)propionic acid;
2-(6-chloro-2-((3-chlorobenzoyl)imino)-7-fluorobenzo[d]thiazol-3(2H)-yl)propionic acid;
2-(6-chloro-2-((3-chlorobenzoyl)imino)-7-fluorobenzo[d]thiazol-3(2H)-yl)butanoic acid;
2-(2-((3-chlorobenzoyl)imino)-6-fluoro-5-(trifluoromethyl)benzo[d]thiazol-3(2H)-yl)propionic acid;
2-(2-((3-chlorobenzoyl)imino)-7-fluoro-6-methoxybenzo[d]thiazol-3(2H)-yl)acetic acid;
2-(2-((3-chlorobenzoyl)imino)-5,6,7-trifluorobenzo[d]thiazol-3(2H)-yl)butanoic acid;
2-(2-((3-chlorobenzoyl)imino)-7-fluoro-6-methylbenzo[d]thiazol-3(2H)-yl)propionic acid;
2-(2-((3-chlorobenzoyl)imino)-6-ethoxy-7-fluorobenzo[d]thiazol-3(2H)-yl)butanoic acid;
2-(5,6-difluoro-2-((3-fluorobenzoyl)imino)benzo[d]thiazol-3(2H)-yl)butanoic acid;
2-(5,7-difluoro-2-((4-methylbenzoyl)imino)benzo[d]thiazol-3(2H)-yl)propionic acid;
2-(5,7-difluoro-2-((3-(trifluoromethyl)benzoyl)imino)benzo[d]thiazol-3(2H)-yl)propionic acid;
2-(2-((3-chlorobenzoyl)imino)-5,7-difluorobenzo[d]thiazol-3(2H)-yl)butanoic acid;
2-(2-((3-chloro-4-fluorobenzoyl)imino)-5,6-difluorobenzo[d]thiazol-3(2H)-yl)propionic acid;
2-(2-((3-chloro-4-fluorobenzoyl)imino)-5,6-difluorobenzo[d]thiazol-3(2H)-yl)butano is acid;
2-(2-((3,5-dichlorobenzoyl)imino)-5,6-difluorobenzo[d]thiazol-3(2H)-yl)butanoic acid;
2-(5,6-difluoro-2-((3-fluoro-5-(trifluoromethyl)benzoyl)iminobenzo[d]thiazol-3(2H)-yl)butanoic acid;
2-(5,6-difluoro-2-((4-fluoro-3-(trifluoromethyl)benzoyl)iminobenzo[d]thiazol-3(2H)-yl)butanoic acid;
2-{6-ethoxy-2-[(4-methylbenzoyl)imino]benzo[d]thiazol-3(2H)-yl}butanoic acid;
2-{4-methoxy-2-[(4-methylbenzoyl)imino]benzo[d]thiazol-3(2H)-yl}butanoic acid;
2-[5-fluoro-2-(4-methylbenzoylimino)benzo[d]thiazol-3(2H)-yl]propionic acid;
2-(6-((4-methylbenzoyl)imino)-[1,3]dioxolo[4′,5′:4,5]benzo[1,2-d]thiazol-7(6H)-yl)butanoic acid;
2-(6,7-difluoro-2-((4-methylbenzoyl)imino)benzo[d]thiazol-3(2H)-yl)butanoic acid;
2-(2-((3-chlorobenzoyl)imino)-6,7-difluorobenzo[d]thiazol-3(2H)-yl)propionic acid;
2-(7-methyl-2-((4-methylbenzoyl)imino)benzo[d]thiazol-3(2H)-yl)butanoic acid;
2-(2-((3-chlorobenzoyl)imino)-7-methylbenzo[d]thiazol-3(2H)-yl)propionic acid;
2-(2-((3-chlorobenzoyl)imino)-7-methylbenzo[d]thiazol-3(2H)-yl)butanoic acid;
2-(5-fluoro-2-((3-(trifluoromethyl)benzoyl)imino)benzo[d]thiazol-3(2H)-yl)propionic acid;
2-(5-chloro-2-((3-(trifluoromethyl)benzoyl)imino)benzo[d]thiazol-3(2H)-yl)propionic acid;
2-(2-((3-chlorobenzoyl)imino)-5,6,7-trifluorobenzo[d]thiazol-3(2H)-yl)propionic acid.

Examples of more preferable compounds or its salts in said embodiment includes:
2-{2-[(3-chlorobenzoyl)imino]-6-ethoxybenzo[d]thiazol-3(2H)-yl}butanoic acid;
2-[7-fluoro-2-(4-methylbenzoylimino)benzo[d]thiazol-3(2H)-yl]butanoic acid;
2-[2-(4-methylbenzoylimino)-7-(trifluoromethyl)benzo[d]thiazol-3(2H)-yl]propionic acid;
2-(2-((3-chlorobenzoyl)imino)-6-(trifluoromethyl)benzo[d]thiazol-3(2H)-yl)butanoic acid;
2-(2-((3-chlorobenzoyl)imino)-6-(trifluoromethoxy)benzo[d]thiazol-3(2H)-yl)butanoic acid;
2-(4-methoxy-2-(3-(trifluoromethyl)benzoylimino)benzo[d]thiazol-3(2H)-yl)propionic acid;
2-(2-((3-chlorobenzoyl)imino)-5,6-difluorobenzo[d]thiazol-3(2H)-yl)propionic acid;
2-(2-((3-chlorobenzoyl)imino)-5,6-difluorobenzo[d]thiazol-3(2H)-yl)butanoic acid;
2-(5,6-difluoro-2-((3-(trifluoromethyl)benzoyl)imino)benzo[d]thiazol-3(2H)-yl)butanoic acid;
2-(7-chloro-2-((3-(trifluoromethyl)benzoyl)imino)benzo[d]thiazol-3(2H)-yl)propionic acid;
2-(7-chloro-2-((3-chlorobenzoyl)imino)benzo[d]thiazol-3(2H)-yl)butanoic acid;
2-(5-chloro-2-((3-chlorobenzoyl)imino)benzo[d]thiazol-3(2H)-yl)butanoic acid;
2-(5,6,7-trifluoro-2-((3-(trifluoromethyl)benzoyl)imino)benzo[d]thiazol-3(2H)-yl)propionic acid;
2-(6-chloro-2-((3-chlorobenzoyl)imino)-7-fluorobenzo[d]thiazol-3(2H)-yl)propionic acid;
2-(6-chloro-2-((3-chlorobenzoyl)imino)-7-fluorobenzo[d]thiazol-3(2H)-yl)butanoic acid;
2-(2-((3-chlorobenzoyl)imino)-6-fluoro-5-(trifluoromethyl)benzo[d]thiazol-3(2H)-yl)propionic acid.

In a second embodiment of the present invention, when the ring A is the benzene ring of the formula A, and R₅ to R₈ are hydrogen, R₂, R₃, and R₄ may be a electron withdrawing group.

In said embodiment, the ring A is the benzene ring of the formula A,

R₅, R₆, R₇, and R₈ are hydrogen,

R₁ is hydrogen; or a C₁˜C₆ alkyl group, and

R₂, R₃, and R₄ are, independently each other, hydrogen; a C₁˜C₃ alkoxy group; or a halogen group, with the provisos that R₂, R₃, and R₄ can not be hydrogen at the same time.

Examples of preferable compounds or its salts in said embodiment includes:
2-[2-(4-methoxybenzoylimino)benzo[d]thiazol-3(2H)-yl]butanoic acid;
2-[2-(3,5-difluorobenzoylimino)benzo[d]thiazol-3(2H)-yl]butanoic acid;
2-[2-(3-bromobenzoylimino)benzo[d]thiazol-3(2H)-yl]butanoic acid;
2-[2-(3-chlorobenzoylimino)benzo[d]thiazol-3(2H)-yl]acetic acid;
2-[2-(3-chlorobenzoylimino)benzo[d]thiazol-3(2H)-yl]butanoic acid.

In another embodiment of the present invention, the compound of Formula 1 or its salt may be a compound or its salt having no aromaticity in the ring A.

In said embodiment, the ring A is the cyclohexene ring of the formula B,

R₁ is a C₁˜C₆ alkyl group, and

R₂, R₃, and R₄ are, independently each other, hydrogen; a trifluoromethyl group; or halogen.

Examples of preferable compounds or its salts in said embodiment includes:
2-(2-(3-(trifluoromethyl)benzoylimino)-4,5,6,7-tetrahydrobenzo[d]thiazol-3(2H)-yl)propionic acid;
2-(2-(3-chlorobenzoylimino)-4,5,6,7-tetrahydrobenzo[d]thiazol-3(2H)-yl)butanoic acid.

Examples of more preferable compound or its salt in said embodiment may be 2-(2-(3-Chlorobenzoylimino)-4,5,6,7-tetrahydrobenzo[d]thiazol-3(2H)-yl)butanoic acid or its pharmaceutically acceptable salt.

The compound of Formula 1 or its pharmaceutically acceptable salt may be in the form of cis- or trans-geometrical isomer, according to the double bond therein (e.g., the imino moiety). The compound of Formula 1 or its pharmaceutically acceptable salt comprises both cis- and trans-geometrical isomers, unless otherwise indicated. The compound of Formula 1 or its pharmaceutically acceptable salt may have substituents containing asymmetric carbon and therefore be in the form of racemic mixture (RS) or in forms of optical isomers, such as (R) or (S) isomer. The compound of Formula 1 or its pharmaceutically acceptable salt comprises both racemic mixture (RS) and optical isomers such as (R) or (S) isomer, unless otherwise indicated.

The compound of Formula 1 of the present invention may be in a pharmaceutically acceptable salt form. The salt may be an acid addition salt form, which includes e.g., salts derived from an inorganic acid such as hydrochloric acid, hydrobromic acid, sulfuric acid, or phosphoric acid; and salts derived from an organic acid such as citric acid, acetic acid, lactic acid, tartaric acid, maleic acid, fumaric acid, formic acid, propionic acid, oxalic acid, trifluoroacetic acid, benzoic acid, glucuronic acid, methanesulfonic acid, glycolic acid, succinic acid, p-toluenesulfonic acid, glutamic acid, or aspartic acid. And also, the pharmaceutically acceptable salt may be a metal salt form, which includes e.g., salts derived from an alkali metal such as lithium, sodium, or potassium; or an alkali earth metal such as calcium or magnesium. The metal salt form also includes a chrome salt. In addition, the pharmaceutically acceptable salt may be an organic ligand-derived salt, e.g., quarternary ammonium salt; an amine salt, e.g., dicyclohexylamine salt or N-methyl-D-glucamine salt; or an amino acid salt derived from arginine, lysine, etc.

The present invention also provides a process for preparing a compound of Formula 1 or its pharmaceutically acceptable salt, which comprises converting a compound of Formula 2 to a compound of Formula 3; reacting the compound of Formula 3 with R₁(CH)X—COOR₅ (X=halogen, R₅═C₁˜C₆ alkyl) to obtain a compound of Formula 4; and converting the compound of Formula 4 to the compound of Formula 1, as shown in the following Reaction Scheme 1:

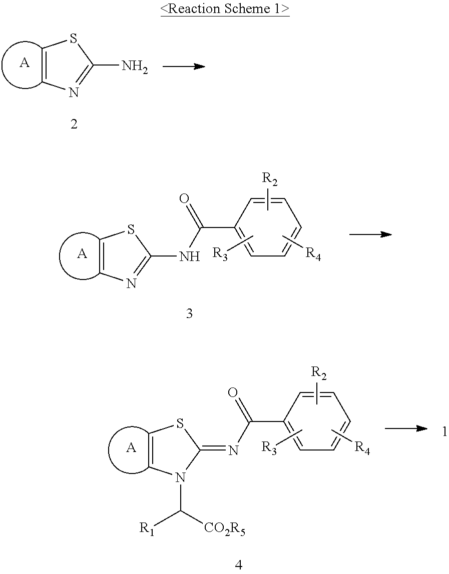

In the Reaction Scheme 1, the ring A, R₁, R₂, R₃, R₄, and R₅ are the same as defined in the above.

The compound of Formula 2 is commercially available and may be prepared according to a known method (for example, Ai Jeng Lin and Sudhaka Kashina, Journal of Heterocyclic chemistry, 1981(18), 759-761; Milos Sedlak, Jiri Hanusek, Michal Holcapek and Vojeslav Sterba, Journal of Physical Organic Chemistry, 2001(14), 187-195). The conversion of the compound of Formula 2 to the compound of Formula 3 may be performed by reacting the compound of Formula 2 with a carboxylic acid or an acyl halide having a phenyl moiety substituted with R₂, R₃, and R₄.

For example, the amide bond may be formed according to known methods, such as an acylation method, an azide method, a carboxylic anhydride reaction method, a carbodiimide method, an active ester method, or a carbonyldiimidazole (for example, Miklos Bodanszky, Principles of Peptide Synthesis, 2nd Ed., 1993). Preferably, an acylating method or a carbodimide method may be used.

The acylating method may be performed in the presence of an organic base such as triethylamine, diisopropylethylamine, pyridine, etc.; and an inorganic base such as potassium carbonate, cesium carbonate, etc. And also, the reaction may be performed in a solvent such as dichloromethane, tetrahydrofuran, N,N-dimethylformamide, etc. Typically, the reaction may be carried out at 0° C. to 80° C. for 10 minutes to 12 hours.

The carbodimide method may be performed by using a coupling agent such as dicyclohexylcarbodimide (DCC), diisopropylcarbodimide and water-soluble N-(3-dimethylaminopropyl)-N′-ethylcarbodimide (EDAC), etc. If necessary, 1-hydroxybenzotriazole (HOBT) may be added for facilitating the reaction. The coupling reaction may be performed in an inert solvent such as dichloromethane, acetonitrile, N,N-dimethylformamide, etc. And also, the reaction may be performed in the presence of an organic base such as triethylamine, diisopropylethylamine, N-methylmorpholine, N,N-dimethylaminopyridine, N-methylpyrrolidine, etc. Typically, the reaction may be carried out at room temperature to 50° C.

The reaction of the compound of Formula 3 and R₁(CH)X—COOR₅ is a reaction of nucleophilic substitution. Preferably, the reaction may be performed in the presence of a base. The base includes an inorganic base such as potassium carbonate (K₂CO₃), cesium carbonate (Cs₂CO₃), sodium tert-butoxide (tert-BuONa), potassium tert-butoxide (tert-BuOK), or sodium hydride (NaH). The reaction may be performed in a solvent including a non-polar organic solvent such as benzene, toluene, etc.; and a polar organic solvent such as N,N-dimethylformamide, acetonitrile, dioxane, tetrahydrofuran, etc. Typically, the reaction may be carried out at 0 to 150° C., preperably 40 to 120° C.

The conversion of the compound of Formula 4 to the compound of Formula 1 may be performed by hydrolyzing the compound of Formula 4. The hydrolysis reaction may be performed under alkaline condition, using sodium hydroxide, lithium hydroxide, potassium hydroxide, etc. The hydrolysis reaction may be performed in water or a mixed solvent of water and a polar solvent (e.g., tetrahydrofuran, ethanol, etc.) as a solvent. Typically, the reaction may be carried out at room temperature to 50° C.

And also, the present invention provides a process for preparing a compound of Formula 1 or its pharmaceutically acceptable salt, which comprises deprotecting a compound of Formula 5 to obtain a compound of Formula 6; converting the compound of Formula 6 to a compound of Formula 4; and converting the compound of Formula 4 to the compound of Formula 1, as shown in the following Reaction Scheme 2:

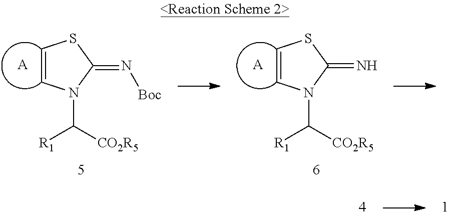

In the Reaction Scheme 2, the ring A, R₁, R₂, R₃, R₄, and R₅ are the same as defined in the above; and Boc is an imine-protecting group.

The compound of Formula 5 may be prepared by introducing a protecting group-containing imine group through an active ester method as in the Reaction Scheme 1. The deprotection of the compound of Formula 5 may be performed according to a known method (for example, Theodora W. Greene and Peter G. M. Wuts, Protective groups in organic synthesis, 3rd Ed., 1999). For example, the deprotection may be performed in an organic solvent (e.g., dichloromethane, dioxane, ethyl acetate, etc.), using trifluoroacetic acid or HCl gas, at room temperature.

The conversion of the compound of Formula 6 to the compound of Formula 4 may be performed by reacting the compound of Formula 6 with a carboxylic acid or an acyl halide having a phenyl moiety substituted with R₂, R₃, and R₄. The reaction may be performed according to an amide coupling reaction, preferably according to an acylating method or a carbodimide method, as described in the Reaction Scheme 1.

The conversion of the compound of Formula 6 to the compound of Formula 4; and the conversion of the compound of Formula 4 to the compound of Formula 1 may be performed according to the same methods as described in the Reaction Scheme 1.

And also, the present invention provides a process for preparing a compound of Formula 1a or its pharmaceutically acceptable salt, which comprises treating a compound of Formula 7 with a benzoyl halide substituted with R₂, R₃, and R₄ and a thiocyanate salt to obtain a compound of Formula 8; cyclizing the compound of Formula 8 to obtain a compound of Formula 9; reacting the compound of Formula 9 with R₁(CH)X—COOR₅ (X=halogen, R₅═C₁˜C₆ alkyl) to obtain a compound of Formula 10; and converting the Formula 10 to the compound of Formula 1a, as shown in the following Reaction Scheme 3:

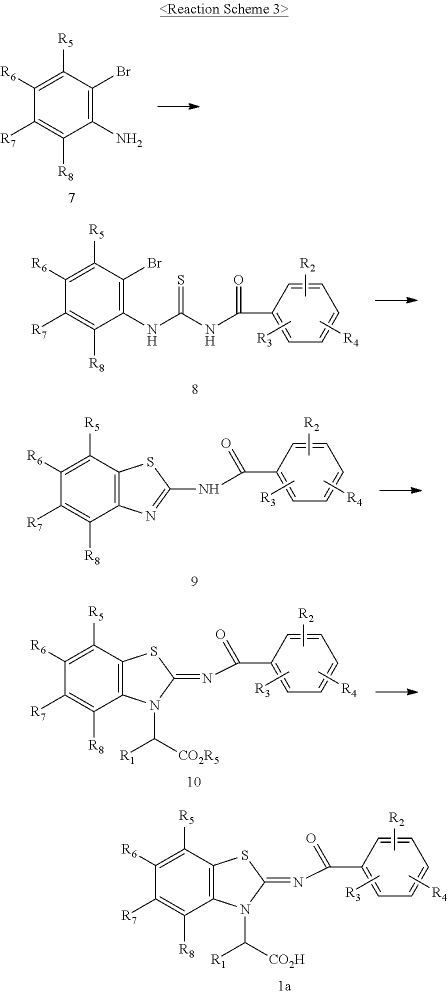

In the Reaction Scheme 3, R₁, R₂, R₃, R₄, R₅, R₆, R₇, and R₈ are the same as defined in the above.

The reaction of the compound of Formula 7 and a benzoyl halide substituted with R₂, R₃, and R₄ and a thiocyanate salt may be performed in a polar organic solvent (e.g., acetone) at room temperature to 80° C., without using a base.

The cyclization of the compound of Formula 8 may be performed in the presence of a base. The base includes an inorganic base such as sodium hydride (NaH), potassium tert-butoxide (tert-BuOK), etc. The reaction may be performed in a polar organic solvent such as N,N-dimethylformamide, acetonitrile, dioxane, tetrahydrofuran, etc. Typically, the reaction may be carried out at room temperature to 100° C. for 1 hour to 12 hours.

The conversion of the compound of Formula 9 to the compound of Formula 10; and the conversion of the compound of Formula 10 to the compound of Formula 1a may be performed according to the same methods as described in the Reaction Scheme 1.

The present invention also provides a pharmaceutical composition for inhibiting cancer cell metastasis, comprising a therapeutically effective amount of the compound of Formula 1 or its pharmaceutically acceptable salt; and a pharmaceutically acceptable carrier. The therapeutically effective amount refers to an amount sufficient for providing an inhibitory activity against cancer cell metastasis. For example, the therapeutically effective amount may be from about 1 mg/kg to about 300 mg/kg per day. Of course, the dosage may be changed according to the patient's age, weight, susceptibility, symptom, or activity of the compound.

And also, the present invention provides a pharmaceutical composition for preventing or treating a disease associated with cancer cell metastasis, comprising a therapeutically effective amount of the compound of Formula 1 or its pharmaceutically acceptable salt; and a pharmaceutically acceptable carrier. The disease associated with cancer cell metastasis is selected from the group consisting of colon cancer, lung cancer, hepatic cancer, gastric cancer, esophageal cancer, pancreatic cancer, gallbladder cancer, kidney cancer, bladder cancer, prostate cancer, testicular cancer, cervical cancer, endometrial cancer, choriocarcinoma, ovarian cancer, breast cancer, thyroid cancer, brain tumor, head and neck cancer, malignant melanoma, lymphoma, and aplastic anemia, but not limited thereto.

The pharmaceutical composition of the present invention may comprise a pharmaceutically acceptable carrier, such as diluents, disintegrants, sweeteners, lubricants, or flavoring agents. The pharmaceutical composition may be formulated to an oral dosage form such as tablets, capsules, powders, granules, suspensions, emulsions, or syrups; or a parenteral dosage form such as injection. The dosage form may be various forms, e.g., dosage forms for single administration or for multiple administrations.

The pharmaceutical composition of the present invention may comprise, for example, a diluent (e.g., lactose, corn starch, etc); a lubricant (e.g., magnesium stearate); an emulsifying agent; a suspending agent; a stabilizer; and/or an isotonic agent. If necessary, the composition further comprises sweeteners and/or flavoring agents.

The composition of the present invention may be administered orally or parenterally, including intravenous, intraperitoneal, subcutaneous, rectal and topical routes of administration. Therefore, the composition of the present invention may be formulated into various forms such as tablets, capsules, aqueous solutions or suspensions. In the case of tablets for oral administration, carriers such as lactose, corn starch, and lubricating agents, e.g. magnesium stearate, are conventionally used. In the case of capsules for oral administration, lactose and/or dried corn starch can be used as a diluent. When an aqueous suspension is required for oral administration, the active ingredient may be combined with emulsifying and/or suspending agents. If desired, certain sweetening and/or flavoring agents may be used. For intramuscular, intraperitoneal, subcutaneous and intravenous administration, sterile solutions of the active ingredient are usually prepared, and the pH of the solutions should be suitably adjusted and buffered. For intravenous administration, the total concentration of solutes should be controlled in order to render the preparation isotonic. The composition of the present invention may be in the form of an aqueous solution containing pharmaceutically acceptable carriers, e.g., saline having a pH level of 7.4. The solutions may be introduced into a patient's intramuscular blood-stream by local bolus injection.

The compound of Formula 1 or its pharmaceutically acceptable salt may be administered in a therapeutically effective amount ranging from about 1 mg/kg to about 300 mg/kg per day to a subject patient. Of course, the dosage may be changed according to the patient's age, weight, susceptibility, symptom, or activity of the compound.

The following examples and experimental examples are provided for illustration purposes only, and are not intended to limit the scope of the invention.

The analyses of the compounds prepared in the following Examples were carried out as follows: Nuclear magnetic resonance (NMR) spectrum analysis was carried out using Bruker 400 MHz spectrometer and chemical shifts thereof were analyzed in ppm. Column chromatography was carried out on silica gel (Merck, 70-230 mesh) (W. C. Still, J. Org. Chem., 1978 (43), 2923-2925). The starting materials in each Example are known compounds, which were synthesized according literatures or obtained from Sigma-Aldrich.

### Preparation 1. 6-trifluoromethoxybenzo[d]thiazol-2-amine

Ammonium thiocyanate (1.70 g, 22.30 mmol) was added to a solution of 4-(trifluoromethoxy)aniline (3.54 g, 20.00 mmol) in acetic acid (30 mL). The reaction mixture was cooled to 0-10° C. A solution of bromine (1.10 mL, 22.00 mmol) in acetic acid (5 mL) was added dropwise to the reaction mixture, which was then stirred at room temperature overnight. The resulting solid was filtered, and then dissolved in water. The aqueous layer was basified to pH 11-12 with solid sodium carbonate. The resulting solid was filtered, washed with water, and then dried in vacuo to give the titled compound (2.84 g) as a white solid. The product was used in the subsequent step without further purification (Yield: 61%).

### Preparation 2. 6-phenoxybenzo[d]thiazol-2-amine

The titled compound (4.27 g) as a white solid was prepared in accordance with the same procedures as in Preparation 1, using 4-phenoxyaniline (3.70 g), instead of 4-(trifluoromethoxy)aniline. The product was used in the next step without further purification (Yield: 88%).

### Preparation 3. 5,6-difluorobenzo[d]thiazol-2-amine

The titled compound (0.73 g) as a white solid was prepared in accordance with the same procedures as in Preparation 1, using 3,4-difluoroaniline (1.29 g), instead of 4-(trifluoromethoxy)aniline. The product was used in the next step without further purification (Yield: 39%).

### Preparation 4. 6-phenylbenzo[d]thiazol-2-amine

The titled compound (3.70 g) as a white solid was prepared in accordance with the same procedures as in Preparation 1, using 4-phenylaniline (3.38 g), instead of 4-(trifluoromethoxy)aniline. The product was used in the next step without further purification (Yield: 82%).

### Preparation 5. 5-fluoro-6-methylbenzo[d]thiazol-2-amine

The titled compound (1.91 g) as a white solid was prepared in accordance with the same procedures as in Preparation 1, using 3-fluoro-4-methylaniline (1.25 g), instead of 4-(trifluoromethoxy)aniline. The product was used in the next step without further purification (Yield: 52%).

### Preparation 6. 6-butylbenzo[d]thiazol-2-amine

The titled compound (2.92 g) as a white solid was prepared in accordance with the same procedures as in Preparation 1, using 4-butylaniline (5.00 g), instead of 4-(trifluoromethoxy)aniline. The product was used in the next step without further purification (Yield: 42%).

### Preparation 7. 6-tert-butylbenzo[d]thiazol-2-amine

The titled compound (2.11 g) as a white solid was prepared in accordance with the same procedures as in Preparation 1, using 4-tert-butylaniline (2.50 g), instead of 4-(trifluoromethoxy)aniline. The product was used in the next step without further purification (Yield: 31%).

### Preparation 8. 6-propylbenzo[d]thiazol-2-amine

The titled compound (4.50 g) as a white solid was prepared in accordance with the same procedures as in Preparation 1, using 4-propylaniline (8.00 g), instead of 4-(trifluoromethoxy)aniline. The product was used in the next step without further purification (Yield: 40%).

### Preparation 9. 6-isopropylbenzo[d]thiazol-2-amine

The titled compound (4.64 g) as a white solid was prepared in accordance with the same procedures as in Preparation 1, using 4-isopropylaniline (8.00 g), instead of 4-(trifluoromethoxy)aniline. The product was used in the next step without further purification (Yield: 41%).

### Preparation 10. 4-methyl-N-(5-nitrobenzo[d]thiazol-2-yl)benzamide

### Step 1: N-[(2-fluoro-3-nitrophenyl)carbamothioyl]-4-methyl-benzamide

p-Toluoyl chloride (4.36 mL, 33.00 mmol) was added to a solution of ammonium thiocyanate (2.74 g, 36.00 mmol) in acetone (60 mL). The reaction mixture was refluxed for 15 minutes. A solution of 2-fluoro-5-nitroaniline (4.68 g, 30.00 mmol) in acetone (20 mL) was added to the reaction mixture, which was then refluxed for 30 minutes. The reaction mixture was poured into ice water. The resulting solid was filtered, washed with water, and then dried under reduced pressure at 50° C. to give the titled compound (9.70 g) as a yellow solid (Yield: 97%). The product was used in the next step without further purification.

### Step 2: 4-methyl-N-(5-nitrobenzo[d]thiazol-2-yl)benzamide

Potassium tert-butoxide (1.10 g, 9.00 mmol) was added to a solution of N-[(2-fluoro-3-nitrophenyl)carbamothioyl]-4-methyl-benzamide (1.00 g, 3.00 mmol) prepared in Step 1 in tetrahydrofuran (50 mL). The reaction mixture was stirred at room temperature for 30 minutes, diluted with ethyl acetate, and then acidified to pH 3-4 with an aqueous 1N HCl solution. The organic layer was separated, washed with brine, dried over anhydrous magnesium sulfate, and then filtered. The filtrate was concentrated under reduced pressure. The residue was suspended with isopropyl ether. The resulting solid was filtered and then dried under reduced pressure to give 0.96 g of the titled compound as a yellow solid (Yield: 98%).

¹H NMR (DMSO-d₆, 400 MHz) δ 13.13 (s, 1H), 8.52 (s, 1H), 8.32 (d, 1H), 8.18 (d, 1H), 8.07 (d, 2H), 7.39 (d, 2H), 2.40 (s, 3H)

### Preparation 11. 4-methyl-N-(5-methoxybenzo[d]thiazol-2-yl)benzamide

### Step 1: 4-methyl-N-[(3-methoxyphenyl)carbamothioyl]benzamide

The titled compound (10.5 g) as a white solid was prepared in accordance with the same procedures as in Step 1 of Preparation 10, using m-anisidine (10.0 g), instead of 2-fluoro-5-nitroaniline. The product was used in the next step without further purification (Yield: 43%).

### Step 2: 4-methyl-N-(5-methoxybenzo[d]thiazol-2-yl)benzamide

To a stirred solution of 4-methyl-N-[(3-methoxyphenyl)carbamothioyl]benzamide (3.00 g, 10.0 mmol) prepared in Step 1 in dichloromethane (40 mL) was added dropwise bromine (0.56 mL, 11.0 mmol) at 0° C. The reaction mixture was stirred at room temperature for 3 hours and then diluted with isopropyl ether. The resulting solid was filtered, washed with ethyl acetate, and then dried under reduced pressure to give 2.10 g of the titled compound as white solid (Yield: 70%).

¹H NMR (DMSO-d₆, 400 MHz) δ 8.02 (d, 2H), 7.86 (d, 1H), 7.37 (d, 2H), 7.28 (s, 1H), 6.97 (d, 1H), 3.82 (s, 3H), 2.38 (s, 3H)

### Preparation 12. 4-methyl-N-(5-benzyloxybenzo[d]thiazol-2-yl)benzamide

### Step 1: 4-methyl-N-[(3-benzyloxyphenyl)carbamothioyl]benzamide

The titled compound (10.98 g) as a white solid was prepared in accordance with the same procedures as in Step 1 of Preparation 10, using 3-benzyloxyaniline (5.98 g), instead of 2-fluoro-5-nitroaniline. The product was used in the next step without further purification (Yield: 97%).

### Step 2: 4-methyl-N-(5-benzyloxybenzo[d]thiazol-2-yl)benzamide

The titled compound as a white solid was prepared in accordance with the same procedures as in Step 2 of Preparation 11, using 4-methyl-N-[(3-benzyloxyphenyl)carbamothioyl]benzamide prepared in Step 1 (Yield: 68%).

¹H NMR (DMSO-d₆, 400 MHz) δ 8.02 (d, 2H), 7.86 (d, 1H), 7.48 (d, 2H), 7.34 (m, 6H), 7.04 (d, 1H), 5.18 (s, 2H), 2.38 (s, 3H)

### Preparation 13. 7-nitrobenzo[d]thiazol-2-amine

### Step 1: 4-methyl-N-[(3-nitrophenyl)carbamothioyl]benzamide

The titled compound (9.46 g) as a yellow solid was prepared in accordance with the same procedures as in Step 1 of Preparation 10, using 3-nitroaniline (4.14 g), instead of 2-fluoro-5-nitroaniline. The product was used in the next step without further purification (Yield: 99%).

### Step 2: 1-(3-nitrophenyl)thiourea

Sodium methoxide (25% in methanol, 7.55 mL, 33.00 mmol) was added dropwise to a solution of 4-methyl-N-[(3-nitrophenyl)carbamothioyl]benzamide (9.46 g, 30.00 mmol) prepared in Step 1 in methanol (150 mL). The reaction mixture was stirred at room temperature overnight, neutralized with an aqueous 1N HCl solution, and then concentrated under reduced pressure. The residue was diluted in ethyl acetate, washed with water and brine, dried over anhydrous magnesium sulfate, and then filtered. The filtrate was concentrated under reduced pressure. The residue was diluted in isopropyl ether and then filtered to obtain the resulting solid. The solid was dried under reduced pressure to give 5.28 g of the titled compound as a yellow solid. The product was used in the next step without further purification (Yield: 89%).

### Step 3: 7-nitrobenzo[d]thiazol-2-amine

To a stirred solution of 1-(3-nitrophenyl)thiourea (5.16 g, 26.20 mmol) prepared in Step 2 in chloroform (130 mL) was added dropwise bromine (2.68 mL, 52.3 mmol) at room temperature. The reaction mixture was refluxed for 3 hours and then cooled to room temperature. The reaction mixture was diluted with isopropyl ether and then filtered. The resulting solid was dissolved in water and then basified to pH 11-12 with sodium carbonate. The aqueous layer was extracted with ethyl acetate. The organic layer was washed with brine, dried over anhydrous magnesium sulfate, and then filtered. The filtrate was concentrated under reduced pressure. The resulting solid was dried under reduced pressure to give 3.52 g of the titled compound as a yellow solid (Yield: 69%).

¹H NMR (CDCl₃, 400 MHz) δ 7.95 (m, 3H), 7.73 (d, 1H), 7.47 (t, 1H)

### Preparation 14. 6-(trifluoromethyl)benzo[d]thiazol-2-amine

The titled compound (11.2 g) as a white solid was prepared in accordance with the same procedures as in Preparation 1, using 4-(trifluoromethyl)aniline (30.0 g), instead of 4-(trifluoromethoxy)aniline. The product was used in the next step without further purification (Yield: 28%).

### Preparation 15. 4,6-difluorobenzo[d]thiazol-2-amine

The titled compound (3.6 g) as a white solid was prepared in accordance with the same procedures as in Preparation 1, using 2,4-difluoroaniline (5.0 g), instead of 4-(trifluoromethoxy)aniline. The product was used in the next step without further purification (Yield: 50%).

### Preparation 16. 6-ethylbenzo[d]thiazol-2-amine

The titled compound (2.22 g) as a white solid was prepared in accordance with the same procedures as in Preparation 1, using 4-ethylaniline (2.42 g), instead of 4-(trifluoromethoxy)aniline. The product was used in the next step without further purification (Yield: 62%).

### Preparation 17. [1,3]dioxolo[4′,5′:4,5]benzo[1,2-d]thiazol-6-amine

The titled compound (3.40 g) as a white solid was prepared in accordance with the same procedures as in Preparation 1, using 3,4-(methylenedioxy)aniline (2.74 g), instead of 4-(trifluoromethoxy)aniline. The product was used in the next step without further purification (Yield: 87%).

### Preparation 18. ethyl 2-(2-iminobenzo[d]thiazol-3(2H)-yl)butanoate

### Step 1: tert-butyl benzo[d]thiazol-2-ylcarbamate

A solution of dimethylaminopyridine (9.7 g, 79.8 mmol) in tetrahydrofuran (15 mL) and di-tert-butyl dicarbonate (19.1 g, 87.8 mmol) were added dropwise at 0° C. to a solution of benzo[d]thiazol-2-amine (12.0 g, 79.8 mmol) in dichloromethane (25 mL). The reaction mixture was stirred at room temperature overnight and then filtered. The filtrate was concentrated under reduced pressure. The resulting residue was recrystallized from ethyl acetate to give 5.8 g of the titled compound as white solid.

### Step 2: ethyl 2-[2-(tert-butoxycarbonylimino)benzo[d]thiazol-3(2H)-yl]butanoate

Ethyl 2-bromobutyrate (2.3 g, 11.98 mmol) and potassium carbonate (2.5 g, 17.97 mmol) were added to a solution of tert-butyl benzo[d]thiazol-2-ylcarbamate (3.0 g, 11.98 mmol) prepared in Step 1 in N,N-dimethylformamide (40.0 mL). The reaction mixture was stirred at room temperature overnight and then quenched with water. The reaction mixture was extracted with ethyl acetate. The organic layer was dried over anhydrous sodium sulfate, filtered, and then evaporated. The residue was purified with silica gel column chromatography (n-hexaneethyl acetate=151) to give 2.3 g of the titled compound as white solid (Yield: 55%).

¹H NMR (CDCl₃, 400 MHz) δ 7.61 (d, 1H), 7.31 (dd, 1H), 7.23 (dd, 1H), 7.09 (d, 1H), 6.07 (brs, 1H), 4.10-4.23 (m, 2H), 2.37-2.42 (m, 1H), 2.21-2.27 (m, 2H), 1.28 (s, 9H), 1.13 (t, 3H), 0.86 (t, 3H)

### Step 3: ethyl 2-(2-iminobenzo[d]thiazol-3(2H)-yl)butanoate

To a stirred solution of ethyl 2-[2-(tert-butoxycarbonylimino)benzo[d]thiazol-3(2H)-yl]butanoate (2.0 g, 5.71 mmol) prepared in Step 2 in ethyl acetate (50 mL) was passed HCl gas at 0° C. for 3 minutes. The reaction mixture was stirred at room temperature overnight and then evaporated. The resulting solid was washed with ethyl acetate and then dried under reduced pressure to give 1.4 g of the titled compound (Yield: 92%).

¹H NMR (CDCl₃, 400 MHz) δ 11.25 (brs, 2H), 7.74 (d, 1H), 7.34-7.42 (m, 2H), 7.17 (d, 1H), 6.24 (brs, 1H), 4.22 (d, 2H), 2.52-2.56 (m, 1H), 2.23-2.30 (m, 1H), 1.15 (t, 3H), 1.04 (t, 3H)

### Preparation 19. ethyl 2-(2-iminobenzo[d]thiazol-3(2H)-yl)propionate

### Step 1: ethyl 2-(2-((tert-butoxycarbonyl)imino)benzo[d]thiazol-3(2H)-yl)propanoate

To a stirred solution of tert-butyl benzo[d]thiazol-2-ylcarbamate (1.0 g, 3.99 mmol) prepared in Step 1 of Preparation 18 in N,N-dimethylformamide (12.0 mL) were added ethyl 2-bromopropionate (868 mg, 4.79 mmol) and potassium carbonate (1.10 g, 7.98 mmol). The reaction mixture was stirred at 80° C. for 3 hours. The reaction mixture was quenched with water and then extracted with ethyl acetate. The organic layer was dried over anhydrous sodium sulfate, filtered, and then evaporated. The residue was purified with silica gel column chromatography (n-hexaneethyl acetate=10/1) to give 501 mg of the titled compound as a white solid (Yield: 36%).

### Step 2: ethyl 2-(2-iminobenzo[d]thiazol-3(2H)-yl)propanoate

To a stirred solution of ethyl 2-(2-((tert-butoxycarbonyl)imino)benzo[d]thiazol-3(2H)-yl)propanoate (501 mg, 1.43 mmol) prepared in Step 1 in ethyl acetate (30 mL) was passed HCl gas at 0° C. for 5 minutes. The reaction mixture was stirred at room temperature overnight and then evaporated. The resulting solid was washed with ethyl acetate and then dried under reduced pressure to give 329 mg of the titled compound as a white solid (Yield: 92%).

### Preparation 20. ethyl 2-(2-iminobenzo[d]thiazol-3(2H)-yl)acetate

To a stirred solution of tert-butyl benzo[d]thiazol-2-ylcarbamate (1.0 g, 3.99 mmol) prepared in Step 1 of Preparation 18 in N,N-dimethylformamide (12.0 mL) were added ethyl bromoacetate (800 mg, 4.79 mmol) and potassium carbonate (1.10 g, 7.98 mmol). The reaction mixture was stirred at 80° C. for 3 hours and then quenched with water. The reaction mixture was extracted with ethyl acetate. The organic layer was dried over anhydrous sodium sulfate, filtered, and then evaporated. The residue was purified with silica gel column chromatography (n-hexaneethyl acetate=10/1) to give 470 mg of the titled compound as a white solid (Yield: 35%).

### Step 2: ethyl 2-(2-iminobenzo[d]thiazol-3(2H)-yl)acetate

To a stirred solution of ethyl 2-(2-((tert-butoxycarbonyl)imino)benzo[d]thiazol-3(2H)-yl)acetate (470 mg, 1.40 mmol) prepared in Step 1 in ethyl acetate (30 mL) was passed HCl gas at 0° C. for 5 minutes. The reaction mixture was stirred at room temperature overnight and then evaporated. The resulting solid was washed with ethyl acetate and then dried under reduced pressure to give 283 mg of the titled compound as a white solid (Yield: 86%).

### Example 1

### 2-[5-bromo-2-(4-methylbenzoylimino)benzo[d]thiazol-3(2H)-yl]butanoic acid

### Step 1: N-(2,5-dibromophenylcarbamothioyl)-4-methylbenzamide

To a stirred solution of 2,5-dibromoaniline (1.77 g, 7.05 mmol) in N,N-dimethylformamide (28 mL) was added portionwise 4-methylbenzoyl isocyanate (500 mg, 2.82 mmol). The reaction mixture was stirred at room temperature overnight. The resulting solid was filtered, washed with methanol, and then dried under reduced pressure to give 560 mg of the titled compound as a white solid (Yield: 46%).

¹H NMR (CDCl₃, 400 MHz) δ 9.12 (s, 1H), 8.59 (s, 1H), 7.81 (d, 2H), 7.51 (d, 1H), 7.35 (d, 2H), 7.29 (s, 1H), 2.45 (s, 3H)

### Step 2: N-(5-bromobenzo[d]thiazol-2-yl)-4-methylbenzamide

To a stirred solution of N-(2,5-dibromophenylcarbamothioyl)-4-methylbenzamide (200 mg, 0.468 mmol) prepared in Step 1 in N,N-dimethylformamide (8.0 mL) was added NaH (60%, 186 mg, 4.68 mmol). The reaction mixture was stirred at 100° C. for 2 hours. The reaction mixture was quenched with water and then extracted with dichloromethane. The organic layer was dried over anhydrous sodium sulfate, filtered, and then evaporated. The residue was used in the next step without further purification.

### Step 3: methyl 2-[5-bromo-2-(4-methylbenzoylimino)benzo[d]thiazol-3(2H)-yl]butanoate

To a stirred solution of N-(5-bromobenzo[d]thiazol-2-yl)-4-methylbenzamide (90 mg, 0.258 mmol) prepared in Step 2 in N,N-dimethylformamide (3.0 mL) were added methyl 2-bromobutyrate (93.9 mg, 0.519 mmol) and potassium carbonate (107 mg, 0.768 mmol). The reaction mixture was stirred at 80° C. for 3 hours. The reaction mixture was quenched with water and then extracted with ethyl acetate. The organic layer was dried over anhydrous sodium sulfate, filtered, and then evaporated. The residue was purified with silica gel column chromatography (n-hexaneethyl acetate=10/1) to give 109.5 mg of the titled compound as a white solid (Yield: 94%)

¹H NMR (CDCl₃, 400 MHz) δ 8.18 (d, 2H), 7.56 (d, 1H), 7.42 (s, 2H), 7.28 (d, 2H), 5.46 (brs, 1H), 3.67 (s, 3H), 2.47-2.54 (m, 2H), 2.42 (s, 3H), 0.92 (t, 3H)

### Step 4: 2-[5-bromo-2-(4-methylbenzoylimino)benzo[d]thiazol-3(2H)-yl]butanoic acid

To a stirred solution of methyl 2-[5-bromo-2-(4-methylbenzoylimino)benzo[d]thiazol-3(2H)-yl]butanoate (41.9 mg, 0.093 mmol) prepared in Step 3 in a mixed solvent of tetrahydrofuran and methanol (1/1, 2.0 mL) was added aqueous 1N sodium hydroxide solution (0.5 mL). The reaction mixture was stirred at room temperature overnight, concentrated under reduced pressure, acidified with an aqueous 1N HCl solution, and then extracted with dichloromethane. The organic layer was washed with brine, dried over anhydrous magnesium sulfate, filtered, and then evaporated. The residue was dried under reduced pressure to give 35.4 mg of the titled compound as a white solid (Yield: 88%).

¹H NMR (CDCl₃, 400 MHz) δ 8.11 (d, 2H), 7.52 (d, 1H), 7.39-7.45 (m, 2H), 7.20 (d, 2H), 5.46 (brs, 1H), 2.48 (dd, 2H), 2.38 (s, 3H), 0.88 (t, 3H)

### Example 2

### 2-[6-(2,3-difluorophenyl)-2-(4-methylbenzoylimino)benzo[d]thiazol-3(2H)-yl]butanoic acid

### Step 1: N-(6-bromobenzo[d]thiazol-2-yl)-4-methylbenzamide

To a stirred solution of 6-bromobenzo[d]thiazol-2-amine (10.0 g, 43.7 mmol) in toluene (146 mL) were added diisopropylethylamine (15.2 mL, 87.3 mmol) and p-toluoyl chloride (10.1 g, 65.5 mmol). The reaction mixture was stirred at 80° C. for 3 hours. The reaction mixture was quenched with water and then extracted with ethyl acetate. The organic layer was washed with an aqueous 1N HCl solution, a saturated sodium bicarbonate solution, and brine, dried over anhydrous magnesium sulfate, filtered, and then evaporated. Isopropyl ether was added to the resulting residue and then filtered. The resulting solid was dried under reduced pressure to give 12.7 g of the titled compound as a white solid (Yield: 84%).

### Step 2: methyl 2-[6-bromo-2-(4-methylbenzoylimino)benzo[d]thiazol-3(2H)-yl]butanoate

To a stirred solution of N-(6-bromobenzo[d]thiazol-2-yl)-4-methylbenzamide (12.7 g, 36.6 mmol) prepared in Step 1 in N,N-dimethylformamide (122 mL) were added potassium carbonate (10.1 g, 73.2 mmol) and methyl 2-bromobutyrate (8.6 g, 47.5 mmol). The reaction mixture was stirred at 80° C. for 2 hours. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=2/1, v/v) to give 12.4 g of the titled compound as a white solid (Yield: 75%).

¹H NMR (CDCl₃, 400 MHz) δ 8.18 (d, 2H), 7.83 (d, 1H), 7.53 (dd, 1H), 7.27 (d, 2H), 7.14 (d, 1H), 5.56 (brs, 1H), 3.66 (s, 3H), 2.42-2.56 (m, 2H), 2.42 (s, 3H), 0.90 (t, 3H)

### Step 3: 2-[6-(2,3-difluorophenyl)-2-(4-methylbenzoylimino)benzo[d]thiazol-3(2H)-yl]butanoic acid

To a stirred solution of methyl 2-[6-bromo-2-(4-methylbenzoylimino)benzo[d]thiazol-3(2H)-yl]butanoate (50 mg, 0.111 mmol) prepared in Step 2 in N,N-dimethylformamide (1.5 mL), were added 2,3-difluorophenyl boronic acid (0.167 mmol, 26.3 mg), tetrakis(triphenylphosphine)palladium(0) (5 mg, 0.004 mmol), and a 2M potassium phosphate (0.5 mL). The reaction mixture was stirred at 80° C. for 4 hours. The reaction mixture was quenched with water, acidified with a 2N HCl solution, and then extracted with dichloromethane. The organic layer was washed with brine, dried over anhydrous magnesium sulfate, filtered, and then evaporated. The residue was purified with silica gel column chromatography (dichloromethanemethanol=30/1) to give 7.7 mg of the titled compound as a white solid (Yield: 15%).

¹H NMR (CDCl₃, 400 MHz) δ 8.18 (d, 2H), 7.79 (s, 1H), 7.52 (d, 1H), 7.35-7.40 (m, 2H), 7.23-7.28 (m, 4H), 5.75 (brs, 1H), 2.46-2.54 (m, 2H), 2.40 (s, 3H), 0.90 (t, 3H)

### Example 3

### 2-[2-(4-methylbenzoylimino)-6-(thiophen-3-yl)benzo[d]thiazol-3(2H)-yl]butanoic acid

The titled compound was prepared in accordance with the same procedures as in Example 2, using thiophen-3-boronic acid, instead of 2,3-difluorophenyl boronic acid (Yield: 25%).

¹H NMR (CDCl₃, 400 MHz) δ 8.17 (d, 2H), 7.85 (s, 1H), 7.60 (d, 1H), 7.37-7.48 (m, 3H), 7.31 (d, 1H), 7.23 (d, 2H), 5.66 (brs, 1H), 2.46-2.52 (m, 2H), 2.39 (s, 3H), 0.88 (t, 3H)

### Example 4

### 2-[6-(4-fluorophenyl)-2-(4-methylbenzoylimino)benzo[d]thiazol-3(2H)-yl]butanoic acid

The titled compound was prepared in accordance with the same procedures as in Example 2, using 4-fluorophenyl boronic acid, instead of 2,3-difluorophenyl boronic acid (Yield: 25%).

¹H NMR (CDCl₃, 400 MHz) δ 8.17 (d, 2H), 7.85 (s, 1H), 7.60 (d, 1H), 7.37-7.48 (m, 3H), 7.31 (d, 1H), 7.23 (d, 2H), 5.66 (brs, 1H), 2.46-2.52 (m, 2H), 2.39 (s, 3H), 0.88 (t, 3H)

### Example 5

### 2-{6-methyl-2-[(4-methylbenzoyl)imino]benzo[d]thiazol-3(2H)-yl}butanoic acid

### Step 1: 4-methyl-N-[6-methylbenzo[d]thiazol-2-yl]benzamide

To a solution of 6-methylbenzo[d]thiazol-2-amine (1.64 g, 10.0 mmol) in tetrahydrofuran (50 mL) were added cesium carbonate (9.0 g, 30.00 mmol) and p-toluoyl chloride (2.0 mL, 15.0 mmol). The reaction mixture was stirred at room temperature overnight. The reaction mixture was quenched with water and then extracted with ethyl acetate. The organic layer was washed with an aqueous 1N HCl solution, saturated sodium bicarbonate solution, and brine. The organic layer was dried over anhydrous magnesium sulfate, filtered, and then evaporated. Isopropyl ether was added to the resulting residue and then filtered. The resulting solid was dried under reduced pressure to give 1.21 g of the titled compound as a white solid (Yield: 43%).

### Step 2: ethyl 2-{2-[(4-methylbenzoyl)imino]-6-methylbenzo[d]thiazol-3(2H)-yl}butanoate

To a stirred solution of 4-methyl-N-[6-methylbenzo[d]thiazol-2-yl]benzamide (217 mg, 0.77 mmol) prepared in Step 1 in N,N-dimethylformamide (3 mL) were added potassium carbonate (387 mg, 2.80 mmol) and ethyl 2-bromobutyrate (207 uL, 1.40 mmol). The reaction mixture was stirred at 50° C. for 5 hours. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=2/1) to give 225 mg of the titled compound as a white solid (Yield: 76%).

¹H NMR (CDCl₃, 400 MHz) δ 8.19 (d, 2H), 7.52 (s, 1H), 7.26 (m, 3H), 7.17 (d, 1H), 5.53 (brs, 1H), 4.20-4.08 (m, 2H), 2.50 (m, 2H), 2.45 (s, 3H), 2.42 (s, 3H), 1.06 (t, 3H), 0.90 (t, 3H)

### Step 3: 2-{6-methyl-2-[(4-methylbenzoyl)imino]benzo[d]thiazol-3(2H)-yl}butanoic acid

To a solution of ethyl 2-{6-methyl-2-[(4-methylbenzoyl)imino]benzo[d]thiazol-3(2H)-yl}butanoate (213 mg, 0.499 mmol) prepared in Step 2 in a mixed solvent of tetrahydrofuran and methanol (1/1, 4.0 mL) was added a 3N sodium hydroxide solution (0.6 mL). The reaction mixture was stirred at room temperature overnight. The reaction mixture was evaporated, diluted with water, and then acidified to pH 3-4 with an aqueous 1N HCl solution. The aqueous layer was extracted with ethyl acetate. The organic layer was washed with brine, dried over anhydrous magnesium sulfate, filtered, and then evaporated. The residue was concentrated under reduced pressure to give 92 mg of the titled compound as a white solid (Yield: 45%).

¹H NMR (DMSO-d₆, 400 MHz) δ 8.07 (d, 2H), 7.26 (s, 1H), 7.64 (brs, 1H), 7.31 (m, 3H), 5.68 (brs, 1H), 2.25-2.50 (m, 2H), 2.40 (s, 3H), 2.38 (s, 3H), 0.72 (s, 3H)

### Example 6

### 2-{5,6-dimethyl-2-[(4-methylbenzoyl)imino]benzo[d]thiazol-3(2H)-yl}butanoic acid

The titled compound as a white solid was prepared in accordance with the same procedures as in Example 5, using 5,6-dimethylbenzo[d]thiazol-2-amine, instead of 6-methylbenzo[d]thiazol-2-amine (Yield: 55%).

¹H NMR (DMSO-d₆, 400 MHz) δ 8.07 (d, 2H), 7.66 (s, 1H), 7.58 (s, 1H), 7.29 (d, 2H), 5.66 (brs, 1H), 2.42-2.55 (m, 2H), 2.37 (s, 3H), 2.34 (s, 3H), 2.30 (s, 3H), 0.73 (t, 3H)

### Example 7

### 2-{6-ethoxy-2-[(4-methylbenzoyl)imino]benzo[d]thiazol-3(2H)-yl}butanoic acid

The titled compound as a white solid was prepared in accordance with the same procedures as in Example 5, using 6-ethoxybenzo[d]thiazol-2-amine, instead of 6-methylbenzo[d]thiazol-2-amine (Yield: 57%).

¹H NMR (DMSO-d₆, 400 MHz) δ 8.07 (d, 2H), 7.66 (brs, 1H), 7.54 (s, 1H), 7.29 (d, 2H), 7.10 (d, 1H), 5.66 (brs, 1H), 4.07 (m, 2H), 2.25-2.50 (m, 2H), 2.37 (s, 3H), 1.35 (s, 3H), 0.73 (s, 3H)

### Example 8

### 2-{4-methoxy-2-[(4-methylbenzoyl)imino]benzo[d]thiazol-3(2H)-yl}butanoic acid

The titled compound as a white solid was prepared in accordance with the same procedures as in Example 5, using 4-methoxybenzo[d]thiazol-2-amine, instead of 6-methylbenzo[d]thiazol-2-amine (Yield: 57%).

¹H NMR (DMSO-d₆, 400 MHz) δ 8.07 (d, 2H), 7.67 (brs, 1H), 7.57 (s, 1H), 7.29 (d, 2H), 7.11 (d, 1H), 5.67 (brs, 1H), 3.82 (s, 3H), 2.25-2.50 (m, 2H), 2.37 (s, 3H), 0.73 (s, 3H)

### Example 9

### 2-{6-bromo-2-[(4-methylbenzoyl)imino]benzo[d]thiazol-3(2H)-yl}butanoic acid

The titled compound as a white solid was prepared in accordance with the same procedures as in Example 5, using 6-bromobenzo[d]thiazol-2-amine, instead of 6-methylbenzo[d]thiazol-2-amine (Yield: 51%).

¹H NMR (DMSO-d₆, 400 MHz) δ 8.21 (s, 1H), 8.07 (d, 2H), 7.70 (m, 2H), 7.31 (d, 2H), 5.72 (brs, 1H), 2.25-2.50 (m, 2H), 2.38 (s, 3H), 0.74 (s, 3H)

### Example 10

### 2-{6-chloro-2-[(4-methylbenzoyl)imino]benzo[d]thiazol-3(2H)-yl}butanoic acid

The titled compound as a white solid was prepared in accordance with the same procedures as in Example 5, using 6-chlorobenzo[d]thiazol-2-amine, instead of 6-methylbenzo[d]thiazol-2-amine (Yield: 43%).

¹H NMR (DMSO-d₆, 400 MHz) δ 8.09 (brs, 3H), 7.77 (brs, 1H), 7.57 (d, 1H), 7.30 (d, 2H), 5.70 (brs, 1H), 2.25-2.50 (m, 2H), 2.34 (s, 3H), 0.74 (s, 3H)

### Example 11

### 2-{6-methoxy-2-[(4-methylbenzoyl)imino]benzo[d]thiazol-3(2H)-yl}butanoic acid

The titled compound as a white solid was prepared in accordance with the same procedures as in Example 5, using 6-methoxybenzo[d]thiazol-2-amine and methyl 2-bromobutyrate, instead of 6-methylbenzo[d]thiazol-2-amine and ethyl 2-bromobutyrate, respectively (Yield: 72%).

¹H NMR (DMSO-d₆, 400 MHz) δ 13.06 (brs, 1H), 8.09 (d, 2H), 7.70 (s, 1H), 7.59 (d, 1H), 7.30 (d, 2H), 7.13 (dd, 1H), 5.71 (brs, 1H), 3.83 (s, 3H), 2.30-2.50 (m, 2H), 2.39 (s, 3H), 0.75 (t, 3H)

### Example 12

### 2-{2-[(4-methylbenzoyl)imino]-6-phenoxybenzo[d]thiazol-3(2H)-yl}butanoic acid

The titled compound as a white solid was prepared in accordance with the same procedures as in Example 5, using 6-phenoxybenzo[d]thiazol-2-amine prepared in Preparation 2 and methyl 2-bromobutyrate, instead of 6-methylbenzo[d]thiazol-2-amine and ethyl 2-bromobutyrate, respectively (Yield: 73%).

¹H NMR (DMSO-d₆, 400 MHz) δ 13.12 (brs, 1H), 8.09 (d, 2H), 7.81 (s, 1H), 7.69 (s, 1H), 7.41 (m, 2H), 7.30 (d, 2H), 7.23 (d, 1H), 7.16 (m, 1H), 7.05 (d, 2H), 5.74 (brs, 1H), 2.30-2.60 (m, 2H), 2.38 (s, 3H), 0.78 (t, 3H)

### Example 13

### 2-{5,6-difluoro-2-[(4-methylbenzoyl)imino]benzo[d]thiazol-3(2H)-yl}butanoic acid

The titled compound as a white solid was prepared in accordance with the same procedures as in Example 5, using 5,6-difluorobenzo[d]thiazol-2-amine prepared in Preparation 3 and methyl 2-bromobutyrate, instead of 6-methylbenzo[d]thiazol-2-amine and ethyl 2-bromobutyrate, respectively (Yield: 53%).

¹H NMR (DMSO-d₆, 400 MHz) δ 13.16 (brs, 1H), 8.17 (m, 2H), 8.07 (d, 2H), 7.31 (d, 2H), 5.62 (brs, 1H), 2.30-2.60 (m, 2H), 2.39 (s, 3H), 0.76 (t, 3H)

### Example 14

### 2-{2-[(4-methylbenzoyl)imino]-6-(trifluoromethoxy)benzo[d]thiazol-3(2H)-yl]butanoic acid

The titled compound as a white solid was prepared in accordance with the same procedures as in Example 5, using 6-trifluoromethoxybenzo[d]thiazol-2-amine prepared in Preparation 1 and methyl 2-bromobutyrate, instead of 6-methylbenzo[d]thiazol-2-amine and ethyl 2-bromobutyrate, respectively (Yield: 71%).

¹H NMR (DMSO-d₆, 400 MHz) δ 13.13 (brs, 1H), 8.14 (s, 1H), 8.10 (m, 2H), 7.91 (s, 1H), 7.57 (d, 1H), 7.32 (d, 2H), 5.76 (brs, 1H), 2.30-2.60 (m, 2H), 2.39 (s, 3H), 0.77 (t, 3H)

### Example 15

### 2-{2-[(4-methylbenzoyl)imino]-6-phenylbenzo[d]thiazol-3(2H)-yl}butanoic acid

The titled compound as a white solid was prepared in accordance with the same procedures as in Example 5, using 6-phenylbenzo[d]thiazol-2-amine prepared in Preparation 4 and methyl 2-bromobutyrate, instead of 6-methylbenzo[d]thiazol-2-amine and ethyl 2-bromobutyrate, respectively (Yield: 65%).

¹H NMR (DMSO-d₆, 400 MHz) δ 8.24 (s, 1H), 8.13 (s, 2H), 7.72 (m, 4H), 7.48 (t, 2H), 7.38 (d, 1H), 7.31 (d, 2H), 5.84 (brs, 1H), 2.30-2.50 (m, 2H), 2.38 (s, 3H), 0.76 (t, 3H)

### Example 16

### 2-{5-fluoro-6-methyl-2-[(4-methylbenzoyl)imino]benzo[d]thiazol-3(2H)-yl}butanoic acid

The titled compound as a white solid was prepared in accordance with the same procedures as in Example 5, using 5-fluoro-6-methylbenzo[d]thiazol-2-amine prepared in Preparation 5 and methyl 2-bromobutyrate, instead of 6-methylbenzo[d]thiazol-2-amine and ethyl 2-bromobutyrate, respectively (Yield: 90%).

¹H NMR (DMSO-d₆, 400 MHz) δ 8.09 (d, 2H), 7.82 (d, 1H), 7.59 (s, 1H), 7.30 (d, 2H), 5.68 (brs, 1H), 2.30-2.50 (m, 2H), 2.37 (s, 3H), 2.30 (s, 3H), 0.73 (t, 3H)

### Example 17

### 2-{6-butyl-2-[(4-methylbenzoyl)imino]benzo[d]thiazol-3(2H)-yl}butanoic acid

The titled compound as a white solid was prepared in accordance with the same procedures as in Example 5, using 6-butylbenzo[d]thiazol-2-amine prepared in Preparation 6 and methyl 2-bromobutyrate, instead of 6-methylbenzo[d]thiazol-2-amine and ethyl 2-bromobutyrate, respectively (Yield: 81%).

¹H NMR (DMSO-d₆, 400 MHz) δ 13.06 (brs, 1H), 8.10 (d, 2H), 7.75 (s, 1H), 7.66 (s, 1H), 7.36 (d, 1H), 7.31 (d, 2H), 5.71 (brs, 1H), 2.67 (t, 2H), 2.31-2.38 (m, 2H), 2.34 (s, 3H), 1.62 (m, 2H), 1.34 (m, 2H), 0.91 (t, 3H), 0.74 (t, 3H)

### Example 18

### 2-{6-(tert-butyl)-2-[(4-methylbenzoyl)imino]benzo[d]thiazol-3(2H)-yl}butanoic acid

The titled compound as a white solid was prepared in accordance with the same procedures as in Example 5, using 6-tert-butylbenzo[d]thiazol-2-amine prepared in Preparation 7 and methyl 2-bromobutyrate, instead of 6-methylbenzo[d]thiazol-2-amine and ethyl 2-bromobutyrate, respectively (Yield: 92%).

¹H NMR (DMSO-d₆, 400 MHz) δ 13.07 (brs, 1H), 8.11 (d, 2H), 7.96 (s, 1H), 7.67 (s, 1H), 7.57 (d, 1H), 7.31 (d, 2H), 5.73 (brs, 1H), 2.38 (s, 3H), 2.30-2.37 (m, 2H), 1.34 (s, 9H), 0.75 (t, 3H)

### Example 19

### 2-{2-[(4-methylbenzoyl)imino]-6-propylbenzo[d]thiazol-3(2H)-yl}butanoic acid

The titled compound as a white solid was prepared in accordance with the same procedures as in Example 5, using 6-propylbenzo[d]thiazol-2-amine prepared in Preparation 8 and methyl 2-bromobutyrate, instead of 6-methylbenzo[d]thiazol-2-amine and ethyl 2-bromobutyrate, respectively (Yield: 97%).

¹H NMR (DMSO-d₆, 400 MHz) δ 13.07 (brs, 1H), 8.11 (d, 2H), 7.76 (s, 1H), 7.67 (s, 1H), 7.36 (d, 1H), 7.31 (d, 2H), 5.71 (brs, 1H), 2.65 (t, 2H), 2.38 (s, 3H), 2.31-2.38 (m, 2H), 1.65 (m, 2H), 0.92 (t, 3H), 0.76 (t, 3H)

### Example 20

### 2-{6-isopropyl-2-[(4-methylbenzoyl)imino]benzo[d]thiazol-3(2H)-yl}butanoic acid

The titled compound as a white solid was prepared in accordance with the same procedures as in Example 5, using 6-isopropylbenzo[d]thiazol-2-amine prepared in Preparation 9 and methyl 2-bromobutyrate, instead of 6-methylbenzo[d]thiazol-2-amine and ethyl 2-bromobutyrate, respectively (Yield: 95%).

¹H NMR (DMSO-d₆, 400 MHz) δ 13.04 (brs, 1H), 8.11 (d, 2H), 7.82 (s, 1H), 7.68 (s, 1H), 7.42 (d, 1H), 7.31 (d, 2H), 5.73 (brs, 1H), 3.00 (m, 1H), 2.38 (s, 3H), 2.31-2.38 (m, 2H), 1.27 (d, 6H), 0.75 (t, 3H)

### Example 21

### 2-{5-methoxy-2-[(4-methylbenzoyl)imino]benzo[d]thiazol-3(2H)-yl}butanoic acid

The titled compound as a white solid was prepared in accordance with the same procedures as in Steps 2 and 3 of Example 5, using 4-methyl-N-(5-methoxybenzo[d]thiazol-2-yl)benzamide prepared in Preparation 11 and methyl 2-bromobutyrate, instead of 4-methyl-N-[6-methylbenzo[d]thiazol-2-yl]benzamide and ethyl 2-bromobutyrate, respectively (Yield: 79%).

¹H NMR (DMSO-d₆, 400 MHz) δ 13.03 (s, 1H), 8.08 (d, 2H), 7.81 (d, 1H), 7.44 (s, 1H), 7.30 (d, 2H), 7.00 (dd, 1H), 5.72 (brs, 1H), 3.86 (s, 3H), 2.30-2.60 (m, 2H), 2.35 (s, 3H), 0.75 (t, 3H)

### Example 22

### 2-{5-(benzyloxy)-2-[(4-methylbenzoyl)imino]benzo[d]thiazol-3(2H)-yl}butanoic acid

The titled compound as a white solid was prepared in accordance with the same procedures as in Steps 2 and 3 of Example 5, using 4-methyl-N-(5-benzyloxybenzo[d]thiazol-2-yl)benzamide prepared in Preparation 12 and methyl 2-bromobutyrate, instead of 4-methyl-N-[6-methylbenzo[d]thiazol-2-yl]benzamide and ethyl 2-bromobutyrate, respectively (Yield: 74%).

¹H NMR (DMSO-d₆, 400 MHz) δ 13.15 (brs, 1H), 8.09 (d, 2H), 7.81 (d, 1H), 7.50 (d, 3H), 7.41 (t, 3H), 7.37 (d, 1H), 7.30 (d, 2H), 7.07 (dd, 1H), 5.69 (d, 1H), 5.21 (s, 2H), 2.28-2.51 (m, 2H), 2.39 (s, 3H), 0.73 (t, 3H)

### Example 23

### 2-{2-[(4-methylbenzoyl)imino]-6-nitrobenzo[d]thiazol-3(2H)-yl}butanoic acid

The titled compound as a white solid was prepared in accordance with the same procedures as in Example 5, using 6-methylbenzo[d]thiazol-2-amine and methyl 2-bromobutyrate, instead of 6-methylbenzo[d]thiazol-2-amine and ethyl 2-bromobutyrate, respectively (Yield: 65%).

¹H NMR (DMSO-d₆, 400 MHz) δ 13.27 (brs, 1H), 9.00 (s, 1H), 8.40 (d, 1H), 8.11 (d, 2H), 8.02 (s, 1H), 7.34 (d, 2H), 5.85 (brs, 1H), 2.60-2.35 (m, 2H), 2.40 (s, 3H), 0.77 (t, 3H)

### Example 24

### 2-{6-acetamido-2-[(4-methylbenzoyl)imino]benzo[d]thiazol-3(2H)-yl}butanoic acid

### Step 1: methyl 2-{6-nitro-2-[(4-methylbenzoyl)imino]benzo[d]thiazol-3(2H)-yl}butanoate

The titled compound as a white solid was prepared in accordance with the same procedures as in Steps 2 and 3 of Example 5, using 6-nitrobenzo[d]thiazol-2-amine and methyl 2-bromobutyrate, instead of 6-methylbenzo[d]thiazol-2-amine and ethyl 2-bromobutyrate, respectively (Yield: 60%).

¹H NMR (CDCl₃, 400 MHz) δ 8.61 (s, 1H), 8.34 (d, 1H), 8.18 (d, 2H), 7.34 (d, 1H), 7.29 (d, 2H), 5.64 (brs, 1H), 3.69 (s, 3H), 2.60-2.40 (m, 2H), 2.44 (s, 3H), 0.93 (t, 3H)

### Step 2: methyl 2-{6-amino-2-[(4-methylbenzoyl)imino]benzo[d]thiazol-3(2H)-yl}butanoate

To a solution of methyl 2-{6-nitro-2-[(4-methylbenzoyl)imino]benzo[d]thiazol-3(2H)-yl}butanoate (10.00 g, 24.20 mmol) prepared in Step 1 in a mixed solvent of N,N-dimethylformamide and ethyl acetate (4/1, 200 mL) was added palladium on activated charcoal (10%, 5.0 g). The reaction mixture was stirred under hydrogen atmosphere (2 atm) overnight. The reaction mixture was filtered through a celite pad. The filtrate was diluted with ethyl acetate, washed with water and brine, dried over anhydrous magnesium sulfate, filtered, and then evaporated. The residue was suspended with isopropyl ether. The resulting solid was filtered and then dried under reduced pressure to give 7.80 g of the titled compound as a white solid (Yield: 84%).

### Step 3: methyl 2-{6-acetamido-2-[(4-methylbenzoyl)imino]benzo[d]thiazol-3(2H)-yl}butanoate

To a solution of 2-{6-amino-2-[(4-methylbenzoyl)imino]benzo[d]thiazol-3(2H)-yl}butanoate (250 mg, 0.65 mmol) prepared in Step 1 in tetrahydrofuran (7 mL), were added diisopropylethylamine (136 uL, 0.78 mmol), 4,4-dimethylaminopyridine (10 mg, 0.07 mmol), and acetic anhydride (68 uL, 0.72 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine, dried over anhydrous magnesium sulfate, filtered, and then evaporated. The residue was purified with silica gel column chromatography (n-hexaneethyl acetate=1/2) to give 235 mg of the titled compound as a white solid (Yield: 85%).

¹H NMR (CDCl₃, 400 MHz) δ 8.19 (d, 2H), 7.97 (s, 1H), 7.51 (d, 1H), 7.37 (s, 1H), 7.28 (d, 2H), 7.21 (d, 1H), 5.76 (brs, 1H), 3.65 (s, 3H), 2.51 (m, 2H), 2.42 (s, 3H), 2.21 (s, 3H), 0.89 (t, 3H)

### Step 4: 2-{6-acetamido-2-[(4-methylbenzoyl)imino]benzo[d]thiazol-3(2H)-yl}butanoic acid

The titled compound as a white solid was prepared in accordance with the same procedures as in Step 3 of Example 5, using methyl 2-{6-acetamido-2-[(4-methylbenzoyl)imino]benzo[d]thiazol-3(2H)-yl}butanoate prepared in Step 3 as a starting material (Yield: 81%).

¹H NMR (DMSO-d₆, 400 MHz) δ 10.23 (s, 1H), 8.15 (s, 1H), 8.11 (d, 2H), 7.49 (s, 2H), 7.31 (d, 2H), 5.75 (brs, 1H), 2.21-2.38 (m, 2H), 2.37 (s, 3H), 2.05 (s, 3H), 0.74 (t, 3H)

### Example 25

### 2-{2-[(4-methylbenzoyl)imino]-6-propionamidobenzo[d]thiazol-3(2H)-yl}butanoic acid

The titled compound as a white solid was prepared in accordance with the same procedures as in Example 24, using propionic anhydride, instead of acetic anhydride (Yield: 83%).

¹H NMR (DMSO-d₆, 400 MHz) δ 10.13 (s, 1H), 8.18 (s, 1H), 8.11 (d, 2H), 7.51 (s, 2H), 7.31 (d, 2H), 5.73 (brs, 1H), 2.38-2.31 (m, 7H), 1.09 (s, 3H), 0.74 (t, 3H)

### Example 26

### 2-{6-isobutyramido-2-[(4-methylbenzoyl)imino]benzo[d]thiazol-3(2H)-yl}butanoic acid

The titled compound as a white solid was prepared in accordance with the same procedures as in Example 24, using isobutyryl chloride, instead of acetic anhydride (Yield: 83%).

¹H NMR (DMSO-d₆, 400 MHz) δ 10.06 (s, 1H), 8.21 (s, 1H), 8.11 (d, 2H), 7.55 (s, 2H), 7.31 (d, 2H), 5.71 (brs, 1H), 2.60 (m, 1H), 2.31-2.38 (m, 5H), 1.12 (d, 6H), 0.73 (t, 3H)

### Example 27

### 2-{2-[(4-methylbenzoyl)imino]-6-pivalamidobenzo[d]thiazol-3(2H)-yl}butanoic acid

The titled compound as a white solid was prepared in accordance with the same procedures as in Example 24, using pivaloyl chloride, instead of acetic anhydride (Yield: 73%).

¹H NMR (DMSO-d₆, 400 MHz) δ 9.44 (s, 1H), 8.19 (s, 1H), 8.11 (d, 2H), 7.66 (d, 1H), 7.56 (s, 1H), 7.31 (d, 2H), 5.73 (brs, 1H), 2.31-2.38 (m, 5H), 1.24 (s, 9H), 0.73 (t, 3H)

### Example 28

### 2-{6-benzamido-2-[(4-methylbenzoyl)imino]benzo[d]thiazol-3(2H)-yl}butanoic acid

The titled compound as a white solid was prepared in accordance with the same procedures as in Example 24, using benzoyl chloride, instead of acetic anhydride (Yield: 70%).

¹H NMR (DMSO-d₆, 400 MHz) δ 10.55 (s, 1H), 8.35 (d, 1H), 8.13 (d, 2H), 8.01 (d, 2H), 7.77 (d, 1H), 7.56 (m, 4H), 7.31 (d, 2H), 5.78 (brs, 1H), 2.29-2.42 (m, 5H), 0.76 (t, 3H)

### Example 29

### 2-{2-[(4-methylbenzoyl)imino]-6-(2-phenylacetamino)benzo[d]thiazol-3(2H)-yl)butanoic acid

The titled compound as a white solid was prepared in accordance with the same procedures as in Example 24, using phenylacetyl chloride, instead of acetic anhydride (Yield: 62%).

¹H NMR (DMSO-d₆, 400 MHz) δ 13.06 (s, 1H), 10.39 (s, 1H), 8.21 (s, 1H), 8.08 (d, 2H), 7.70 (s, 1H), 7.62 (d, 1H), 7.32 (m, 7H), 5.68 (brs, 1H), 3.66 (s, 2H), 2.29-2.50 (m, 5H), 0.73 (t, 3H)

### Example 30

### 2-{2-[(4-methylbenzoyl)imino]-6-(phenylsulfonamido)benzo[d]thiazol-3(2H)-yl}butanoic acid

The titled compound as a white solid was prepared in accordance with the same procedures as in Example 24, using benzenesulfonyl chloride, instead of acetic anhydride (Yield: 65%).

¹H NMR (DMSO-d₆, 400 MHz) δ 10.45 (s, 1H), 8.06 (d, 2H), 7.79 (d, 2H), 7.58 (m, 5H), 7.29 (d, 2H), 7.15 (d, 1H), 5.59 (brs, 1H), 2.24-2.39 (m, 5H), 0.69 (t, 3H)

### Example 31

### 2-{6-(cyclopentanecarboxamido)-2-[(4-methylbenzoyl)imino]benzo[d]thiazol-3(2H)-yl}butanoic acid

The titled compound as a white solid was prepared in accordance with the same procedures as in Example 24, using cyclopentyl acetyl chloride, instead of acetic anhydride (Yield: 71%).

¹H NMR (DMSO-d₆, 400 MHz) δ 10.08 (s, 1H), 8.22 (s, 1H), 8.08 (d, 2H), 7.68 (s, 1H), 7.61 (d, 1H), 7.30 (d, 2H), 5.67 (brs, 1H), 2.79 (m, 1H), 2.29-2.50 (m, 5H), 1.86 (m, 2H), 1.71 (m, 4H), 1.55 (m, 2H), 0.75 (t, 3H)

### Example 32

### 2-{6-(2-fluorobenzamido)-2-[(4-methylbenzoyl)imino]benzo[d]thiazol-3(2H)-yl}butanoic acid

The titled compound as a white solid was prepared in accordance with the same procedures as in Example 24, using 2-fluorobenzoyl chloride, instead of acetic anhydride (Yield: 67%).

¹H NMR (DMSO-d₆, 400 MHz) δ 10.63 (s, 1H), 8.35 (s, 1H), 8.09 (d, 2H), 7.70 (m, 3H), 7.60 (m, 1H), 7.34 (m, 4H), 5.69 (brs, 1H), 2.29-2.50 (m, 5H), 0.75 (t, 3H)

### Example 33

### 2-{6-[3-(4-fluorophenyl)ureido]-2-[(4-methylbenzoyl)imino]benzo[d]thiazol-3(2H)-yl}butanoic acid

The titled compound as a white solid was prepared in accordance with the same procedures as in Example 24, using 4-fluorophenyl isocyanate, instead of acetic anhydride (Yield: 74%).

¹H NMR (DMSO-d₆, 400 MHz) δ 8.86 (s, 1H), 8.77 (s, 1H), 8.07 (m, 3H), 7.67 (s, 1H), 7.49 (m, 3H), 7.30 (d, 2H), 7.11 (t, 2H), 5.67 (brs, 1H), 2.28-2.50 (m, 5H), 0.75 (t, 3H)

### Example 34

### 2-{6-[3-(3-chlorophenyl)ureido]-2-[(4-methylbenzoyl)imino]benzo[d]thiazol-3(2H)-yl}butanoic acid

The titled compound as a white solid was prepared in accordance with the same procedures as in Example 24, using 3-chlorophenyl isocyanate, instead of acetic anhydride (Yield: 67%).

¹H NMR (DMSO-d₆, 400 MHz) δ 8.96 (d, 2H), 8.07 (m, 3H), 7.72 (s, 1H), 7.71 (s, 1H), 7.50 (t, 1H), 7.30 (m, 4H), 7.01 (t, 1H), 5.71 (brs, 1H), 2.33 (m, 5H), 0.75 (t, 3H)

### Example 35

### 2-{6-(4-fluorophenylsulfonamido)-2-[(4-methylbenzoyl)imino]benzo[d]thiazol-3(2H)-yl}butanoic acid

The titled compound as a white solid was prepared in accordance with the same procedures as in Example 24, using 4-fluorobenzenesulfonyl chloride, instead of acetic anhydride (Yield: 61%).

¹H NMR (DMSO-d₆, 400 MHz) δ 10.45 (s, 1H), 8.06 (d, 2H), 7.84 (m, 2H), 7.64 (m, 2H), 7.38 (t, 2H), 7.29 (d, 2H), 7.14 (t, 1H), 5.60 (brs, 1H), 2.24-2.39 (m, 5H), 0.69 (t, 3H)

### Example 36

### 2-{2-[(4-methylbenzoyl)imino]-6-[4-(trifluoromethyl)phenylsulfonamido]benzo[d]thiazol-3(2H)-yl}butanoic acid

The titled compound as a white solid was prepared in accordance with the same procedures as in Example 24, using 4-trifluoromethylbenzenesulfonyl chloride, instead of acetic anhydride (Yield: 65%).

¹H NMR (DMSO-d₆, 400 MHz) δ 10.66 (s, 1H), 8.06 (d, 2H), 7.97 (m, 4H), 7.67 (m, 2H), 7.29 (d, 2H), 7.16 (dd, 1H), 5.61 (brs, 1H), 2.26-2.36 (m, 5H), 0.69 (t, 3H)

### Example 37

### 2-{2-[(4-methylbenzoyl)imino]-5-nitrobenzo[d]thiazol-3(2H)-yl}butanoic acid

The titled compound as a white solid was prepared in accordance with the same procedures as in Steps 2 and 3 of Example 5, using 4-methyl-N-(5-nitrobenzo[d]thiazol-2-yl)benzamide prepared in Preparation 10 and methyl 2-bromobutyrate, instead of 4-methyl-N-[6-methylbenzo[d]thiazol-2-yl]benzamide and ethyl 2-bromobutyrate, respectively (Yield: 79%).

¹H NMR (DMSO-d₆, 400 MHz) δ 8.34 (s, 1H), 8.18 (d, 2H), 8.12 (d, 2H), 7.32 (d, 2H), 5.87 (brs, 1H), 2.30-2.50 (m, 2H), 2.39 (s, 3H), 0.78 (t, 3H)

### Example 38

### 2-{5-acetamido-2-[(4-methylbenzoyl)imino]benzo[d]thiazol-3(2H)-yl}butanoic acid

### Step 1: methyl 2-{5-nitro-2-[(4-methylbenzoyl)imino]benzo[d]thiazol-3(2H)-yl}butanoate

To a stirred solution of 4-methyl-N-(5-nitrobenzo[d]thiazol-2-yl)benzamide (0.96 g, 3.06 mmol) prepared in Preparation 10 in N,N-dimethylformamide (30 mL), were added potassium carbonate (930 mg, 6.73 mmol) and methyl 2-bromobutyrate (0.76 mL, 6.13 mmol). The reaction mixture was stirred at 50° C. for 5 hours. The reaction mixture was and quenched with water and then extracted with ethyl acetate. The organic layer was washed with brine, dried over anhydrous magnesium sulfate, filtered, and then evaporated. The residue was purified with silica gel column chromatography (n-hexaneethyl acetatedichloromethane=512) to give 830 mg of the titled compound as a yellow solid (Yield: 66%).

¹H NMR (CDCl₃, 400 MHz) δ 8.20 (m, 4H), 7.85 (d, 1H), 7.30 (d, 2H), 5.54 (brs, 1H), 3.71 (s, 3H), 2.57 (m, 2H), 2.44 (s, 3H), 0.94 (t, 3H)

### Step 2: 2-{5-amino-2-[(4-methylbenzoyl)imino]benzo[d]thiazol-3(2H)-yl}butanoate

To a solution of methyl 2-{5-nitro-2-[(4-methylbenzoyl)imino]benzo[d]thiazol-3(2H)-yl}butanoate (150 mg, 0.36 mmol) prepared in Step 1 in a mixed solvent of N,N-dimethylformamide and ethyl acetate (4/1, 4 mL) was added palladium on activated charcoal (10%, 75 mg). The reaction mixture was stirred under hydrogen atmosphere (2 atm) overnight. The reaction mixture was filtered through a celite pad. The filtrate was diluted with ethyl acetate. The organic layer was washed with water and brine, dried over anhydrous magnesium sulfate, filtered, and then evaporated. The residue was suspended with isopropyl ether. The resulting solid was filtered and then dried under reduced pressure to give 150 mg of the titled compound as a white solid (Yield: 100%).

### Step 3: methyl 2-{5-acetamido-2-[(4-methylbenzoyl)imino]benzo[d]thiazol-3(2H)-yl}butanoate

To a solution of 2-{5-amino-2-[(4-methylbenzoyl)imino]benzo[d]thiazol-3(2H)-yl}butanoate (300 mg, 0.78 mmol) prepared in Step 2 in tetrahydrofuran (10 mL), were added diisopropylethylamine (163 uL, 0.94 mmol), 4,4-dimethylaminopyridine (10 mg, 0.07 mmol), and acetic anhydride (81 uL, 0.86 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine. The organic layer was dried over anhydrous magnesium sulfate, filtered, and then evaporated. The residue was purified with silica gel column chromatography (n-hexaneethyl acetate=1/2) to give 275 mg of the titled compound as a white solid (Yield: 83%).

¹H NMR (CDCl₃, 400 MHz) δ 8.19 (d, 2H), 8.10 (s, 1H), 7.60 (d, 1H), 7.33 (s, 1H), 7.28 (d, 2H), 7.06 (d, 1H), 5.49 (brs, 1H), 3.66 (s, 3H), 2.51 (m, 2H), 2.41 (s, 3H), 2.22 (s, 3H), 0.91 (t, 3H)

### Step 4: 2-{5-acetamido-2-[(4-methylbenzoyl)imino]benzo[d]thiazol-3(2H)-yl}butanoic acid

The titled compound as a white solid was prepared in accordance with the same procedures as in Step 3 of Example 5, using methyl 2-{5-acetamido-2-[(4-methylbenzoyl)imino]benzo[d]thiazol-3(2H)-yl}butanoate prepared in Step 3 as a starting material (Yield: 81%).

¹H NMR (DMSO-d₆, 400 MHz) δ 10.28 (s, 1H), 8.11 (d, 2H), 7.69 (d, 2H), 7.51 (d, 1H), 7.30 (d, 2H), 5.86 (brs, 1H), 2.37 (m, 4H), 2.21 (m, 1H), 1.95 (s, 3H), 0.73 (t, 3H)

### Example 39

### 2-{2-[(4-methylbenzoyl)imino]-5-propionamidobenzo[d]thiazol-3(2H)-yl}butanoic acid

The titled compound as a white solid was prepared in accordance with the same procedures as in Example 38, using propionic anhydride, instead of acetic anhydride (Yield: 82%).

¹H NMR (DMSO-d₆, 400 MHz) δ 10.17 (s, 1H), 8.12 (d, 2H), 8.02 (s, 1H), 7.80 (d, 2H), 7.48 (d, 1H), 7.31 (d, 2H), 5.81 (brs, 1H), 2.35 (m, 7H), 1.09 (t, 3H), 0.76 (t, 3H)

### Example 40

### 2-{5-isobutyramido-2-[(4-methylbenzoyl)imino]benzo[d]thiazol-3(2H)-yl}butanoic acid

The titled compound as a white solid was prepared in accordance with the same procedures as in Example 38, using isobutyryl chloride, instead of acetic anhydride (Yield: 80%).

¹H NMR (DMSO-d₆, 400 MHz) δ 10.13 (s, 1H), 8.13 (d, 3H), 7.84 (d, 1H), 7.47 (d, 1H), 7.32 (d, 2H), 5.76 (brs, 1H), 2.63 (m, 1H), 2.38 (m, 5H), 1.14 (d, 6H), 0.77 (t, 3H)

### Example 41

### 2-{2-[(4-methylbenzoyl)imino]-5-pivalamidobenzo[d]thiazol-3(2H)-yl}butanoic acid

The titled compound as a white solid was prepared in accordance with the same procedures as in Example 38, using pivaloyl chloride, instead of acetic anhydride (Yield: 76%).

¹H NMR (DMSO-d₆, 400 MHz) δ 9.47 (s, 1H), 8.12 (d, 3H), 7.83 (d, 1H), 7.60 (d, 1H), 7.32 (d, 2H), 5.77 (brs, 1H), 2.38 (m, 5H), 1.25 (s, 9H), 0.77 (t, 3H)

### Example 42

### 2-{5-benzamido-2-[(4-methylbenzoyl)imino]benzo[d]thiazol-3(2H)-yl}butanoic acid

The titled compound as a white solid was prepared in accordance with the same procedures as in Example 38, using benzoyl chloride, instead of acetic anhydride (Yield: 80%).

¹H NMR (DMSO-d₆, 400 MHz) δ 10.53 (s, 1H), 8.23 (s, 1H), 8.14 (d, 2H), 8.00 (d, 2H), 7.91 (d, 1H), 7.72 (d, 1H), 7.58 (m, 3H), 7.33 (d, 2H), 5.82 (brs, 1H), 2.42 (m, 5H), 0.87 (t, 3H)

### Example 43

### 2-{2-[(4-methylbenzoyl)imino]-5-(2-phenylacetamido)benzo[d]thiazol-3(2H)-yl}butanoic acid

The titled compound as a white solid was prepared in accordance with the same procedures as in Example 38, using phenylacetyl chloride, instead of acetic anhydride (Yield: 63%).

¹H NMR (DMSO-d₆, 400 MHz) δ 10.16 (s, 1H), 8.11 (m, 3H), 7.82 (d, 1H), 7.45 (d, 1H), 7.30 (d, 2H), 5.73 (brs, 1H), 2.81 (m, 1H), 2.37 (m, 5H), 1.87 (m, 2H), 1.70 (m, 4H), 1.57 (m, 2H), 0.75 (t, 3H)

### Example 44

### 2-{2-[(4-methylbenzoyl)imino]-5-(phenylsulfonamido)benzo[d]thiazol-3(2H)-yl}butanoic acid

The titled compound as a white solid was prepared in accordance with the same procedures as in Example 38, using benzenesulfonyl chloride, instead of acetic anhydride (Yield: 70%).

¹H NMR (DMSO-d₆, 400 MHz) δ 10.70 (s, 1H), 8.12 (m, 3H), 7.86 (d, 1H), 7.63 (m, 3H), 7.35 (m, 4H), 5.85 (brs, 1H), 2.37-2.50 (m, 5H), 0.75 (t, 3H)

### Example 45

### 2-{5-(cyclopentanecarboxamido)-2-[(4-methylbenzoyl)imino]benzo[d]thiazol-3(2H)-yl}butanoic acid

The titled compound as a white solid was prepared in accordance with the same procedures as in Example 38, using cyclopentyl acetyl chloride, instead of acetic anhydride (Yield: 67%).

¹H NMR (DMSO-d₆, 400 MHz) δ 10.47 (s, 1H), 8.10 (m, 3H), 7.84 (d, 1H), 7.45 (d, 1H), 7.28 (m, 7H), 5.71 (brs, 1H), 3.67 (s, 2H), 2.36 (m, 5H), 0.73 (t, 3H)

### Example 46

### 2-{5-(2-fluorobenzamido)-2-[(4-methylbenzoyl)imino]benzo[d]thiazol-3(2H)-yl}butanoic acid

The titled compound as a white solid was prepared in accordance with the same procedures as in Example 38, using 2-fluorobenzoyl chloride, instead of acetic anhydride (Yield: 72%).

¹H NMR (DMSO-d₆, 400 MHz) δ 10.57 (s, 1H), 8.09 (d, 2H), 7.79 (m, 3H), 7.61 (m, 1H), 7.55 (m, 2H), 7.34 (s, 1H), 7.30 (d, 2H), 5.72 (brs, 1H), 2.37 (s, 3H), 2.31 (m, 2H), 0.63 (t, 3H)

### Example 47

### 2-{5-[3-(4-fluorophenyl)ureido]-2-[(4-methylbenzoyl)imino]benzo[d]thiazol-3(2H)-yl}butanoic acid

The titled compound as a white solid was prepared in accordance with the same procedures as in Example 38, using 4-fluorophenyl isocyanate, instead of acetic anhydride (Yield: 68%).

¹H NMR (DMSO-d₆, 400 MHz) δ 10.55 (s, 1H), 8.09 (d, 2H), 7.82 (m, 2H), 7.69 (d, 1H), 7.32 (m, 5H), 6.95 (d, 1H), 5.83 (brs, 1H), 2.36 (m, 4H), 2.36 (m, 1H), 0.63 (t, 3H)

### Example 48

### 2-{5-[3-(3-chlorophenyl)ureido]-2-[(4-methylbenzoyl)imino]benzo[d]thiazol-3(2H)-yl}butanoic acid

The titled compound as a white solid was prepared in accordance with the same procedures as in Example 38, using 3-chlorophenyl isocyanate, instead of acetic anhydride (Yield: 61%).

¹H NMR (DMSO-d₆, 400 MHz) δ 8.09 (d, 2H), 7.96 (d, 2H), 7.88 (d, 2H), 7.66 (d, 1H), 7.29 (m, 3H), 5.91 (brs, 1H), 2.36 (m, 4H), 2.07 (m, 1H), 0.61 (t, 3H)

### Example 49

### 2-{5-(4-fluorophenylsulfonamido)-2-[(4-methylbenzoyl)imino]benzo[d]thiazol-3(2H)-yl}butanoic acid

The titled compound as a white solid was prepared in accordance with the same procedures as in Example 38, using 4-fluorobenzenesulfonyl chloride, instead of acetic anhydride (Yield: 69%).

¹H NMR (DMSO-d₆, 400 MHz) δ 8.99 (s, 1H), 8.83 (s, 1H), 8.11 (d, 2H), 7.90 (s, 1H), 7.81 (d, 2H), 7.46 (m, 2H), 7.30 (m, 3H), 7.12 (t, 2H), 5.72 (brs, 1H), 2.37 (m, 5H), 0.76 (t, 3H)

### Example 50

### 2-{2-[(4-methylbenzoyl)imino]-5-[4-(trifluoromethyl)phenylsulfonamido]benzo[d]thiazol-3(2H)-yl}butanoic acid

The titled compound as a white solid was prepared in accordance with the same procedures as in Example 38, using 4-trifluoromethylbenzenesulfonyl chloride, instead of acetic anhydride (Yield: 72%).

¹H NMR (DMSO-d₆, 400 MHz) δ 9.10 (s, 1H), 9.03 (s, 1H), 8.11 (d, 2H), 7.92 (s, 1H), 7.82 (d, 1H), 7.73 (s, 1H), 7.28 (m, 5H), 7.02 (t, 1H), 5.78 (brs, 1H), 2.37 (m, 5H), 0.76 (t, 3H)

### Example 51

### 2-{2-[(4-methylbenzoyl)imino]-7-nitrobenzo[d]thiazol-3(2H)-yl}butanoic acid

The titled compound as a white solid was prepared in accordance with the same procedures as in Example 5, using 7-nitrobenzo[d]thiazol-2-amine prepared in Preparation 13 and methyl 2-bromobutyrate, instead of 6-methylbenzo[d]thiazol-2-amine and ethyl 2-bromobutyrate, respectively (Yield: 77%).

¹H NMR (DMSO-d₆, 400 MHz) δ 13.26 (brs, 1H), 8.30 (m, 2H), 8.12 (d, 2H), 7.83 (t, 1H), 7.33 (d, 2H), 5.84 (brs, 1H), 2.35-2.60 (m, 2H), 2.40 (s, 3H), 0.77 (t, 3H)

### Example 52

### 2-{2-[(3-chlorobenzoyl)imino]-6-methoxybenzo[d]thiazol-3(2H)-yl}acetic acid

### Step 1: 3-chloro-N-[6-(methoxy)benzo[d]thiazol-2-yl]benzamide

To a solution of 6-methoxybenzo[d]thiazol-2-amine (1.80 g, 10.00 mmol) in tetrahydrofuran (100 mL), were added diisopropylethylamine (2.61 mL, 15.00 mmol), 4,4-dimethylaminopyridine (122 mg, 1.00 mmol), and 3-chlorobenzoyl chloride (1.54 mL, 12.00 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine. The organic layer was dried over anhydrous magnesium sulfate, filtered, and then evaporated. The residue was diluted with isopropyl ether. The resulting solid was filtered and then dried under reduced pressure to give 2.18 g of the titled compound as a white solid (Yield: 68%).

### Step 2: ethyl 2-{2-[(3-chlorobenzoyl)imino]-6-methoxybenzo[d]thiazol-3(2H)-yl}acetate

To a solution of 3-chloro-N-[6-(methoxy)benzo[d]thiazol-2-yl]benzamide (300 mg, 0.94 mmol) prepared in Step 1 in N,N-dimethylformamide (10 mL), potassium carbonate (286 mg, 2.07 mmol) and ethyl bromoacetate (208 uL, 1.88 mmol). The reaction mixture was stirred at 50° C. overnight. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=10/1˜1/1) to give 280 mg of the titled compound as a white solid (Yield: 73%).

¹H NMR (CDCl₃, 400 MHz) δ 8.29 (s, 1H), 8.19 (d, 1H), 7.48 (d, 1H), 7.38 (t, 1H), 7.26 (d, 1H), 7.17 (d, 1H), 7.05 (d, 1H), 7.03 (d, 1H), 5.18 (s, 2H), 4.29 (m, 2H), 3.87 (s, 3H), 1.29 (t, 3H)

### Step 3: 2-{2-[(3-chlorobenzoyl)imino]-6-methoxybenzo[d]thiazol-3(2H)-yl}acetic acid

To a solution of ethyl 2-{2-[(3-chlorobenzoyl)imino]-6-methoxybenzo[d]thiazol-3(2H)-yl}acetate (250 mg, 0.62 mmol) prepared in Step 2 in a mixed solvent of tetrahydrofuran and methanol (1/1, 6.0 mL) was added a 1N sodium hydroxide solution (3.0 mL). The reaction mixture was stirred at room temperature overnight and then evaporated. The aqueous layer was acidified to pH 3-4 with an aqueous 1N HCl solution, and then extracted with ethyl acetate. The organic layer was washed with brine, dried over anhydrous magnesium sulfate, filtered, and then evaporated. The residue was dried under reduced pressure to give 180 mg of the titled compound as a white solid (Yield: 78%).

¹H NMR (DMSO-d₆, 400 MHz) δ 13.36 (s, 1H), 8.17 (s, 1H), 8.15 (d, 1H), 7.65 (m, 2H), 7.59 (s, 1H), 7.54 (t, 1H), 7.11 (dd, 1H), 5.29 (s, 2H), 3.81 (s, 3H)

### Example 53

### 2-{2-[(3-chlorobenzoyl)imino]-6-methoxybenzo[d]thiazol-3(2H)-yl}propionic acid

The titled compound as a white solid was prepared in accordance with the same procedures as in Example 52, using ethyl 2-bromopropionate, instead of ethyl bromoacetate (Yield: 81%).

¹H NMR (DMSO-d₆, 400 MHz) δ 13.10 (s, 1H), 8.18 (s, 1H), 8.13 (d, 1H), 7.72 (d, 1H), 7.61 (m, 2H), 7.53 (t, 1H), 7.15 (t, 1H), 5.83 (s, 1H), 3.82 (s, 3H), 1.69 (d, 3H)

### Example 54

### 2-{2-[(3-chlorobenzoyl)imino]-6-methoxybenzo[d]thiazol-3(2H)-yl}butanoic acid

The titled compound as a white solid was prepared in accordance with the same procedures as in Example 52, using methyl 2-bromobutyrate, instead of ethyl bromoacetate (Yield: 82%).

¹H NMR (DMSO-d₆, 400 MHz) δ 13.09 (s, 1H), 8.16 (d, 1H), 8.12 (d, 1H), 7.73 (s, 1H), 7.64 (m, 2H), 7.53 (t, 1H), 7.14 (dd, 1H), 5.71 (s, 1H), 3.82 (s, 3H), 2.28-2.44 (m, 2H), 0.73 (d, 3H)

### Example 55

### 2-{2-[(3-chlorobenzoyl)imino]-6-ethoxybenzo[d]thiazol-3(2H)-yl}propionic acid

### Step 1: 3-chloro-N-[6-(ethoxy)benzo[d]thiazol-2-yl]benzamide

To a solution of 6-ethoxybenzo[d]thiazol-2-amine (1.94 g, 10.00 mmol) in tetrahydrofuran (100 mL), were added diisopropylethylamine (2.61 mL, 15.00 mmol), 4,4-dimethylaminopyridine (122 mg, 1.00 mmol), and 3-chlorobenzoyl chloride (1.54 mL, 12.00 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine. The organic layer was dried over anhydrous magnesium sulfate, filtered, and then evaporated. The residue was diluted with isopropyl ether. The resulting solid was filtered and then dried under reduced pressure to give 2.33 g of the titled compound as a white solid (Yield: 70%).

### Step 2: ethyl 2-{2-[(3-chlorobenzoyl)imino]-6-ethoxybenzo[d]thiazol-3(2H)-yl}propionate

To a solution of 3-chloro-N-[6-(ethoxy)benzo[d]thiazol-2-yl]benzamide (300 mg, 0.90 mmol) prepared in Step 1 in N,N-dimethylformamide (10 mL), were added potassium carbonate (280 mg, 1.98 mmol) and ethyl 2-bromopropionate (234 uL, 1.80 mmol). The reaction mixture was stirred at 50° C. for 5 hours. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=31) to give 286 mg of the titled compound as a white solid (Yield: 73%).

¹H NMR (CDCl₃, 400 MHz) δ 8.25 (s, 1H), 8.17 (d, 1H), 7.49 (d, 1H), 7.40 (m, 1H), 7.23 (m, 2H), 7.04 (d, 1H), 5.65 (s, 1H), 4.20 (m, 2H), 4.11 (m, 2H), 1.84 (d, 3H), 1.45 (t, 3H), 1.10 (t, 3H)

### Step 3: 2-{2-[(3-chlorobenzoyl)imino]-6-ethoxybenzo[d]thiazol-3(2H)-yl}propionic acid

The titled compound as a white solid was prepared in accordance with the same procedures as in Step 3 of Example 5, using ethyl 2-{2-[(3-chlorobenzoyl)imino]-6-ethoxybenzo[d]thiazol-3(2H)-yl}propionate prepared in Step 2 as a starting material (Yield: 82%).

¹H NMR (DMSO-d₆, 400 MHz) δ 13.10 (s, 1H), 8.18 (s, 1H), 8.13 (d, 1H), 7.70 (d, 1H), 7.64 (t, 1H), 7.59 (d, 1H), 7.53 (t, 1H), 7.15 (dd, 1H), 5.82 (s, 1H), 4.09 (q, 2H), 1.69 (d, 3H), 1.34 (t, 3H)

### Example 56

### 2-{2-[(3-chlorobenzoyl)imino]-6-ethoxybenzo[d]thiazol-3(2H)-yl}butanoic acid

The titled compound as a white solid was prepared in accordance with the same procedures as in Example 55, using methyl 2-bromobutyrate, instead of ethyl 2-bromopropionate (Yield: 78%).

¹H NMR (DMSO-d₆, 400 MHz) δ 12.95 (s, 1H), 8.16 (s, 1H), 8.12 (d, 1H), 7.71 (s, 1H), 7.64 (t, 1H), 7.59 (d, 1H), 7.53 (t, 1H), 7.14 (dd, 1H), 5.70 (s, 1H), 4.10 (m, 2H), 2.45-2.27 (m, 2H), 1.35 (t, 3H), 0.70 (t, 3H)

### Example 57

### 2-{2-[(3-chlorobenzoyl)imino]-6-methylbenzo[d]thiazol-3(2H)-yl}acetic acid

### Step 1: 3-chloro-N-(6-methylbenzo[d]thiazol-2-yl)benzamide

To a solution of 6-methylbenzo[d]thiazol-2-amine (1.64 g, 10.00 mmol) in tetrahydrofuran (100 mL), were added diisopropylethylamine (2.61 mL, 15.00 mmol), 4,4-dimethylaminopyridine (122 mg, 1.00 mmol), and 3-chlorobenzoyl chloride (1.54 mL, 12.00 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine, dried over anhydrous magnesium sulfate, filtered, and then evaporated. The residue was diluted with isopropyl ether. The resulting solid was filtered and then dried under reduced pressure to give 1.72 g of the titled compound as a white solid (Yield: 56%).

### Step 2: ethyl 2-{2-[(3-chlorobenzoyl)imino]-6-methylbenzo[d]thiazol-3(2H)-yl}acetate

To a solution of 3-chloro-N-(6-methylbenzo[d]thiazol-2-yl)benzamide (300 mg, 0.99 mmol) prepared in Step 1 in N,N-dimethylformamide (10 mL), were added potassium carbonate (300 mg, 2.18 mmol) and ethyl bromoacetate (220 uL, 1.98 mmol). The reaction mixture was stirred at 50° C. for 5 hours. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=2/1) to give 296 mg of the titled compound as a white solid (Yield: 77%).

¹H NMR (CDCl₃, 400 MHz) δ 8.25 (s, 1H), 8.17 (d, 1H), 7.52 (s, 1H), 7.48 (d, 1H), 7.37 (m, 1H), 7.26 (m, 1H), 7.12 (m, 1H), 5.19 (s, 2H), 4.27 (m, 2H), 2.45 (s, 3H), 1.28 (t, 3H)

### Step 3: 2-{2-[(3-chlorobenzoyl)imino]-6-methylbenzo[d]thiazol-3(2H)-yl}acetic acid

The titled compound as a white solid was prepared in accordance with the same procedures as in Step 3 of Example 5, using ethyl 2-{2-[(3-chlorobenzoyl)imino]-6-methylbenzo[d]thiazol-3(2H)-yl}acetate prepared in Step 2 as a starting material (Yield: 74%).

¹H NMR (DMSO-d₆, 400 MHz) δ 13.33 (s, 1H), 8.18 (s, 1H), 8.16 (d, 1H), 7.75 (s, 1H), 7.63 (m, 2H), 7.54 (t, 1H), 7.35 (d, 1H), 5.30 (s, 2H), 2.40 (s, 3H)

### Example 58

### 2-{2-[(3-chlorobenzoyl)imino]-6-methylbenzo[d]thiazol-3(2H)-yl}propionic acid

The titled compound as a white solid was prepared in accordance with the same procedures as in Example 57, using ethyl 2-bromopropionate, instead of ethyl bromoacetate (Yield: 79%).

¹H NMR (DMSO-d₆, 400 MHz) δ 13.09 (s, 1H), 8.19 (s, 1H), 8.14 (d, 1H), 7.77 (s, 1H), 7.65 (m, 2H), 7.54 (t, 1H), 7.39 (d, 1H), 5.84 (s, 1H), 2.41 (s, 3H), 1.69 (d, 3H)

### Example 59

### 2-{2-[(3-chlorobenzoyl)imino]-6-methylbenzo[d]thiazol-3(2H)-yl}butanoic acid

The titled compound as a white solid was prepared in accordance with the same procedures as in Example 57, using methyl 2-bromobutyrate, instead of ethyl bromoacetate (Yield: 68%).

¹H NMR (DMSO-d₆, 400 MHz) δ 13.09 (s, 1H), 8.17 (d, 1H), 8.13 (d, 1H), 7.76 (s, 1H), 7.70 (s, 1H), 7.62 (t, 1H), 7.53 (t, 1H), 7.37 (d, 1H), 5.72 (s, 1H), 2.28-2.40 (m, 5H), 0.73 (t, 3H)

### Example 60

### 2-[2-(4-methoxybenzoylimino)benzo[d]thiazol-3(2H)-yl]butanoic acid

### Step 1: methyl 2-[2-(4-methoxybenzoylimino)benzo[d]thiazol-3(2H)-yl]butanoate

To a solution of 2-aminobenzothiazole (100 mg, 0.666 mmol) in tetrahydrofuran (3.3 mL), were added 4-methoxybenzoyl chloride (114 mg, 0.866 mmol) and potassium carbonate (183 mg, 1.33 mmol). The reaction mixture was stirred at 80° C. overnight, cooled to room temperature, and then concentrated under reduced pressure. N,N-dimethylformamide (3.3 mL) and methyl 2-bromobutyrate (0.15 mL, 1.33 mmol) were added to the resulting residue. The reaction mixture was stirred at 80° C. for 1 hour. The reaction mixture was quenched with water and then extracted with ethyl acetate. The organic layer was dried over anhydrous sodium sulfate, filtered, and then evaporated. The residue was purified with silica gel column chromatography (n-hexaneethyl acetate=10/1) to give 50.0 mg of the titled compound as a white solid (Yield: 55%).

¹H NMR (CDCl₃, 400 MHz) δ 8.27 (d, 2H), 7.70 (d, 1H), 7.43 (t, 1H), 7.30 (t, 2H), 6.97 (d, 1H), 5.53 (brs, 1H), 3.87 (s, 3H), 3.64 (s, 3H), 2.46-2.55 (m, 2H), 0.91 (t, 3H)

### Step 2: 2-[2-(4-methoxybenzoylimino)benzo[d]thiazol-3(2H)-yl]butanoic acid

To a solution of methyl 2-[2-(4-methoxybenzoylimino)benzo[d]thiazol-3(2H)-yl]butanoate (20 mg, 0.052 mmol) prepared in Step 1 in a mixed solvent of tetrahydrofuran and methanol (1/1, 1.0 mL) was added a 1N sodium hydroxide solution (0.2 mL). The reaction mixture was stirred at room temperature overnight. The reaction mixture was evaporated, acidified with an aqueous 1N HCl solution, and then extracted with dichloromethane. The organic layer was washed with brine, dried over anhydrous magnesium sulfate, filtered, evaporated and then dried under reduced pressure to give 17 mg of the titled compound as a white solid (Yield: 88%).

¹H NMR (CDCl₃, 400 MHz) δ 8.17 (d, 2H), 7.59 (s, 1H), 7.22-7.32 (m, 3H), 6.83 (d, 2H), 5.56 (brs, 1H), 3.79 (s, 3H), 2.41 (brs, 2H), 0.81 (brs, 3H)

### Example 61

### 2-[2-(3-methoxybenzoylimino)benzo[d]thiazol-3(2H)-yl]butanoic acid

The titled compound was prepared in accordance with the same procedures as in Example 60, using 3-methoxybenzoyl chloride, instead of 4-methoxybenzoyl chloride (Yield: 88%)

¹H NMR (CDCl₃, 400 MHz) δ 7.86 (d, 1H), 7.81 (s, 1H), 7.70 (d, 1H), 7.41 (t, 1H), 7.26-7.33 (m, 3H), 7.04 (d, 1H), 5.52 (brs, 1H), 3.84 (s, 3H), 2.45-2.49 (m, 2H), 0.87 (t, 3H)

### Example 62

### 2-[2-(3-fluorobenzoylimino)benzo[d]thiazol-3(2H)-yl]butanoic acid

The titled compound was prepared in accordance with the same procedures as in Example 60, using 3-fluorobenzoyl chloride, instead of 4-methoxybenzoyl chloride (Yield: 93%)

¹H NMR (CDCl₃, 400 MHz) δ 8.04 (d, 1H), 7.94 (d, 1H), 7.73 (d, 1H), 7.44 (dd, 1H), 7.28-7.36 (m, 3H), 7.16 (t, 1H), 5.66 (brs, 1H), 2.49-2.57 (m, 2H), 0.91 (t, 3H)

### Example 63

### 2-[2-(2-methylbenzoylimino)benzo[d]thiazol-3(2H)-yl]butanoic acid

The titled compound was prepared in accordance with the same procedures as in Example 60, using 2-toluoyl chloride, instead of 4-methoxybenzoyl chloride (Yield: 78%)

¹H NMR (CDCl₃, 400 MHz) δ 8.11 (d, 1H), 7.65 (d, 1H), 7.21-7.33 (m, 6H), 5.63 (brs, 1H), 2.69 (t, 3H), 2.38 (brs, 2H), 0.83 (t, 3H)

### Example 64

### 2-{2-[4-(trifluoromethyl)benzoylimino]benzo[d]thiazol-3(2H)-yl}butanoic acid

The titled compound was prepared in accordance with the same procedures as in Example 60, using 4-(trifluoromethyl)benzoyl chloride, instead of 4-methoxybenzoyl chloride (Yield: 94%).

¹H NMR (CDCl₃, 400 MHz) δ 8.25 (d, 2H), 7.58 (d, 3H), 7.22-7.33 (m, 3H), 5.51 (brs, 1H), 2.39 (brs, 2H), 0.81 (brs, 3H)

### Example 65

### 2-[2-(2,6-dichlorobenzoylimino)benzo[d]thiazol-3(2H)-yl]butanoic acid

The titled compound was prepared in accordance with the same procedures as in Example 60, using 2,6-dichlorobenzoyl chloride, instead of 4-methoxybenzoyl chloride (Yield: 35%).

¹H NMR (CDCl₃, 400 MHz) δ 7.79 (d, 1H), 7.51 (d, 1H), 7.33-7.44 (m, 5H), 5.71 (brs, 1H), 2.43-2.46 (m, 2H), 0.86 (brs, 3H)

### Example 66

### 2-[2-(2-methoxybenzoylimino)benzo[d]thiazol-3(2H)-yl]butanoic acid

The titled compound was prepared in accordance with the same procedures as in Example 60, using 2-methoxybenzoyl chloride, instead of 4-methoxybenzoyl chloride (Yield: 94%)

¹H NMR (CDCl₃, 400 MHz) δ 8.07 (d, 1H), 7.69 (d, 1H), 7.41-7.45 (m, 3H), 7.32 (d, 1H), 6.94-7.00 (m, 2H), 5.64 (brs, 1H), 3.88 (s, 3H), 2.49 (dd, 2H), 0.85 (t, 3H)

### Example 67

### 2-{2-[3-(benzyloxy)benzoylimino]benzo[d]thiazol-3(2H)-yl}butanoic acid

The titled compound was prepared in accordance with the same procedures as in Example 60, using 3-(benzyloxy)benzoyl chloride and ethyl 2-bromobutyrate, instead of 4-methoxybenzoyl chloride and methyl 2-bromobutyrate, respectively (Yield: 93%).

¹H NMR (CDCl₃, 400 MHz) δ 10.60 (brs, 1H), 7.87 (d, 2H), 7.67 (d, 1H), 7.26-7.43 (m, 9H), 7.11 (d, 1H), 5.47 (brs, 1H), 5.10 (s, 2H), 2.44 (brs, 2H), 0.83 (t, 3H)

### Example 68

### 2-[2-(2-fluorobenzoylimino)benzo[d]thiazol-3(2H)-yl]butanoic acid

The titled compound was prepared in accordance with the same procedures as in Example 60, using 2-fluorobenzoyl chloride and ethyl 2-bromobutyrate, instead of 4-methoxybenzoyl chloride and methyl 2-bromobutyrate, respectively (Yield: 93%).

¹H NMR (CDCl₃, 400 MHz) δ 10.76 (brs, 1H), 8.10 (t, 1H), 7.66 (d, 1H), 7.28-7.38 (m, 4H), 7.03-7.14 (m, 2H), 5.73 (brs, 1H), 2.44 (brb, 2H), 0.84 (t, 3H)

### Example 69

### 2-[2-(5-fluoro-2-methylbenzoylimino)benzo[d]thiazol-3(2H)-yl]butanoic acid

The titled compound was prepared in accordance with the same procedures as in Example 60, using 5-fluoro-2-methylbenzoyl chloride and ethyl 2-bromobutyrate, instead of 4-methoxybenzoyl chloride and methyl 2-bromobutyrate, respectively (Yield: 98%).

¹H NMR (CDCl₃, 400 MHz) δ 10.60 (brs, 1H), 8.86 (d, 1H), 7.69 (d, 1H), 7.40 (dd, 1H), 7.26-7.32 (m, 2H), 7.17 (dd, 1H), 7.05 (dd, 1H), 5.72 (brs, 1H), 2.66 (s, 3H), 2.42-2.49 (m, 2H), 0.87 (t, 3H)

### Example 70

### 2-[2-(4-(dimethylamino)benzoylimino)benzo[d]thiazol-3(2H)-yl]butanoic acid

The titled compound was prepared in accordance with the same procedures as in Example 60, using 4-(dimethylamino)benzoyl chloride and ethyl 2-bromobutyrate, instead of 4-methoxybenzoyl chloride and methyl 2-bromobutyrate, respectively (Yield: 77%).

¹H NMR (CDCl₃, 400 MHz) δ 8.10 (s, 2H), 7.72 (d, 1H), 7.34-7.48 (m, 3H), 7.12 (brs, 2H), 5.54 (brs, 1H), 3.10 (s, 6H), 2.48 (brs, 2H), 0.86 (brs, 3H)

### Example 71

### 3-{[3-(1-carboxypropyl)benzo[d]thiazol-2(3H)-ylidene]carbamoyl}benzoic acid

The titled compound was prepared in accordance with the same procedures as in Example 60, using 4-(dimethylamino)benzoyl chloride and ethyl 2-bromobutyrate, instead of 4-methoxybenzoyl chloride and methyl 2-bromobutyrate, respectively (Yield: 93%).

¹H NMR (CD₃OD, 400 MHz) δ 8.92 (s, 1H), 8.52 (d, 1H), 8.20 (d, 1H), 7.83 (d, 1H), 7.51-7.65 (m, 3H), 7.37 (dd, 1H), 5.83 (brs, 1H), 2.54 (t, 2H), 0.88 (t, 3H)

### Example 72

### 2-{2-[2-(trifluoromethyl)benzoylimino]benzo[d]thiazol-3(2H)-yl}butanoic acid

The titled compound was prepared in accordance with the same procedures as in Example 60, using 2-(trifluoromethyl)benzoyl chloride, instead of 4-methoxybenzoyl chloride (Yield: 92%).

¹H NMR (CD₃OD, 400 MHz) δ 9.96 (brs, 1H), 7.88 (d, 1H), 7.71 (d, 2H), 7.47-7.56 (m, 2H), 7.41 (dd, 1H), 7.26-7.35 (m, 2H), 5.95 (brs, 1H), 2.35-2.44 (m, 2H), 0.86 (t, 3H)

### Example 73

### 2-{[3-(1-carboxypropyl)benzo[d]thiazol-2(3H)-ylidene]carbamoyl}benzoic acid

The titled compound was prepared in accordance with the same procedures as in Example 60, using methyl phthaloyl chloride, instead of 4-methoxybenzoyl chloride (Yield: 85%).

¹H NMR (CDCl₃, 400 MHz) δ 11.83 (brs, 2H), 8.20 (d, 1H), 7.91 (s, 1H), 7.70 (d, 1H), 7.54 (s, 1H), 7.27-7.44 (m, 3H), 5.30 (brs, 1H), 2.35-2.50 (m, 2H), 0.83 (t, 3H)

### Example 74

### 2-{2-[(1,1′-biphenyl)-3-carbonylimino]benzo[d]thiazol-3(2H)-yl}butanoic acid

The titled compound was prepared in accordance with the same procedures as in Example 60, using biphenyl 3-carbonyl chloride, instead of 4-methoxybenzoyl chloride (Yield: 89%).

¹H NMR (CDCl₃, 400 MHz) δ 11.13 (brs, 2H), 8.59 (s, 1H), 8.27 (d, 1H), 7.68-7.74 (m, 4H), 7.26-7.50 (m, 7H), 5.57 (brs, 1H), 2.50 (brs, 2H), 0.87 (t, 3H)

### Example 75

### 2-(5,6-difluoro-2-((3-fluoro-4-(trifluoromethyl)benzoyl)imino)benzo[d]thiazol-3(2H)-yl)butanoic acid

The titled compound was prepared in accordance with the same procedures as in Example 52, using, 6-difluorobenzo[d]thiazol-2-amine prepared in Preparation 3, 3-fluoro-4-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 26%).

¹H NMR (MeOD, 400 MHz) δ 8.22 (d, 1H), 8.13 (d, 1H), 7.95-7.75 (m, 3H), 5.75-5.60 (brs, 1H), 2.65-2.45 (m, 2H), 0.89 (t, 3H)

### Example 76

### 2-[2-(2,4-dichloro-5-fluorobenzoylimino)benzo[d]thiazol-3(2H)-yl]butanoic acid

The titled compound was prepared in accordance with the same procedures as in Example 60, using 2,4-dichloro-5-fluorobenzoyl chloride, instead of 4-methoxybenzoyl chloride (Yield: 77%).

¹H NMR (CDCl₃, 400 MHz) δ 11.07 (brs, 1H), 7.93 (d, 1H), 7.73 (d, 1H), 7.43-7.50 (m, 2H), 7.32-7.38 (m, 2H), 5.83 (brs, 1H), 2.40-2.48 (m, 2H), 0.87 (t, 3H)

### Example 77

### 2-{2-[5-fluoro-2-(trifluoromethyl)benzoylimino]benzo[d]thiazol-3(2H)-yl}butanoic acid

The titled compound was prepared in accordance with the same procedures as in Example 60, using 5-fluoro-2-(trifluoromethyl)benzoyl chloride, instead of 4-methoxybenzoyl chloride (Yield: 86%).

¹H NMR (CDCl₃, 400 MHz) δ 10.53 (brs, 1H), 7.73 (d, 2H), 7.65 (d, 1H), 7.43 (d, 1H), 7.35 (t, 2H), 7.20 (t, 1H), 5.96 (brs, 1H), 2.36-2.46 (m, 2H), 0.87 (t, 3H)

### Example 78

### 2-[2-(3,5-difluorobenzoylimino)benzo[d]thiazol-3(2H)-yl]butanoic acid

The titled compound was prepared in accordance with the same procedures as in Example 60, using 3,5-difluorobenzoyl chloride, instead of 4-methoxybenzoyl chloride (Yield: 81%).

¹H NMR (CDCl₃, 400 MHz) δ 10.05 (brs, 1H), 7.72-7.77 (m, 3H), 7.45 (d, 1H), 7.35 (d, 2H), 6.93 (t, 1H), 5.63 (brs, 1H), 2.49 (brs, 2H), 0.88 (t, 3H)

### Example 79

### 2-[2-(4-tert-butylbenzoylimino)benzo[d]thiazol-3(2H)-yl]butanoic acid

The titled compound was prepared in accordance with the same procedures as in Example 60, using 4-tert-butylbenzoyl chloride, instead of 4-methoxybenzoyl chloride (Yield: 66%).

¹H NMR (CDCl₃, 400 MHz) δ 8.22 (d, 2H), 7.61 (d, 1H), 7.45 (d, 2H), 7.22-7.36 (m, 3H), 5.67 (brs, 1H), 2.48 (brd, 2H), 1.31 (s, 9H), 0.85 (t, 3H)

### Example 80

### 2-[2-(4-(methoxymethyl)benzoylimino)benzo[d]thiazol-3(2H)-yl]butanoic acid

The titled compound was prepared in accordance with the same procedures as in Example 60, using 4-chloromethylbenzoyl chloride, instead of 4-methoxybenzoyl chloride (Yield: 66%).

¹H NMR (CDCl₃, 400 MHz) δ 8.20 (d, 2H), 7.90 (d, 1H), 7.34-7.47 (m, 5H), 5.54 (brs, 1H), 4.52 (s, 2H), 3.40 (s, 3H), 2.48-2.55 (m, 2H), 0.89 (t, 3H)

### Example 81

### 2-[2-(4-nitrobenzoylimino)benzo[d]thiazol-3(2H)-yl]butanoic acid

The titled compound was prepared in accordance with the same procedures as in Example 60, using 4-nitrobenzoyl chloride, instead of 4-methoxybenzoyl chloride (Yield: 94%).

¹H NMR (DMSO-d₆, 400 MHz) δ 8.32-8.41 (m, 4H), 8.00 (d, 1H), 7.59 (dd, 1H), 7.43 (dd, 1H), 5.82 (brs, 1H), 2.34-2.53 (m, 2H), 0.75 (t, 3H)

### Example 82

### 2-[2-(3-nitrobenzoylimino)benzo[d]thiazol-3(2H)-yl]butanoic acid

The titled compound was prepared in accordance with the same procedures as in Example 60, using 3-nitrobenzoyl chloride, instead of 4-methoxybenzoyl chloride (Yield: 83%).

¹H NMR (DMSO-d₆, 400 MHz) δ 8.90 (s, 1H), 8.56 (d, 1H), 8.42 (d, 1H), 8.00 (d, 1H), 7.82 (dd, 2H), 7.58 (dd, 1H), 7.43 (dd, 1H), 5.81 (brs, 1H), 2.30-2.53 (m, 2H), 0.75 (t, 3H)

### Example 83

### 2-[2-(3-bromobenzoylimino)benzo[d]thiazol-3(2H)-yl]butanoic acid

The titled compound was prepared in accordance with the same procedures as in Example 60, using 3-bromobenzoyl chloride, instead of 4-methoxybenzoyl chloride (Yield: 86%).

¹H NMR (DMSO-d₆, 400 MHz) δ 8.32 (s, 1H), 8.16 (d, 1H), 7.97 (d, 1H), 7.77-7.91 (m, 2H), 7.57 (dd, 1H), 7.48 (dd, 1H), 7.40 (dd, 1H), 5.81 (brs, 1H), 2.33-2.63 (m, 2H), 0.73 (t, 3H)

### Example 84

### 2-[2-(4-cyanobenzoylimino)benzo[d]thiazol-3(2H)-yl]butanoic acid

The titled compound was prepared in accordance with the same procedures as in Example 60, using 4-cyanobenzoyl chloride, instead of 4-methoxybenzoyl chloride (Yield: 81%).

¹H NMR (CDCl₃, 400 MHz) δ 8.20 (s, 2H), 7.67 (d, 1H), 7.57 (s, 2H), 7.28-7.44 (m, 3H), 5.35 (brs, 1H), 2.40 (brs, 2H), 0.79 (t, 3H)

### Example 85

### 4-{[3-(1-carboxypropyl)benzo[d]thiazol-2(3H)-ylidene]carbamoyl}benzoic acid

The titled compound was prepared in accordance with the same procedures as in Example 60, using methyl 4-(chloroformyl)benzoate, instead of 4-methoxybenzoyl chloride (Yield: 91%).

¹H NMR (CD₃OD, 400 MHz) δ 8.35 (d, 2H), 8.12 (d, 2H), 7.84 (d, 1H), 7.68 (d, 1H), 7.54 (t, 1H), 7.39 (t, 1H), 5.77 (brs, 1H), 2.49-2.59 (m, 2H), 0.87 (t, 3H)

### Example 86

### 2-[2-(4-phenoxybenzoylimino)benzo[d]thiazol-3(2H)-yl]butanoic acid

### Step 1: ethyl 2-[2-(4-phenoxybenzoylimino)benzo[d]thiazol-3(2H)-yl]butanoate

A solution of ethyl 2-(2-iminobenzo[d]thiazol-3(2H)-yl)butanoate (50 mg, 0.19 mmol) prepared in Step 3 of Preparation 18, 4-phenoxybenzoic acid (61.1 mg, 0.29 mmol), (benzotriazol-1-yloxy)tris(dimethylamino)phosphonium hexafluorophosphate (126 mg, 0.29 mmol), and diisopropylethylamine (0.10 mL, 0.57 mmol) in N,N-dimethylformamide (1.9 mL) was stirred at room temperature overnight. The reaction mixture was quenched with water and then extracted with ethyl acetate. The organic layer was dried over anhydrous sodium sulfate and then evaporated. The residue was purified with silica gel column chromatography (n-hexaneethyl acetate=41) to give 34 mg of the titled compound as a white solid (Yield: 39%).

¹H NMR (CDCl₃, 400 MHz) 8.29 (d, 1H), 7.72 (d, 1H), 6.99-7.46 (m, 11H), 5.50 (brs, 1H), 4.06-4.22 (m, 2H), 2.46-2.56 (m, 2H), 1.06 (t, 3H), 0.91 (t, 3H)

### Step 2: 2-[2-(4-phenoxybenzoylimino)benzo[d]thiazol-3(2H)-yl]butanoic acid

The titled compound was prepared in accordance with the same procedures as in Step 2 of Example 60, using ethyl 2-[2-(4-phenoxybenzoylimino)benzo[d]thiazol-3(2H)-yl]butanoate prepared in Step 1 as a starting material (Yield: 95%).

¹H NMR (CDCl₃, 400 MHz) δ 8.23 (d, 1H), 7.68 (d, 1H), 7.26-7.41 (m, 5H), 7.17 (dd, 1H), 7.06 (d, 2H), 6.99 (d, 2H), 5.56 (brs, 1H), 2.43-2.50 (m, 2H), 0.87 (t, 3H)

### Example 87

### 2-{2-[(1,1′-biphenyl)-2-carbonylimino]benzo[d]thiazol-3(2H)-yl}butanoic acid

The titled compound was prepared in accordance with the same procedures as in Example 86, using 2-biphenylcarboxylic acid, instead of 4-phenoxybenzoic acid (Yield: 99%).

¹H NMR (CD₃OD, 400 MHz) δ 7.93 (d, 1H), 7.76 (d, 1H), 7.25-7.52 (m, 11H), 5.22-5.26 (m, 1H), 2.03-2.16 (m, 2H), 0.58 (t, 3H)

### Example 88

### 2-[2-(4-aminobenzoylimino)benzo[d]thiazol-3(2H)-yl]butanoic acid

### Step 1: methyl 2-{[2-(4-nitrobenzoyl)imino]benzo[d]thiazol-3(2H)-yl}butanoate

The titled compound was prepared in accordance with the same procedures as in Step 1 of Example 60, using 4-nitrobenzoyl chloride, instead of 4-methoxybenzoyl chloride (Yield: 43%).

¹H NMR (CDCl₃, 400 MHz) δ 8.46 (d, 2H), 8.32 (m, 2H), 7.78 (d, 1H), 7.52 (dd, 1H), 7.37-7.41 (m, 2H), 5.61 (brs, 1H), 3.59 (s, 3H), 2.48-2.62 (m, 2H), 0.93 (t, 3H)

### Step 2: methyl 2-[2-(4-aminobenzoylimino)benzo[d]thiazol-3(2H)-yl]butanoate

To a solution of methyl 2-[2-(4-nitrobenzoylimino)benzo[d]thiazol-3(2H)-yl]butanoate (55 mg, 0.137 mmol) prepared in Step 1 in ethyl acetate (2.0 mL) was added palladium on activated charcoal (10 wt %, 5.5 mg). The reaction mixture was stirred at room temperature under hydrogen atmosphere for 6 hours. The reaction mixture was filtered through a celite pad. The filtrate was evaporated and purified with silica gel column chromatography (n-hexaneethyl acetate=11) to give 47 mg of the titled compound as a yellow solid (Yield: 93%).

¹H NMR (CDCl₃, 400 MHz) δ 8.13 (d, 2H), 7.68 (d, 1H), 7.41 (dd, 1H), 7.24-7.30 (m, 2H), 6.69 (d, 2H), 5.52 (brs, 1H), 4.07-4.15 (m, 2H), 3.67 (s, 3H), 2.44-2.52 (m, 2H), 0.90 (t, 3H)

### Step 3: 2-[2-(4-aminobenzoylimino)benzo[d]thiazol-3(2H)-yl]butanoic acid

The titled compound was prepared in accordance with the same procedures as in Step 2 of Example 60, using methyl 2-[2-(4-aminobenzoylimino)benzo[d]thiazol-3(2H)-yl]butanoate prepared in Step 2 as a starting material (Yield: 71%).

¹H NMR (CDCl₃, 400 MHz) δ 8.04 (d, 2H), 7.66 (d, 1H), 7.41 (dd, 1H), 7.26-7.33 (m, 2H), 6.61 (d, 2H), 5.91 (brs, 3H), 5.48 (brs, 1H), 2.48 (brd, 2H), 0.86 (t, 3H)

### Example 89

### 2-[2-(3-aminobenzoylimino)benzo[d]thiazol-3(2H)-yl]butanoic acid

The titled compound was prepared in accordance with the same procedures as in Example 88, using 3-nitrobenzoyl chloride, instead of 4-methoxybenzoyl chloride (Yield: 86%).

¹H NMR (CDCl₃, 400 MHz) δ 7.60-7.70 (m, 3H), 7.36 (d, 1H), 7.25 (dd, 2H), 7.11 (dd, 1H), 6.78 (d, 1H), 6.36 (brs, 3H), 5.40 (brs, 1H), 2.38 (brd, 2H), 0.77 (t, 3H)

### Example 90

### 2-[5-fluoro-2-(4-methylbenzoylimino)benzo[d]thiazol-3(2H)-yl]butanoic acid

Methyl 2-[5-fluoro-2-(4-methylbenzoylimino)benzo[d]thiazol-3(2H)-yl]butanoate was prepared in accordance with the same procedures as in Step 1, 2, and 3 of Example 1, using 2,5-difluoroaniline, instead of 2,5-dibromoaniline. The titled compound was prepared in accordance with the same procedures as in Step 2 of Example 60, using methyl 2-[5-fluoro-2-(4-methylbenzoylimino)benzo[d]thiazol-3(2H)-yl]butanoate as a starting material (Yield: 92%).

¹H NMR (CDCl₃, 400 MHz) δ 8.31 (d, 2H), 7.93 (dd, 1H), 7.23 (d, 2H), 7.04-7.08 (m, 2H), 5.56 (brs, 1H), 2.49 (brs, 2H), 2.39 (s, 3H), 0.89 (t, 3H)

### Example 91

### 2-[5-fluoro-2-(4-methylbenzoylimino)benzo[d]thiazol-3(2H)-yl]propionic acid

The titled compound was prepared in accordance with the same procedures as in Example 90, using ethyl 2-bromopropionate, instead of methyl 2-bromobutyrate (Yield: 92%).

¹H NMR (CDCl₃, 400 MHz) δ 8.19 (d, 2H), 7.66 (dd, 1H), 7.27 (d, 2H), 7.05-7.14 (m, 2H), 5.82 (brs, 1H), 2.42 (s, 3H), 1.85 (d, 3H)

### Example 92

### 2-[5-fluoro-2-(4-methylbenzoylimino)benzo[d]thiazol-3(2H)-yl]acetic acid

The titled compound was prepared in accordance with the same procedures as in Example 90, using ethyl bromoacetate, instead of methyl 2-bromobutyrate (Yield: 58%).

¹H NMR (CDCl₃, 400 MHz) δ 8.20 (d, 2H), 7.65 (dd, 1H), 7.27 (d, 2H), 7.05-7.19 (m, 2H), 5.21 (s, 2H), 2.43 (s, 3H)

### Example 93

### 2-[7-fluoro-2-(4-methylbenzoylimino)benzo[d]thiazol-3(2H)-yl]butanoic acid

The titled compound was prepared in accordance with the same procedures as in Example 90, using 2,3-difluoroaniline, instead of 2,5-dibromoaniline (Yield: 100%).

¹H NMR (CDCl₃, 400 MHz) δ 8.19 (d, 2H), 7.37-7.43 (m, 1H), 7.28 (d, 2H), 7.18 (d, 1H), 7.05 (dd, 2H), 5.74 (brs, 1H), 2.48-2.53 (m, 2H), 2.43 (s, 3H), 0.90 (t, 3H)

### Example 94

### 2-[7-fluoro-2-(4-methylbenzoylimino)benzo[d]thiazol-3(2H)-yl]propionic acid

The titled compound was prepared in accordance with the same procedures as in Example 90, using 2,3-difluoroaniline and ethyl 2-bromopropionate, instead of 2,5-dibromoaniline and methyl 2-bromobutyrate, respectively (Yield: 93%).

¹H NMR (CDCl₃, 400 MHz) δ 8.19 (d, 2H), 7.42-7.48 (m, 1H), 7.28 (d, 2H), 7.22 (d, 1H), 7.06 (dd, 2H), 5.85 (brs, 1H), 2.43 (s, 3H), 1.85 (d, 3H)

### Example 95

### 2-[7-fluoro-2-(4-methylbenzoylimino)benzo[d]thiazol-3(2H)-yl]acetic acid

The titled compound was prepared in accordance with the same procedures as in Example 90, using 2,3-difluoroaniline and ethyl bromoacetate, instead of 2,5-dibromoaniline and methyl 2-bromobutyrate, respectively (Yield: 75%).

¹H NMR (DMSO-d₆, 400 MHz) δ 8.15 (d, 2H), 7.55-7.63 (m, 2H), 7.27-7.36 (m, 3H), 5.34 (s, 2H), 2.40 (s, 3H)

### Example 96

### 2-[2-(4-methylbenzoylimino)-5-(trifluoromethyl)benzo[d]thiazol-3(2H)-yl]butanoic acid

The titled compound was prepared in accordance with the same procedures as in Example 90, using 2-fluoro-5-(trifluoro-methyl)aniline, instead of 2,5-dibromoaniline (Yield: 85%).

¹H NMR (CDCl₃, 400 MHz) δ 8.15 (d, 2H), 7.79 (d, 1H), 7.55 (d, 2H), 7.21-7.26 (m, 2H), 5.58 (brs, 1H), 2.50-2.55 (m, 2H), 2.39 (s, 3H), 0.90 (t, 3H)

### Example 97

### 2-[2-(4-methylbenzoylimino)-7-(trifluoromethyl)benzo[d]thiazol-3(2H)-yl]butanoic acid

The titled compound was prepared in accordance with the same procedures as in Example 90, using 2-fluoro-3-(trifluoromethyl)aniline, instead of 2,5-dibromoaniline (Yield: 96%).

¹H NMR (DMSO-d₆, 400 MHz) δ 8.15 (d, 2H), 7.62-7.76 (m, 3H), 7.34 (d, 2H), 5.74 (brs, 1H), 2.39-2.50 (m, 1H), 2.39 (s, 3H), 2.15-2.19 (m, 1H), 0.76 (t, 3H)

### Example 98

### 2-[2-(4-methylbenzoylimino)-7-(trifluoromethyl)benzo[d]thiazol-3(2H)-yl]propionic acid

The titled compound was prepared in accordance with the same procedures as in Example 90, using 2-fluoro-3-(trifluoromethyl)aniline and ethyl 2-bromopropionate, instead of 2,5-dibromoaniline and methyl 2-bromobutyrate, respectively (Yield: 94%).

¹H NMR (DMSO-d₆, 400 MHz) δ 8.12 (d, 3H), 7.79 (d, 2H), 7.34 (d, 2H), 5.98 (brs, 1H), 2.40 (s, 3H), 1.75 (d, 3H)

### Example 99

### 2-[2-(4-methylbenzoylimino)-7-(trifluoromethyl)benzo[d]thiazol-3(2H)-yl]acetic acid

The titled compound was prepared in accordance with the same procedures as in Example 90, using 2-fluoro-3-(trifluoromethyl)aniline and ethyl bromoacetate, instead of 2,5-dibromoaniline and methyl 2-bromobutyrate, respectively (Yield: 100%).

¹H NMR (DMSO-d₆, 400 MHz) δ 8.12 (d, 2H), 8.03 (d, 1H), 7.73-7.78 (m, 2H), 7.30 (d, 2H), 5.34 (s, 2H), 2.37 (s, 3H)

### Example 100

### 2-[5-methyl-2-(4-methylbenzoylimino)benzo[d]thiazol-3(2H)-yl]butanoic acid

The titled compound was prepared in accordance with the same procedures as in Example 90, using 2-fluoro-5-methylaniline, instead of 2,5-dibromoaniline (Yield: 78%).

¹H NMR (CDCl₃, 400 MHz) δ 8.13 (d, 2H), 7.57 (d, 1H), 7.22 (d, 2H), 7.14 (d, 2H), 5.59 (brs, 1H), 2.45-2.51 (m, 2H), 2.46 (s, 3H), 2.39 (s, 3H), 0.88 (t, 3H)

### Example 101

### 2-[5-methyl-2-(4-methylbenzoylimino)benzo[d]thiazol-3(2H)-yl]propionic acid

The titled compound was prepared in accordance with the same procedures as in Example 90, using 2-fluoro-5-methylaniline and ethyl 2-bromopropionate, instead of 2,5-dibromoaniline and methyl 2-bromobutyrate, respectively (Yield: 84%).

¹H NMR (CDCl₃, 400 MHz) δ 8.13 (d, 2H), 7.56 (d, 1H), 7.20 (d, 2H), 7.11-7.14 (m, 2H), 5.60 (brs, 1H), 2.46 (s, 3H), 2.38 (s, 3H), 1.83 (d, 3H)

### Example 102

### 2-[5-methyl-2-(4-methylbenzoylimino)benzo[d]thiazol-3(2H)-yl)acetic acid

The titled compound was prepared in accordance with the same procedures as in Example 90, using 2-fluoro-5-methylaniline and ethyl bromoacetate, instead of 2,5-dibromoaniline and methyl 2-bromobutyrate, respectively (Yield: 76%).

¹H NMR (DMSO-d₆, 400 MHz) δ 8.10 (d, 2H), 7.76 (d, 1H), 7.41 (s, 1H), 7.27 (d, 2H), 7.17 (d, 1H), 5.11 (s, 2H), 2.41 (s, 3H), 2.36 (s, 3H)

### Example 103

### 2-[7-methyl-2-(4-methylbenzoylimino)benzo[d]thiazol-3(2H)-yl]propionic acid

The titled compound was prepared in accordance with the same procedures as in Example 90, using 2-fluoro-3-methylaniline and ethyl 2-bromopropionate, instead of 2,5-dibromoaniline and methyl 2-bromobutyrate, respectively (Yield: 44%).

¹H NMR (CD₃OD, 400 MHz) δ 8.20 (d, 2H), 7.46 (brs, 2H), 7.31 (d, 2H), 7.22 (d, 1H), 5.91 (brs, 1H), 2.56 (s, 3H), 2.44 (s, 3H), 1.82 (d, 3H)

### Example 104

### 2-[7-methyl-2-(4-methylbenzoylimino)benzo[d]thiazol-3(2H)-yl]acetic acid

The titled compound was prepared in accordance with the same procedures as in Example 90, using 2-fluoro-3-methylaniline and ethyl bromoacetate, instead of 2,5-dibromoaniline and methyl 2-bromobutyrate, respectively (Yield: 60%).

¹H NMR (CD₃OD, 400 MHz) δ 8.16 (d, 2H), 7.25-7.39 (m, 4H), 7.16 (d, 1H), 5.19 (s, 2H), 2.51 (s, 3H), 2.41 (s, 3H)

### Example 105

### 2-[5-chloro-2-(4-methylbenzoylimino)benzo[d]thiazol-3(2H)-yl]butanoic acid

The titled compound was prepared in accordance with the same procedures as in Example 90, using 5-chloro-2-fluoroaniline, instead of 2,5-dibromoaniline (Yield: 96%).

¹H NMR (DMSO-d₆, 400 MHz) δ 8.08 (d, 2H), 7.96 (d, 2H), 7.40 (dd, 1H), 7.30 (d, 2H), 5.69 (brs, 1H), 2.36-2.50 (m, 2H), 2.38 (s, 3H), 0.75 (t, 3H)

### Example 106

### 2-[5-chloro-2-(4-methylbenzoylimino)benzo[d]thiazol-3(2H)-yl]propionic acid

The titled compound was prepared in accordance with the same procedures as in Example 90, using 5-chloro-2-fluoroaniline and ethyl 2-bromopropionate, instead of 2,5-dibromoaniline and methyl 2-bromobutyrate, respectively (Yield: 100%).

¹H NMR (DMSO-d₆, 400 MHz) δ 8.09 (d, 2H), 7.96 (d, 2H), 7.42 (dd, 1H), 7.31 (d, 2H), 5.80 (brd, 1H), 2.38 (s, 3H), 1.71 (d, 3H)

### Example 107

### 2-[5-chloro-2-(4-methylbenzoylimino)benzo[d]thiazol-3(2H)-yl]acetic acid

The titled compound was prepared in accordance with the same procedures as in Example 90, using 5-chloro-2-fluoroaniline and ethyl bromoacetate, instead of 2,5-dibromoaniline and methyl 2-bromobutyrate, respectively (Yield: 95%).

¹H NMR (DMSO-d₆, 400 MHz) δ 8.11 (d, 2H), 7.95 (d, 2H), 7.41 (dd, 1H), 7.31 (d, 2H), 5.30 (s, 2H), 2.38 (s, 3H)

### Example 108

### 2-[7-chloro-2-(4-methylbenzoylimino)benzo[d]thiazol-3(2H)-yl]butanoic acid

The titled compound was prepared in accordance with the same procedures as in Example 90, using 3-chloro-2-fluoroaniline, instead of 2,5-dibromoaniline (Yield: 97%).

¹H NMR (DMSO-d₆, 400 MHz) δ 8.09 (d, 2H), 7.80 (s, 1H), 7.58 (dd, 1H), 7.49 (d, 1H), 7.32 (d, 2H), 5.74 (brs, 1H), 2.36-2.50 (m, 2H), 2.38 (s, 3H), 0.76 (t, 3H)

### Example 109

### 2-[7-chloro-2-(4-methylbenzoylimino)benzo[d]thiazol-3(2H)-yl]propionic acid

The titled compound was prepared in accordance with the same procedures as in Example 90, using 3-chloro-2-fluoroaniline and ethyl 2-bromopropionate, instead of 2,5-dibromoaniline and methyl 2-bromobutyrate, respectively (Yield: 97%).

¹H NMR (DMSO-d₆, 400 MHz) δ 8.11 (d, 2H), 7.77 (s, 1H), 7.59 (dd, 1H), 7.49 (d, 1H), 7.32 (d, 2H), 5.87 (brd, 1H), 2.39 (s, 3H), 1.72 (d, 3H)

### Example 110

### 2-[7-chloro-2-(4-methylbenzoylimino)benzo[d]thiazol-3(2H)-yl]acetic acid

The titled compound was prepared in accordance with the same procedures as in Example 90, using 3-chloro-2-fluoroaniline and ethyl bromoacetate, instead of 2,5-dibromoaniline and methyl 2-bromobutyrate, respectively (Yield: 91%).

¹H NMR (DMSO-d₆, 400 MHz) δ 8.13 (d, 2H), 7.73 (d, 1H), 7.56 (dd, 1H), 7.48 (d, 1H), 7.33 (d, 2H), 5.32 (s, 2H), 2.38 (s, 3H)

### Example 111

### 2-[2-(4-methylbenzoylimino)-5-propoxybenzo[d]thiazol-3(2H)-yl]butanoic acid

### Step 1: methyl 2-[5-hydroxy-2-(4-methylbenzoylimino)benzo[d]thiazol-3(2H)-yl]butanoate

A solution of methyl 2-{5-(benzyloxy)-2-[(4-methylbenzoyl)imino]benzo[d]thiazol-3(2H)-yl}butanoate (1.00 g, 2.11 mmol) prepared in Example 22 and palladium/carbon (10 wt %, 100 mg) in a mixed solvent of ethyl acetate and methanol (1/1, 42 mL) was stirred at room temperature under hydrogen atmosphere for 3 hours. The reaction mixture was filtered through a celite pad. The resulting filtrate was concentrated under reduced pressure to give 733 mg of the titled compound as a colorless liquid (Yield: 90%).

¹H NMR (400 MHz, CDCl₃)₈8.16 (d, 2H), 7.52 (d, 1H), 7.28 (d, 2H), 7.02 (s, 1H), 6.97 (d, 1H), 5.61 (brs, 1H), 3.60 (s, 3H), 2.47 (brs, 2H), 2.42 (s, 3H), 0.88 (t, 3H)

### Step 2: methyl 2-[2-(4-methylbenzoylimino)-5-propoxybenzo[d]thiazol-3(2H)-yl]butanoate

To a solution of methyl 2-[5-hydroxy-2-(4-methylbenzoylimino)benzo[d]thiazol-3(2H)-yl]butanoate (50 mg, 0.13 mmol) prepared in Step 1 in N,N-dimethylformamide (1.3 mL), were added 1-iodopropane (44.2 mg, 0.26 mmol) and potassium carbonate (35.9 mg, 0.26 mmol). The reaction mixture was stirred at room temperature for 3 hours. The reaction mixture was quenched with water and then extracted with ethyl acetate. The organic layer was dried over anhydrous sodium sulfate, filtered and then evaporated. The residue was purified with silica gel column chromatography (n-hexaneethyl acetate=10/1) to give 43.2 mg of the titled compound as a white solid (Yield: 23%)

¹H NMR (CDCl₃, 400 MHz) δ 8.19 (d, 2H), 7.56 (d, 1H), 7.27 (d, 2H), 6.90 (d, 1H), 6.82 (s, 1H), 5.49 (brs, 1H), 3.94-4.00 (m, 2H), 3.65 (s, 3H), 2.48-2.55 (m, 2H), 2.42 (s, 3H), 1.82-1.88 (m, 2H), 1.07 (t, 3H), 0.90 (t, 3H)

### Step 3: 2-[2-(4-methylbenzoylimino)-5-propoxybenzo[d]thiazol-3(2H)-yl]butanoic acid

The titled compound was prepared in accordance with the same procedures as in Step 2 of Example 60, using methyl 2-[2-(4-methylbenzoylimino)-5-propoxybenzo[d]thiazol-3(2H)-yl]butanoate prepared in Step 2 as a starting material (Yield: 76%).

¹H NMR (CDCl₃, 400 MHz) δ 8.15 (d, 2H), 7.51 (d, 1H), 7.21 (d, 2H), 6.86-6.88 (m, 2H), 5.57 (brs, 1H), 3.93 (brs, 2H), 2.45 (brs, 2H), 2.38 (s, 3H), 1.79-1.84 (m, 2H), 1.04 (t, 3H), 0.85 (t, 3H)

### Example 112

### 2-[5-butoxy-2-(4-methylbenzoylimino)benzo[d]thiazol-3(2H)-yl]butanoic acid

The titled compound was prepared in accordance with the same procedures as in Example 111, using iodobutane, instead of 1-iodopropane (Yield: 65%).

¹H NMR (CDCl₃, 400 MHz) δ 8.16 (d, 2H), 7.54 (d, 1H), 7.22-7.67 (m, 2H), 6.88 (s, 2H), 5.45 (brs, 1H), 4.00 (brs, 2H), 2.50 (brs, 2H), 2.39 (s, 3H), 1.79 (brs, 2H), 1.51 (brs, 2H), 1.00 (t, 3H), 0.88 (brs, 3H)

### Example 113

### 2-[5-isopropoxy-2-(4-methylbenzoylimino)benzo[d]thiazol-3(2H)-yl]butanoic acid

The titled compound was prepared in accordance with the same procedures as in Example 111, using 2-iodopropane, instead of 1-iodopropane (Yield: 84%).

¹H NMR (CDCl₃, 400 MHz) δ 8.16 (d, 2H), 7.51 (d, 1H), 7.21 (d, 2H), 6.86 (s, 2H), 5.57 (brs, 1H), 4.54-4.58 (m, 1H), 2.45 (brs, 2H), 2.39 (s, 3H), 1.35 (d, 6H), 0.86 (brs, 3H)

### Example 114

### 2-[5-(cyclohexyloxy)-2-(4-methylbenzoylimino)benzo[d]thiazol-3(2H)-yl]butanoic acid

The titled compound was prepared in accordance with the same procedures as in Example 111, using cyclohexyl iodide, instead of 1-iodopropane (Yield: 68%).

¹H NMR (CDCl₃, 400 MHz) δ 8.16 (d, 2H), 7.55 (d, 1H), 7.25 (d, 2H), 6.91 (s, 2H), 5.55 (brs, 1H), 4.35-4.40 (m, 1H), 2.51 (brs, 2H), 2.41 (s, 3H), 1.99 (brs, 2H), 1.81 (brs, 2H), 1.54-1.60 (m, 3H), 1.36-1.44 (m, 3H), 0.90 (t, 3H)

### Example 115

### 2-[5-hydroxy-2-(4-methylbenzoylimino)benzo[d]thiazol-3(2H)-yl]butanoic acid

The titled compound was prepared in accordance with the same procedures as in Step 2 of Example 60, using methyl 2-[5-hydroxy-2-(4-methylbenzoylimino)benzo[d]thiazol-3(2H)-yl]butanoate prepared in Step 1 of Example 111 as a starting material (Yield: 80%).

¹H NMR (CD₃OD, 400 MHz) δ 8.04 (d, 2H), 7.46 (d, 1H), 7.16 (d, 2H), 6.92 (s, 1H), 6.75 (d, 1H), 5.55 (brs, 1H), 2.34-2.44 (m, 2H), 2.28 (s, 3H), 0.76 (t, 3H)

### Example 116

### 2-[6-(4-fluorophenylamino)-2-(4-methylbenzoylimino)benzo[d]thiazol-3(2H)-yl]butanoic acid

### Step 1: N-(6-bromobenzo[d]thiazol-2-yl)-4-methylbenzamide

To a solution of 6-bromobenzo[d]thiazol-2-amine (10.0 g, 43.7 mmol) in toluene (146 mL), were added diisopropylethylamine (15.2 mL, 87.3 mmol) and p-toluoyl chloride (10.1 g, 65.5 mmol). The reaction mixture was stirred at 80° C. for 3 hours. The reaction mixture was quenched with water and then extracted with ethyl acetate. The organic layer was washed with an aqueous 1N HCl solution, a saturated sodium bicarbonate solution, and brine. The organic layer was dried over anhydrous magnesium sulfate, filtered, and then evaporated. The residue was diluted with isopropyl ether. The resulting solid was filtered and then dried under reduced pressure to give 12.7 g of the titled compound as a white solid (Yield: 84%).

### Step 2: methyl 2-[6-bromo-2-(4-methylbenzoylimino)benzo[d]thiazol-3(2H)-yl]butanoate

To a solution of N-(6-bromobenzo[d]thiazol-2-yl)-4-methylbenzamide (12.7 g, 36.6 mmol) prepared in Step 1 in N,N-dimethylformamide (122 mL), were added potassium carbonate (10.1 g, 73.2 mmol) and methyl 2-bromobutyrate (8.6 g, 47.5 mmol). The reaction mixture was stirred at 80° C. for 2 hours. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=2/1) to give 12.4 g of the titled compound as a white solid (Yield: 75%).

¹H NMR (CDCl₃, 400 MHz) δ 8.18 (d, 2H), 7.83 (d, 1H), 7.53 (dd, 1H), 7.27 (d, 2H), 7.14 (d, 1H), 5.56 (brs, 1H), 3.66 (s, 3H), 2.42-2.56 (m, 2H), 2.42 (s, 3H), 0.90 (t, 3H)

### Step 3: methyl 2-[6-(4-fluorophenylamino)-2-(4-methylbenzoylimino)benzo[d]thiazol-3(2H)-yl]butanoate

To a solution of methyl 2-[6-bromo-2-(4-methylbenzoylimino)benzo[d]thiazol-3(2H)-yl]butanoate (50 mg, 0.111 mmol) prepared in Step 2 in 1,4-dioxane (0.7 mL), were added 4-fluoroaniline (11.1 mg, 0.10 mmol), palladium acetate (1.5 mg, 0.007 mmol), XANTPHOS (4.1 mg, 0.007 mmol), and cesium carbonate (43.7 mg, 0.134 mmol). The reaction mixture was reacted in a microwave reactor (300 W, 100° C.) for 20 minutes, cooled to room temperature, and then concentrated under reduced pressure. The resulting residue was purified with silica gel column chromatography (n-hexane/ethyl acetate=10/1) to give 20.8 mg of the titled compound as a yellow solid (Yield: 65%).

¹H NMR (CDCl₃, 400 MHz) δ 8.18 (d, 2H), 7.30 (s, 1H), 7.26 (d, 2H), 7.15 (d, 1H), 6.98-7.06 (m, 5H), 5.81 (s, 1H), 5.54 (brs, 1H), 3.66 (s, 3H), 2.44-2.51 (m, 2H), 2.42 (s, 3H), 0.91 (t, 3H)

### Step 4: 2-[6-(4-fluorophenylamino)-2-(4-methylbenzoylimino)benzo[d]thiazol-3(2H)-yl]butanoic acid

The titled compound was prepared in accordance with the same procedures as in Step 2 of Example 60, using methyl 2-[6-(4-fluorophenylamino)-2-(4-methylbenzoylimino)benzo[d]thiazol-3(2H)-yl]butan oate prepared in Step 3 as a starting material (Yield: 93%).

¹H NMR (CDCl₃, 400 MHz) δ 8.07 (d, 2H), 6.86-7.25 (m, 9H), 5.32 (brs, 2H), 2.32 (s, 3H), 2.23-2.42 (m, 2H), 0.77 (t, 3H)

### Example 117

### 2-[2-(4-methylbenzoylimino)-6-(pyrimidin-2-ylamino)benzo[d]thiazol-3(2H)-yl]butanoic acid

The titled compound was prepared in accordance with the same procedures as in Example 116, using 2-aminopyrimidine, instead of 4-fluoroaniline (Yield: 79%).

¹H NMR (CDCl₃, 400 MHz) δ 9.41 (s, 1H), 8.40 (d, 2H), 8.30 (s, 1H), 8.18 (d, 2H), 7.18 (brs, 4H), 6.76 (dd, 1H), 5.55 (brs, 1H), 2.38-2.56 (m, 2H), 2.37 (s, 3H), 0.91 (t, 3H)

### Example 118

### 2-[7-bromo-2-(4-methylbenzoylimino)benzo[d]thiazol-3(2H)-yl]butanoic acid

### Step 1: a mixture of 5-bromobenzo[d]thiazol-2-amine and 7-bromobenzo[d]thiazol-2-amine

To a stirred solution of 1-(3-bromophenyl)thiourea (1.00 g, 4.33 mmol) in dichloromethane (15 mL) was added dropwise a solution of bromine (345 mg, 4.33 mmol) in dichloromethane (15 mL). The reaction mixture was stirred at room temperature overnight. The resulting solid was filtered, washed with dichloromethane, and then dried under reduced pressure to give 809 mg of the titled compound as a yellow solid (Yield: 82%).

### Step 2: a mixture of N-(5-bromobenzo[d]thiazol-2-yl)-4-methylbenzamide and N-(7-bromobenzo[d]thiazol-2-yl)-4-methylbenzamide

To a solution of the mixture of 5-bromobenzo[d]thiazol-2-amine and 7-bromobenzo[d]thiazol-2-amine (100 mg, 0.436 mmol) prepared in Step 1 in toluene (2.2 mL), were added p-toluoyl chloride (135 mg, 0.872 mmol) and diisopropylethylamine (0.22 mL, 1.38 mmol). The reaction mixture was stirred at 80° C. for 2 hours. The reaction mixture was quenched with water and then extracted with dichloromethane. The extract was dried over anhydrous sodium sulfate, filtered, and then evaporated. The residue was used in the next step without further purification.

### Step 3: methyl 2-[7-bromo-2-(4-methylbenzoylimino)benzo[d]thiazol-3(2H)-yl]butanoate

To a solution of the mixture of N-(5-bromobenzo[d]thiazol-2-yl)-4-methylbenzamide and N-(7-bromobenzo[d]thiazol-2-yl)-4-methylbenzamide (90 mg, 0.258 mmol) prepared in Step 2 in N,N-dimethylformamide (3.0 mL), were added methyl 2-bromobutyrate (93.9 mg, 0.519 mmol) and potassium carbonate (107 mg, 0.768 mmol). The reaction mixture was stirred at 80° C. for 3 hours. The reaction mixture was quenched with water and then extracted with ethyl acetate. The extract was dried over anhydrous sodium sulfate, filtered, and then evaporated. The residue was purified with silica gel column chromatography (n-hexaneethyl acetate=10/1) to give 26.7 mg of the titled compound as a white solid (Yield: 23%).

¹H NMR (CDCl₃, 400 MHz) δ 8.19 (d, 2H), 7.45 (d, 1H), 7.20-7.34 (m, 4H), 5.53 (brs, 1H), 3.67 (s, 3H), 2.47-2.54 (m, 2H), 2.43 (s, 3H), 0.91 (t, 3H)

### Step 4: 2-[7-bromo-2-(4-methylbenzoylimino)benzo[d]thiazol-3(2H)-yl]butanoic acid

To a solution of methyl 2-[7-bromo-2-(4-methylbenzoylimino)benzo[d]thiazol-3(2H)-yl]butanoate (9.1 mg, 0.020 mmol) prepared in Step 3 in a mixed solvent of tetrahydrofuran and methanol (1/1, 2.0 mL) was added a 1N sodium hydroxide solution (0.5 mL) and the reaction mixture was stirred at room temperature overnight. The reaction mixture was evaporated, acidified with an aqueous 1N HCl solution, and then extracted with dichloromethane. The extract was washed with brine, dried over anhydrous magnesium sulfate, filtered, and then evaporated. The residue was dried under reduced pressure to give 5.8 mg of the titled compound as a white solid (Yield: 67%).

¹H NMR (CDCl₃, 400 MHz) δ 8.14 (d, 2H), 7.45 (d, 1H), 7.22-7.28 (m, 4H), 5.55 (brs, 1H), 2.48 (brs, 2H), 2.41 (s, 3H), 0.90 (t, 3H)

### Example 119

### 2-[2-(benzoylimino)benzo[d]thiazol-3(2H)-yl]acetic acid

### Step 1: N-(benzo[d]thiazol-2-yl)benzamide

To a solution of benzo[d]thiazol-2-amine (2.0 g, 13.3 mmol) in tetrahydrofuran (25 mL), were added cesium carbonate (13.0 g, 20.0 mmol) and benzoyl chloride (3.7 mL, 39.9 mmol). The reaction mixture was stirred at room temperature overnight and then concentrated under reduced pressure. The residue was dissolved in ethyl acetate. The solution was washed with distilled water, dried over anhydrous magnesium sulfate, filtered, and then evaporated. The resulting solid was washed with ethyl acetate and then dried under reduced pressure to give 2.4 g of the titled compound as a white solid (Yield: 71%).

### Step 2: ethyl 2-[2-(benzoylimino)benzo[d]thiazol-3(2H)-yl]acetate

To a solution of N-(benzo[d]thiazol-2-yl)benzamide (300 mg, 1.18 mmol) prepared in Step 1 in N,N-dimethylformamide (4.00 mL) was added sodium hydride (60%, 71 mg, 1.77 mmol). The reaction mixture was stirred at room temperature for 1 minute and then ethyl bromoacetate (144 uL, 1.30 mmol) was added dropwise thereto. The reaction mixture was stirred at room temperature for 1 hour and then refluxed under stirring at 80° C. overnight. The reaction mixture was quenched with water and then extracted with ethyl acetate. The extract was dried over anhydrous magnesium sulfate, filtered, and then evaporated. The residue was purified with silica gel column chromatography (n-hexaneethyl acetate=10/1) to give 350 mg of the titled compound as a white solid (Yield: 87%)

¹H NMR (CDCl₃, 400 MHz) δ 8.34 (d, 2H), 7.72 (d, 1H), 7.53 (t, 1H), 7.44-7.48 (m, 3H), 7.33 (t, 1H), 7.23-7.26 (m, 1H), 5.24 (s, 2H), 4.27 (q, 2H), 1.27 (t, 3H)

### Step 3: 2-[2-(benzoylimino)benzo[d]thiazol-3(2H)-yl]acetic acid

To a solution of ethyl 2-[2-(benzoylimino)benzo[d]thiazol-3(2H)-yl]acetate (100 mg, 0.29 mmol) prepared in Step 2 in methanol (4.0 mL) was added a 1N sodium hydroxide solution (2.0 mL). The reaction mixture was stirred at 30° C. overnight. The reaction mixture was evaporated, diluted with water, washed with diethyl ether, and then acidified with an aqueous 1N HCl solution. The resulting solid was filtered, washed with water, and then dried under reduced pressure to give 76 mg of the titled compound as a white solid (Yield: 84%).

¹H NMR (DMSO-d₆, 400 MHz) δ 13.37 (s, 1H), 8.25 (d, 2H), 7.96 (d, 1H), 7.74 (d, 1H), 7.51-7.62 (m, 4H), 7.39 (t, 1H), 5.34 (s, 2H)

### Example 120

### 2-[2-(4-methylbenzoylimino)benzo[d]thiazol-3(2H)-yl]acetic acid

The titled compound was prepared in accordance with the same procedures as in Example 119, using p-toluoyl chloride, instead of benzoyl chloride (Yield: 95%).

¹H NMR (CD₃OD, 400 MHz) δ 8.18 (d, 2H), 7.81 (d, 1H), 7.52-7.54 (m, 2H), 7.38 (t, 1H), 7.29 (d, 2H), 5.33 (s, 2H), 2.42 (s, 3H)

### Example 121

### 2-[2-(3-methylbenzoylimino)benzo[d]thiazol-3(2H)-yl]acetic acid

### Step 1: N-(benzo[d]thiazol-2-yl)-3-methylbenzamide

The titled compound was prepared in accordance with the same procedures as in Step 1 of Example 119, using m-toluoyl chloride, instead of benzoyl chloride (Yield: 35%).

### Step 2: ethyl 2-[2-(3-methylbenzoylimino)benzo[d]thiazol-3(2H)-yl]acetate

To a solution of N-(benzo[d]thiazol-2-yl)-3-methylbenzamide (100 mg, 0.37 mmol) prepared in Step 1 in N,N-dimethylformamide (1.00 mL), were added ethyl bromoacetate (61 mg, 0.56 mmol) and potassium carbonate (153 mg, 1.11 mmol). The reaction mixture was refluxed under stirring at 80° C. overnight. The reaction mixture was quenched with water and then extracted with ethyl acetate. The extract was dried over anhydrous magnesium sulfate, filtered, and then evaporated. The residue was washed with n-hexaneethyl acetate (10/1) and then dried under reduced pressure to give 103 mg of the titled compound as a pale yellow solid (Yield: 78%).

¹H NMR (CDCl₃, 400 MHz) δ 8.14 (m, 2H), 7.71 (d, 1H), 7.45 (t, 1H), 7.30-7.34 (m, 3H), 7.22-7.26 (m, 1H), 5.23 (s, 2H), 4.27 (q, 2H), 2.43 (s, 3H), 1.26 (t, 3H)

### Step 3: 2-[2-(3-methylbenzoylimino)benzo[d]thiazol-3(2H)-yl]acetic acid

The titled compound was prepared in accordance with the same procedures as in Step 3 of Example 119, using ethyl 2-[2-(3-methylbenzoylimino)benzo[d]thiazol-3(2H)-yl]acetate prepared in Step 2 as a starting material (Yield: 92%).

¹H NMR (DMSO-d₆, 400 MHz) δ 13.37 (m, 1H), 8.05 (s, 2H), 7.95 (d, 1H), 7.74 (d, 1H), 7.53 (t, 1H), 7.36-7.40 (m, 3H), 5.33 (s, 2H), 2.40 (s, 3H)

### Example 122

### 2-[2-(4-chlorobenzoylimino)benzo[d]thiazol-3(2H)-yl]acetic acid

The titled compound was prepared in accordance with the same procedures as in Example 119, using 4-chlorobenzoyl chloride, instead of benzoyl chloride (Yield: 92%).

¹H NMR (DMSO-d₆, 400 MHz) δ 13.40 (s, 1H), 8.24 (d, 2H), 7.97 (d, 1H), 7.74 (d, 1H), 7.52-7.60 (m, 3H), 7.39 (t, 1H), 5.35 (s, 2H)

### Example 123

### 2-[2-(3-chlorobenzoylimino)benzo[d]thiazol-3(2H)-yl]acetic acid

The titled compound was prepared in accordance with the same procedures as in Example 121, using 3-chlorobenzoyl chloride, instead of m-toluoyl chloride (Yield: 67%).

¹H NMR (DMSO-d₆, 400 MHz) δ 13.29-13.55 (m, 1H), 8.08-8.12 (m, 2H), 7.89 (d, 1H), 7.68 (d, 1H), 7.58 (d, 1H), 7.46-7.49 (m, 2H), 7.32 (t, 1H), 5.26 (s, 2H)

### Example 124

### 2-[2-(2-chlorobenzoylimino)benzo[d]thiazol-3(2H)-yl]acetic acid

The titled compound was prepared in accordance with the same procedures as in Example 119, using 2-chlorobenzoyl chloride, instead of benzoyl chloride (Yield: 67%).

¹H NMR (DMSO-d₆, 400 MHz) δ 7.97 (t, 2H), 7.71 (d, 1H), 7.48-7.56 (m, 3H), 7.38-7.46 (m, 2H), 5.26 (s, 2H)

### Example 125

### 2-[2-(3-(trifluoromethyl)benzoylimino)benzo[d]thiazol-3(2H)-yl]acetic acid

The titled compound was prepared in accordance with the same procedures as in Example 119, using 3-(trifluoromethyl)benzoyl chloride, instead of benzoyl chloride (Yield: 91%).

¹H NMR (DMSO-d₆, 400 MHz) δ 8.49 (m, 2H), 7.97 (m, 2H), 7.75 (m, 2H), 7.40-7.54 (m, 2H), 5.29 (s, 2H)

### Example 126

### 2-[2-(4-(trifluoromethoxy)benzoylimino)benzo[d]thiazol-3(2H)-yl]acetic acid

The titled compound was prepared in accordance with the same procedures as in Example 119, using 4-trifluoromethoxybenzoyl chloride, instead of benzoyl chloride (Yield: 87%).

¹H NMR (DMSO-d₆, 400 MHz) δ 13.40 (s, 1H), 8.35 (d, 2H), 7.97 (d, 1H), 7.75 (d, 1H), 7.50-7.57 (m, 3H), 7.40 (t, 1H), 5.36 (s, 2H)

### Example 127

### 2-[2-(2,6-dimethoxybenzoylimino)benzo[d]thiazol-3(2H)-yl]acetic acid

The titled compound was prepared in accordance with the same procedures as in Example 119, using 2,6-dimethoxybenzoyl chloride, instead of benzoyl chloride (Yield: 71%).

¹H NMR (DMSO-d₆, 400 MHz) δ 12.88 (s, 1H), 8.04 (d, 1H), 7.80 (d, 1H), 7.48 (t, 2H), 7.37 (t, 1H), 6.80 (d, 2H), 4.63 (s, 2H), 3.75 (s, 6H)

### Example 128

### 2-[2-(4-methylbenzoylimino)benzo[d]thiazol-3(2H)-yl]propionic acid

The titled compound was prepared in accordance with the same procedures as in Example 119, using p-toluoyl chloride and ethyl 2-bromopropionate, instead of benzoyl chloride and ethyl bromoacetate, respectively (Yield: 89%).

¹H NMR (DMSO-d₆, 400 MHz) δ 8.12 (d, 2H), 7.95 (d, 1H), 7.77 (d, 1H), 7.56 (t, 1H), 7.39 (t, 1H), 7.32 (d, 2H), 5.88 (m, 1H), 2.40 (s, 3H), 1.72 (d, 3H)

### Example 129

### 2-[2-(3-methylbenzoylimino)benzo[d]thiazol-3(2H)-yl]propionic acid

The titled compound was prepared in accordance with the same procedures as in Example 121, using ethyl 2-bromopropionate, instead of ethyl bromoacetate (Yield: 50%).

¹H NMR (DMSO-d₆, 400 MHz) δ 13.08 (m, 1H), 8.03-8.05 (m, 2H), 7.95 (d, 1H), 7.78 (d, 1H), 7.55 (t, 1H), 7.36-7.40 (m, 3H), 5.88 (m, 1H), 2.40 (s, 3H), 1.72 (d, 3H)

### Example 130

### 2-[2-(4-chlorobenzoylimino)benzo[d]thiazol-3(2H)-yl]propionic acid

The titled compound was prepared in accordance with the same procedures as in Example 119, using 4-chlorobenzoyl chloride and ethyl 2-bromopropionate, instead of benzoyl chloride and ethyl bromoacetate, respectively (Yield: 85%).

¹H NMR (DMSO-d₆, 400 MHz) δ 13.13 (s, 1H), 8.22 (d, 2H), 7.98 (d, 1H), 7.79 (d, 1H), 7.55-7.59 (m, 3H), 7.40 (t, 1H), 5.91 (m, 1H), 1.72 (d, 3H)

### Example 131

### 2-[2-(3-chlorobenzoylimino)benzo[d]thiazol-3(2H)-yl]propionic acid

The titled compound was prepared in accordance with the same procedures as in Example 121, using ethyl p-toluoyl chloride and ethyl 2-bromopropionate, instead of 111-toluoyl chloride and ethyl bromoacetate, respectively (Yield: 30%).

¹H NMR (DMSO-d₆, 400 MHz) δ 13.05 (m, 1H), 8.21 (s, 1H), 8.15 (d, 1H), 7.99 (d, 1H), 7.81 (d, 1H), 7.66 (d, 1H), 7.56 (dd, 2H), 7.41 (t, 1H), 5.90 (m, 1H), 1.71 (d, 3H)

### Example 132

### 2-[2-(2-chlorobenzoylimino)benzo[d]thiazol-3(2H)-yl]propionic acid

The titled compound was prepared in accordance with the same procedures as in Example 119, using 2-chlorobenzoyl chloride and ethyl 2-bromopropionate, instead of benzoyl chloride and ethyl bromoacetate, respectively (Yield: 92%).

¹H NMR (DMSO-d₆, 400 MHz) δ 8.04 (d, 1H), 7.96 (d, 1H), 7.72 (d, 1H), 7.49-7.58 (m, 3H), 7.39-7.45 (m, 2H), 5.89 (m, 1H), 1.69 (d, 3H)

### Example 133

### 2-{2-[3-(trifluoromethyl)benzoylimino]benzo[d]thiazol-3(2H)-yl}propionic acid

The titled compound was prepared in accordance with the same procedures as in Example 119, using 3-(trifluoromethyl)benzoyl chloride and ethyl 2-bromopropionate, instead of benzoyl chloride and ethyl bromoacetate, respectively (Yield: 100%).

¹H NMR (CD₃OD, 400 MHz) δ 8.56 (s, 1H), 8.50 (d, 1H), 7.82 (d, 1H), 7.76 (d, 1H), 7.65 (t, 2H), 7.52 (t, 1H), 7.33 (t, 1H), 5.81-5.82 (m, 1H), 1.85 (d, 3H)

### Example 134

### 2-{2-[4-(trifluoromethoxy)benzoylimino]benzo[d]thiazol-3(2H)-yl}propionic acid

The titled compound was prepared in accordance with the same procedures as in Example 119, using 4-trifluoromethoxybenzoyl chloride and ethyl 2-bromopropionate, instead of benzoyl chloride and ethyl bromoacetate, respectively (Yield: 78%).

¹H NMR (DMSO-d₆, 400 MHz) δ 13.12 (s, 1H), 8.33 (d, 2H), 7.98 (d, 1H), 7.80 (d, 1H), 7.58 (t, 1H), 7.50 (d, 2H), 7.41 (t, 1H), 5.90-5.92 (m, 1H), 1.72 (d, 3H)

### Example 135

### 2-[2-(2,6-dimethoxybenzoylimino)benzo[d]thiazol-3(2H)-yl]propionic acid

The titled compound was prepared in accordance with the same procedures as in Example 119, using 2,6-dimethoxybenzoyl chloride and ethyl 2-bromopropionate, instead of benzoyl chloride and ethyl bromoacetate, respectively (Yield: 65%).

¹H NMR (DMSO-d₆, 400 MHz) δ 7.94 (d, 1H), 7.58 (m, 1H), 7.52 (t, 1H), 7.38 (t, 1H), 7.29 (t, 1H), 6.69 (d, 2H), 5.90 (m, 1H), 3.71 (s, 6H), 1.64 (d, 3H)

### Example 136

### 2-[2-(benzoylimino)benzo[d]thiazol-3(2H)-yl]butanoic acid

The titled compound was prepared in accordance with the same procedures as in Example 119, using methyl 2-bromobutyrate instead ethyl bromoacetate (Yield: 41%).

¹H NMR (DMSO-d₆, 400 MHz) δ 13.13 (s, 1H), 8.21 (d, 2H), 7.97 (d, 1H), 7.83 (m, 1H), 7.50-7.62 (m, 4H), 7.39 (t, 1H), 5.63-5.78 (m, 1H), 2.34-2.50 (m, 2H), 0.75 (t, 3H)

### Example 137

### 2-(2-((3,5-bis(trifluoromethyl)benzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine prepared in Preparation 3, 3,5-bis(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 36%).

¹H NMR (MeOD, 400 MHz) δ 8.76 (s, 2H), 7.89 (s, 1H), 7.95-7.75 (m, 2H), 5.70-5.50 (brs, 1H), 2.70-2.45 (m, 2H), 0.90 (t, 3H)

### Example 138

### 2-[2-(4-chlorobenzoylimino)benzo[d]thiazol-3(2H)-yl]butanoic acid

The titled compound was prepared in accordance with the same procedures as in Example 119, using 4-chlorobenzoyl chloride and methyl 2-bromobutyrate, instead of benzoyl chloride and ethyl bromoacetate, respectively (Yield: 52%).

¹H NMR (DMSO-d₆, 400 MHz) δ 8.20 (d, 2H), 7.98 (d, 1H), 7.82 (m, 1H), 7.55-7.60 (m, 3H), 7.40 (t, 1H), 5.79 (m, 1H), 2.34-2.36 (m, 2H), 0.75 (t, 3H)

### Example 139

### 2-[2-(3-chlorobenzoylimino)benzo[d]thiazol-3(2H)-yl]butanoic acid

The titled compound was prepared in accordance with the same procedures as in Example 119, using 3-chlorobenzoyl chloride and methyl 2-bromobutyrate, instead of benzoyl chloride and ethyl bromoacetate, respectively (Yield: 46%).

¹H NMR (DMSO-d₆, 400 MHz) δ 8.18 (s, 1H), 8.13 (d, 1H), 7.97 (d, 1H), 7.83 (m, 1H), 7.65 (d, 1H), 7.55-7.57 (m, 2H), 7.41 (t, 1H), 5.74 (m, 1H), 2.34-2.50 (m, 2H), 0.74 (t, 3H)

### Example 140

### 2-[2-(2-chlorobenzoylimino)benzo[d]thiazol-3(2H)-yl]butanoic acid

The titled compound was prepared in accordance with the same procedures as in Example 119, using 2-chlorobenzoyl chloride and methyl 2-bromobutyrate, instead of benzoyl chloride and ethyl bromoacetate, respectively (Yield: 22%).

¹H NMR (CD₃OD, 400 MHz) δ 8.03 (d, 1H), 7.85 (d, 1H), 7.60 (m, 1H), 7.52-7.53 (m, 1H), 7.43-7.48 (m, 2H), 7.36-7.41 (m, 2H), 5.90 (m, 1H), 2.47 (m, 2H), 0.84 (m, 3H)

### Example 141

### 2-[2-(2,6-difluorobenzoylimino)benzo[d]thiazol-3(2H)-yl]butanoic acid

The titled compound was prepared in accordance with the same procedures as in Example 119, using 2,6-difluorobenzoyl chloride and methyl 2-bromobutyrate, instead of benzoyl chloride and ethyl bromoacetate, respectively (Yield: 36%).

¹H NMR (CD₃OD, 400 MHz) δ 7.85 (d, 1H), 7.58 (m, 1H), 7.53 (t, 1H), 7.38-7.47 (m, 2H), 7.02 (t, 2H), 5.94 (m, 1H), 2.39-2.50 (m, 2H), 0.82 (t, 3H)

### Example 142

### 2-[2-(3-(trifluoromethyl)benzoylimino)benzo[d]thiazol-3(2H)-yl]butanoic acid

The titled compound was prepared in accordance with the same procedures as in Example 119, using 3-(trifluoromethyl)benzoyl chloride and methyl 2-bromobutyrate, instead of benzoyl chloride and ethyl bromoacetate, respectively (Yield: 80%).

¹H NMR (CD₃OD, 400 MHz) δ 8.57 (s, 1H), 8.52 (d, 1H), 7.83 (t, 2H), 7.68 (dd, 2H), 7.54 (t, 1H), 7.37 (t, 1H), 5.73 (m, 1H), 2.51-2.61 (m, 2H), 0.87 (t, 3H)

### Example 143

### 2-[2-(4-(trifluoromethoxy)benzoylimino)benzo[d]thiazol-3(2H)-yl]butanoic acid

The titled compound was prepared in accordance with the same procedures as in Example 119, using 4-trifluoromethoxybenzoyl chloride and methyl 2-bromobutyrate, instead of benzoyl chloride and ethyl bromoacetate, respectively (Yield: 7%).

¹H NMR (CD₃OD, 400 MHz) δ 8.36 (d, 2H), 7.83 (d, 1H), 7.66 (m, 1H), 7.53 (t, 1H), 7.36-7.40 (m, 3H), 5.76 (m, 1H), 2.52 (m, 2H), 0.86 (t, 3H)

### Example 144

### 2-[2-(2,6-dimethoxybenzoylimino)benzo[d]thiazol-3(2H)-yl]butanoic acid

The titled compound was prepared in accordance with the same procedures as in Example 119, using 2,6-dimethoxybenzoyl chloride and methyl 2-bromobutyrate, instead of benzoyl chloride and ethyl bromoacetate, respectively (Yield: 82%).

¹H NMR (CD₃OD, 400 MHz) δ 7.84 (d, 1H), 7.49-7.50 (m, 2H), 7.38 (t, 1H), 7.30 (t, 1H), 6.68 (d, 2H), 5.96-6.03 (m, 1H), 3.77 (s, 6H), 2.33-2.42 (m, 2H), 0.81 (t, 3H)

### Example 145

### 2-[2-(4-methylbenzoylimino)benzo[d]thiazol-3(2H)-yl]pentanoic acid

The titled compound was prepared in accordance with the same procedures as in Example 119, using p-toluoyl chloride and ethyl 2-bromovalerate, instead of benzoyl chloride and ethyl bromoacetate, respectively (Yield: 84%).

¹H NMR (DMSO-d₆, 400 MHz) δ 8.11 (d, 2H), 7.95 (d, 1H), 7.78 (m, 1H), 7.54 (t, 1H), 7.38 (t, 1H), 7.32 (d, 2H), 5.75 (m, 1H), 2.39 (s, 3H), 2.28 (m, 1H), 1.23-1.27 (m, 1H), 1.09 (t, 1H), 0.99 (m, 1H), 0.82 (t, 3H)

### Example 146

### 2-[2-(4-methylbenzoylimino)benzo[d]thiazol-3(2H)-yl]hexanoic acid

The titled compound was prepared in accordance with the same procedures as in Example 119, using p-toluoyl chloride and methyl 2-bromohexanoate, instead of benzoyl chloride and ethyl bromoacetate, respectively (Yield: 93%).

¹H NMR (DMSO-d₆, 400 MHz) δ 8.12 (d, 2H), 7.89 (d, 1H), 7.55 (m, 1H), 7.46 (t, 1H), 7.32 (d, 3H), 5.80 (m, 1H), 2.39 (m, 4H), 1.23-1.32 (m, 4H), 0.93 (m, 1H), 0.72 (t, 3H)

### Example 147

### 2-[2-(4-methylbenzoylimino)benzo[d]thiazol-3(2H)-yl]octanoic acid

The titled compound was prepared in accordance with the same procedures as in Example 119, using p-toluoyl chloride and ethyl 2-bromooctanoate, instead of benzoyl chloride and ethyl bromoacetate, respectively (Yield: 91%).

¹H NMR (DMSO-d₆, 400 MHz) δ 8.10 (d, 2H), 7.95 (d, 1H), 7.80-7.90 (m, 1H), 7.54 (t, 1H), 7.38 (t, 1H), 7.32 (d, 2H), 5.66 (m, 1H), 2.39 (s, 3H), 2.28 (m, 1H), 1.24 (m, 4H), 1.09 (m, 4H), 0.95 (m, 1H), 0.71 (t, 3H)

### Example 148

### 3-methyl-2-[2-(4-methylbenzoylimino)benzo[d]thiazol-3(2H)-yl]butanoic acid

The titled compound was prepared in accordance with the same procedures as in Example 119, using p-toluoyl chloride and ethyl 2-bromo-3-methylbutyrate, instead of benzoyl chloride and ethyl bromoacetate, respectively (Yield: 6%).

¹H NMR (CD₃OD, 400 MHz) δ 8.17 (d, 2H), 7.81 (d, 1H), 7.69 (d, 1H), 7.52 (t, 1H), 7.37 (t, 1H), 7.30 (d, 2H), 3.13 (m, 1H), 2.86 (m, 1H), 2.42 (s, 3H), 1.40 (d, 3H), 0.66 (d, 3H)

### Example 149

### 2-[2-(4-methylbenzoylimino)benzo[d]thiazol-3(2H)-yl]-3-butenoic acid

The titled compound was prepared in accordance with the same procedures as in Example 119, using p-toluoyl chloride and methyl 4-bromocrotonate, instead of benzoyl chloride and ethyl bromoacetate, respectively (Yield: 12%).

¹H NMR (CD₃OD, 400 MHz) δ 8.17 (d, 2H), 7.81 (d, 1H), 7.52 (d, 2H), 7.35-7.39 (m, 1H), 7.28 (d, 2H), 7.05-7.08 (m, 1H), 5.73-5.77 (m, 1H), 5.39-5.40 (m, 2H), 2.40 (s, 3H)

### Example 150

### 2-fluoro-2-[2-(4-methylbenzoylimino)benzo[d]thiazol-3(2H)-yl]acetic acid

The titled compound was prepared in accordance with the same procedures as in Example 119, using p-toluoyl chloride and ethyl bromofluoroacetate, instead of benzoyl chloride and ethyl bromoacetate, respectively (Yield: 22%).

¹H NMR (CD₃OD, 400 MHz) δ 8.20 (d, 2H), 7.45-7.77 (m, 4H), 7.28-7.36 (m, 3H), 2.40 (s, 3H)

### Example 151

### 3,3,3-trifluoro-2-[2-(4-methylbenzoylimino)benzo[d]thiazol-3(2H)-yl]propionic acid

The titled compound was prepared in accordance with the same procedures as in Example 119, using p-toluoyl chloride and methyl 2-bromo-3,3,3-trifluoropropanoate, instead of benzoyl chloride and ethyl bromoacetate, respectively (Yield: 48%).

¹H NMR (CD₃OD, 400 MHz) δ 8.15 (d, 2H), 7.73 (d, 1H), 7.47 (t, 1H), 7.35 (t, 2H), 7.28 (d, 2H), 2.41 (s, 3H)

### Example 152

### 2-[(2-(4-methylbenzoylimino)benzo[d]thiazol-3(2H)-yl]-2-phenylacetic acid

The titled compound was prepared in accordance with the same procedures as in Example 119, using p-toluoyl chloride and alpha-bromophenylacetate, instead of benzoyl chloride and ethyl bromoacetate, respectively (Yield: 38%).

¹H NMR (DMSO-d₆, 400 MHz) δ 8.14 (d, 2H), 7.86 (d, 1H), 7.50 (d, 2H), 7.20-7.32 (m, 9H), 2.38 (s, 3H)

### Example 153

### 2-[6-fluoro-2-(4-methylbenzoylimino)benzo[d]thiazol-3(2H)-yl]butanoic acid

The titled compound was prepared in accordance with the same procedures as in Example 119, using 2-amino-6-fluorobenzothiazole, p-toluoyl chloride, and methyl 2-bromobutyrate, instead of 2-aminobenzothiazole, benzoyl chloride, and ethyl bromoacetate, respectively (Yield: 66%).

¹H NMR (DMSO-d₆, 400 MHz) δ 8.08 (d, 2H), 7.89 (d, 1H), 7.78 (m, 1H), 7.40 (t, 1H), 7.30 (d, 2H), 5.71 (m, 1H), 2.37-2.50 (m, 5H), 0.74 (t, 3H)

### Example 154

### 2-[4,6-difluoro-2-(4-methylbenzoylimino)benzo[d]thiazol-3(2H)-yl]butanoic acid

The titled compound was prepared in accordance with the same procedures as in Example 119, using 2-amino-4,6-difluorobenzothiazole, p-toluoyl chloride, and methyl 2-bromobutyrate, instead of 2-aminobenzothiazole, benzoyl chloride, and ethyl bromoacetate, respectively (Yield: 35%).

¹H NMR (CD₃OD, 400 MHz) δ 8.15 (d, 2H), 7.51 (d, 1H), 7.18-7.30 (m, 3H), 2.55 (m, 1H), 2.41 (m, 4H), 0.92 (t, 3H)

### Example 155

### 2-[2-(4-methylbenzoylimino)-6-(trifluoromethyl)benzo[d]thiazol-3(2H)-yl]butanoic acid

The titled compound was prepared in accordance with the same procedures as in Example 119, using 2-amino-6-trifluoromethylbenzothiazole, p-toluoyl chloride, and methyl 2-bromobutyrate, instead of 2-aminobenzothiazole, benzoyl chloride, and ethyl bromoacetate, respectively (Yield: 58%).

¹H NMR (DMSO-d₆, 400 MHz) δ 8.43 (s, 1H), 8.09 (d, 2H), 7.93 (m, 1H), 7.87 (d, 1H), 7.32 (d, 2H), 5.80 (m, 1H), 2.38-2.50 (m, 5H), 0.75 (t, 3H)

### Example 156

### 3-(1-carboxypropyl)-2-(4-methylbenzoylimino)-2,3-dihydrobenzo[d]thiazol-6-carboxylic acid

### Step 1: methyl 2-(4-methylbenzamido)benzo[d]thiazol-6-carboxylate

To a solution of 2-amino-benzothiazol-6-carboxylic acid methyl ester (5 g, 24.0 mmol) in tetrahydrofuran (50.0 mL), were added dropwise diisopropylethylamine (5.0 mL, 28.8 mmol) and p-toluoyl chloride (3.3 mL, 24.8 mmol). The reaction mixture was stirred at room temperature for 30 minutes and then dimethylaminopyridine (586 mg, 4.8 mmol) was added thereto. The reaction mixture was stirred at room temperature for 1 day. An aqueous 1N HCl solution (30.0 mL) and ethyl acetate (60.0 mL) were added to the reaction mixture. The reaction mixture was stirred for 5 minutes and then filtered. The resulting solid was washed with ethyl acetate and then dried under reduced pressure to give 5.0 g of the titled compound as a pale yellow solid (Yield: 64%).

### Step 2: a mixture of methyl 2-(4-methylbenzamido)benzo[d]thiazol-6-carboxylate and 2-(4-methylbenzamido)benzo[d]thiazol-6-carboxylic acid

To a solution of methyl 2-(4-methylbenzamido)benzo[d]thiazol-6-carboxylate (5.0 g, 15.3 mmol) prepared in Step 1 in methanol (50.0 mL) was added a 1N sodium hydroxide solution (20.0 mL). The reaction mixture was stirred at room temperature overnight. The reaction mixture was evaporated and diluted with water. The aqueous layer was washed with diethyl ether and then acidified with an aqueous 1N HCl solution. The resulting solid was filtered, washed with water, and then dried under reduced pressure to give 4.8 g of the titled compound as a white solid.

### Step 3: methyl 3-(1-methoxy-1-oxobutan-2-yl)-2-(4-methylbenzoylimino)-2,3-dihydrobenzo[d]thiazol 6-carboxylate

To the mixture of methyl 2-(4-methylbenzamido)benzo[d]thiazol-6-carboxylate and 2-(4-methylbenzamido)benzo[d]thiazol-6-carboxylic acid (100 mg, 0.32 mmol) prepared in Step 2 in dimethylformamide (1.5 mL) were added potassium carbonate (133 mg, 0.96 mmol) and methyl 2-bromobutyrate (55 uL, 0.48 mmol). The reaction mixture was refluxed under stirring at 80° C. overnight. The reaction mixture was quenched with water and then extracted with ethyl acetate. The organic layer was dried over anhydrous magnesium sulfate, filtered, and then evaporated. The residue was purified with preparative thin layer chromatography (n-hexaneethyl acetate=41) to give 50 mg of the titled compound as a white solid (Yield: 37%).

¹H NMR (DMSO-d₆, 400 MHz) δ 8.61 (s, 1H), 8.12 (d, 1H), 8.06 (d, 2H), 7.93 (d, 1H), 7.34 (d, 2H), 5.90 (m, 1H), 3.90 (s, 3H), 3.59 (s, 3H), 2.40-2.50 (m, 5H), 0.77 (t, 3H)

### Step 4: 3-(1-carboxypropyl)-2-(4-methylbenzoylimino)-2,3-dihydrobenzo[d]thiazol-6-carboxylic acid

The titled compound was prepared in accordance with the same procedures as in Step 3 of Example 119, using methyl 3-(1-methoxy-1-oxobutan-2-yl)-2-(4-methylbenzoylimino)-2,3-dihydrobenzo[d]thiazol-6-carboxylate prepared in Step 3 as a starting material (Yield: 65%).

¹H NMR (DMSO-d₆, 400 MHz) δ 13.11 (m, 2H), 8.54 (s, 1H), 8.06-8.11 (m, 3H), 7.84-7.88 (m, 1H), 7.32 (d, 2H), 5.79-5.81 (m, 1H), 2.39-2.50 (m, 5H), 0.76 (t, 3H)

### Example 157

### 2-[2-(4-methylbenzoylimino)-6-(phenylcarbamoyl)benzo[d]thiazol-3(2H)-yl]butanoic acid

### Step 1: ethyl 2-(4-methylbenzamido)benzo[d]thiazol-6-carboxylate

To a solution of 2-amino-benzothiazol-6-carboxylic acid ethyl ester (5 g, 22.5 mmol) in tetrahydrofuran (50.0 mL), were added dropwise diisopropylethylamine (4.7 mL, 27.0 mmol) and p-toluoyl chloride (3.3 mL, 24.8 mmol). The reaction mixture was stirred at room temperature for 30 minutes and then dimethylaminopyridine (550 mg, 4.5 mmol) was added thereto. The reaction mixture was stirred at room temperature for 2 days. An aqueous 1N HCl solution (30.0 mL) and ethyl acetate (60.0 mL) were added to the reaction mixture. The reaction mixture was stirred for 5 minutes and then filtered. The resulting solid was washed with ethyl acetate and then dried under reduced pressure to give 4.5 g of the titled compound as a yellow solid (Yield: 59%).

¹H NMR (DMSO-d₆, 400 MHz) δ 13.01 (brs, 1H), 8.67 (s, 1H), 8.05 (t, 3H), 7.85 (d, 1H), 7.38 (d, 2H), 4.35 (q, 2H), 2.40 (s, 3H), 1.35 (t, 3H)

### Step 2: 2-(4-methylbenzamido)benzo[d]thiazol-6-carboxylic acid

To a solution of ethyl 2-(4-methylbenzamido)benzo[d]thiazol-6-carboxylate (2.0 g, 5.9 mmol) prepared in Step 1 in methanol (15.0 mL) was added a 1N sodium hydroxide solution (9.0 mL). The reaction mixture was stirred at room temperature overnight, concentrated under reduced pressure, and then diluted with water. The aqueous layer was washed with diethyl ether and then acidified with an aqueous 1N HCl solution. The resulting solid was filtered, washed with water, and then dried under reduced pressure to give 2.1 g of the titled compound as a white solid (Yield: 100%).

¹H NMR (DMSO-d₆, 400 MHz) δ 12.97 (brs, 1H), 8.65 (d, 1H), 8.00-8.06 (m, 3H), 7.83 (t, 1H), 7.37 (d, 2H), 3.87 (s, 1H), 2.39 (s, 3H)

### Step 3: 2-(4-methylbenzamido)-N-phenylbenzo[d]thiazol-6-carboxamide

To a solution of 2-(4-methylbenzamido)benzo[d]thiazol-6-carboxylic acid (300 mg, 0.96 mmol), aniline (96 uL, 1.06 mmol) and (benzotriazol-1-yloxy)tris(dimethylamino)phosphonium hexafluorophosphate (510 mg, 1.15 mmol) in N,N-dimethylformamide (3.0 mL) was added dropwise diisopropylethylamine (669 uL, 3.84 mmol). The reaction mixture was stirred at room temperature overnight. The reaction mixture was quenched with water and then added to ethyl acetate. The reaction mixture was stirred for 5 minutes. The resulting solid was filtered, washed with water and ethyl acetate, and then dried under reduced pressure to give 205 mg of the titled compound as a yellow solid (Yield: 55%).

¹H NMR (DMSO-d₆, 400 MHz) δ 12.95 (m, 1H), 10.28 (s, 1H), 8.63-8.66 (m, 1H), 8.02-8.09 (m, 3H), 7.78-7.88 (m, 3H), 7.34-7.39 (m, 4H), 7.10 (t, 1H), 2.40 (s, 3H)

### Step 4: methyl 2-[2-(4-methylbenzoylimino)-6-(phenylcarbamoyl)benzo[d]thiazol-3(2H)-yl]butanoate

To a solution of 2-(4-methylbenzamido)-N-phenylbenzo[d]thiazol-6-carboxamide (200 mg, 0.52 mmol) prepared in Step 3 in N,N-dimethylformamide (2.0 mL) was added dropwise potassium carbonate (216 mg, 1.56 mmol) and methyl 2-bromobutyrate (90 uL, 0.78 mmol). The reaction mixture was refluxed under stirring at 80° C. overnight. The reaction mixture was quenched with water and then extracted with ethyl acetate. The organic layer was dried over anhydrous magnesium sulfate, filtered, and then evaporated. The residue was purified with silica gel column chromatography (n-hexaneethyl acetate=2/1, v/v) to give 200 mg of the titled compound as a white solid (Yield: 79%).

¹H NMR (CDCl₃, 400 MHz) δ 8.23 (s, 1H), 8.17 (d, 2H), 7.97 (d, 1H), 7.88 (s, 1H), 7.65 (d, 2H), 7.34-7.41 (m, 3H), 7.28 (d, 2H), 7.18 (t, 1H), 5.63 (m, 1H), 3.68 (s, 3H), 2.46-2.57 (m, 2H), 2.42 (s, 3H), 0.92 (t, 3H)

### Step 5: 2-[2-(4-methylbenzoylimino)-6-(phenylcarbamoyl)benzo[d]thiazol-3(2H)-yl]butanoic acid

The titled compound was prepared in accordance with the same procedures as in Step 3 of Example 119, using methyl 2-[2-(4-methylbenzoylimino)-6-(phenylcarbamoyl)benzo[d]thiazol-3(2H)-yl]butanoate prepared in Step 4 as a starting material (Yield: 71%).

¹H NMR (DMSO-d₆, 400 MHz) δ 10.39 (s, 1H), 8.56 (s, 1H), 8.11-8.19 (m, 3H), 7.85 (d, 3H), 7.37-7.44 (m, 4H), 7.16 (t, 1H), 5.85 (m, 1H), 2.44-2.55 (m, 5H), 0.81 (t, 3H)

### Example 158

### 2-[6-(benzylcarbamoyl)-2-(4-methylbenzoylimino)benzo[d]thiazol-3(2H)-yl]butanoic acid

The titled compound was prepared in accordance with the same procedures as in Example 157, using benzylamine, instead of aniline (Yield: 47%).

¹H NMR (DMSO-d₆, 400 MHz) δ 9.10-9.12 (m, 1H), 8.39-8.40 (m, 1H), 8.10-8.12 (m, 2H), 7.87-8.05 (m, 1H), 7.65 (m, 1H), 7.31-7.33 (m, 6H), 7.23 (m, 1H), 5.73 (m, 1H), 4.50 (s, 2H), 2.10-2.45 (m, 5H), 0.72 (t, 3H)

### Example 159

### 2-[2-(4-methylbenzoylimino)-6-(phenethylcarbamoyl)benzo[d]thiazol-3(2H)-yl]butanoic acid

The titled compound was prepared in accordance with the same procedures as in Example 157, using phenethylamine, instead of aniline (Yield: 100%).

¹H NMR (DMSO-d₆, 400 MHz) δ 8.63-8.64 (m, 1H), 8.29 (s, 1H), 8.11 (d, 2H), 7.86 (d, 1H), 7.54 (m, 1H), 7.17-7.31 (m, 7H), 5.70 (m, 1H), 3.49 (q, 2H), 2.85 (t, 2H), 2.37-2.48 (m, 4H), 2.16 (m, 1H), 0.72 (t, 3H)

### Example 160

### 2-[2-(4-methylbenzoylimino)-4,5,6,7-tetrahydrobenzo[d]thiazol-3(2H)-yl]butanoic acid

The titled compound was prepared in accordance with the same procedures as in Example 119, using 2-amino-4,5,6,7-tetrahydrobenzothiazole, p-toluoyl chloride, and methyl 2-bromobutyrate, instead of 2-aminobenzothiazole, benzoyl chloride, and ethyl bromoacetate, respectively (Yield: 80%).

¹H NMR (DMSO-d₆, 400 MHz) δ 8.00 (d, 2H), 7.24 (d, 2H), 2.50-2.55 (m, 4H), 2.35-2.40 (m, 1H), 2.35 (s, 3H), 2.08 (m, 1H), 1.77-1.82 (m, 4H), 0.74 (t, 3H)

### Example 161

### 2-(5,6-dimethyl-2-((4-methylbenzoyl)imino)benzo[d]thiazol-3(2H)-yl)propionic acid

The titled compound was prepared in accordance with the same procedures as in Example 52, using 5,6-dimethylbenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 34%).

¹H NMR (DMSO-d₆, 400 MHz); 13.00 (brs, 1H), 8.09 (d, 2H), 7.68 (s, 1H), 7.60 (s, 1H), 7.29 (d, 2H), 5.79 (brs, 1H), 2.39 (s, 3H), 2.36 (s, 3H), 2.32 (s, 3H), 1.70 (d, 3H)

### Example 162

### 2-(6-ethoxy-2-((4-methylbenzoyl)imino)benzo[d]thiazol-3(2H)-yl)propionic acid

The titled compound was prepared in accordance with the same procedures as in Example 52, using 6-ethoxybenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 34%).

¹H NMR (DMSO-d₆, 400 MHz); 13.04, (s, 1H), 8.10 (d, 2H), 7.66 (d, 1H), 7.57 (d, 1H), 7.30 (d, 2H), 7.12 (dd, 1H), 5.81 (brs, 1H), 4.09 (q, 2H), 2.39 (s, 3H), 1.70 (d, 3H), 1.36 (s, 3H)

### Example 163

### 2-(4-methoxy-2-((4-methylbenzoyl)imino)benzo[d]thiazol-3(2H)-yl)propionic acid

The titled compound was prepared in accordance with the same procedures as in Example 52, using 4-methoxybenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 28%).

¹H NMR (DMSO-d₆, 400 MHz); 13.04 (s, 1H), 8.10 (d, 2H), 7.67 (d, 1H), 7.59 (s, 1H), 7.30 (d, 2H), 7.14 (d, 1H), 5.83 (brs, 1H), 3.80 (s, 3H), 2.39 (s, 3H), 1.70 (d, 3H)

### Example 164

### 2-(6-methyl-2-((4-methylbenzoyl)imino)benzo[d]thiazol-3(2H)-yl)propionic acid

The titled compound was prepared in accordance with the same procedures as in Example 52, using 6-methylbenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 31%).

¹H NMR (DMSO-d₆, 400 MHz); 13.05 (s, 1H), 8.10 (d, 2H), 7.75 (s, 1H), 7.64 (d, 1H), 7.36 (d, 1H), 7.31 (d, 2H), 5.85 (brs, 1H), 2.42 (s, 3H), 2.39 (s, 3H), 1.70 (d, 3H)

### Example 165

### 2-(6-(hydroxymethyl)-2-((4-methylbenzoyl)imino)benzo[d]thiazol-3(2H)-yl)butanoic acid

### Step 1: ethyl 4-aminobenzo[d]thiazol-6-carboxylate

To a solution of ethyl 4-aminobenzoate (16.5 g, 100.0 mmol) and potassium thiocyanate (10.7 g, 110.0 mmol) in acetic acid (150 mL) was added dropwise a solution of bromine (5.1 mL, 100.0 mmol) in acetic acid (50 mL) at below 10° C. The reaction mixture was stirred at room temperature overnight. The resulting solid was filtered and then dissolved in warm water (50° C.). The aqueous layer was washed with chloroform and then basified to pH 11 with solid sodium carbonate. The resulting solid was filtered, washed with water, and then dried under reduced pressure to give 10.2 g of the titled compound as a white solid (Yield: 46%).

### Step 2: (2-aminobenzo[d]thiazol-6-yl)methanol

To a solution of ethyl 2-aminobenzo[d]thiazol-6-carboxylate (10.10 g, 45.44 mmol) prepared in Step 1 in THF (320 mL) was added portionwise LAH (7.59 g, 199.94 mmol) at 0° C. The reaction mixture was stirred at room temperature for 7 hours. Water (8.0 mL), 20% sodium hydroxide (8.0 mL), and water (24 mL) were sequentially added to the reaction mixture, which was then stirred for 2 hours. The resulting solid was filtered and washed with ethyl acetate. The filtrate was concentrated under reduced pressure. The resulting solid was suspended with diethyl ether, filtered, and then dried under reduced pressure to give 5.52 g of the titled compound as a white solid (Yield: 67%).

### Step 3: N-(6-(hydroxymethyl)benzo[d]thiazol-2(3H)-ylidene)-4-methylbenzamide

To a solution of (2-aminobenzo[d]thiazol-6-yl)methanol (5.34 g, 29.63 mmol) in tetrahydrofuran (300 mL), were added triethylamine (6.16 mL, 44.45 mmol), 4,4-dimethylaminopyridine (362 mg, 2.963 mmol), and p-toluoyl chloride (5.48 mL, 41.48 mmol). The reaction mixture was stirred at 80° C. for 4 hours. A 3N sodium hydroxide solution (20 mL) was added to the reaction mixture, which was then stirred at room temperature overnight. The reaction mixture was extracted with ethyl acetate. The organic layer was washed with brine, dried over anhydrous magnesium sulfate, filtered, and then evaporated. Isopropyl ether was added to the resulting residue and then filtered. The resulting solid was dried under reduced pressure to give 4.84 g of the titled compound as a white solid (Yield: 55%).

¹H NMR (DMSO-d₆, 400 MHz); 8.02 (d, 2H), 7.89 (s, 1H), 7.71 (d, 1H), 7.36 (m, 3H), 5.34 (brs, 1H), 4.59 (s, 2H), 2.50 (s, 1H), 2.38 (s, 3H)

### Step 4: methyl 2-(6-(hydroxymethyl)-2-((4-methylbenzoyl)imino)benzo[d]thiazol-3(2H)-yl)butanoate

To a solution of N-(6-(hydroxymethyl)benzo[d]thiazol-2(3H)-ylidene)-4-methylbenzamide (500 mg, 1.676 mmol) prepared in Step 3 in N,N-dimethylformamide (10 mL) were added potassium carbonate (695 mg, 5.028 mmol) and methyl 2-bromobutyrate (289 uL, 2.514 mmol). The reaction mixture was stirred at 50° C. overnight. The reaction mixture was quenched with water and then extracted with ethyl acetate. The organic layer was washed with brine, dried over anhydrous magnesium sulfate, filtered, and then evaporated. Isopropyl ether was added to the resulting residue and then filtered. The resulting solid was dried under reduced pressure to give 470 mg of the titled compound as a white solid (Yield: 70%).

¹H NMR (CDCl₃, 400 MHz); 8.19 (d, 2H), 7.76 (s, 1H), 7.44 (d, 1H), 7.26 (m, 3H), 5.60 (brs, 1H), 4.79 (s, 2H), 3.65 (s, 3H), 2.51 (m, 2H), 2.42 (s, 3H), 1.97 (s, 1H), 1.57 (s, 2H), 0.89 (t, 3H).

### Step 5: 2-(6-(hydroxymethyl)-2-((4-methylbenzoyl)imino)benzo[d]thiazol-3(2H)-yl)butanoic acid

To a solution of methyl 2-(6-(hydroxymethyl)-2-((4-methylbenzoyl)imino)benzo[d]thiazol-3(2H)-yl)butanoate (150 mg, 0.376 mmol) prepared in Step 4 in a mixed solvent of tetrahydrofuran and methanol (1/1, 4.0 mL) was added a 3N sodium hydroxide solution (1.3 mL). The reaction mixture was stirred at room temperature overnight. The reaction mixture was evaporated and then diluted with water. The aqueous layer was acidified to pH 3-4 with an aqueous 1N HCl solution and then extracted with ethyl acetate. The organic layer was washed with brine, dried over anhydrous magnesium sulfate, filtered, and then evaporated. The residue was recrystallized from n-hexaneethyl acetate to give 62 mg of the titled compound as a white solid (Yield: 43%).

¹H NMR (DMSO-d₆, 400 MHz); 13.04 (s, 1H), 8.10 (d, 2H), 7.88 (s, 1H), 7.74 (s, 1H), 7.47 (d, 1H), 7.31 (d, 2H), 5.75 (brs, 1H), 5.32 (brs, 1H), 4.60 (s, 2H), 2.50-2.30 (m, 2H), 2.39 (s, 3H), 0.74 (t, 3H)

### Example 166

### 2-(6-(benzyloxy)-2-((4-methylbenzoyl)imino)benzo[d]thiazol-3(2H)-yl)butanoic acid

### Step 1: 2-aminobenzo[d]thiazol-6-ol

A mixture of 6-methoxy-2-aminobenzothiazole (5.0 g, 27.74 mmol) in a bromic acid solution (48%, 41 mL) was refluxed for 5 hours and then cooled to room temperature. The reaction mixture was diluted with water and then neutralized with sodium carbonate. The resulting solid was filtered, washed with water, and then dried under reduced pressure to give 5.81 g of the titled compound as a gray solid.

### Step 2: 2-((4-methylbenzoyl)imino)-2,3-dihydrobenzo[d]thiazol-6-yl 4-methylbenzoate

To a solution of 2-aminobenzo[d]thiazol-6-ol (4.61 g, 27.74 mmol) prepared in Step 1 in tetrahydrofuran (250 mL), were sequentially added triethylamine (5.77 mL, 41.61 mmol), dimethylaminopyridine (339 mg, 2.77 mmol), and p-toluoyl chloride (5.13 mL, 38.84 mmol. The reaction mixture was stirred at room temperature overnight. The reaction mixture was diluted with ethyl acetate and then washed with an aqueous 1N HCl solution, a saturated sodium hydrogen carbonate solution, and brine. The organic layer was dried over magnesium sulfate and then filtered. The filtrate was evaporated, suspended with isopropyl ether and then filtered. The resulting solid was dried under reduced pressure to give 5.81 g of the titled compound as a gray solid.

¹H NMR (DMSO-d₆, 400 MHz); 8.04 (t, 4H), 7.54 (s, 1H), 7.47 (d, 1H), 7.42 (d, 2H), 7.20 (d, 2H), 7.06 (d, 1H), 2.42 (s, 3H), 2.34 (s, 3H)

### Step 3: N-(6-hydroxybenzo[d]thiazol-2(3H)-ylidene)-4-methylbenzamide

A 3N sodium hydroxide solution (16 mL) was added to 2-((4-methylbenzoyl)imino)-2,3-dihydrobenzo[d]thiazol-6-yl 4-methylbenzoate (3.81 g, 9.47 mmol) prepared in Step 2 in a mixed solvent of tetrahydrofuran and methanol (1/1, 40 mL). The reaction mixture was stirred at room temperature overnight and then evaporated. The residue was diluted with water, acidified to pH 3-4 with an aqueous 1N HCl solution and then extracted with ethyl acetate. The organic layer was washed with a saturated sodium hydrogen carbonate solution and brine, dried over magnesium sulfate, and then filtered. The filtrate was concentrated under reduced pressure. The resulting solid was suspended with isopropyl ether. The resulting solid was filtered, and then dried under reduced pressure to give 2.69 g of the titled compound as a gray solid.

¹H NMR (DMSO-d₆, 400 MHz); 9.67 (s, 1H), 8.00 (d, 2H), 7.59 (d, 1H), 7.36 (d, 2H), 7.30 (s, 1H), 6.92 (d, 1H), 2.38 (s, 3H)

### Step 4: N-(6-((tert-butyldimethylsilyl)oxy)benzo[d]thiazol-2(3H)-ylidene)-4-methylbenzamide

To a solution of N-(6-hydroxybenzo[d]thiazol-2(3H)-ylidene)-4-methylbenzamide (2.69 g, 9.46 mmol) prepared in Step 3 in N,N-dimethylformamide (50 mL), were added imidazole (1.93 g, 28.35 mmol) and tert-butyldimethylsilyl chloride (2.14 g, 14.20 mmol). The reaction mixture was stirred at room temperature overnight. The reaction mixture was diluted with ethyl acetate, and then washed with water and brine. The organic layer was dried over magnesium sulfate and then filtered. The filtrate was concentrated under reduced pressure. The resulting residue was purified with silica gel column chromatography (n-hexaneethyl acetate=31) to give 4.53 g of the titled compound as a white solid.

### Step 5: methyl 2-(6-((tert-butyldimethylsilyl)oxy)-2-((4-methylbenzoyl)imino)benzo[d]thiazol-3(2H)-yl)butanoate

To a solution of N-(6-((tert-butyldimethylsilyl)oxy)benzo[d]thiazol-2(3H)-ylidene)-4-methylbenzamide (4.50 g, 11.29 mmol) prepared in Step 4 in N,N-dimethylformamide (40 mL), were added potassium carbonate (6.24 g, 45.16 mmol) and methyl 2-bromobutyrate (2.80 mL, 22.58 mmol). The reaction mixture was stirred at 30° C. overnight. The reaction mixture was quenched with water and then extracted with ethyl acetate twice. The combined organic layer was washed with brine, dried over magnesium sulfate, and then filtered. The filtrate was concentrated under reduced pressure. The residue was purified with silica gel column chromatography (n-hexaneethyl acetate=31) to give 4.83 g of the titled compound as a white solid.

### Step 6: methyl 2-(6-hydroxy-2-((4-methylbenzoyl)imino)benzo[d]thiazol-3(2H)-yl)butanoate

To a solution of 2-(6-((tert-butyldimethylsilyl)oxy)-2-((4-methylbenzoyl)imino)benzo[d]thiazol-3(2H)-yl)butanoate (3.0 g, 6.02 mmol) prepared in Step 5 in tetrahydrofuran (25 mL) was added tetrabutylammonium fluoride (1M solution of tetrahydrofuran, 7.0 mL, 7.0 mmol). The reaction mixture was stirred at room temperature for 1 hour, diluted with ethyl acetate, and then washed with an aqueous 1N HCl solution, a saturated sodium hydrogen carbonate solution, and brine. The organic layer was dried over magnesium sulfate and then filtered. The filtrate was concentrated under reduced pressure. The resulting residue was purified with silica gel column chromatography (dichloromethanemethanol=20/1) to give 2.10 g of the titled compound as a white solid.

### Step 7: methyl 2-(6-(benzyloxy)-2-((4-methylbenzoyl)imino)benzo[d]thiazol-3(2H)-yl)butanoate

To a solution of methyl 2-(6-hydroxy-2-((4-methylbenzoyl)imino)benzo[d]thiazol-3(2H)-yl)butanoate (200 mg, 0.52 mmol) prepared in Step 6 in N,N-dimethylformamide (3 mL), were added potassium carbonate (216 mg, 1.56 mmol) and benzyl bromide (93 uL, 0.78 mmol). The reaction mixture was stirred at room temperature overnight. The reaction mixture was quenched with water and then extracted with ethyl acetate twice. The combined organic layer was washed with brine, dried over magnesium sulfate, and then filtered. The filtrate was concentrated under reduced pressure. The residue was purified with silica gel column chromatography (n-hexaneethyl acetate=31) to give 220 mg of the titled compound as a white solid.

### Step 8: 2-(6-(benzyloxy)-2-((4-methylbenzoyl)imino)benzo[d]thiazol-3(2H)-yl)butanoic acid

To a solution of 2-(6-(benzyloxy)-2-((4-methylbenzoyl)imino)benzo[d]thiazol-3(2H)-yl)butanoate (210 mg, 0.44 mmol) prepared in Step 7 in a mixed solvent of tetrahydrofuran and methanol (1/1, 5 mL) was added a 3N sodium hydroxide solution (1 mL). The reaction mixture was stirred at room temperature overnight and then evaporated. The residue was diluted with water and then acidified to pH 3-4 with an aqueous 1N HCl solution. The resulting solid was filtered, washed with water and diisopropyl ether, and then dried under reduced pressure to give 160 mg of the titled compound as a gray solid.

¹H NMR (DMSO-d₆, 400 MHz); 8.12 (d, 2H), 7.59 (d, 1H), 7.48 (d, 2H), 7.42 (m, 3H), 7.37 (d, 1H), 7.30 (d, 2H), 7.09 (dd, 1H), 5.71 (d, 1H), 5.16 (s, 2H), 2.41 (m, 1H), 2.38 (s, 3H), 2.07 (m, 1H), 0.73 (t, 3H)

### Example 167

### 2-(2-((4-methylbenzoyl)imino)-6-(2-morpholinoethoxy)benzo[d]thiazol-3(2H)-yl)butanoic acid

### Step 1: methyl 2-(2-((4-methylbenzoyl)imino)-6-(2-morpholinoethoxy)benzo[d]thiazol-3(2H)-yl)butanoate

To a solution of triphenylphosphine (177 mg, 0.676 mmol) and diisopropyl azodicarboxylate (133 uL, 0.676 mmol) in tetrahydrofuran (3 mL), were added methyl 2-(6-hydroxy-2-((4-methylbenzoyl)imino)benzo[d]thiazol-3(2H)-yl)butanoate (200 mg, 0.52 mmol) prepared in Step 6 of Example 166 and 2-morpholinoethanol (82 uL, 0.676 mmol). The reaction mixture was stirred at room temperature overnight. The reaction mixture was quenched with water and then extracted with ethyl acetate twice. The combined organic layers were washed with brine, dried over magnesium sulfate, and then filtered. The filtrate was concentrated under reduced pressure. The residue was purified with silica gel column chromatography (dichloromethanemethanol=20/1) to give 220 mg of the titled compound as a white solid.

### Step 2: 2-(2-((4-methylbenzoyl)imino)-6-(2-morpholinoethoxy)benzo[d]thiazol-3(2H)-yl)butanoic acid

To a solution of methyl 2-(2-((4-methylbenzoyl)imino)-6-(2-morpholinoethoxy)benzo[d]thiazol-3(2H)-yl)butanoate (220 mg, 0.44 mmol) prepared in Step 1 in a mixed solvent of tetrahydrofuran and methanol (1/1, 5 mL) was added a 3N sodium hydroxide solution (1 mL). The reaction mixture was stirred at room temperature overnight and then evaporated. The residue was diluted with water and then acidified to pH 3-4 with an aqueous 1N HCl solution. The resulting solid was filtered, washed with water and diisopropyl ether, and then dried under reduced pressure to give 124 mg of the titled compound as a gray solid.

¹H NMR (DMSO-d₆, 400 MHz); 8.09 (d, 2H), 7.67 (s, 1H), 7.59 (s, 1H), 7.30 (d, 2H), 7.13 (d, 1H), 5.70 (brs, 1H), 4.16 (t, 2H), 3.59 (t, 4H), 2.74 (t, 2H), 2.50-2.25 (m, 2H), 2.39 (s, 3H), 0.75 (t, 3H)

### Example 168

### 2-(6-hydroxy-2-((4-methylbenzoyl)imino)benzo[d]thiazol-3(2H)-yl)butanoic acid

To a solution of methyl 2-(6-hydroxy-2-((4-methylbenzoyl)imino)benzo[d]thiazol-3(2H)-yl)butanoate (200 mg, 0.52 mmol) prepared in Step 6 of Example 166 in a mixed solvent of tetrahydrofuran and methanol (1/1, 4 mL) was added a 3N sodium hydroxide solution (1 mL). The reaction mixture was stirred at room temperature overnight. The reaction mixture was evaporated and diluted with water. The aqueous layer was acidified to pH 3-4 with an aqueous 1N HCl solution. The resulting solid was filtered, washed with water and diisopropyl ether, and then dried under reduced pressure to give 114 mg of the titled compound as a gray solid.

¹H NMR (DMSO-d₆, 400 MHz); 13.05 (brs, 1H), 9.76 (brs, 1H), 8.09 (d, 2H), 7.58 (s, 1H), 7.29 (m, 3H), 6.95 (m, 1H), 5.66 (brs, 1H), 2.50-2.30 (m, 2H), 2.38 (s, 3H), 0.75 (t, 3H)

### Example 169

### 2-[2-(4-methylbenzoylimino)-4-methoxybenzo[d]thiazol-3(2H)-yl]acetic acid

### Step 1: ethyl 2-[2-(4-methylbenzoylimino)-4-methoxybenzo[d]thiazol-3(2H)-yl]acetate

To a solution of 4-methyl-N-(4-methoxybenzo[d]thiazol-2-yl)benzamide (264 mg, 0.88 mmol), prepared in Example 8, in N,N-dimethylformamide (3.0 mL) were added potassium carbonate (365 mg, 2.64 mmol) and ethyl bromoacetate (147 uL, 1.32 mmol). The reaction mixture was refluxed overnight. The reaction mixture was quenched with water and then extracted with ethyl acetate. The organic layer was dried over anhydrous magnesium sulfate, filtered, and then evaporated. The residue was washed with isopropyl ether and then dried under reduced pressure to give 200 mg of the titled compound as a yellow solid (Yield: 72%).

¹H NMR (CDCl₃, 400 MHz) δ 8.21 (d, 2H), 7.22-7.26 (m, 3H), 7.12 (d, 1H), 7.01 (d, 1H), 5.18 (s, 2H), 4.25 (q, 2H), 3.87 (s, 3H), 2.41 (s, 3H), 1.25 (t, 3H)

### Step 2: 2-[2-(4-methylbenzoylimino)-4-methoxybenzo[d]thiazol-3(2H)-yl]acetic acid

To a solution of ethyl 2-[2-(4-methylbenzoylimino)-4-methoxybenzo[d]thiazol-3(2H)-yl]acetate (243 mg, 0.63 mmol) prepared in Step 1 in methanol (2.5 mL) was added a 10% sodium hydroxide solution (1.5 mL). The reaction mixture was stirred at room temperature overnight. The reaction mixture was evaporated, diluted with water, and then acidified with an aqueous 1N HCl solution. The resulting solid was filtered, washed with water, and then dried under reduced pressure to give 220 mg of the titled compound as a white solid (Yield: 98%).

¹H NMR (DMSO-d₆, 400 MHz) δ 8.12 (d, 2H), 7.63 (d, 1H), 7.58 (s, 1H), 7.32 (d, 2H), 7.12 (d, 1H), 5.28 (s, 2H), 3.83 (s, 3H), 2.39 (s, 3H)

### Example 170

### 2-(6-(azetidin-1-carbonyl)-2-(4-methylbenzoylimino)benzo[d]thiazol-3(2H)-yl)butanoic acid

### Step 1: ethyl 2-((4-methylbenzoyl)imino)-2,3-dihydroxybenzo[d]thiazol-6-carboxylate

To a solution of ethyl 2-aminobenzo[d]thiazol-6-carboxylate (5 g, 22.5 mmol) in tetrahydrofuran (50.0 mL) were added diisopropylethylamine (4.7 mL, 27.0 mmol) and p-toluoyl chloride (3.3 mL, 24.8 mmol). The reaction mixture was stirred at room temperature for 30 minutes and then N,N-dimethylaminopyridine (550 mg, 4.5 mmol) were added thereto. The reaction mixture was stirred at 40° C. for 6 hours and then at room temperature for 2 days. The reaction mixture was diluted with an aqueous 1N HCl solution (30.0 mL) and ethyl acetate (60.0 mL) and then stirred for 5 minutes. The resulting solid was filtered, washed with ethyl acetate, and then dried under reduced pressure to give 4.5 g of the titled compound as a yellow solid (Yield: 59%).

¹H NMR (DMSO-d₆, 400 MHz) δ 13.01 (bs, 1H), 8.67 (s, 1H), 8.05 (t, 3H), 7.85 (d, 1H), 7.38 (d, 2H), 4.35 (q, 2H), 2.40 (s, 3H), 1.35 (t, 3H)

### Step 2: 2-((4-methylbenzoyl)imino)-2,3-dihydrobenzo[d]thiazol-6-carboxylic acid

To a solution of ethyl 2-((4-methylbenzoyl)imino)-2,3-dihydroxybenzo[d]thiazol-6-carboxylate (2.0 g, 5.9 mmol) prepared in Step 1 in methanol (15.0 mL) was added a 10% sodium hydroxide solution (9.0 mL). The reaction mixture was stirred at room temperature for 5 days. The resulting solid was filtered, washed with methanol and ether, and then added to water. The reaction mixture was acidified with 1N HCl solution. The resulting solid was filtered, washed with water, and then dried under reduced pressure to give 2.15 g of the titled compound as a white solid (Yield: 100%).

¹H NMR (DMSO-d₆, 400 MHz) δ 12.97 (bs, 1H), 8.65 (d, 1H), 8.00-8.06 (m, 3H), 7.83 (t, 1H), 7.37 (d, 2H), 3.87 (s, 1H), 2.39 (s, 3H)

### Step 3: N-(6-(azetidin-1-carbonyl)benzo[d]thiazol-2(3H)-ylidene)-4-methylbenzamide

To a solution of 2-((4-methylbenzoyl)imino)-2,3-dihydrobenzo[d]thiazol-6-carboxylic acid (120 mg, 0.38 mmol), prepared in Step 2, azetidine hydrochloride (39 mg, 0.42 mmol) and BOP (202 mg, 0.46 mmol) in N,N-dimethylformamide (1.0 mL) was added diisopropylethylamine (265 uL, 1.52 mmol). The reaction mixture was stirred at room temperature overnight. The reaction mixture was diluted with ethyl acetate and water and then stirred for 5 minutes. The resulting solid was filtered, washed with water and ethyl acetate, and then dried under reduced pressure to give 91 mg of the titled compound as a yellow solid (Yield: 68%).

¹H NMR (DMSO-d₆, 400 MHz) δ 8.28 (s, 1H), 7.99-8.08 (m, 3H), 7.77 (d, 1H), 7.69 (d, 1H), 7.36 (d, 2H), 4.35 (m, 2H), 4.05 (m, 2H), 2.38 (s, 3H), 2.24-2.28 (m, 2H)

### Step 4: methyl 2-(6-(azetidin-1-carbonyl)-2-((4-methylbenzoyl)imino)benzo[d]thiazol-3(2H)-yl)butanoate

To a solution of N-(6-(azetidin-1-carbonyl)benzo[d]thiazol-2(3H)-ylidene)-4-methylbenzamide (91 mg, 0.26 mmol), prepared in Step 3, in N,N-dimethylformamide (1.0 mL) were added potassium carbonate (108 mg, 0.78 mmol) and methyl 2-bromobutyrate (45 uL, 0.39 mmol). The reaction mixture was stirred at 80° C. overnight and then cooled to room temperature. The reaction mixture was and quenched with water and then extracted with ethyl acetate. The organic layer was dried over anhydrous magnesium sulfate, filtered, and then evaporated. Diisopropyl ether and ethyl acetate were added to the filtrate. The resulting mixture was stirred for 5 minutes and then filtered. The resulting solid was washed with diisopropyl ether, and then dried under reduced pressure to give 61 mg of the titled compound as a white solid (Yield: 52%).

¹H NMR (CDCl₃, 400 MHz) δ 8.18 (d, 2H), 8.01 (s, 1H), 7.73-7.88 (m, 2H), 7.26-7.31 (m, 2H), 5.54-5.62 (m, 1H), 4.27-4.38 (m, 3H), 3.66 (s, 3H), 3.13 (m, 1H), 2.37-2.54 (m, 7H), 0.90 (t, 3H)

### Step 5: 2-(6-(azetidin-1-carbonyl)-2-((4-methylbenzoyl)imino)benzo[d]thiazol-3(2H)-yl)butanoic acid

To a solution of methyl 2-(6-(azetidin-1-carbonyl)-2-((4-methylbenzoyl)imino)benzo[d]thiazol-3(2H)-yl)butanoate (61 mg, 0.14 mmol), prepared in Step 4, in methanol (2.0 mL) was added a 10% sodium hydroxide solution (1.0 mL). The reaction mixture was stirred at room temperature overnight. The reaction mixture was diluted with water and then evaporated. The aqueous layer was acidified with an aqueous 1N HCl solution. The resulting solid was filtered, dried under reduced pressure, and then purified with Prep. TLC (dichloromethanemethanol=10/1) to give 10 mg of the titled compound as a white solid (Yield: 16%).

¹H NMR (MeOD, 400 MHz) δ 8.18 (m, 2H), 7.89-8.07 (m, 1H), 7.49-7.69 (m, 2H), 7.28 (m, 2H), 6.14 (m, 1H), 4.20-4.42 (m, 2H), 3.03-3.11 (m, 3H), 2.56 (m, 1H), 2.36-2.40 (m, 5H), 0.82 (m, 3H)

### Example 171

### 2-(2-((3-chlorobenzoyl)imino)-6-(trifluoromethyl)benzo[d]thiazol-3(2H)-yl)acetic acid

The titled compound was prepared in accordance with the same procedures as in Example 52, using 6-trifluoromethylbenzo[d]thiazol-2-amine prepared in Preparation 14, instead of 6-methoxybenzo[d]thiazol-2-amine (Yield: 41%).

¹H NMR (DMSO-d₆, 400 MHz); 13.46 (s, 1H), 8.47 (s, 1H), 8.20 (m, 2H), 7.96 (d, 1H), 7.89 (d, 1H), 7.68 (d, 1H), 7.58 (m, 1H), 5.39 (s, 2H)

### Example 172

### 2-(2-((3-chlorobenzoyl)imino)-6-(trifluoromethyl)benzo[d]thiazol-3(2H)-yl)propionic acid

The titled compound was prepared in accordance with the same procedures as in Example 52, using 6-trifluoromethylbenzo[d]thiazol-2-amine prepared in Preparation 14 and ethyl 2-bromopropionate, instead of 6-methoxybenzo[d]thiazol-2-amine and ethyl bromoacetate, respectively (Yield: 45%).

¹H NMR (DMSO-d₆, 400 MHz); 13.21 (s, 1H), 8.48 (s, 1H), 8.20 (s, 1H), 8.15 (d, 1H), 8.00 (d, 1H), 7.92 (d, 1H), 7.68 (d, 1H), 7.54 (m, 1H), 5.96 (s, 1H), 1.73 (d, 3H)

### Example 173

### 2-(2-((3-chlorobenzoyl)imino)-6-(trifluoromethyl)benzo[d]thiazol-3(2H)-yl)butanoic acid

The titled compound was prepared in accordance with the same procedures as in Example 52, using 6-trifluoromethylbenzo[d]thiazol-2-amine prepared in Preparation 14 and methyl 2-bromobutyrate, instead of 6-methoxybenzo[d]thiazol-2-amine and ethyl bromoacetate, respectively (Yield: 56%).

¹H NMR (DMSO-d₆, 400 MHz); 13.24 (s, 1H), 8.49 (s, 1H), 8.18 (s, 1H), 8.14 (d, 1H), 8.02 (s, 1H), 7.92 (d, 1H), 7.68 (d, 1H), 7.56 (t, 1H), 5.84 (s, 1H), 2.50-2.31 (m, 2H), 0.75 (t, 3H)

### Example 174

### 2-(2-((3-chlorobenzoyl)imino)-6-(trifluoromethoxy)benzo[d]thiazol-3(2H)-yl)acetic acid

The titled compound was prepared in accordance with the same procedures as in Example 52, using 6-trifluoromethoxybenzo[d]thiazol-2-amine prepared in Preparation 1, instead of 6-methoxybenzo[d]thiazol-2-amine (Yield: 52%).

¹H NMR (DMSO-d₆, 400 MHz); 13.46 (s, 1H), 8.18-8.13 (m, 3H), 7.87 (d, 1H), 7.66 (d, 1H), 7.56 (m, 2H), 5.35 (s, 2H)

### Example 175

### 2-(2-((3-chlorobenzoyl)imino)-6-(trifluoromethoxy)benzo[d]thiazol-3(2H)-yl)propionic acid

The titled compound was prepared in accordance with the same procedures as in Example 52, using 6-trifluoromethoxybenzo[d]thiazol-2-amine prepared in Preparation 1 and ethyl 2-bromopropionate, instead of 6-methoxybenzo[d]thiazol-2-amine and ethyl bromoacetate, respectively (Yield: 28%).

¹H NMR (DMSO-d₆, 400 MHz); 13.18 (s, 1H), 8.19 (s, 1H), 8.14 (m, 2H), 7.91 (d, 1H), 7.66 (d, 1H), 7.56 (m, 2H), 5.89 (s, 1H), 1.71 (d, 3H)

### Example 176

### 2-(2-((3-chlorobenzoyl)imino)-6-(trifluoromethoxy)benzo[d]thiazol-3(2H)-yl)butanoic acid

The titled compound was prepared in accordance with the same procedures as in Example 52, using 6-trifluoromethoxybenzo[d]thiazol-2-amine prepared in Preparation 1 and methyl 2-bromobutyrate, instead of 6-methoxybenzo[d]thiazol-2-amine and ethyl bromoacetate, respectively (Yield: 34%).

¹H NMR (DMSO-d₆, 400 MHz); 13.25 (s, 1H), 8.17-8.12 (m, 3H), 7.92 (s, 1H), 7.66 (d, 1H), 7.56 (m, 2H), 5.78 (s, 1H), 2.48-2.32 (m, 2H), 0.75 (t, 3H)

### Example 177

### 2-(6-chloro-2-((3-chlorobenzoyl)imino)benzo[d]thiazol-3(2H)-yl)acetic acid

The titled compound was prepared in accordance with the same procedures as in Example 52, using 6-chlorobenzo[d]thiazol-2-amine, instead of 6-methoxybenzo[d]thiazol-2-amine (Yield: 22%).

¹H NMR (DMSO-d₆, 400 MHz); 13.41 (s, 1H), 8.18-8.12 (m, 3H), 7.79 (d, 1H), 7.66 (m, 1H), 7.59-7.53 (m, 2H), 5.32 (s, 2H)

### Example 178

### 2-(6-chloro-2-((3-chlorobenzoyl)imino)benzo[d]thiazol-3(2H)-yl)propionic acid

The titled compound was prepared in accordance with the same procedures as in Example 52, using 6-chlorobenzo[d]thiazol-2-amine and ethyl 2-bromopropionate, instead of 6-methoxybenzo[d]thiazol-2-amine and ethyl bromoacetate, respectively (Yield: 49%).

¹H NMR (DMSO-d₆, 400 MHz); 13.17 (s, 1H), 8.19 (s, 1H), 8.18-8.12 (m, 2H), 7.83 (d, 1H), 7.66-7.61 (m, 2H), 7.54 (t, 1H), 5.87 (s, 1H), 1.70 (d, 3H)

### Example 179

### 2-(6-chloro-2-((3-chlorobenzoyl)imino)benzo[d]thiazol-3(2H)-yl)butanoic acid

The titled compound was prepared in accordance with the same procedures as in Example 52, using 6-chlorobenzo[d]thiazol-2-amine and methyl 2-bromobutyrate, instead of 6-methoxybenzo[d]thiazol-2-amine and ethyl bromoacetate, respectively (Yield: 36%).

¹H NMR (DMSO-d₆, 400 MHz); 13.17 (s, 1H), 8.17-8.11 (m, 3H), 7.84 (s, 1H), 7.66-7.60 (m, 2H), 7.54 (t, 1H), 5.75 (s, 1H), 2.48-2.30 (m, 2H), 0.74 (t, 3H)

### Example 180

### 2-(2-((3-bromobenzoyl)imino)benzo[d]thiazol-3(2H)-yl)acetic acid

The titled compound was prepared in accordance with the same procedures as in Example 52, using benzo[d]thiazol-2-amine and 3-bromobenzoyl chloride, instead of 6-methoxybenzo[d]thiazol-2-amine and 3-chlorobenzoyl chloride, respectively (Yield: 44%).

¹H NMR (DMSO-d₆, 400 MHz) δ 8.34 (s, 1H), 8.20 (d, 1H), 7.96 (d, 1H), 7.76 (dd, 2H), 7.46-7.54 (m, 2H), 7.39 (dd, 1H), 5.33 (brs, 2H).

### Example 181

### 2-(2-((3-bromobenzoyl)imino)benzo[d]thiazol-3(2H)-yl)propionic acid

The titled compound was prepared in accordance with the same procedures as in Example 52, using benzo[d]thiazol-2-amine, 3-bromobenzoyl chloride, and ethyl 2-bromopropionate, instead of 6-methoxybenzo[d]thiazol-2-amine, 3-chlorobenzoyl chloride, and ethyl bromoacetate, respectively (Yield: 45%).

¹H NMR (DMSO-d₆, 400 MHz) δ 8.34 (s, 1H), 8.18 (d, 1H), 7.97 (d, 1H), 7.79 (dd, 2H), 7.56 (dd, 1H), 7.45 (dd, 1H), 7.40 (dd, 1H), 5.87 (brs, 1H), 1.71 (d, 3H).

### Example 182

### 2-(2-(4-methylbenzoylimino)-6-(methylcarbamoyl)benzo[d]thiazol-3(2H)-yl)butanoic acid

### Step 1: N-methyl-2-(4-methylbenzamido)benzo[d]thiazol-6-carboxamide

To a solution of 2-((4-methylbenzoyl)imino)-2,3-dihydrobenzo[d]thiazol-6-carboxylic acid (300 mg, 0.96 mmol), prepared in Step 2 of Example 170, methylamine hydrochloride (71 mg, 1.06 mmol) and BOP (510 mg, 1.15 mmol) in N,N-dimethylformamide (3.0 mL) was added diisopropylethylamine (836 uL, 4.80 mmol). The reaction mixture was stirred at room temperature overnight. The reaction mixture was diluted with ethyl acetate and water and then stirred for 5 minutes. The resulting solid was filtered, washed with water and ethyl acetate, and then dried under reduced pressure to give 185 mg of the titled compound as a pale yellow solid (Yield: 59%).

¹H NMR (DMSO-d₆, 400 MHz) δ 12.94 (s, 1H), 8.47 (d, 1H), 8.04 (d, 2H), 7.78-7.92 (m, 2H), 7.36 (d, 2H), 3.87 (s, 1H), 2.79-2.97 (m, 3H), 2.39 (s, 3H)

### Step 2: methyl 2-(2-((4-methylbenzoyl)imino-6-(methylcarbamoyl(2H)-yl)butanoate

To a solution of N-methyl-2-(4-methylbenzamido)benzo[d]thiazol-6-carboxamide (185 mg, 0.57 mmol) prepared in Step 1 in N,N-dimethylformamide (2.0 mL) were added potassium carbonate (236 mg, 1.71 mmol) and methyl 2-bromobutyrate (98 uL, 0.86 mmol). The reaction mixture was stirred at 80° C. overnight and cooled to room temperature. The reaction mixture was quenched with water and then extracted with ethyl acetate. The organic layer was dried over anhydrous magnesium sulfate, filtered, and then evaporated. The residue was purified with silica gel column chromatography (n-hexaneethyl acetate=8/1˜1/1, v/v) to give 160 mg of the titled compound as a white solid (Yield: 66%).

¹H NMR (CDCl₃, 400 MHz) δ 8.20 (s, 1H), 8.08 (d, 2H), 7.90 (d, 1H), 7.27 (d, 1H), 7.18-7.25 (m, 2H), 6.94-6.95 (m, 1H), 5.53 (m, 1H), 3.57 (s, 3H), 2.91 (d, 3H), 2.39-2.46 (m, 2H), 2.34 (s, 3H), 0.82 (t, 3H)

### Step 3: 2-(2-(4-methylbenzoylimino)-6-(methylcarbamoyl)benzo[d]thiazol-3(2H)-yl)butanoic acid

To a solution of methyl 2-(2-((4-methylbenzoyl)imino-6-(methylcarbamoyl(2H)-yl)butanoate (160 mg, 0.38 mmol) prepared in Step 2 in methanol (3.0 mL) was added a 10% sodium hydroxide solution (1.0 mL). The reaction mixture was stirred at room temperature for 5 days. The reaction mixture was evaporated, diluted with water, washed with ether, and then acidified with an aqueous 1N HCl solution. The aqueous layer was added sodium chloride and extracted with acetonitrile. The organic layer was dried over anhydrous magnesium sulfate, filtered, and then evaporated. The residue was filtered, washed with methanol, and then dried under reduced pressure to give 84 mg of the titled compound as a white solid (Yield: 54%).

¹H NMR (DMSO-d₆, 400 MHz) δ 8.49-8.52 (m, 1H), 8.36 (s, 1H), 8.08 (d, 2H), 7.96 (d, 1H), 7.85 (m, 1H), 7.30 (d, 2H), 5.76 (m, 1H), 2.81 (d, 3H), 2.31-2.37 (m, 5H), 0.74 (t, 3H)

### Example 183

### 2-(2-(4-methylbenzoylimino)-6-(propylcarbamoyl)benzo[d]thiazol-3(2H)-yl)butanoic acid

The titled compound was prepared in accordance with the same procedures as in Example 182, using propylamine, instead of methylamine hydrochloride (Yield: 12%).

¹H NMR (DMSO-d₆, 400 MHz) δ 8.53 (t, 1H), 8.39 (s, 1H), 8.11 (d, 2H), 8.00 (d, 1H), 7.87 (m, 1H), 7.33 (d, 2H), 5.78 (m, 1H), 3.23-3.27 (m, 2H), 2.40-2.50 (m, 5H), 1.52-1.59 (m, 2H), 0.92 (t, 3H), 0.76 (t, 3H)

### Example 184

### 2-(2-(4-(trifluoromethyl)benzoylimino)benzo[d]thiazol-3(2H)-yl)acetic acid

### Step 1: ethyl 2-(2-((4-(trifluoromethyl(benzoyl)imino)benzo[d]thiazol-3(2H)-yl)acetate

To a solution of N-(benzo[d]thiazol-2-yl)-4-(trifluoromethyl)benzamide (300 mg, 0.93 mmol) and potassium carbonate (386 mg, 2.79 mmol) in N,N-dimethylformamide (3.0 mL) was added ethyl bromoacetate (154 uL, 1.40 mmol). The reaction mixture was refluxed overnight. The reaction mixture was quenched with water and then extracted with ethyl acetate. The organic layer was dried over anhydrous magnesium sulfate, filtered, and then evaporated. The residue was stirred with isopropyl ether for 5 minute, filtered, washed with a mixed solvent of n-hexaneethyl acetate (81), and then dried under reduced pressure to give 298 mg of the titled compound as a white solid (Yield: 78%).

¹H NMR (CDCl₃, 400 MHz) δ 8.42 (d, 2H), 7.70-7.75 (m, 3H), 7.49 (t, 1H), 7.36 (t, 1H), 7.26-7.28 (m, 1H), 5.24 (s, 2H), 4.27 (q, 2H), 1.27 (t, 3H)

### Step 2: 2-(2-((4-(trifluoromethyl)benzoyl)imino)benzo[d]thiazol-3(2H)-yl)acetic acid

To a solution of ethyl 2-(2-((4-(trifluoromethyl(benzoyl)imino)benzo[d]thiazol-3(2H)-yl)acetate (298 mg, 0.73 mmol) prepared in Step 1 in methanol (6.0 mL) was added a 10% sodium hydroxide solution (3.0 mL). The reaction mixture was stirred at room temperature overnight. The reaction mixture was evaporated, diluted with water, and then acidified with an aqueous 1N HCl solution. The resulting solid was filtered, washed with water, and then dried under reduced pressure to give 244 mg of the titled compound as a white solid (Yield: 88%).

¹H NMR (DMSO-d₆, 400 MHz) δ 8.40 (d, 2H), 7.97 (d, 1H), 7.88 (d, 2H), 7.75 (d, 1H), 7.54 (t, 1H), 7.39 (t, 1H), 5.36 (s, 2H)

### Example 185

### 2-(2-(4-methylbenzoylimino)-4,5,6,7-tetrahydrobenzo[d]thiazol-3(2H)-yl)acetic acid

The titled compound was prepared in accordance with the same procedures as in Example 184, using 4-methyl-N-(4,5,6,7-tetrahydrobenzo[d]thiazol-2-yl)benzamide, instead of N-(benzo[d]thiazol-2-yl)-4-(trifluoromethyl)benzamide (Yield: 95%).

¹H NMR (DMSO-d₆, 400 MHz) δ 8.03 (d, 2H), 7.24 (d, 2H), 4.95 (s, 2H), 2.48-2.53 (m, 4H), 2.34 (s, 3H), 1.78 (m, 4H)

### Example 186

### 2-(2-(4-(trifluoromethyl)benzoylimino)benzo[d]thiazol-3(2H)-yl)propionic acid

The titled compound was prepared in accordance with the same procedures as in Example 184, using ethyl 2-bromopropionate, instead of ethyl bromoacetate (Yield: 93%).

¹H NMR (DMSO-d₆, 400 MHz) δ 12.97-13.30 (m, 1H), 8.38 (d, 2H), 7.99 (d, 1H), 7.87 (d, 2H), 7.80 (d, 1H), 7.57 (t, 1H), 7.40 (t, 1H), 5.92 (m, 1H), 1.72 (d, 3H)

### Example 187

### 2-(2-(4-methylbenzoylimino)-4,5,6,7-tetrahydrobenzo[d]thiazol-3(2H)-yl)propionic acid

The titled compound was prepared in accordance with the same procedures as in Example 52, using 4-methyl-N-(4,5,6,7-tetrahydrobenzo[d]thiazol-2-yl)benzamide and ethyl 2-bromopropionate, instead of N-(benzo[d]thiazol-2-yl)-4-(trifluoromethyl)benzamide and ethyl bromoacetate, respectively (Yield: 95%).

¹H NMR (DMSO-d₆, 400 MHz) δ 8.01 (d, 2H), 7.23 (d, 2H), 5.18 (m, 1H), 2.53-2.58 (m, 4H), 2.34 (s, 3H), 1.76-1.82 (m, 4H), 1.62 (d, 3H)

### Example 188

### 2-(6-methoxy-2-((3-(trifluoromethyl)benzoyl)imino)benzo[d]thiazol-3(2H)-yl)acetic acid

The titled compound was prepared in accordance with the same procedures as in Example 52, using 3-(trifluoromethyl)benzoyl chloride, instead of 3-chlorobenzoyl chloride (Yield: 34%).

¹H NMR (DMSO-d₆, 400 MHz); 8.51 (d, 1H), 8.49 (s, 1H), 7.97 (d, 1H), 7.78 (t, 1H), 7.72 (d, 1H), 7.63 (s, 1H), 7.17 (d, 1H), 5.33 (s, 2H), 3.84 (s, 3H)

### Example 189

### 2-(6-methoxy-2-((3-(trifluoromethyl)benzoyl)imino)benzo[d]thiazol-3(2H)-yl)propionic acid

The titled compound was prepared in accordance with the same procedures as in Example 52, using 3-(trifluoromethyl)benzoyl chloride and ethyl 2-bromopropionate, instead of 3-chlorobenzoyl chloride and ethyl bromoacetate, respectively (Yield: 45%).

¹H NMR (DMSO-d₆, 400 MHz); 8.51 (s, 1H), 8.48 (d, 1H), 7.97 (d, 1H), 7.77 (t, 2H), 7.64 (s, 1H), 7.19 (d, 1H), 5.85 (s, 1H), 3.84 (s, 3H), 1.73 (d, 3H)

### Example 190

### 2-(6-methoxy-2-((3-(trifluoromethyl)benzoyl)imino)benzo[d]thiazol-3(2H)-yl)butanoic acid

The titled compound was prepared in accordance with the same procedures as in Example 52, using 3-(trifluoromethyl)benzoyl chloride and methyl 2-bromobutyrate, instead of 3-chlorobenzoyl chloride and ethyl bromoacetate, respectively (Yield: 51%).

¹H NMR (DMSO-d₆, 400 MHz); 8.49 (s, 1H), 8.47 (d, 1H), 7.96 (d, 1H), 7.77 (t, 2H), 7.64 (s, 1H), 7.18 (d, 1H), 5.73 (s, 1H), 3.84 (s, 3H), 2.50 (m, 1H), 2.34 (m, 1H), 0.76 (t, 3H)

### Example 191

### 2-(6-ethoxy-2-((3-(trifluoromethyl)benzoyl)imino)benzo[d]thiazol-3(2H)-yl)acetic acid

The titled compound was prepared in accordance with the same procedures as in Example 52, using 6-ethoxybenzo[d]thiazol-2-amine and 3-(trifluoromethyl)benzoyl chloride, instead of 6-methoxybenzo[d]thiazol-2-amine and 3-chlorobenzoyl chloride, respectively (Yield: 42%).

¹H NMR (DMSO-d₆, 400 MHz); 8.51 (d, 1H), 8.49 (s, 1H), 7.97 (d, 1H), 7.78 (t, 1H), 7.70 (d, 1H), 7.61 (s, 1H), 7.15 (d, 1H), 5.32 (s, 2H), 4.11 (q, 2H), 1.36 (t, 3H)

### Example 192

### 2-(6-ethoxy-2-((3-(trifluoromethyl)benzoyl)imino)benzo[d]thiazol-3(2H)-yl)propionic acid

The titled compound was prepared in accordance with the same procedures as in Example 52, using 6-ethoxybenzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 26%).

¹H NMR (DMSO-d₆, 400 MHz); 8.50 (s, 1H), 8.48 (d, 1H), 7.96 (d, 1H), 7.77 (m, 2H), 7.62 (s, 1H), 7.18 (d, 1H), 5.85 (s, 1H), 4.11 (q, 2H), 1.73 (d, 3H), 1.36 (t, 3H)

### Example 193

### 2-(6-ethoxy-2-((3-(trifluoromethyl)benzoyl)imino)benzo[d]thiazol-3(2H)-yl)butanoic acid

The titled compound was prepared in accordance with the same procedures as in Example 52, using 6-ethoxybenzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 42%).

¹H NMR (DMSO-d₆, 400 MHz); 8.49 (s, 1H), 8.47 (d, 1H), 7.96 (d, 1H), 7.77 (t, 2H), 7.62 (s, 1H), 7.17 (d, 1H), 5.72 (s, 1H), 4.12 (q, 2H), 2.50 (m, 1H), 2.35 (m, 1H), 1.36 (t, 3H), 0.80 (t, 3H)

### Example 194

### 2-(6-methyl-2-((3-(trifluoromethyl)benzoyl)imino)benzo[d]thiazol-3(2H)-yl)acetic acid

The titled compound was prepared in accordance with the same procedures as in Example 52, using 6-methylbenzo[d]thiazol-2-amine and 3-(trifluoromethyl)benzoyl chloride, instead of 6-methoxybenzo[d]thiazol-2-amine and 3-chlorobenzoyl chloride, respectively (Yield: 36%).

¹H NMR (DMSO-d₆, 400 MHz); 8.51 (d, 1H), 8.49 (s, 1H), 7.97 (d, 1H), 7.78 (m, 2H), 7.68 (d, 1H), 7.38 (d, 1H), 5.33 (s, 2H), 2.42 (s, 3H)

### Example 195

### 2-(6-methyl-2-((3-(trifluoromethyl)benzoyl)imino)benzo[d]thiazol-3(2H)-yl)propionic acid

The titled compound was prepared in accordance with the same procedures as in Example 52, using 6-methylbenzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 18%).

¹H NMR (DMSO-d₆, 400 MHz); 8.51 (s, 1H), 8.49 (d, 1H), 7.97 (d, 1H), 7.79-7.72 (m, 3H), 7.41 (d, 1H), 5.87 (s, 1H), 2.43 (s, 3H), 1.73 (d, 3H)

### Example 196

### 2-(6-methyl-2-((3-(trifluoromethyl)benzoyl)imino)benzo[d]thiazol-3(2H)-yl)butanoic acid

The titled compound was prepared in accordance with the same procedures as in Example 52, using 6-methylbenzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 26%).

¹H NMR (DMSO-d₆, 400 MHz); 8.50 (s, 1H), 8.47 (d, 1H), 7.97 (d, 1H), 7.80-7.75 (m, 3H), 5.74 (s, 1H), 2.50 (m, 1H), 2.43 (s, 3H), 2.35 (m, 1H), 0.76 (t, 3H)

### Example 197

### 2-(6-(trifluoromethoxy)-2-((3-(trifluoromethyl)benzoyl)imino)benzo[d]thiazol-3(2H)-yl)acetic acid

The titled compound was prepared in accordance with the same procedures as in Example 52, using 6-(trifluoromethoxy)benzo[d]thiazol-2-amine and 3-(trifluoromethyl)benzoyl chloride, instead of 6-methoxybenzo[d]thiazol-2-amine and 3-chlorobenzoyl chloride, respectively (Yield: 26%).

¹H NMR (DMSO-d₆, 400 MHz); 8.52 (d, 1H), 8.50 (s, 1H), 8.16 (s, 1H), 7.99 (d, 1H), 7.91 (d, 1H), 7.81 (t, 1H), 7.59 (d, 1H), 5.38 (s, 2H)

### Example 198

### 2-(6-(trifluoromethoxy)-2-((3-(trifluoromethyl)benzoyl)imino)benzo[d]thiazol-3(2H)-yl)propionic acid

The titled compound was prepared in accordance with the same procedures as in Example 52, using 6-(trifluoromethoxy)benzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 40%).

¹H NMR (DMSO-d₆, 400 MHz); 8.52 (s, 1H), 8.49 (d, 1H), 8.18 (s, 1H), 7.97 (m, 2H), 7.79 (t, 1H), 7.63 (d, 1H), 5.93 (s, 1H), 1.76 (d, 3H)

### Example 199

### 2-(6-(trifluoromethoxy)-2-((3-(trifluoromethyl)benzoyl)imino)benzo[d]thiazol-3(2H)-yl)butanoic acid

The titled compound was prepared in accordance with the same procedures as in Example 52, using 6-(trifluoromethoxy)benzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 38%).

¹H NMR (DMSO-d₆, 400 MHz); 8.50 (s, 1H), 8.48 (d, 1H), 8.19 (s, 1H), 7.99 (d, 2H), 7.81 (t, 1H), 7.62 (d, 1H), 5.81 (s, 1H), 2.50 (m, 1H), 2.37 (m, 1H), 0.78 (t, 3H)

### Example 200

### 2-(4,6-difluoro-2-(4-methylbenzoylimino)benzo[d]thiazol-3(2H)-yl)acetic acid

### Step 1: N-(4,6-difluorobenzo[d]thiazol-2-yl)-4-methylbenzamide

To a solution of 4,6-difluorobenzo[d]thiazol-2-amine (1.80 g, 10.00 mmol) in tetrahydrofuran (100 mL) were added diisopropylethylamine (2.61 mL, 15.00 mmol), 4,4-dimethylaminopyridine (122 mg, 1.00 mmol), and p-toluoyl chloride (1.54 mL, 12.00 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine. The organic layer was dried over anhydrous magnesium sulfate, filtered, and then evaporated. The residue was washed with isopropyl ether and ethyl acetate and then dried under reduced pressure to give 4.4 g of the titled compound as a white solid (Yield: 75%).

¹H NMR (CDCl₃, 400 MHz) δ 10.57-10.76 (m, 1H), 7.88 (d, 2H), 7.26-7.36 (m, 3H), 6.93 (t, 1H), 2.43 (s, 3H)

### Step 2: ethyl 2-(4,6-difluoro-2-((4-methylbenzoyl)imino)benzo[d]thiazol-3(2H)-yl)acetate

To a solution of N-(4,6-difluorobenzo[d]thiazol-2-yl)-4-methylbenzamide (300 mg, 0.98 mmol) prepared in Step 1 in N,N-dimethylformamide (3.0 mL) were added potassium carbonate (406 mg, 2.94 mmol) and ethyl bromoacetate (163 uL, 1.47 mmol). The reaction mixture was stirred at 80° C. overnight. The reaction mixture was quenched with water and then extracted with ethyl acetate. The organic layer was washed with brine, dried over anhydrous magnesium sulfate, filtered, and then evaporated. The residue was washed with a mixed solvent of n-hexane and ethyl acetate (81) and then dried under reduced pressure to give 249 mg of the titled compound as a white solid (Yield: 65%).

¹H NMR (CDCl₃, 400 MHz) δ 8.20 (d, 2H), 7.22-7.27 (m, 3H), 6.94 (t, 1H), 5.42 (s, 2H), 4.28 (q, 2H), 2.42 (s, 3H), 1.29 (t, 3H)

### Step 3: 2-(4,6-difluoro-2-(4-methylbenzoylimino)benzo[d]thiazol-3(2H)-yl)acetic acid

To a solution of ethyl 2-(4,6-difluoro-2-((4-methylbenzoyl)imino)benzo[d]thiazol-3(2H)-yl)acetate (249 mg, 0.64 mmol) prepared in Step 2 in methanol (5.0 mL) was added a 10% sodium hydroxide solution (2.0 mL). The reaction mixture was stirred at room temperature overnight. The reaction mixture was evaporated, diluted with water, and then washed with ether. The aqueous layer was acidified with an aqueous 1N HCl solution. The resulting solid was filtered, washed with water, and then dried under reduced pressure to give 216 mg of the titled compound as a white solid (Yield: 93%).

¹H NMR (DMSO-d₆, 400 MHz) δ 8.12 (d, 2H), 7.79 (d, 1H), 7.50 (t, 1H), 7.31 (d, 2H), 5.39 (s, 2H), 2.37 (s, 3H)

### Example 201

### 2-(4,6-difluoro-2-(4-methylbenzoylimino)benzo[d]thiazol-3(2H)-yl)propionic acid

The titled compound was prepared in accordance with the same procedures as in Example 200, using ethyl 2-bromopropionate instead ethyl bromoacetate (Yield: 51%).

¹H NMR (DMSO-d₆, 400 MHz) δ 8.13 (d, 2H), 7.81 (d, 1H), 7.52 (t, 1H), 7.31 (d, 2H), 6.30 (m, 1H), 2.37 (s, 3H), 1.72 (d, 3H)

### Example 202

### 2-(2-(4-methylbenzoylimino)-6-(pyrrolidin-1-carbonyl)benzo[d]thiazol-3(2H)-yl)butanoic acid

The titled compound was prepared in accordance with the same procedures as in Example 170, using pyrrolidine instead azetidine hydrochloride (Yield: 48%).

¹H NMR (DMSO-d₆, 400 MHz) δ 13.13-13.14 (m, 1H), 8.16 (s, 1H), 8.11 (d, 2H), 7.83 (m, 1H), 7.70 (d, 1H), 7.33 (d, 2H), 5.78 (m, 1H), 3.50 (d, 4H), 2.40 (m, 5H), 1.83-1.89 (m, 4H), 0.77 (t, 3H)

### Example 203

### 2-(2-(4-methylbenzoylimino)-6-(piperidin-1-carbonyl)benzo[d]thiazol-3(2H)-yl)butanoic acid

The titled compound was prepared in accordance with the same procedures as in Example 170, using piperidine instead azetidine hydrochloride (Yield: 69%).

¹H NMR (DMSO-d₆, 400 MHz) δ 13.06 (m, 1H), 8.14 (d, 2H), 8.03 (d, 1H), 7.75 (m, 1H), 7.46 (d, 1H), 7.25 (d, 2H), 5.69-5.70 (m, 1H), 3.25-3.51 (m, 4H), 2.32-2.43 (m, 5H), 1.46-1.55 (m, 6H), 0.70 (t, 3H)

### Example 204

### 2-(2-(4-methylbenzoylimino)-6-(morpholine-4-carbonyl)benzo[d]thiazol-3(2H)-yl)butanoic acid

The titled compound was prepared in accordance with the same procedures as in Example 170, using morpholine instead azetidine hydrochloride (Yield: 8%).

¹H NMR (MeOD, 400 MHz) δ 8.18 (d, 2H), 7.91 (s, 1H), 7.51-7.60 (m, 2H), 7.29 (d, 2H), 6.00 (m, 1H), 3.59-3.70 (m, 8H), 2.41-2.53 (m, 5H), 0.83 (m, 3H)

### Example 205

### 2-(6-chloro-2-((3-(trifluoromethyl)benzoyl)imino)benzo[d]thiazol-3(2H)-yl)acetic acid

The titled compound was prepared in accordance with the same procedures as in Example 52, using 6-chlorobenzo[d]thiazol-2-amine and 3-(trifluoromethyl)benzoyl chloride, instead of 6-methoxybenzo[d]thiazol-2-amine and 3-chlorobenzoyl chloride, respectively (Yield: 16%).

¹H NMR (DMSO-d₆, 400 MHz); 8.52 (d, 1H), 8.49 (s, 1H), 8.16 (s, 1H), 7.99 (d, 1H), 7.81 (m, 2H), 7.62 (d, 1H), 5.36 (s, 2H)

### Example 206

### 2-(6-chloro-2-((3-(trifluoromethyl)benzoyl)imino)benzo[d]thiazol-3(2H)-yl)propionic acid

The titled compound was prepared in accordance with the same procedures as in Example 52, using 6-chlorobenzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride and ethyl bromoacetate, respectively (Yield: 18%).

¹H NMR (DMSO-d₆, 400 MHz); 8.51 (s, 1H), 8.49 (d, 1H), 8.17 (s, 1H), 7.99 (d, 1H), 7.87 (d, 1H), 7.78 (t, 1H), 7.66 (d, 1H), 5.89 (s, 1H), 1.74 (d, 3H)

### Example 207

### 2-(6-chloro-2-((3-(trifluoromethyl)benzoyl)imino)benzo[d]thiazol-3(2H)-yl)butanoic acid

The titled compound was prepared in accordance with the same procedures as in Example 52, using 6-chlorobenzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 24%).

¹H NMR (DMSO-d₆, 400 MHz); 8.49 (s, 1H), 8.47 (d, 1H), 8.18 (s, 1H), 7.99 (d, 1H), 7.90 (s, 1H), 7.79 (t, 1H), 7.66 (d, 1H), 5.78 (s, 1H), 2.51 (m, 1H), 2.35 (m, 1H), 0.77 (t, 3H)

### Example 208

### 2-(2-((3-chlorobenzoyl)imino)-6-fluorobenzo[d]thiazol-3(2H)-yl)acetic acid

The titled compound was prepared in accordance with the same procedures as in Example 52, using 6-fluorobenzo[d]thiazol-2-amine, instead of 6-methoxybenzo[d]thiazol-2-amine (Yield: 33%).

¹H NMR (DMSO-d₆, 400 MHz); 8.21 (s, 1H), 8.18 (d, 1H), 7.96 (d, 1H), 7.83 (dd, 1H), 7.68 (d, 1H), 7.59 (t, 1H), 7.44 (t, 1H), 5.35 (s, 2H)

### Example 209

### 2-(2-((3-chlorobenzoyl)imino)-6-fluorobenzo[d]thiazol-3(2H)-yl)propionic acid

The titled compound was prepared in accordance with the same procedures as in Example 52, using 6-fluorobenzo[d]thiazol-2-amine and ethyl 2-bromopropionate, instead of 6-methoxybenzo[d]thiazol-2-amine and ethyl bromoacetate, respectively (Yield: 25%).

¹H NMR (DMSO-d₆, 400 MHz); 8.16 (s, 1H), 8.14 (d, 1H), 7.97 (d, 1H), 7.86 (d, 1H), 7.68 (d, 1H), 7.56 (t, 1H), 7.47 (t, 1H), 5.90 (s, 1H), 1.73 (d, 3H)

### Example 210

### 2-(2-((3-chlorobenzoyl)imino)-6-fluorobenzo[d]thiazol-3(2H)-yl)butanoic acid

The titled compound was prepared in accordance with the same procedures as in Example 52, using 6-fluorobenzo[d]thiazol-2-amine and methyl 2-bromobutyrate, instead of 6-methoxybenzo[d]thiazol-2-amine and ethyl bromoacetate, respectively (Yield: 28%).

¹H NMR (DMSO-d₆, 400 MHz); 8.16 (s, 1H), 8.13 (d, 1H), 7.97 (d, 1H), 7.89 (s, 1H), 7.68 (d, 1H), 7.56 (t, 1H), 7.47 (t, 1H), 5.77 (s, 1H), 2.51 (m, 1H), 2.34 (m, 1H), 0.76 (t, 3H)

### Example 211

### 2-(2-((3-chlorobenzoyl)imino)-5,6-dimethylbenzo[d]thiazol-3(2H)-yl)acetic acid

The titled compound was prepared in accordance with the same procedures as in Example 52, using 5,6-dimethylbenzo[d]thiazol-2-amine, instead of 6-methoxybenzo[d]thiazol-2-amine (Yield: 36%).

¹H NMR (DMSO-d₆, 400 MHz); 8.19 (s, 1H), 8.17 (d, 1H), 7.71 (s, 1H), 7.66 (d, 1H), 7.58 (m, 2H), 5.30 (s, 2H), 2.35 (s, 3H), 2.32 (s, 3H)

### Example 212

### 2-(2-((3-chlorobenzoyl)imino)-5,6-dimethylbenzo[d]thiazol-3(2H)-yl)propionic acid

The titled compound was prepared in accordance with the same procedures as in Example 52, using 5,6-dimethylbenzo[d]thiazol-2-amine and ethyl 2-bromopropionate, instead of 6-methoxybenzo[d]thiazol-2-amine and ethyl bromoacetate, respectively (Yield: 38%).

¹H NMR (DMSO-d₆, 400 MHz); 8.20 (s, 1H), 8.15 (d, 1H), 7.72 (s, 1H), 7.66 (m, 2H), 7.55 (t, 1H), 5.82 (s, 1H), 2.37 (s, 3H), 2.32 (s, 3H), 1.72 (d, 3H)

### Example 213

### 2-(2-((3-chlorobenzoyl)imino)-5,6-dimethylbenzo[d]thiazol-3(2H)-yl)butanoic acid

The titled compound was prepared in accordance with the same procedures as in Example 52, using 5,6-dimethylbenzo[d]thiazol-2-amine and methyl 2-bromobutyrate, instead of 6-methoxybenzo[d]thiazol-2-amine and ethyl bromoacetate, respectively (Yield: 12%).

¹H NMR (DMSO-d₆, 400 MHz); 8.18 (s, 1H), 8.14 (d, 1H), 7.72-7.64 (m, 3H), 7.55 (t, 1H), 5.69 (s, 1H), 2.50 (m, 1H), 2.37-2.33 (m, 1H), 2.37 (s, 3H), 2.33 (s, 3H), 0.75 (t, 3H)

### Example 214

### 2-(6-(trifluoromethyl)-2-((3-(trifluoromethyl)benzoyl)imino)benzo[d]thiazol-3(2H)-yl)acetic acid

The titled compound was prepared in accordance with the same procedures as in Example 52, using 6-(trifluoromethyl)benzo[d]thiazol-2-amine and 3-(trifluoromethyl)benzoyl chloride, instead of 6-methoxybenzo[d]thiazol-2-amine and 3-chlorobenzoyl chloride, respectively (Yield: 8%).

¹H NMR (DMSO-d₆, 400 MHz); 8.53-8.50 (m, 3H), 8.00 (d, 2H), 7.91 (d, 1H), 7.79 (t, 1H), 5.41 (s, 2H)

### Example 215

### 2-(6-(trifluoromethyl)-2-((3-(trifluoromethyl)benzoyl)imino)benzo[d]thiazol-3(2H)-yl)propionic acid

The titled compound was prepared in accordance with the same procedures as in Example 52, using 6-(trifluoromethyl)benzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 15%).

¹H NMR (DMSO-d₆, 400 MHz); 8.52-8.48 (m, 3H), 8.05-7.93 (m, 3H), 7.80 (t, 1H), 5.99 (s, 1H), 1.77 (d, 3H)

### Example 216

### 2-(6-(trifluoromethyl)-2-((3-(trifluoromethyl)benzoyl)imino)benzo[d]thiazol-3(2H)-yl)butanoic acid

The titled compound was prepared in accordance with the same procedures as in Example 52, using 6-(trifluoromethyl)benzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 24%).

¹H NMR (DMSO-d₆, 400 MHz); 8.53-8.47 (m, 3H), 8.07 (s, 1H), 8.00 (d, 1H), 7.95 (d, 1H), 7.80 (t, 1H), 5.87 (s, 1H), 2.50 (m, 1H), 2.38 (m, 1H), 0.78 (t, 3H)

### Example 217

### 2-(5,6-dimethyl-2-((3-(trifluoromethyl)benzoyl)imino)benzo[d]thiazol-3(2H)-yl)acetic acid

The titled compound was prepared in accordance with the same procedures as in Example 52, using 5,6-dimethylbenzo[d]thiazol-2-amine and 3-(trifluoromethyl)benzoyl chloride, instead of 6-methoxybenzo[d]thiazol-2-amine and 3-chlorobenzoyl chloride, respectively (Yield: 16%).

¹H NMR (DMSO-d₆, 400 MHz); 8.50 (m, 2H), 7.97 (d, 1H), 7.78 (t, 1H), 7.72 (s, 1H), 7.61 (s, 1H), 5.30 (s, 2H), 2.35 (s, 3H), 2.32 (s, 3H)

### Example 218

### 2-(5,6-dimethyl-2-((3-(trifluoromethyl)benzoyl)imino)benzo[d]thiazol-3(2H)-yl)propionic acid

The titled compound was prepared in accordance with the same procedures as in Example 52, using 5,6-dimethylbenzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 23%).

¹H NMR (DMSO-d₆, 400 MHz); 8.51 (s, 1H), 8.48 (d, 1H), 7.96 (d, 1H), 7.77 (m, 2H), 7.68 (s, 1H), 5.82 (s, 1H), 2.37 (s, 3H), 2.33 (s, 3H), 1.74 (d, 3H)

### Example 219

### 2-(5,6-dimethyl-2-((3-(trifluoromethyl)benzoyl)imino)benzo[d]thiazol-3(2H)-yl)butanoic acid

The titled compound was prepared in accordance with the same procedures as in Example 52, using 5,6-dimethylbenzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 24%).

¹H NMR (DMSO-d₆, 400 MHz); 8.49 (s, 1H), 8.46 (d, 1H), 7.96 (d, 1H), 7.79-7.74 (m, 3H), 5.71 (s, 1H), 2.50 (m, 1H), 2.37-2.33 (m, 1H), 2.37 (s, 3H), 2.33 (s, 3H), 0.76 (t, 3H)

### Example 220

### 2-(6-fluoro-2-((3-(trifluoromethyl)benzoyl)imino)benzo[d]thiazol-3(2H)-yl)acetic acid

The titled compound was prepared in accordance with the same procedures as in Example 52, using 6-fluorobenzo[d]thiazol-2-amine and 3-(trifluoromethyl)benzoyl chloride, instead of 6-methoxybenzo[d]thiazol-2-amine and 3-chlorobenzoyl chloride, respectively (Yield: 16%).

¹H NMR (DMSO-d₆, 400 MHz); 8.49 (m, 2H), 7.97 (m, 2H), 7.85-7.77 (m, 2H), 7.44 (t, 1H), 5.36 (s, 2H)

### Example 221

### 2-(6-fluoro-2-((3-(trifluoromethyl)benzoyl)imino)benzo[d]thiazol-3(2H)-yl)propionic acid

The titled compound was prepared in accordance with the same procedures as in Example 52, using 6-fluorobenzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 20%).

¹H NMR (DMSO-d₆, 400 MHz) 8.51 (s, 1H), 8.49 (d, 1H), 7.98 (d, 2H), 7.89 (d, 1H), 7.78 (t, 1H), 7.48 (t, 1H), 5.89 (s, 1H), 1.74 (d, 3H)

### Example 222

### 2-(6-fluoro-2-((3-(trifluoromethyl)benzoyl)imino)benzo[d]thiazol-3(2H)-yl)butanoic acid

The titled compound was prepared in accordance with the same procedures as in Example 52, using 6-fluorobenzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 25%).

¹H NMR (DMSO-d₆, 400 MHz); 8.50 (s, 1H), 8.47 (d, 1H), 7.99 (d, 2H), 7.91 (s, 1H), 7.78 (t, 1H), 7.48 (t, 1H), 5.78 (s, 1H), 2.51 (m, 1H), 2.35 (m, 1H), 0.77 (t, 3H)

### Example 223

### 2-(2-(3,5-difluorobenzoylimino)benzo[d]thiazol-3(2H)-yl)propionic acid

### Step 1: ethyl 2-(2-((3,5-difluorobenzoyl)imino)benzo[d]thiazol-3(2H)-yl)propionate

To a solution of N-(benzo[d]thiazol-2-yl)-3,5-difluorobenzamide (100 mg, 0.344 mmol) in N,N-dimethylformamide (3.0 mL) were added potassium carbonate (95 mg, 0.688 mmol) and ethyl 2-bromopropionate (93 mg, 0.516 mmol). The reaction mixture was stirred at 80° C. overnight and then diluted with water (15 mL). The resulting solid was filtered and then dried under reduced pressure to give 110 mg of the titled compound as a white solid (Yield: 82%).

¹H NMR (CDCl₃, 400 MHz) δ 7.74-7.82 (m, 3H), 7.49 (dd, 1H), 7.32-7.38 (m, 2H), 6.96 (dd, 1H), 5.67 (brs, 1H), 4.15-4.23 (m, 2H), 1.86 (d, 3H), 1.12 (t, 3H)

### Step 2: 2-(2-(3,5-difluorobenzoylimino)benzo[d]thiazol-3(2H)-yl)propionic acid

To a solution of ethyl 2-(2-((3,5-difluorobenzoyl)imino)benzo[d]thiazol-3(2H)-yl)propionate (100 mg, 0.256 mmol) prepared in Step 1 in a mixed solvent of tetrahydrofuran and methanol (1/1, 4.0 mL) was added a 1N sodium hydroxide solution (1.0 mL). The reaction mixture was stirred at room temperature overnight. The reaction mixture was evaporated, diluted with water, and then acidified to pH 3-4 with an aqueous 1N HCl solution. The resulting solid was filtered and then dried under reduced pressure to give 74.2 mg of the titled compound as a white solid (Yield: 80%).

¹H NMR (DMSO-d₆, 400 MHz) δ 13.18 (brs, 1H), 8.02 (d, 1H), 7.83 (d, 3H), 7.42-7.62 (m, 3H), 5.94 (s, 1H), 1.71 (d, 3H).

### Example 224

### 2-(2-(3,5-difluorobenzoylimino)benzo[d]thiazol-3(2H)-yl)acetic acid

The titled compound was prepared in accordance with the same procedures as in Example 223, using ethyl bromoacetate, instead of ethyl 2-bromopropionate (Yield: 89%).

¹H NMR (DMSO-d₆, 400 MHz) δ 8.10 (d, 1H), 7.85 (d, 2H), 7.79 (d, 1H), 7.50-7.59 (m, 2H), 7.42 (dd, 1H), 5.38 (s, 2H).

### Example 225

### 2-(5,6-difluoro-2-((3-methyl-5-(trifluoromethoxy)benzoyl)imino)benzo[d]thiazol-3(2H)-yl)butanoic acid

The titled compound was prepared in accordance with the same procedures as in Example 52, using 5,6-difluorobenzo[d]thiazol-2-amine prepared in Preparation 3, 3-methyl-5-(trifluoromethoxy)benzoyl chloride, and methyl 2-bromobutyrate, instead of 6-methoxybenzo[d]thiazol-2-amine, 3-chlorobenzoyl chloride, and ethyl bromoacetate, respectively (Yield: 21%).

¹H NMR (MeOD, 400 MHz) δ 8.09 (s, 1H), 7.93 (s, 1H), 7.90-7.85 (m, 1H), 7.85-7.70 (m, 1H), 7.31 (s, 1H), 5.75-5.55 (brs, 1H), 2.70-2.40 (m, 2H), 2.48 (s, 3H), 0.89 (t, 3H)

### Example 226

### 2-(6-ethyl-2-((4-methylbenzoyl)imino)benzo[d]thiazol-3(2H)-yl)acetic acid

The titled compound was prepared in accordance with the same procedures as in Example 52, using 6-ethylbenzo[d]thiazol-2-amine and p-toluoyl chloride, instead of 6-methoxybenzo[d]thiazol-2-amine and 3-chlorobenzoyl chloride, respectively (Yield: 32%).

¹H NMR (DMSO-d₆, 400 MHz); 13.36 (s, 1H), 8.14 (d, 2H), 7.78 (s, 1H), 7.64 (d, 1H), 7.38 (d, 1H), 7.33 (d, 2H), 5.30 (s, 2H), 2.73 (q, 2H), 2.39 (s, 3H), 1.24 (t, 3H)

### Example 227

### 2-(6-ethyl-2-((4-methylbenzoyl)imino)benzo[d]thiazol-3(2H)-yl)propionic acid

The titled compound was prepared in accordance with the same procedures as in Example 52, using 6-ethylbenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 26%).

¹H NMR (DMSO-d₆, 400 MHz); 13.04 (s, 1H), 8.13 (d, 2H), 7.79 (s, 1H), 7.68 (d, 1H), 7.41 (d, 1H), 7.32 (d, 2H), 5.86 (s, 1H), 2.73 (q, 2H), 2.39 (s, 3H), 1.74 (d, 3H), 1.24 (t, 3H)

### Example 228

### 2-(6-ethyl-2-((4-methylbenzoyl)imino)benzo[d]thiazol-3(2H)-yl)butanoic acid

The titled compound was prepared in accordance with the same procedures as in Example 52, using 6-ethylbenzo[d]thiazol-2-amine, p-toluoyl chloride, and methyl 2-bromobutyrate, instead of 6-methoxybenzo[d]thiazol-2-amine, 3-chlorobenzoyl chloride, and ethyl bromoacetate, respectively (Yield: 41%).

¹H NMR (DMSO-d₆, 400 MHz); 13.04 (s, 1H), 8.11 (d, 2H), 7.79 (s, 1H), 7.69 (s, 1H), 7.40 (d, 1H), 7.32 (d, 2H), 5.73 (s, 1H), 2.73 (q, 2H), 2.51 (m, 1H), 2.39 (s, 3H), 2.33 (m, 1H), 1.24 (t, 3H), 0.75 (t, 3H)

### Example 229

### 2-(2-((3-chlorobenzoyl)imino)-6-ethylbenzo[d]thiazol-3(2H)-yl)acetic acid

The titled compound was prepared in accordance with the same procedures as in Example 52, using 6-ethylbenzo[d]thiazol-2-amine, instead of 6-methoxybenzo[d]thiazol-2-amine (Yield: 32%).

¹H NMR (DMSO-d₆, 400 MHz); 13.38 (s, 1H), 8.21 (s, 1H), 8.18 (d, 1H), 7.81 (s, 1H), 7.67 (t, 2H), 7.56 (t, 1H), 7.41 (d, 1H), 5.33 (s, 2H), 2.73 (q, 2H), 1.24 (t, 3H)

### Example 230

### 2-(2-((3-chlorobenzoyl)imino)-6-ethylbenzo[d]thiazol-3(2H)-yl)propionic acid

The titled compound was prepared in accordance with the same procedures as in Example 52, using 6-ethylbenzo[d]thiazol-2-amine and ethyl 2-bromopropionate, instead of 6-methoxybenzo[d]thiazol-2-amine and ethyl bromoacetate, respectively (Yield: 36%).

¹H NMR (DMSO-d₆, 400 MHz); 13.09 (s, 1H), 8.21 (s, 1H), 8.16 (d, 1H), 7.83 (s, 1H), 7.74 (d, 1H), 7.67 (d, 1H), 7.55 (t, 1H), 7.44 (d, 1H), 5.88 (s, 1H), 2.73 (q, 2H), 1.72 (d, 3H), 1.24 (t, 3H)

### Example 231

### 2-(2-((3-chlorobenzoyl)imino)-6-ethylbenzo[d]thiazol-3(2H)-yl)butanoic acid

The titled compound was prepared in accordance with the same procedures as in Example 52, using 6-ethylbenzo[d]thiazol-2-amine and methyl 2-bromobutyrate, instead of 6-methoxybenzo[d]thiazol-2-amine and ethyl bromoacetate, respectively (Yield: 24%).

¹H NMR (DMSO-d₆, 400 MHz); 13.12 (s, 1H), 8.19 (s, 1H), 8.15 (d, 1H), 7.83 (s, 1H), 7.76 (s, 1H), 7.67 (d, 1H), 7.55 9t, 1H), 7.43 (d, 1H), 5.75 (s, 1H), 2.74 (q, 2H), 2.47 (m, 1H), 2.34 (m, 1H), 1.25 (t, 3H), 0.75 (t, 3H)

### Example 232

### 2-(6-ethyl-2-((3-(trifluoromethyl)benzoyl)imino)benzo[d]thiazol-3(2H)-yl)acetic acid

The titled compound was prepared in accordance with the same procedures as in Example 52, using 6-ethylbenzo[d]thiazol-2-amine and 3-(trifluoromethyl)benzoyl chloride, instead of 6-methoxybenzo[d]thiazol-2-amine and 3-chlorobenzoyl chloride, respectively (Yield: 25%).

¹H NMR (DMSO-d₆, 400 MHz); 13.39 (s, 1H), 8.52 (d, 1H), 8.50 (s, 1H), 7.98 (d, 1H), 7.83 (s, 1H), 7.80 (t, 1H), 7.71 (d, 1H), 7.42 (d, 1H), 5.34 (s, 2H), 2.74 (q, 2H), 1.24 (t, 3H)

### Example 233

### 2-(6-ethyl-2-((3-(trifluoromethyl)benzoyl)imino)benzo[d]thiazol-3(2H)-yl)propionic acid

The titled compound was prepared in accordance with the same procedures as in Example 52, using 6-ethylbenzo[d]thiazol-2-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 25%).

¹H NMR (DMSO-d₆, 400 MHz); 13.10 (s, 1H), 8.51 (s, 1H), 8.49 (d, 1H), 7.97 (d, 1H), 7.85 (s, 1H), 7.78 (m, 2H), 7.45 (d, 1H), 5.88 (s, 1H), 2.74 (q, 2H), 1.73 (d, 3H), 1.24 (t, 3H)

### Example 234

### 2-(6-ethyl-2-((3-(trifluoromethyl)benzoyl)imino)benzo[d]thiazol-3(2H)-yl)butanoic acid

The titled compound was prepared in accordance with the same procedures as in Example 52, using 6-ethylbenzo[d]thiazol-2-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 22%).

¹H NMR (DMSO-d₆, 400 MHz); 13.11 (s, 1H), 8.50 (s, 1H), 8.48 (d, 1H), 7.98 (d, 1H), 7.85 (s, 1H), 7.78 (m, 2H), 7.45 (d, 1H), 5.77 (s, 1H), 2.75 (q, 2H), 2.51 (m, 1H), 2.36 (m, 1H), 1.25 (t, 3H), 0.77 (t, 3H)

### Example 235

### 2-(6-((4-methylbenzoyl)imino)-[1,3]dioxolo[4′,5′:4,5]benzo[1,2-d]thiazol-7(6H)-yl)acetic acid

The titled compound was prepared in accordance with the same procedures as in Example 52, using [1,3]dioxolo[4′,5′:4,5]benzo[1,2-d]thiazol-6-amine and p-toluoyl chloride, instead of 6-methoxybenzo[d]thiazol-2-amine and 3-chlorobenzoyl chloride, respectively (Yield: 32%).

¹H NMR (DMSO-d₆, 400 MHz); 13.41 (s, 1H), 8.18 (d, 2H), 7.60 (d, 2H), 7.38 (d, 2H), 6.19 (s, 2H), 5.33 (s, 2H), 2.45 (s, 3H)

### Example 236

### 2-(6-((4-methylbenzoyl)imino)-[1,3]dioxolo[4′,5′:4,5]benzo[1,2-d]thiazol-7(6H)-yl)propionic acid

The titled compound was prepared in accordance with the same procedures as in Example 52, using [1,3]dioxolo[4′,5′:4,5]benzo[1,2-d]thiazol-6-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 36%).

¹H NMR (DMSO-d₆, 400 MHz); 13.03 (s, 1H), 8.10 (d, 2H), 7.55 (s, 1H), 7.31 (d, 2H), 6.14 (s, 2H), 5.73 (s, 1H), 2.39 (s, 3H), 1.70 (d, 3H)

### Example 237

### 2-(6-((4-methylbenzoyl)imino)-[1,3]dioxolo[4′,5′:4,5]benzo[1,2-d]thiazol-7(6H)-yl)butanoic acid

The titled compound was prepared in accordance with the same procedures as in Example 52, using [1,3]dioxolo[4′,5′:4,5]benzo[1,2-d]thiazol-6-amine, p-toluoyl chloride, and methyl 2-bromobutyrate, instead of 6-methoxybenzo[d]thiazol-2-amine, 3-chlorobenzoyl chloride, and ethyl bromoacetate, respectively (Yield: 18%).

¹H NMR (DMSO-d₆, 400 MHz); 13.02 (s, 1H), 8.08 (d, 2H), 7.58 (s, 1H), 7.54 (s, 1H), 7.30 (d, 2H), 6.14 (d, 2H), 5.59 (s, 1H), 2.51 (m, 1H), 2.38 (s, 3H), 2.31 (m, 1H), 0.75 (t, 3H)

### Example 238

### 2-(6-((3-chlorobenzoyl)imino)-[1,3]dioxolo[4′,5′:4,5]benzo[1,2-d]thiazol-7(6H)-yl)acetic acid

The titled compound was prepared in accordance with the same procedures as in Example 52, using [1,3]dioxolo[4′,5′:4,5]benzo[1,2-d]thiazol-6-amine, instead of 6-methoxybenzo[d]thiazol-2-amine (Yield: 19%).

¹H NMR (DMSO-d₆, 400 MHz); 13.36 (s, 1H), 8.18 (s, 1H), 8.15 (d, 1H), 7.66 (d, 1H), 7.55 (m, 3H), 6.14 (s, 2H), 5.29 (s, 2H)

### Example 239

### 2-(6-((3-chlorobenzoyl)imino)-[1,3]dioxolo[4′,5′:4,5]benzo[1,2-d]thiazol-7(6H)-yl)propionic acid

The titled compound was prepared in accordance with the same procedures as in Example 52, using [1,3]dioxolo[4′,5′:4,5]benzo[1,2-d]thiazol-6-amine and ethyl 2-bromopropionate, instead of 6-methoxybenzo[d]thiazol-2-amine and ethyl bromoacetate, respectively (Yield: 12%).

¹H NMR (DMSO-d₆, 400 MHz); 13.10 (s, 1H), 8.18 (s, 1H), 8.13 (d, 1H), 7.65-7.52 (m, 4H), 6.15 (s, 2H), 5.75 (s, 1H), 1.70 (d, 3H)

### Example 240

### 2-(6-((3-chlorobenzoyl)imino)-[1,3]dioxolo[4′,5′:4,5]benzo[1,2-d]thiazol-7(6H)-yl)butanoic acid

The titled compound was prepared in accordance with the same procedures as in Example 52, using [1,3]dioxolo[4′,5′:4,5]benzo[1,2-d]thiazol-6-amine and methyl 2-bromobutyrate, instead of 6-methoxybenzo[d]thiazol-2-amine and ethyl bromoacetate, respectively (Yield: 23%).

¹H NMR (DMSO-d₆, 400 MHz); 12.98 (s, 1H), 8.05 (s, 1H), 8.00 (d, 1H), 7.54 (d, 2H), 7.45 (m, 2H), 6.04 (d, 2H), 5.51 (s, 1H), 2.40 (m, 1H), 2.21 (m, 1H), 0.64 (t, 3H)

### Example 241

### 2-(6-((3-(trifluoromethyl)benzoyl)imino)-[1,3]dioxolo[4′,5′:4,5]benzo[1,2-d]thiazol-7(6H)-yl)acetic acid

The titled compound was prepared in accordance with the same procedures as in Example 52, using [1,3]dioxolo[4′,5′:4,5]benzo[1,2-d]thiazol-6-amine and 3-(trifluoromethyl)benzoyl chloride, instead of 6-methoxybenzo[d]thiazol-2-amine and 3-chlorobenzoyl chloride, respectively (Yield: 18%).

¹H NMR (DMSO-d₆, 400 MHz); 13.37 (s, 1H), 8.49 (d, 1H), 8.47 (s, 1H), 7.96 (d, 1H), 7.77 (t, 1H), 7.58 (s, 2H), 6.14 (s, 2H), 5.30 (s, 2H)

### Example 242

### 2-(6-((3-(trifluoromethyl)benzoyl)imino)-[1,3]dioxolo[4′,5′:4,5]benzo[1,2-d]thiazol-7(6H)-yl)propionic acid

The titled compound was prepared in accordance with the same procedures as in Example 52, using [1,3]dioxolo[4′,5′:4,5]benzo[1,2-d]thiazol-6-amine, 3-(trifluoromethyl)benzoyl chloride, and ethyl 2-bromopropionate, instead of 6-methoxybenzo[d]thiazol-2-amine, 3-chlorobenzoyl chloride, and ethyl bromoacetate, respectively (Yield: 16%).

¹H NMR (DMSO-d₆, 400 MHz); 13.07 (s, 1H), 8.49 (s, 1H), 8.46 (d, 1H), 7.95 (d, 1H), 7.76 (t, 1H), 7.62 (d, 1H), 7.59 (s, 1H), 6.15 (s, 2H), 5.76 (s, 1H), 1.72 (d, 3H)

### Example 243

### 2-(6-((3-(trifluoromethyl)benzoyl)imino)-[1,3]dioxolo[4′,5′:4,5]benzo[1,2-d]thiazol-7(6H)-yl)butanoic acid

The titled compound was prepared in accordance with the same procedures as in Example 52, using [1,3]dioxolo[4′,5′:4,5]benzo[1,2-d]thiazol-6-amine, 3-(trifluoromethyl)benzoyl chloride, and methyl 2-bromobutyrate, instead of 6-methoxybenzo[d]thiazol-2-amine, 3-chlorobenzoyl chloride, and ethyl bromoacetate, respectively (Yield: 15%).

¹H NMR (DMSO-d₆, 400 MHz); 13.08 (s, 1H), 8.48 (s, 1H), 8.44 (d, 1H), 7.95 (d, 1H), 7.76 (t, 1H), 7.65 (s, 1H), 7.59 (s, 1H), 6.16 (s, 2H), 5.63 (s, 1H), 2.51 (m, 1H), 2.32 (m, 1H), 0.76 (t, 3H)

### Example 244

### 2-(6-fluoro-2-((4-methylbenzoyl)imino)benzo[d]thiazol-3(2H)-yl)acetic acid

The titled compound was prepared in accordance with the same procedures as in Example 52, using 6-fluorobenzo[d]thiazol-2-amine and p-toluoyl chloride, instead of 6-methoxybenzo[d]thiazol-2-amine and 3-chlorobenzoyl chloride, respectively (Yield: 26%).

¹H NMR (DMSO-d₆, 400 MHz); 8.12 (d, 2H), 7.92 (d, 1H), 7.75 (dd, 1H), 7.40 (t, 1H), 7.33 (d, 2H), 5.30 (s, 2H), 2.39 (s, 3H)

### Example 245

### 2-(6-fluoro-2-((4-methylbenzoyl)imino)benzo[d]thiazol-3(2H)-yl)propionic acid

The titled compound was prepared in accordance with the same procedures as in Example 52, using 6-fluorobenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 30%).

¹H NMR (DMSO-d₆, 400 MHz); 8.13 (d, 2H), 7.91 (d, 1H), 7.69 (s, 1H), 7.40 (t, 1H), 7.32 (d, 2H), 5.88 (s, 1H), 2.39 (s, 3H), 1.69 (d, 3H)

### Example 246

### 2-(2-((4-methylbenzoyl)imino)-6-(trifluoromethyl)benzo[d]thiazol-3(2H)-yl)acetic acid

The titled compound was prepared in accordance with the same procedures as in Example 52, using 6-(trifluoromethyl)benzo[d]thiazol-2-amine and p-toluoyl chloride, instead of 6-methoxybenzo[d]thiazol-2-amine and 3-chlorobenzoyl chloride, respectively (Yield: 29%).

¹H NMR (DMSO-d₆, 400 MHz); 8.41 (s, 1H), 8.13 (d, 2H), 7.83 (d, 1H), 7.76 (d, 1H), 7.29 (d, 2H), 5.20 (s, 2H), 2.36 (s, 3H)

### Example 247

### 2-(2-((4-methylbenzoyl)imino)-6-(trifluoromethyl)benzo[d]thiazol-3(2H)-yl)propionic acid

The titled compound was prepared in accordance with the same procedures as in Example 52, using 6-(trifluoromethyl)benzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 31%).

¹H NMR (DMSO-d₆, 400 MHz); 8.34 (s, 1H), 8.03 (d, 2H), 7.78 (m, 2H), 7.23 (d, 2H), 5.84 (s, 1H), 2.29 (s, 3H), 1.62 (d, 3H)

### Example 248

### 2-(2-((4-methylbenzoyl)imino)-6-(trifluoromethoxy)benzo[d]thiazol-3(2H)-yl)acetic acid

The titled compound was prepared in accordance with the same procedures as in Example 52, using 6-(trifluoromethoxy)benzo[d]thiazol-2-amine and p-toluoyl chloride, instead of 6-methoxybenzo[d]thiazol-2-amine and 3-chlorobenzoyl chloride, respectively (Yield: 21%).

¹H NMR (DMSO-d₆, 400 MHz); 8.12 (d, 2H), 8.09 (s, 1H), 7.70 (d, 1H), 7.51 (d, 1H), 7.29 (d, 2H), 5.19 (s, 2H), 2.37 (s, 3H)

### Example 249

### 2-(2-((4-methylbenzoyl)imino)-6-(trifluoromethoxy)benzo[d]thiazol-3(2H)-yl)propionic acid

The titled compound was prepared in accordance with the same procedures as in Example 52, using 6-(trifluoromethoxy)benzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 45%).

¹H NMR (DMSO-d₆, 400 MHz); 8.13 (m, 3H), 7.84 (d, 1H), 7.57 (d, 1H), 7.34 (d, 2H), 5.90 (s, 1H), 2.40 (s, 3H), 1.72 (d, 3H)

### Example 250

### 2-(6-ethoxy-2-((4-methylbenzoyl)imino)benzo[d]thiazol-3(2H)-yl)acetic acid

The titled compound was prepared in accordance with the same procedures as in Example 52, using 6-ethoxybenzo[d]thiazol-2-amine and p-toluoyl chloride, instead of 6-methoxybenzo[d]thiazol-2-amine and 3-chlorobenzoyl chloride, respectively (Yield: 12%).

¹H NMR (DMSO-d₆, 400 MHz); 8.12 (d, 2H), 7.60 (d, 1H), 7.55 (s, 1H), 7.31 (d, 2H), 7.10 (d, 1H), 5.24 (s, 2H), 4.10 (q, 2H), 2.38 (s, 3H), 1.36 (t, 3H)

### Example 251

### 2-(5,6-dimethyl-2-((4-methylbenzoyl)imino)benzo[d]thiazol-3(2H)-yl)acetic acid

The titled compound was prepared in accordance with the same procedures as in Example 52, using 5,6-dimethylbenzo[d]thiazol-2-amine and p-toluoyl chloride, instead of 6-methoxybenzo[d]thiazol-2-amine and 3-chlorobenzoyl chloride, respectively (Yield: 16%).

¹H NMR (DMSO-d₆, 400 MHz); 13.23 (s, 1H), 8.04 (d, 2H), 7.59 (s, 1H), 7.46 (s, 1H), 7.24 (d, 2H), 5.18 (s, 2H), 2.30 (s, 3H), 2.26 (s, 3H), 2.23 (s, 3H)

### Example 252

### 2-(5,6-difluoro-2-((4-methylbenzoyl)imino)benzo[d]thiazol-3(2H)-yl)acetic acid

The titled compound was prepared in accordance with the same procedures as in Example 52, using 5,6-difluorobenzo[d]thiazol-2-amine and p-toluoyl chloride, instead of 6-methoxybenzo[d]thiazol-2-amine and 3-chlorobenzoyl chloride, respectively (Yield: 31%).

¹H NMR (DMSO-d₆, 400 MHz); 8.09-8.03 (m, 3H), 7.96 (m, 1H), 7.25 (d, 2H), 5.19 (s, 2H), 2.31 (s, 3H)

### Example 253

### 2-(5,6-difluoro-2-((4-methylbenzoyl)imino)benzo[d]thiazol-3(2H)-yl)propionic acid

The titled compound was prepared in accordance with the same procedures as in Example 52, using 5,6-difluorobenzo[d]thiazol-2-amine, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 9%).

¹H NMR (DMSO-d₆, 400 MHz); 8.15 (t, 1H), 8.11 (d, 2H), 8.00 (s, 1H), 7.33 (d, 2H), 5.76 (s, 1H), 2.39 (s, 3H), 1.71 (d, 3H)

### Example 254

### 3-hydroxy-2-(2-(4-methylbenzoylimino)benzo[d]thiazol-3(2H)-yl)propionic acid

### Step 1: N-(benzo[d]thiazol-2-yl)-4-methylbenzamide

To a solution of benzo[d]thiazol-2-amine (5.0 g, 33.3 mmol) in tetrahydrofuran (50.0 mL) were added diisopropylethylamine (7.0 mL, 40.0 mmol), 4,4-dimethylaminopyridine (814 mg, 6.66 mmol), and p-toluoyl chloride (4.8 mL, 36.6 mmol). The reaction mixture was stirred at room temperature overnight. The reaction mixture was quenched with water and then extracted with ethyl acetate. The organic layer was washed with an aqueous 1N HCl solution, a saturated sodium bicarbonate solution, and brine. The organic layer was dried over anhydrous magnesium sulfate, filtered, and then evaporated. The residue was washed with isopropyl ether and ethyl acetate and then dried under reduced pressure to give 8.5 g of the titled compound as a white solid (Yield: 95%).

¹H NMR (CDCl₃, 400 MHz) δ 11.12 (bs, 1H), 7.83-7.89 (m, 3H), 7.38 (d, 1H), 7.28-7.32 (m, 2H), 7.22-7.25 (m, 2H), 2.39 (s, 3H)

### Step 2: methyl 2-bromo-3-hydroxypropanoate

To a solution of L-serine (5.0 g, 47.6 mmol), a 48 w/w % bromic acid solution (13.0 mL, 109.5 mmol), and potassium bromide (20.0 g, 157.1 mmol) in water (44.0 mL) was added portionwise sodium nitrite (6.0 g, 80.9 mmol) at −10° C. The reaction mixture was stirred at room temperature overnight. The reaction mixture was saturated with sodium chloride and then extracted with ethyl acetate. The organic layer was dried over anhydrous magnesium sulfate, filtered, and then evaporated. The residue was dissolved in methanol (50.0 mL) and then 48 w/w % bromic acid (0.2 mL) was added thereto. The reaction mixture was stirred at 65° C. for 2 days and then concentrated under reduced pressure to discard excess methanol. The resulting dark yellow residue was dissolved in dichloromethane (100.0 mL) and then washed with a saturated sodium hydrogen carbonate solution (50.0 mL) and brine. The organic layer was dried over anhydrous sodium sulfate, filtered, and then evaporated. The residue was dried under reduced pressure to give 5.7 g of the titled compound (Yield: 65%).

¹H NMR (CDCl₃, 400 MHz) δ 4.36 (t, 1H), 4.01-4.08 (m, 1H), 3.93-3.97 (m, 1H), 3.82 (s, 3H), 2.56 (t, 1H)

### Step 3: methyl 3-hydroxy-2-(2-((4-methylbenzoyl)imino)benzo[d]thiazol-3(2H)-yl)propanoate

To a solution of N-(benzo[d]thiazol-2-yl)-4-methylbenzamide (100 mg, 0.37 mmol) prepared in Step 1 in N,N-dimethylformamide (1.0 mL) were added potassium carbonate (153 mg, 1.11 mmol) and methyl 2-bromo-3-hydroxypropanoate (102 mg, 0.56 mmol) prepared in Step 2. The reaction mixture was stirred at 80° C. for 6.5 hours and then cooled to room temperature. The reaction mixture was quenched with water and then extracted with ethyl acetate. The organic layer was dried over anhydrous magnesium sulfate, filtered, and then evaporated. The residue was purified with silica gel column chromatography (n-hexaneethyl acetate=8/1˜1/1) to give 66 mg of the titled compound as a white solid (Yield: 48%).

¹H NMR (CDCl₃, 400 MHz) δ 8.13-8.18 (m, 2H), 7.56-7.62 (m, 1H), 7.42-7.49 (m, 2H), 7.20-7.29 (m, 3H), 4.83-4.94 (m, 2H), 4.67-4.74 (m, 1H), 4.47-4.59 (m, 1H), 3.67 (d, 3H), 2.40 (s, 3H)

### Step 4: 3-hydroxy-2-(2-(4-methylbenzoylimino)benzo[d]thiazol-3(2H)-yl)propionic acid

To a solution of methyl 3-hydroxy-2-(2-((4-methylbenzoyl)imino)benzo[d]thiazol-3(2H)-yl)propanoate (65 mg, 0.18 mmol) prepared in Step 3 in methanol (2.0 mL) was added a 10% sodium hydroxide solution (1.0 mL). The reaction mixture was stirred at room temperature overnight. The reaction mixture was evaporated, diluted with water, and then washed with ether. The aqueous layer was acidified with an aqueous 1N HCl solution. The resulting solid was filtered, washed with water, and then dried under reduced pressure to give 51 mg of the titled compound as a white solid (Yield: 79%).

¹H NMR (DMSO-d₆, 400 MHz) δ 12.83-13.07 (m, 1H), 8.19 (d, 2H), 7.91 (d, 1H), 7.74 (d, 1H), 7.52 (t, 1H), 7.31-7.37 (m, 3H), 5.76-5.78 (m, 1H), 4.89 (d, 1H), 4.58-4.68 (m, 2H), 2.40 (s, 3H)

### Example 255

### 3-hydroxy-2-(2-(3-(trifluoromethyl)benzoylimino)benzo[d]thiazol-3(2H)-yl)propionic acid

### Step 1: methyl 3-hydroxy-2-(2-((3-(trifluoromethyl)benzoyl)imino)benzo[d]thiazol-3(2H)-yl)propanoate

To a solution of N-(benzo[d]thiazol-2-yl)-3-(trifluoromethyl)benzamide (100 mg, 0.31 mmol) in N,N-dimethylformamide (1.0 mL) were added potassium carbonate (128 mg, 0.93 mmol) and methyl 2-bromo-3-hydroxypropanoate (85 mg, 0.46 mmol) prepared in Step 2 of Example 254. The reaction mixture was stirred at 80° C. overnight and cooled to room temperature. The reaction mixture was quenched with water and then extracted with ethyl acetate. The organic layer was dried over anhydrous magnesium sulfate, filtered, and then evaporated. The residue was purified with silica gel column chromatography (n-hexaneethyl acetate=81-11) to give 43 mg of the titled compound as a white solid (Yield: 33%).

¹H NMR (CDCl₃, 400 MHz) δ 8.52 (m, 1H), 8.30 (m, 1H), 7.67 (d, 1H), 7.55 (d, 1H), 7.46-7.51 (m, 2H), 7.36 (t, 1H), 7.17-7.22 (m, 1H), 4.93-4.97 (m, 1H), 4.82-4.90 (m, 1H), 4.56-4.61 (m, 1H), 3.67 (d, 3H)

### Step 2: 3-hydroxy-2-(2-(3-(trifluoromethyl)benzoylimino)benzo[d]thiazol-3(2H)-yl)propionic acid

To a solution of methyl 3-hydroxy-2-(2-((3-(trifluoromethyl)benzoyl)imino)benzo[d]thiazol-3(2H)-yl)propanoate (43 mg, 0.10 mmol) prepared in Step 1 in methanol (2.0 mL) was added a 10% sodium hydroxide solution (0.5 mL). The reaction mixture was stirred at room temperature overnight. The reaction mixture was evaporated, diluted with water, and then washed with ether. The aqueous layer was acidified with an aqueous 1N HCl solution. The resulting solid was filtered, washed with water, and then dried under reduced pressure to give 22 mg of the titled compound as a white solid (Yield: 54%).

¹H NMR (MeOD, 400 MHz) δ 8.68 (s, 1H), 8.62 (d, 1H), 7.75-7.85 (m, 3H), 7.68 (t, 1H), 7.54 (t, 1H), 7.38 (t, 1H), 5.11 (dd, 1H), 4.85 (m, 1H), 4.68 (dd, 1H)

### Example 256

### 2-(2-(3-chlorobenzoylimino)benzo[d]thiazol-3(2H)-yl)-3-hydroxypropionic acid

### Step 1: methyl 2-(2-((3-chlorobenzoyl)imino)benzo[d]thiazol-3(2H)-yl)-3-hydroxypropanoate

To a solution of N-(benzo[d]thiazol-2-yl)-3-chlorobenzamide (100 mg, 0.35 mmol) in N,N-dimethylformamide (1.0 mL) were added potassium carbonate (145 mg, 1.05 mmol) and methyl 2-bromo-3-hydroxypropanoate (96 mg, 0.52 mmol) prepared in Step 2 of Example 254. The reaction mixture was stirred at 80° C. overnight and cooled to room temperature. The reaction mixture was quenched with water and then extracted with ethyl acetate. The organic layer was dried over anhydrous magnesium sulfate, filtered, and then evaporated. The residue was purified with silica gel column chromatography (n-hexaneethyl acetate=81-11) to give 36 mg of the titled compound as a white solid (Yield: 26%).

¹H NMR (CDCl₃, 400 MHz) δ 8.22 (m, 1H), 8.09-8.11 (m, 1H), 7.56-7.61 (m, 1H), 7.47 (d, 1H), 7.38-7.42 (m, 2H), 7.32 (t, 1H), 7.24 (t, 1H), 4.93 (dd, 1H), 4.77-4.81 (m, 1H), 4.62 (dd, 1H), 3.68 (s, 3H)

### Step 2: 2-(2-(3-chlorobenzoylimino)benzo[d]thiazol-3(2H)-yl)-3-hydroxypropionic acid

To a solution of methyl 2-(2-((3-chlorobenzoyl)imino)benzo[d]thiazol-3(2H)-yl)-3-hydroxypropanoate (36 mg, 0.09 mmol) prepared in Step 1 in methanol (2.0 mL) was added a 10% sodium hydroxide solution (0.5 mL). The reaction mixture was stirred at room temperature overnight. The reaction mixture was evaporated, diluted with water, and then washed with ether. The aqueous layer was acidified with an aqueous 1N HCl. The resulting solid was filtered, washed with water, and then dried under reduced pressure to give 14 mg of the titled compound as a white solid (Yield: 41%).

¹H NMR (MeOD, 400 MHz) δ 8.34 (s, 1H), 8.27 (d, 1H), 7.75-7.81 (m, 2H), 7.51-7.55 (m, 2H), 7.46 (t, 1H), 7.37 (t, 1H), 5.06 (dd, 1H), 4.84 (m, 1H), 4.69 (dd, 1H)

### Example 257

### 2-(2-(4-methylbenzoylimino)benzo[d]thiazol-3(2H)-yl)-3-phenylpropionic acid

### Step 1: methyl 2-bromo-3-phenylpropanoate

To a solution of D,L-phenylalanine (5.0 g, 30.3 mmol), a 48 w/w % bromic acid solution (8.3 mL, 69.7 mmol), and potassium bromide (11.9 g, 100.0 mmol) in water (44.0 mL) was added portionwise sodium nitrite (6.0 g, 80.9 mmol) at −10° C. The reaction mixture was stirred at room temperature overnight. The reaction mixture was saturated with sodium chloride and extracted with ethyl acetate. The organic layer was dried over anhydrous magnesium sulfate, filtered, and then evaporated. The residue was dissolved in methanol (60.0 mL) and then 48 w/w % bromic acid (0.5 mL) was added thereto. The reaction mixture was stirred at 65° C. for 2 days and then concentrated under reduced pressure to discard excess methanol. The resulting dark yellow residue was dissolved in dichloromethane (100.0 mL) and washed with a saturated sodium hydrogen carbonate solution (50.0 mL) and brine. The organic layer was dried over anhydrous sodium sulfate, filtered, and then evaporated. The residue was dried under reduced pressure to give 6.5 g of the titled compound (Yield: 88%).

¹H NMR (CDCl₃, 400 MHz) δ 7.25-7.32 (m, 3H), 7.20 (d, 2H), 4.40 (t, 1H), 3.71 (s, 3H), 3.46 (dd, 1H), 3.24 (dd, 1H)

### Step 2: 2-(2-((4-methylbenzoyl)imino)benzo[d]thiazol-3(2H)-yl)-3-phenylpropionic acid

To a solution of N-(benzo[d]thiazol-2-yl)-4-methylbenzamide (100 mg, 0.37 mmol) prepared in Step 1 of Example 254 in N,N-dimethylformamide (1.0 mL) were added potassium carbonate (153 mg, 1.11 mmol) and methyl 2-bromo-3-phenylpropanoate (135 mg, 0.56 mmol). The reaction mixture was stirred at 80° C. overnight and cooled to room temperature. The reaction mixture was quenched with water and then extracted with ethyl acetate. The organic layer was dried over anhydrous magnesium sulfate, filtered, and then evaporated. The residue was purified with silica gel column chromatography (n-hexaneethyl acetate=8/1˜1/1). Methanol (2.0 mL) and a 10% sodium hydroxide solution (0.5 mL) were added to the resulting product. The reaction mixture was stirred at room temperature overnight and then concentrated under reduced pressure. The residue was diluted with water, washed with ether, and then acidified with an aqueous 1N HCl solution. The aqueous layer was saturated with sodium chloride and extracted with acetonitrile. The organic layer was dried over anhydrous magnesium sulfate, filtered, and then evaporated. The residue was purified with Prep. TLC (dichloromethanemethanol=10/1) to give 11 mg of the titled compound as a white solid (Yield: 7%).

¹H NMR (MeOD, 400 MHz) δ 8.18 (m, 2H), 7.64 (d, 1H), 7.20-7.31 (m, 5H), 6.92-6.98 (m, 5H), 5.82 (m, 1H), 3.72-3.89 (m, 2H), 2.42 (s, 3H)

### Example 258

### 3-phenyl-2-(2-(3-(trifluoromethyl)benzoylimino)benzo[d]thiazol-3(2H)-yl)propionic acid

The titled compound as a white solid was prepared in accordance with the same procedures as in Example 255, using methyl 2-bromo-3-phenylpropanoate, instead of methyl 2-bromo-3-hydroxypropanoate (Yield: 10%).

¹H NMR (MeOD, 400 MHz) δ 8.52-8.57 (m, 2H), 7.85 (d, 1H), 7.68-7.70 (m, 2H), 7.24-7.38 (m, 3H), 6.96-6.98 (m, 5H), 5.90 (m, 1H), 3.75-3.85 (m, 2H)

### Example 259

### 2-(2-(3-chlorobenzoylimino)benzo[d]thiazol-3(2H)-yl)-3-phenylpropionic acid

The titled compound as a white solid was prepared in accordance with the same procedures as in Example 256, using methyl 2-bromo-3-phenylpropanoate, instead of methyl 2-bromo-3-hydroxypropanoate (Yield: 9%).

¹H NMR (MeOD, 400 MHz) δ 8.20-8.25 (m, 2H), 7.68 (m, 1H), 7.55 (d, 1H), 7.47 (m, 1H), 7.24-7.29 (m, 3H), 6.97-6.98 (m, 5H), 6.07 (m, 1H), 3.75 (m, 2H)

### Example 260

### 2-(2-(3-chlorobenzoylimino)benzo[d]thiazol-3(2H)-yl)octanoic acid

The titled compound as a white solid was prepared in accordance with the same procedures as in Example 256, using ethyl 2-bromooctanoate, instead of methyl 2-bromo-3-hydroxypropanoate (Yield: 7%).

¹H NMR (DMSO-d₆, 400 MHz) δ 8.20 (s, 1H), 8.15 (d, 1H), 7.99 (d, 1H), 7.86 (m, 1H), 7.67 (d, 1H), 7.54-7.58 (m, 2H), 7.41 (t, 1H), 5.78 (m, 1H), 2.31-2.51 (m, 2H), 1.25 (m, 4H), 1.10 (m, 4H), 0.72 (t, 3H)

### Example 261

### 2-(2-(3-chlorobenzoylimino)benzo[d]thiazol-3(2H)-yl)-3-methylbutanoic acid

The titled compound as a white solid was prepared in accordance with the same procedures as in Example 256, using ethyl 2-bromo-3-methylbutyrate, instead of methyl 2-bromo-3-hydroxypropanoate (Yield: 12%).

¹H NMR (DMSO-d₆, 400 MHz) δ 8.21 (s, 1H), 8.15 (d, 1H), 7.99 (d, 1H), 7.94-7.98 (m, 1H), 7.67 (d, 1H), 7.55-7.60 (m, 2H), 7.42 (t, 1H), 5.27 (m, 1H), 3.05 (m, 1H), 1.35 (d, 3H), 0.58 (d, 3H)

### Example 262

### 2-(2-(3-(trifluoromethyl)benzoylimino)benzo[d]thiazol-3(2H)-yl)octanoic acid

The titled compound as a white solid was prepared in accordance with the same procedures as in Example 255, using ethyl 2-bromooctanoate, instead of methyl 2-bromo-3-hydroxypropanoate (Yield: 15%).

¹H NMR (DMSO-d₆, 400 MHz) δ 8.50 (s, 1H), 8.47 (d, 2H), 7.96-8.02 (m, 2H), 7.86 (m, 1H), 7.78 (t, 1H), 7.58 (t, 1H), 7.42 (t, 1H), 5.80 (m, 1H), 2.32-2.51 (m, 2H), 1.25-1.26 (m, 4H), 1.09 (m, 4H), 0.70 (t, 3H)

### Example 263

### 3-methyl-2-(2-(3-(trifluoromethyl)benzoylimino)benzo[d]thiazol-3(2H)-yl)butanoic acid

The titled compound as a white solid was prepared in accordance with the same procedures as in Example 255, using ethyl 2-bromo-3-methylbutyrate, instead of methyl 2-bromo-3-hydroxypropanoate (Yield: 16%).

¹H NMR (DMSO-d₆, 400 MHz) δ 8.54 (s, 1H), 8.50 (d, 1H), 7.96-8.02 (m, 3H), 7.79 (t, 1H), 7.59 (t, 1H), 7.43 (t, 1H), 5.31 (m, 1H), 3.07 (m, 1H), 1.35 (d, 3H), 0.59 (d, 3H)

### Example 264

### 2-(2-(3-chlorobenzoylimino)benzo[d]thiazol-3(2H)-yl)-2-phenylacetic acid

The titled compound as a yellow solid was prepared in accordance with the same procedures as in Example 256, using methyl 2-bromo-2-phenylacetate, instead of methyl 2-bromo-3-hydroxypropanoate (Yield: 8%).

¹H NMR (MeOD, 400 MHz) δ 8.23 (m, 2H), 7.41-7.71 (m, 6H), 7.21-7.28 (m, 6H)

### Example 265

### 2-phenyl-2-(2-(3-(trifluoromethyl)benzoylimino)benzo[d]thiazol-3(2H)-yl)acetic acid

The titled compound as a white solid was prepared in accordance with the same procedures as in Example 255, using methyl 2-bromo-2-phenylacetate, instead of methyl 2-bromo-3-hydroxypropanoate (Yield: 15%).

¹H NMR (MeOD, 400 MHz) δ 8.52-8.54 (m, 2H), 7.82 (d, 1H), 7.75 (d, 1H), 7.65 (t, 1H), 7.54 (d, 3H), 7.23-7.32 (m, 6H)

### Example 266

### 2-(7-(trifluoromethyl)-2-(3-(trifluoromethyl)benzoylimino)benzo[d]thiazol-3(2H)-yl)acetic acid

### Step 1: N-((2-fluoro-3-(trifluoromethyl)phenyl)carbamothionyl)-3-(trifluoromethyl)benzamide

To a solution of ammonium thiocyanate (512 mg, 6.7 mmol) in acetone (6.2 mL) was added dropwise 3-(trifluoromethyl)benzoyl chloride (911 uL, 6.2 mmol) over 5 minutes. The reaction mixture was refluxed under stirring for 15 minutes and then a solution of 2-fluoro-3-(trifluoromethyl)aniline (1.0 g, 5.6 mmol) in acetone (5.0 mL) was added thereto. The reaction mixture was refluxed under stirring for 1 hour, poured into ice water, and then filtered. The resulting solid was washed with water and then dried under reduced pressure to give 2.2 g of the titled compound as a yellow solid (Yield: 96%).

¹H NMR (CDCl₃, 400 MHz) δ 12.73 (s, 1H), 9.15 (s, 1H), 8.71 (t, 1H), 8.19 (s, 1H), 8.09 (d, 1H), 7.94 (d, 1H), 7.73 (t, 1H), 7.53 (t, 1H), 7.32 (t, 1H)

### Step 2: 3-(trifluoromethyl)-N-(7-(trifluoromethyl)benzo[d]thiazol-2-yl)benzamide

To a solution of N-((2-fluoro-3-(trifluoromethyl)phenyl)carbamothionyl)-3-(trifluoromethyl)benzamide (2.2 g, 5.4 mmol) prepared in Step 1 in N,N-dimethylformamide (20.0 mL) was added sodium hydride (60%, 1.1 g, 27.0 mmol) at 0° C. The reaction mixture was stirred at room temperature for 4 hours. The reaction mixture was quenched with water and then extracted with ethyl acetate. The organic layer was washed with brine, dried over anhydrous magnesium sulfate, filtered, and then evaporated. Ether and n-hexane was added to the resulting residue and then filtered. The resulting solid was dried under reduced pressure to give 1.5 g of the titled compound as a yellow solid (Yield: 71%).

¹H NMR (CDCl₃, 400 MHz) δ 8.27 (s, 1H), 8.20 (d, 1H), 7.87 (d, 2H), 7.61-7.71 (m, 3H), 7.47 (t, 1H)

### Step 3: ethyl 2-(7-(trifluoromethyl)-2-((3-(trifluoromethyl)benzoyl)imino)benzo[d]thiazol-3(2H)-yl)acetate

To a solution of 3-(trifluoromethyl)-N-(7-(trifluoromethyl)benzo[d]thiazol-2-yl)benzamide (200 mg, 0.51 mmol) prepared in Step 2 in N,N-dimethylformamide (2.0 mL), were added potassium carbonate (211 mg, 1.53 mmol) and ethyl bromoacetate (85 uL, 0.76 mmol). The reaction mixture was stirred at 80° C. overnight. The reaction mixture was quenched with water and then extracted with ethyl acetate. The organic layer was washed with brine, dried over anhydrous magnesium sulfate, filtered, and then evaporated. The residue was washed with isopropyl ether. The resulting solid was filtered and then dried under reduced pressure to give 182 mg of the titled compound as a white solid (Yield: 75%).

¹H NMR (CDCl₃, 400 MHz) δ 8.59 (s, 1H), 8.49 (d, 1H), 7.79 (d, 1H), 7.57-7.64 (m, 3H), 7.44 (d, 1H), 5.26 (s, 2H), 4.30 (q, 2H), 1.30 (t, 3H)

### Step 4: 2-(7-(trifluoromethyl)-2-(3-(trifluoromethyl)benzoylimino)benzo[d]thiazol-3(2H)-yl)acetic acid

To a solution of ethyl 2-(7-(trifluoromethyl)-2-((3-(trifluoromethyl)benzoyl)imino)benzo[d]thiazol-3(2H)-yl)acetate (182 mg, 0.38 mmol) prepared in Step 3 in methanol (3.0 mL) was added a 10% sodium hydroxide solution (1.0 mL). The reaction mixture was stirred at room temperature overnight. The reaction mixture was evaporated, diluted with water, and then washed with ether. The aqueous layer was acidified with an aqueous 1N HCl solution. The resulting solid was filtered, washed with water, and then dried under reduced pressure to give 162 mg of the titled compound as a white solid (Yield: 95%).

¹H NMR (DMSO-d₆, 400 MHz) δ 8.50 (d, 1H), 8.44 (s, 1H), 8.00 (d, 1H), 7.93 (d, 1H), 7.70-7.80 (m, 3H), 5.26 (s, 2H)

### Example 267

### 2-(7-(trifluoromethyl)-2-(3-(trifluoromethyl)benzoylimino)benzo[d]thiazol-3(2H)-yl)propionic acid

The titled compound as a pale yellow solid was prepared in accordance with the same procedures as in Example 266, using ethyl 2-bromopropionate, instead of ethyl bromoacetate (Yield: 36%).

¹H NMR (DMSO-d₆, 400 MHz) δ 13.25 (bs, 1H), 8.53 (s, 1H), 8.49 (d, 1H), 8.19-8.21 (m, 1H), 8.01 (d, 1H), 7.79-7.84 (m, 3H), 6.01-6.02 (m, 1H), 1.77 (d, 3H)

### Example 268

### 2-(7-(trifluoromethyl)-2-(3-(trifluoromethyl)benzoylimino)benzo[d]thiazol-3(2H)-yl)butanoic acid

The titled compound as a pale yellow solid was prepared in accordance with the same procedures as in Example 266, using methyl 2-bromobutyrate, instead of ethyl bromoacetate (Yield: 39%).

¹H NMR (DMSO-d₆, 400 MHz) δ 13.13-13.25 (m, 1H), 8.51 (s, 1H), 8.48 (d, 1H), 8.23 (m, 1H), 8.01 (d, 1H), 7.79-7.84 (m, 3H), 5.90-5.94 (m, 1H), 2.37-2.51 (m, 2H), 0.82 (t, 3H)

### Example 269

### 2-(2-(3-chlorobenzoylimino)-7-(trifluoromethyl)benzo[d]thiazol-3(2H)-yl)acetic acid

The titled compound as a white solid was prepared in accordance with the same procedures as in Example 266, using 3-chlorobenzoyl chloride, instead of 3-(trifluoromethyl)benzoyl chloride (Yield: 42%).

¹H NMR (DMSO-d₆, 400 MHz) δ 13.54 (bs, 1H), 8.23 (s, 1H), 8.20 (d, 1H), 8.13 (d, 1H), 7.77-7.83 (m, 2H), 7.21 (d, 1H), 7.58-7.62 (m, 1H), 5.44 (s, 2H)

### Example 270

### 2-(2-(3-chlorobenzoylimino)-7-(trifluoromethyl)benzo[d]thiazol-3(2H)-yl)propionic acid

The titled compound as a pale yellow solid was prepared in accordance with the same procedures as in Example 266, using 3-chlorobenzoyl chloride and ethyl 2-bromopropionate, instead of 3-(trifluoromethyl)benzoyl chloride and ethyl bromoacetate, respectively (Yield: 35%).

¹H NMR (DMSO-d₆, 400 MHz) δ 13.26 (bs, 1H), 8.22 (s, 1H), 8.17 (d, 2H), 7.80-7.83 (m, 2H), 7.70 (d, 1H), 7.57-7.63 (m, 1H), 6.01 (m, 1H), 1.76 (d, 3H)

### Example 271

### 2-(2-(3-chlorobenzoylimino)-7-(trifluoromethyl)benzo[d]thiazol-3(2H)-yl)butanoic acid

The titled compound as a pale yellow solid was prepared in accordance with the same procedures as in Example 266, using 3-chlorobenzoyl chloride and methyl 2-bromobutyrate, instead of 3-(trifluoromethyl)benzoyl chloride and ethyl bromoacetate, respectively (Yield: 32%).

¹H NMR (DMSO-d₆, 400 MHz) δ 13.13-13.28 (m, 1H), 8.14-8.20 (m, 3H), 7.83 (m, 2H), 7.70 (d, 1H), 7.59 (t, 1H), 5.89 (m, 1H), 2.37-2.51 (m, 2H), 0.79 (t, 3H)

### Example 272

### 2-(2-(2-amino-4-fluorobenzoylimino)benzo[d]thiazol-3(2H)-yl)butanoic acid

### Step 1: ethyl 2-(2-((2-amino-4-fluorobenzoyl)imino)benzo[d]thiazol-3(2H)-yl)butanoate

To a solution of ethyl 2-(2-iminobenzo[d]thiazol-3(2H)-yl)butanoate (30 mg, 0.12 mmol) prepared in Preparation 18 in N,N-dimethylformamide (1.0 mL) were added 4-fluoroanthranilic acid (24.8 mg, 0.16 mmol), diisopropylethylamine (31 mg, 0.24 mmol), and (benzotriazol-1-yloxy)tris-(dimethylamino)phosphonium hexafluorophosphate (53 mg, 0.12 mmol). The reaction mixture was stirred at room temperature overnight. The reaction mixture was quenched with water and then extracted with ethyl acetate. The organic layer was washed with brine, dried over anhydrous sodium sulfate, filtered, and then evaporated. The residue was purified with silica gel column chromatography (n-hexaneethyl acetate=10/1) to give 37.7 mg of the titled compound as a yellow solid (Yield: 81%).

¹H NMR (CDCl₃, 400 MHz) δ 8.27 (dd, 1H), 7.69 (d, 1H), 7.43 (dd, 1H), 7.26-7.32 (m, 2H), 6.40 (dd, 1H), 6.31-6.34 (m, 3H), 5.49 (brs, 1H), 3.66 (s, 3H), 2.44-2.53 (m, 2H), 0.90 (t, 3H).

### Step 2: 2-(2-(2-amino-4-fluorobenzoylimino)benzo[d]thiazol-3(2H)-yl)butanoic acid

To a solution of ethyl 2-(2-((2-amino-4-fluorobenzoyl)imino)benzo[d]thiazol-3(2H)-yl)butanoate (37.7 mg, 0.097 mmol) prepared in Step 1 in a mixed solvent of tetrahydrofuran and methanol (2/1, 3.0 mL) was added a 1N sodium hydroxide solution (1.0 mL). The reaction mixture was stirred at room temperature overnight. The reaction mixture was evaporated, diluted with water, and then washed with ether. The aqueous layer was acidified to pH 3-4 with an aqueous 1N HCl solution, added sodium chloride and extracted with dichloromethane. The organic layer was dried over anhydrous magnesium sulfate, filtered, and then evaporated. The residue was dried under reduced pressure to give 36 mg of the titled compound as a yellow solid (Yield: 99%).

¹H NMR (CD₃OD, 400 MHz) δ 8.31 (dd, 1H), 7.75 (d, 1H), 7.57 (d, 1H), 7.49 (dd, 1H), 7.31 (dd, 1H), 6.42 (d, 1H), 6.31 (dd, 1H), 5.62 (brs, 1H), 2.41-2.57 (m, 2H), 0.84 (t, 3H).

### Example 273

### 2-(2-(4-(benzyloxy)benzoylimino)benzo[d]thiazol-3(2H)-yl)butanoic acid

The titled compound as a white solid was prepared in accordance with the same procedures as in Example 272, using 4-benzyloxybenzoic acid, instead of 4-fluoroanthranilic acid (Yield: 90%).

¹H NMR (CD₃OD, 400 MHz) δ 8.23 (d, 2H), 7.75 (d, 1H), 7.59 (d, 1H), 7.48 (dd, 1H), 7.43 (d, 2H), 7.27-7.37 (m, 4H), 7.04 (d, 2H), 5.68 (brs, 1H), 5.12 (s, 2H), 2.45-2.56 (m, 2H), 0.83 (t, 3H)

### Example 274

### 2-(2-(2-amino-4-fluorobenzoylimino)benzo[d]thiazol-3(2H)-yl)propionic acid

The titled compound as a yellow solid was prepared in accordance with the same procedures as in Example 272, using ethyl 2-(2-iminobenzo[d]thiazol-3(2H)-yl)propionate prepared in Preparation 19, instead of ethyl 2-(2-iminobenzo[d]thiazol-3(2H)-yl)butanoate and, respectively (Yield: 78%).

¹H NMR (CD₃OD, 400 MHz) δ 8.33 (dd, 1H), 7.73 (d, 1H), 7.55 (d, 1H), 7.47 (dd, 1H), 7.30 (dd, 1H), 6.41 (d, 1H), 6.31 (dd, 1H), 5.71 (brd, 1H), 1.78 (d, 3H).

### Example 275

### 2-(2-(4-(benzyloxy)benzoylimino)benzo[d]thiazol-3(2H)-yl)propionic acid

The titled compound as a white solid was prepared in accordance with the same procedures as in Example 272, using ethyl 2-(2-iminobenzo[d]thiazol-3(2H)-yl)propionate prepared in Preparation 19 and 4-benzyloxybenzoic acid, instead of ethyl 2-(2-iminobenzo[d]thiazol-3(2H)-yl)butanoate and 4-fluoroanthranilic acid, respectively (Yield: 68%).

¹H NMR (DMSO-d₆, 400 MHz) δ 8.19 (d, 2H), 7.94 (d, 1H), 7.77 (d, 1H), 7.55 (dd, 1H), 7.36-7.50 (m, 6H), 7.12 (d, 2H), 5.87 (brs, 1H), 5.22 (s, 2H), 1.71 (d, 3H).

### Example 276

### 2-(2-(3-fluorobenzoylimino)benzo[d]thiazol-3(2H)-yl)propionic acid

### Step 1: ethyl 2-(2-((3-fluorobenzoyl)imino)benzo[d]thiazol-3(2H)-yl)propionate

To a solution of ethyl 2-(2-iminobenzo[d]thiazol-3(2H)-yl)propionate (30 mg, 0.12 mmol) prepared in Preparation 19 in tetrahydrofuran (1 mL) were added diisopropylethylamine (31 mg, 0.24 mmol) and 3-fluorobenzoyl chloride (28.5 mg, 0.18 mmol). The reaction mixture was stirred at room temperature overnight. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=10/1) to give 32.6 mg of the titled compound as a white solid (Yield: 73%).

¹H NMR (CDCl₃, 400 MHz) δ 8.11 (d, 1H), 8.00 (d, 1H), 7.74 (d, 1H), 7.20-7.50 (m, 5H), 5.67 (brs, 1H), 4.11-4.23 (m, 2H), 1.86 (d, 3H), 1.09 (t, 3H).

### Step 2: 2-(2-(3-fluorobenzoylimino)benzo[d]thiazol-3(2H)-yl)propionic acid

To a solution of ethyl 2-(2-((3-fluorobenzoyl)imino)benzo[d]thiazol-3(2H)-yl)propionate (32.6 mg, 0.088 mmol) prepared in Step 1 in a mixed solvent of tetrahydrofuran and methanol (21, 3.0 mL) was added a 1N sodium hydroxide solution (1.0 mL). The reaction mixture was stirred at room temperature overnight and then concentrated under reduced pressure. The residue was diluted with water and then washed with ether. The aqueous layer was acidified to pH 3-4 with an aqueous 1N HCl solution. Sodium chloride was added to the solution, which was then extracted with dichloromethane. The organic layer was dried over anhydrous magnesium sulfate, filtered, and then evaporated. The residue was dried under reduced pressure to give 30.3 mg of the titled compound as a yellow solid (Yield: 100%).

¹H NMR (CD₃OD, 400 MHz) d8.10 (d, 1H), 7.95 (d, 1H), 7.79 (d, 1H), 7.54 (d, 2H), 7.44-7.50 (m, 1H), 7.38-7.41 (m, 1H), 7.25 (dd, 1H), 5.84 (brs, 1H), 1.86 (d, 3H).

### Example 277

### 2-(2-(4-butylbenzoylimino)benzo[d]thiazol-3(2H)-yl)butanoic acid

The titled compound as a white solid was prepared in accordance with the same procedures as in Example 276, using ethyl 2-(2-iminobenzo[d]thiazol-3(2H)-yl)butanoate prepared in Preparation 18 and 4-n-butylbenzoyl chloride, instead of ethyl 2-(2-iminobenzo[d]thiazol-3(2H)-yl)propionate and 3-fluorobenzoyl chloride, respectively (Yield: 81%).

¹H NMR (CDCl₃, 400 MHz) δ 8.14 (d, 2H), 7.59 (brs, 1H), 7.18-7.26 (m, 5H), 5.65 (brs, 1H), 2.62 (dd, 2H), 2.37 (brs, 2H), 1.54-1.60 (m, 2H), 1.30-1.37 (m, 2H), 0.91 (t, 3H), 0.78 (brs, 3H).

### Example 278

### 2-(2-(3-fluorobenzoylimino)benzo[d]thiazol-3(2H)-yl)acetic acid

The titled compound as a white solid was prepared in accordance with the same procedures as in Example 276, using ethyl 2-(2-iminobenzo[d]thiazol-3(2H)-yl)acetate prepared in Preparation 20, instead of ethyl 2-(2-iminobenzo[d]thiazol-3(2H)-yl)propionate (Yield: 70%).

¹H NMR (CD₃OD, 400 MHz) d8.10 (d, 1H), 7.97 (d, 1H), 7.77 (d, 1H), 7.37-7.55 (m, 4H), 7.24 (dd, 1H), 5.31 (s, 2H).

### Example 279

### 2-(2-(4-butylbenzoylimino)benzo[d]thiazol-3(2H)-yl)propionic acid

The titled compound as a white solid was prepared in accordance with the same procedures as in Example 276, using 4-n-butylbenzoyl chloride, instead of 3-fluorobenzoyl chloride (Yield: 34%).

¹H NMR (CD₃OD, 400 MHz) d8.19 (d, 2H), 7.79 (d, 1H), 7.60 (d, 1H), 7.50 (dd, 1H), 7.35 (dd, 1H), 7.28 (d, 2H), 5.89 (brs, 1H), 2.68 (dd, 2H), 1.82 (d, 3H), 1.60-1.67 (m, 2H), 1.35-1.42 (m, 2H), 0.95 (t, 3H).

### Example 280

### 2-(2-(3-chlorobenzoylimino)-4-methoxybenzo[d]thiazol-3(2H)-yl)acetic acid

The titled compound as a white solid was prepared in accordance with the same procedures as in Example 200, using 4-methoxybenzo[d]thiazol-2-amine and 3-chlorobenzoyl chloride, instead of 4,6-difluorobenzo[d]thiazol-2-amine and p-toluoyl chloride, respectively (Yield: 45%).

¹H NMR (DMSO-d₆, 400 MHz) δ 8.15-8.17 (m, 2H), 7.63 (d, 1H), 7.51-7.54 (m, 2H), 7.34 (t, 1H), 7.17 (d, 1H), 5.52 (s, 2H), 3.89 (s, 3H)

### Example 281

### 2-(4-methoxy-2-(3-(trifluoromethyl)benzoylimino)benzo[d]thiazol-3(2H)-yl)acetic acid

The titled compound as a white solid was prepared in accordance with the same procedures as in Example 200, using 4-methoxybenzo[d]thiazol-2-amine and 3-(trifluoromethyl)benzoyl chloride, instead of 4,6-difluorobenzo[d]thiazol-2-amine and p-toluoyl chloride, respectively (Yield: 40%).

¹H NMR (DMSO-d₆, 400 MHz) δ 8.53 (d, 1H), 8.47 (s, 1H), 7.97 (d, 1H), 7.78 (t, 1H), 7.57 (d, 1H), 7.36 (t, 1H), 7.20 (d, 1H), 5.58 (s, 2H), 3.92 (s, 3H)

### Example 282

### 2-(2-(3-chlorobenzoylimino)-4-methoxybenzo[d]thiazol-3(2H)-yl)propionic acid

The titled compound as a white solid was prepared in accordance with the same procedures as in Example 200, using 4-methoxybenzo[d]thiazol-2-amine, 3-chlorobenzoyl chloride, and ethyl 2-bromopropionate, instead of 4,6-difluorobenzo[d]thiazol-2-amine, p-toluoyl chloride, and ethyl bromoacetate, respectively (Yield: 38%).

¹H NMR (DMSO-d₆, 400 MHz) δ 12.97-12.98 (m, 1H), 8.22 (m, 2H), 7.66 (d, 1H), 7.56 (d, 2H), 7.37 (t, 1H), 7.23 (d, 1H), 6.49-6.53 (m, 1H), 3.94 (bs, 3H), 1.74 (d, 3H)

### Example 283

### 2-(4-methoxy-2-(3-(trifluoromethyl)benzoylimino)benzo[d]thiazol-3(2H)-yl)propionic acid

The titled compound as a white solid was prepared in accordance with the same procedures as in Example 200, using 4-methoxybenzo[d]thiazol-2-amine, 3-(trifluoromethyl)benzoyl chloride, and ethyl 2-bromopropionate, instead of 4,6-difluorobenzo[d]thiazol-2-amine, p-toluoyl chloride, and ethyl bromoacetate, respectively (Yield: 39%).

¹H NMR (MeOD, 400 MHz) δ 8.53 (m, 2H), 7.83 (d, 1H), 7.68 (t, 1H), 7.41 (d, 1H), 7.34 (t, 1H), 7.16 (d, 1H), 6.57 (m, 1H), 3.97 (s, 3H), 1.85 (bs, 3H)

### Example 284

### 2-(2-(3-chlorobenzoylimino)-4-methoxybenzo[d]thiazol-3(2H)-yl)butanoic acid

The titled compound as a white solid was prepared in accordance with the same procedures as in Example 200, using 4-methoxybenzo[d]thiazol-2-amine, 3-chlorobenzoyl chloride, and methyl 2-bromobutyrate, instead of 4,6-difluorobenzo[d]thiazol-2-amine, p-toluoyl chloride, and ethyl bromoacetate, respectively (Yield: 42%).

¹H NMR (DMSO-d₆, 400 MHz) δ 8.07-8.26 (m, 2H), 7.67 (d, 1H), 7.54-7.58 (m, 2H), 7.39 (t, 1H), 7.24 (m, 1H), 6.47-6.66 (m, 1H), 3.59-3.67 (m, 3H), 2.08-2.45 (m, 2H), 0.80 (m, 3H)

### Example 285

### 2-(4-methoxy-2-(3-(trifluoromethyl)benzoylimino)benzo[d]thiazol-3(2H)-yl)butanoic acid

The titled compound as a white solid was prepared in accordance with the same procedures as in Example 200, using 4-methoxybenzo[d]thiazol-2-amine, 3-(trifluoromethyl)benzoyl chloride, and methyl 2-bromobutyrate, instead of 4,6-difluorobenzo[d]thiazol-2-amine, p-toluoyl chloride, and ethyl bromoacetate, respectively (Yield: 36%).

¹H NMR (DMSO-d₆, 400 MHz) δ 8.39-8.63 (m, 2H), 7.97 (d, 1H), 7.78 (t, 1H), 7.59 (d, 1H), 7.40 (t, 1H), 7.25 (m, 1H), 6.48-6.67 (m, 1H), 3.56-3.67 (m, 3H), 2.07-2.45 (m, 2H), 0.82 (t, 3H)

### Example 286

### 2-(5,6-difluoro-2-((4-methylbenzoyl)imino)benzo[d]thiazol-3(2H)-yl)acetic acid

The titled compound as a white solid was prepared in accordance with the same procedures as in Example 52, using 5,6-difluorobenzo[d]thiazol-2-amine prepared in Preparation 3 and p-toluoyl chloride, instead of 6-methoxybenzo[d]thiazol-2-amine and 3-chlorobenzoyl chloride, respectively (Yield: 32%).

¹H NMR (MeOD, 400 MHz): 8.15 (d, 2H), 7.83-7.77 (m, 1H), 7.66-7.61 (m, 1H), 7.28 (d, 2H), 5.29 (s, 2H), 2.41 (s, 3H)

### Example 287

### 2-(5,6-difluoro-2-((4-methylbenzoyl)imino)benzo[d]thiazol-3(2H)-yl)propionic acid

The titled compound as a white solid was prepared in accordance with the same procedures as in Example 52, using 5,6-difluorobenzo[d]thiazol-2-amine prepared in Preparation 3, p-toluoyl chloride, and ethyl 2-bromopropionate, instead of 6-methoxybenzo[d]thiazol-2-amine, 3-chlorobenzoyl chloride, and ethyl bromoacetate, respectively (Yield: 26%).

¹H NMR (MeOD, 400 MHz): 8.15 (d, 2H), 7.85-7.79 (m, 1H), 7.74-7.69 (m, 1H), 7.28 (d, 2H), 5.69 (m, 1H), 2.41 (s, 3H), 1.82 (d, 3H)

### Example 288

### 2-(2-((3-chlorobenzoyl)imino)-5,6-difluorobenzo[d]thiazol-3(2H)-yl)acetic acid

The titled compound as a white solid was prepared in accordance with the same procedures as in Example 52, using 5,6-difluorobenzo[d]thiazol-2-amine prepared in Preparation 3, instead of 6-methoxybenzo[d]thiazol-2-amine (Yield: 23%).

¹H NMR (MeOD, 400 MHz): 8.22 (s, 1H), 8.18 (d, 1H), 7.83 (m, 1H), 7.68 (m, 1H), 7.55 (m, 1H), 7.46 (m, 1H), 5.30 (s, 2H), 2.4 (s, 3H)

### Example 289

### 2-(2-((3-chlorobenzoyl)imino)-5,6-difluorobenzo[d]thiazol-3(2H)-yl)propionic acid

The titled compound as a white solid was prepared in accordance with the same procedures as in Example 52, using 5,6-difluorobenzo[d]thiazol-2-amine prepared in Preparation 3 and ethyl 2-bromopropionate, instead of 6-methoxybenzo[d]thiazol-2-amine and ethyl bromoacetate, respectively (Yield: 30%).

¹H NMR (MeOD, 400 MHz); 8.24 (s, 1H), 8.17 (d, 1H), 7.84 (m, 1H), 7.76 (m, 1H), 7.55 (m, 1H), 7.46 (m, 1H), 5.69 (m, 1H), 1.83 (s, 3H)

### Example 290

### 2-(2-((3-chlorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine prepared in Preparation 3 and methyl 2-bromobutyrate, instead of 6-methoxybenzo[d]thiazol-2-amine and ethyl bromoacetate, respectively (Yield: 32%).

¹H NMR (MeOD, 400 MHz): 8.23 (s, 1H), 8.18 (d, 1H), 7.85 (m, 1H), 7.74 (m, 1H), 7.56 (m, 1H), 7.47 (m, 1H), 5.66 (m, 1H), 2.60-2.40 (m, 2H), 0.86 (t, 3H)

### Example 291

### 2-(5,6-difluoro-2-((3-(trifluoromethyl)benzoyl)imino)benzo[d]thiazol-3(2H)-yl)acetic acid

The titled compound as a white solid was prepared in accordance with the same procedures as in Example 52, using 5,6-difluorobenzo[d]thiazol-2-amine prepared in Preparation 3 and 3-(trifluoromethyl)benzoyl chloride, instead of 6-methoxybenzo[d]thiazol-2-amine and 3-chlorobenzoyl chloride, respectively (Yield: 22%).

¹H NMR (MeOD, 400 MHz): 8.52 (m, 2H), 7.85 (m, 2H), 7.71 (m, 2H), 5.32 (s, 2H)

### Example 292

### 2-(5,6-difluoro-2-((3-(trifluoromethyl)benzoyl)imino)benzo[d]thiazol-3(2H)-yl)propionic acid

The titled compound as a white solid was prepared in accordance with the same procedures as in Example 52, using 5,6-difluorobenzo[d]thiazol-2-amine prepared in Preparation 3,3-(trifluoromethyl)benzoyl chloride, and ethyl 2-bromopropionate, instead of 6-methoxybenzo[d]thiazol-2-amine, 3-chlorobenzoyl chloride and ethyl bromoacetate, respectively (Yield: 28%).

¹H NMR (MeOD, 400 MHz): 8.55 (s, 1H), 8.50 (d, 1H), 7.83 (m, 2H), 7.78 (m, 1H), 7.69 (m, 1H), 5.69 (m, 1H), 1.84 (d, 3H)

### Example 293

### 2-(5,6-difluoro-2-((3-(trifluoromethyl)benzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine prepared in Preparation 3,3-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 16%).

¹H NMR (MeOD, 400 MHz): 8.54 (s, 1H), 8.49 (d, 1H), 7.95-7.75 (m, 3H), 7.69 (m, 1H), 5.61 (m, 1H), 2.70-2.40 (m, 2H), 0.88 (t, 3H)

### Example 294

### 2-(2-(3-(trifluoromethyl)benzoylimino)-4,5,6,7-tetrahydrobenzo[d]thiazol-3(2H)-yl)acetic acid

### Step 1: ethyl 2-(2-((3-(trifluoromethyl)benzoyl)imino)-4,5,6,7-tetrahydrobenzo[d]thiazol-3(2H)-yl)acetate

To a solution of 4,5,6,7-tetrahydrobenzo[d]thiazol-2-amine (200 mg, 1.297 mmol) in tetrahydrofuran (4.3 mL) were added 3-(trifluoromethyl)benzoyl chloride (324 mg, 1.56 mmol) and potassium carbonate (359 mg, 2.59 mmol). The reaction mixture was stirred at 80° C. for 5 hours and then concentrated under reduced pressure. To the resulting intermediate (0.43 mmol) were added N,N-dimethylformamide (1.5 mL) and ethyl bromoacetate (93 mg, 0.559 mmol). The reaction mixture was stirred at 80° C. for 3 hours and then cooled to room temperature. The reaction mixture was quenched with water and then extracted with ethyl acetate. The extract was dried over anhydrous magnesium sulfate, filtered, and then evaporated. The residue was purified with silica gel column chromatography (n-hexaneethyl acetate=41) to give 103 mg of the titled compound as a white solid (Yield: 58%).

¹H NMR (CDCl₃, 400 MHz) δ 12.40 (brs, 1H), 8.54 (s, 1H), 8.43 (d, 1H), 7.71 (d, 1H), 7.54 (dd, 1H), 4.90 (s, 2H), 4.24-4.30 (m, 2H), 2.59 (brs, 2H), 2.50 (brs, 2H), 1.87-1.92 (m, 4H), 1.30 (t, 3H).

### Step 2: 2-(2-(3-(trifluoromethyl)benzoylimino)-4,5,6,7-tetrahydrobenzo[d]thiazol-3(2H)-yl)acetic acid

A 1N sodium hydroxide solution (2.0 mL) was added to a solution of ethyl 2-(2-((3-(trifluoromethyl)benzoyl)imino)-4,5,6,7-tetrahydrobenzo[d]thiazol-3(2H)-yl)acetate (103 mg, 0.25 mmol) prepared in Step 1 in a mixed solvent of tetrahydrofuran and methanol (21, 6.0 mL). The reaction mixture was stirred at room temperature overnight, concentrated under reduced pressure, diluted with water, acidified to pH 3-4 with an aqueous 1N HCl solution, and then saturated with sodium chloride. The separated aqueous layer was extracted with dichloromethane three times. The combined organic layer was dried over anhydrous magnesium sulfate and then filtered. The filtrate was concentrated under reduced pressure to give 85 mg of the titled compound as a white solid (Yield: 89%).

¹H NMR (CD₃OD, 400 MHz) δ 8.50 (s, 1H), 8.44 (d, 1H), 7.76 (d, 1H), 7.61 (dd, 1H), 5.02 (s, 2H), 2.59-2.64 (m, 4H), 1.90-1.95 (m, 4H).

### Example 295

### 2-(2-(3-(trifluoromethyl)benzoylimino)-4,5,6,7-tetrahydrobenzo[d]thiazol-3(2H)-yl)propionic acid

The titled compound as a white solid was prepared in accordance with the same procedures as in Example 294, using ethyl 2-bromopropionate, instead of ethyl bromoacetate (Yield: 49%).

¹H NMR (CDCl₃, 400 MHz) δ 8.46 (s, 1H), 8.33 (d, 1H), 7.64 (d, 1H), 7.44 (dd, 1H), 4.92 (brs, 1H), 2.40-2.56 (m, 4H), 1.83-1.91 (m, 4H), 1.72 (d, 3H).

### Example 296

### 2-(2-(3-(trifluoromethyl)benzoylimino)-4,5,6,7-tetrahydrobenzo[d]thiazol-3(2H)-yl)butanoic acid

The titled compound as a white solid was prepared in accordance with the same procedures as in Example 294, using methyl 2-bromobutyrate, instead of ethyl bromoacetate (Yield: 52%).

¹H NMR (DMSO-d₆, 400 MHz) δ 8.44 (s, 1H), 8.38 (d, 1H), 7.89 (d, 1H), 7.72 (dd, 1H), 5.07 (brs, 1H), 2.44-2.64 (m, 5H), 2.20-2.26 (m, 1H), 1.78-1.86 (m, 4H), 0.77 (t, 3H).

### Example 297

### 2-(2-(3-chlorobenzoylimino)-4,5,6,7-tetrahydrobenzo[d]thiazol-3(2H)-yl)acetic acid

The titled compound as a white solid was prepared in accordance with the same procedures as in Example 294, using 3-chlorobenzoyl chloride, instead of 3-(trifluoromethyl)benzoyl chloride (Yield: 66%).

¹H NMR (CD₃OD, 400 MHz) δ 8.21 (s, 1H), 8.12 (d, 1H), 7.45 (d, 1H), 7.37 (dd, 1H), 4.97 (s, 2H), 2.63 (brs, 2H), 2.56 (brs, 2H), 1.90-2.00 (m, 4H).

### Example 298

### 2-(2-(3-chlorobenzoylimino)-4,5,6,7-tetrahydrobenzo[d]thiazol-3(2H)-yl)propionic acid

The titled compound as a white solid was prepared in accordance with the same procedures as in Example 294, using 3-chlorobenzoyl chloride and ethyl 2-bromopropionate, instead of 3-(trifluoromethyl)benzoyl chloride and ethyl bromoacetate, respectively (Yield: 52%).

¹H NMR (DMSO-d₆, 400 MHz) δ 8.13 (s, 1H), 8.07 (d, 1H), 7.59 (d, 1H), 7.50 (dd, 1H), 5.23 (brs, 1H), 2.51-2.63 (m, 4H), 1.78-1.87 (m, 4H), 1.64 (d, 3H).

### Example 299

### 2-(2-(3-chlorobenzoylimino)-4,5,6,7-tetrahydrobenzo[d]thiazol-3(2H)-yl)butanoic acid

The titled compound as a white solid was prepared in accordance with the same procedures as in Example 294, using 3-chlorobenzoyl chloride and methyl 2-bromobutyrate, instead of 3-(trifluoromethyl)benzoyl chloride and ethyl bromoacetate, respectively (Yield: 50%).

¹H NMR (CDCl₃, 400 MHz) δ 8.13 (s, 1H), 8.03 (d, 1H), 7.39 (d, 1H), 7.30 (dd, 1H), 4.82 (brs, 1H), 2.33-2.58 (m, 6H), 1.81-1.90 (m, 4H), 0.85 (t, 3H).

### Example 300

### 2-(6-methoxy-2-(4-methylbenzoylimino)benzo[d]thiazol-3(2H)-yl)acetic acid

The titled compound as a white solid was prepared in accordance with the same procedures as in Example 294, using 6-methoxybenzo[d]thiazol-2-amine and p-toluoyl chloride, instead of 4,5,6,7-tetrahydrobenzo[d]thiazol-2-amine and 3-(trifluoromethyl)benzoyl chloride, respectively (Yield: 61%).

¹H NMR (CD₃OD, 400 MHz) δ 8.17 (d, 2H), 7.44 (s, 1H), 7.38-7.46 (m, 2H), 7.26 (d, 2H), 5.23 (s, 2H), 3.38 (s, 3H), 2.42 (s, 3H).

### Example 301

### 2-(6-methoxy-2-(4-methylbenzoylimino)benzo[d]thiazol-3(2H)-yl)propionic acid

The titled compound as a white solid was prepared in accordance with the same procedures as in Example 294, using 6-methoxybenzo[d]thiazol-2-amine, p-toluoyl chloride, and ethyl 2-bromopropionate, instead of 4,5,6,7-tetrahydrobenzo[d]thiazol-2-amine, 3-(trifluoromethyl)benzoyl chloride, and ethyl bromoacetate, respectively (Yield: 61%).

¹H NMR (DMSO-d₆, 400 MHz) δ 8.11 (d, 2H), 7.64 (d, 1H), 7.58 (s, 1H), 7.31 (d, 2H), 7.13 (, 2H), 5.83 (brs, 1H), 3.83 (s, 3H), 2.39 (s, 3H), 1.69 (d, 3H).

### Example 302

### 2-(6-chloro-2-(4-methylbenzoylimino)benzo[d]thiazol-3(2H)-yl)acetic acid

The titled compound as a white solid was prepared in accordance with the same procedures as in Example 294, using 6-chlorobenzo[d]thiazol-2-amine and p-toluoyl chloride, instead of 4,5,6,7-tetrahydrobenzo[d]thiazol-2-amine and 3-(trifluoromethyl)benzoyl chloride, respectively (Yield: 64%).

¹H NMR (CD₃OD, 400 MHz) δ 8.19 (d, 2H), 7.72 (s, 1H), 7.43 (d, 1H), 7.35 (d, 1H), 7.25 (d, 2H), 5.16 (s, 2H), 2.42 (s, 3H).

### Example 303

### 2-(6-chloro-2-(4-methylbenzoylimino)benzo[d]thiazol-3(2H)-yl)propionic acid

The titled compound as a white solid was prepared in accordance with the same procedures as in Example 294, using 6-chlorobenzo[d]thiazol-2-amine, p-toluoyl chloride, and ethyl 2-bromopropionate, instead of 4,5,6,7-tetrahydrobenzo[d]thiazol-2-amine, 3-(trifluoromethyl)benzoyl chloride, and ethyl bromoacetate, respectively (Yield: 75%).

¹H NMR (CD₃OD, 400 MHz) δ 8.19 (d, 2H), 7.75 (s, 1H), 7.44 (dd, 2H), 7.28 (d, 2H), 5.92 (brs, 1H), 2.42 (s, 3H), 1.83 (d, 3H).

### Example 304

### 2-(4,6-difluoro-2-((3-(trifluoromethyl)benzoyl)imino)benzo[d]thiazol-3(2H)-yl)acetic acid

The titled compound as a white solid was prepared in accordance with the same procedures as in Example 200, using 3-(trifluoromethyl)benzoyl chloride, instead of p-toluoyl chloride (Yield: 18%).

¹H NMR (MeOD, 400 MHz): 8.52 (m, 2H), 7.86 (d, 1H), 7.69 (t, 1H), 7.53 (d, 1H), 7.21 (t, 1H), 5.49 (s, 2H)

### Example 305

### 2-(2-((3-chlorobenzoyl)imino)-4,6-difluorobenzo[d]thiazol-3(2H)-yl)acetic acid

The titled compound as a white solid was prepared in accordance with the same procedures as in Example 200, using 3-chlorobenzoyl chloride, instead of p-toluoyl chloride (Yield: 24%).

¹H NMR (DMSO-d₆, 400 MHz): 13.54 (brs, 1H), 8.22 (s, 1H), 8.21 (d, 1H), 7.86 (d, 1H), 7.69 (d, 1H), 7.60-7.54 (m, 2H), 5.46 (s, 2H)

### Example 306

### 2-(2-((3-chlorobenzoyl)imino)-4,6-difluorobenzo[d]thiazol-3(2H)-yl)propionic acid

The titled compound as a white solid was prepared in accordance with the same procedures as in Example 200, using 3-chlorobenzoyl chloride and ethyl 2-bromopropionate, instead of p-toluoyl chloride and ethyl bromoacetate, respectively (Yield: 29%).

¹H NMR (MeOD, 400 MHz): 8.22 (s, 1H), 8.18 (d, 1H), 7.65-7.50 (m, 2H), 7.47 (t, 1H), 7.24 (t, 1H), 6.23 (m, 1H), 1.86 (d, 3H)

### Example 307

### 2-(7-chloro-2-((3-(trifluoromethyl)benzoyl)imino)benzo[d]thiazol-3(2H)-yl)acetic acid

The titled compound as a white solid was prepared in accordance with the same procedures as in Example 266, using 33-chloro-2-fluoroaniline, instead of 2-fluoro-3-(trifluoromethyl)aniline (Yield: 33%).

¹H NMR (MeOD, 400 MHz): 8.55-8.51 (m, 2H), 7.85 (d, 1H), 7.69 (t, 1H), 7.60-7.49 (m, 2H), 7.41 (d, 1H), 5.35 (s, 2H)

### Example 308

### 2-(7-chloro-2-((3-(trifluoromethyl)benzoyl)imino)benzo[d]thiazol-3(2H)-yl)propionic acid

The titled compound as a white solid was prepared in accordance with the same procedures as in Example 266, using 3-chloro-2-fluoroaniline and ethyl 2-bromopropionate, instead of 2-fluoro-3-(trifluoromethyl)aniline and ethyl bromoacetate, respectively (Yield: 18%).

¹H NMR (MeOD, 400 MHz): 8.57 (s, 1H), 8.53 (d, 1H), 7.86 (d, 1H), 7.75-7.60 (m, 2H), 7.44 (d, 1H), 5.80 (m, 1H), 1.86 (d, 3H)

### Example 309

### 2-(7-chloro-2-((3-chlorobenzoyl)imino)benzo[d]thiazol-3(2H)-yl)propionic acid

The titled compound as a white solid was prepared in accordance with the same procedures as in Example 266, using 3-chloro-2-fluoroaniline, 3-chlorobenzoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 16%).

¹H NMR (MeOD, 400 MHz): 8.25 (s, 1H), 8.20 (d, 1H), 7.63 (d, 1H), 7.60-7.50 (m, 2H), 7.50-7.40 (m. 2H), 5.79 (m. 1H), 1.85 (d, 3H)

### Example 310

### 2-(7-chloro-2-((3-chlorobenzoyl)imino)benzo[d]thiazol-3(2H)-yl)butanoic acid

The titled compound as a white solid was prepared in accordance with the same procedures as in Example 266, using 3-chloro-2-fluoroaniline, 3-chlorobenzoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 32%).

¹H NMR (MeOD, 400 MHz): 8.25 (s, 1H), 8.19 (d, 1H), 7.66 (d, 1H), 7.60-7.50 (m, 2H), 7.50-7.40 (m. 2H), 5.69 (m. 1H), 2.70-2.40 (m, 2H), 0.87 (t, 3H)

### Example 311

### 2-(5-chloro-2-(3-(trifluoromethyl)benzoylimino)benzo[d]thiazol-3(2H)-yl)acetic acid

The titled compound as a white solid was prepared in accordance with the same procedures as in Example 266, using 5-chloro-2-fluoroaniline, instead of 2-fluoro-3-(trifluoromethyl)aniline (Yield: 14%).

¹H NMR (DMSO-d₆, 400 MHz) δ 8.50 (m, 2H), 7.99-8.04 (m, 3H), 7.80 (t, 1H), 7.47 (d, 2H), 5.36 (s, 2H)

### Example 312

### 2-(5-chloro-2-(3-chlorobenzoylimino)benzo[d]thiazol-3(2H)-yl)acetic acid

The titled compound as a white solid was prepared in accordance with the same procedures as in Example 266, using 5-chloro-2-fluoroaniline and 3-chlorobenzoyl chloride, instead of 2-fluoro-3-(trifluoromethyl)aniline and 3-(trifluoromethyl)benzoyl chloride, respectively (Yield: 16%).

¹H NMR (DMSO-d₆, 400 MHz) δ 8.15-8.18 (m, 2H), 7.99 (d, 1H), 7.90 (s, 1H), 7.65 (d, 1H), 7.54 (t, 1H), 7.44 (d, 1H), 5.26 (s, 2H)

### Example 313

### 2-(7-methyl-2-(3-(trifluoromethyl)benzoylimino)benzo[d]thiazol-3(2H)-yl)acetic acid

The titled compound as a white solid was prepared in accordance with the same procedures as in Example 266, using 2-fluoro-3-methylaniline, instead of 2-fluoro-3-(trifluoromethyl)aniline (Yield: 21%).

¹H NMR (DMSO-d₆, 400 MHz) δ 8.32-8.34 (m, 2H), 7.79 (d, 1H), 7.60 (t, 1H), 7.45 (d, 1H), 7.29 (t, 1H), 7.07 (d, 1H), 5.17 (s, 2H), 2.33 (s, 3H)

### Example 314

### 2-(7-methyl-2-(3-(trifluoromethyl)benzoylimino)benzo[d]thiazol-3(2H)-yl)propionic acid

The titled compound as a white solid was prepared in accordance with the same procedures as in Example 266, using 2-fluoro-3-methylaniline and ethyl 2-bromopropionate, instead of 2-fluoro-3-(trifluoromethyl)aniline and ethyl bromoacetate, respectively (Yield: 25%).

¹H NMR (MeOD, 400 MHz) δ 8.55 (s, 1H), 8.50 (d, 1H), 7.82 (d, 1H), 7.66 (t, 1H), 7.43-7.51 (m, 2H), 7.18 (d, 1H), 5.78 (m, 1H), 2.50 (s, 3H), 1.85 (d, 3H)

### Example 315

### 2-(2-(4-methylbenzoylimino)-5,6,7,8-tetrahydro-2H-cyclohepta[d]thiazol-3(4H)-yl)acetic acid

The titled compound as a white solid was prepared in accordance with the same procedures as in Example 200, using 5,6,7,8-tetrahydro-4H-cyclohepta[d][1,3]thiazol-2-amine, instead of 4,6-difluorobenzo[d]thiazol-2-amine (Yield: 44%).

¹H NMR (DMSO-d₆, 400 MHz) δ 13.29-13.45 (m, 1H), 8.05 (d, 2H), 7.26 (d, 2H), 5.14 (s, 2H), 2.69 (m, 4H), 2.36 (s, 3H), 1.80 (m, 2H), 1.68 (m, 4H)

### Example 316

### 2-(2-(4-methylbenzoylimino)-5,6,7,8-tetrahydro-2H-cyclohepta[d]thiazol-3(4H)-yl)propionic acid

The titled compound as a white solid was prepared in accordance with the same procedures as in Example 200, using 5,6,7,8-tetrahydro-4H-cyclohepta[d][1,3]thiazol-2-amine and ethyl 2-bromopropionate, instead of 4,6-difluorobenzo[d]thiazol-2-amine and ethyl bromoacetate, respectively (Yield: 39%).

¹H NMR (DMSO-d₆, 400 MHz) δ 12.97-13.01 (m, 1H), 8.04 (d, 2H), 7.25 (d, 2H), 5.55-5.80 (m, 1H), 2.77 (m, 2H), 2.67 (m, 2H), 2.36 (s, 3H), 1.81 (m, 2H), 1.68 (m, 4H), 1.61 (d, 3H)

### Example 317

### 2-(2-(4-methylbenzoylimino)-5,6,7,8-tetrahydro-2H-cyclohepta[d]thiazol-3(4H)-yl)butanoic acid

The titled compound as a white solid was prepared in accordance with the same procedures as in Example 200, using 5,6,7,8-tetrahydro-4H-cyclohepta[d][1,3]thiazol-2-amine and methyl 2-bromobutyrate, instead of 4,6-difluorobenzo[d]thiazol-2-amine and ethyl bromoacetate, respectively (Yield: 35%).

¹H NMR (DMSO-d₆, 400 MHz) δ 12.66-12.95 (m, 1H), 8.01 (d, 2H), 7.23 (d, 2H), 2.66-2.76 (m, 4H), 2.34 (s, 3H), 2.23 (m, 2H), 1.80 (m, 2H), 1.64 (m, 4H), 0.73 (m, 3H)

### Example 318

### 2-(2-(3-chlorobenzoylimino)-5,6,7,8-tetrahydro-2H-cyclohepta[d]thiazol-3(4H)-yl)acetic acid

The titled compound as a white solid was prepared in accordance with the same procedures as in Example 200, using 5,6,7,8-tetrahydro-4H-cyclohepta[d][1,3]thiazol-2-amine and 3-chlorobenzoyl chloride, instead of 4,6-difluorobenzo[d]thiazol-2-amine and p-toluoyl chloride, respectively (Yield: 31%).

¹H NMR (DMSO-d₆, 400 MHz) δ 13.38-13.41 (m, 1H), 8.08-8.12 (m, 2H), 7.59 (d, 1H), 7.51 (t, 1H), 5.16 (s, 2H), 2.71 (m, 4H), 1.80 (m, 2H), 1.68 (m, 4H)

### Example 319

### 2-(2-(3-chlorobenzoylimino)-5,6,7,8-tetrahydro-2H-cyclohepta[d]thiazol-3(4H)-yl)propionic acid

The titled compound as a white solid was prepared in accordance with the same procedures as in Example 200, using 5,6,7,8-tetrahydro-4H-cyclohepta[d][1,3]thiazol-2-amine, 3-chlorobenzoyl chloride, and ethyl 2-bromopropionate, instead of 4,6-difluorobenzo[d]thiazol-2-amine, p-toluoyl chloride, and ethyl bromoacetate, respectively (Yield: 38%).

¹H NMR (DMSO-d₆, 400 MHz) δ 13.01 (m, 1H), 8.14 (s, 1H), 8.08 (d, 1H), 7.58 (d, 1H), 7.49 (t, 1H), 5.61-5.66 (m, 1H), 2.80 (m, 2H), 2.69 (m, 2H), 1.81 (m, 2H), 1.69 (m, 4H), 1.61 (d, 3H)

### Example 320

### 2-(2-(3-chlorobenzoylimino)-5,6,7,8-tetrahydro-2H-cyclohepta[d]thiazol-3(4H)-yl)butanoic acid

The titled compound as a white solid was prepared in accordance with the same procedures as in Example 200, using 5,6,7,8-tetrahydro-4H-cyclohepta[d][1,3]thiazol-2-amine, 3-chlorobenzoyl chloride, and methyl 2-bromobutyrate, instead of 4,6-difluorobenzo[d]thiazol-2-amine, p-toluoyl chloride, and ethyl bromoacetate, respectively (Yield: 49%).

¹H NMR (DMSO-d₆, 400 MHz) δ 12.83-12.98 (m, 1H), 8.12 (s, 1H), 8.06-8.08 (m, 1H), 7.58 (d, 1H), 7.49 (t, 1H), 4.95-5.58 (m, 1H), 2.71-2.81 (m, 4H), 2.25-2.33 (m, 2H), 1.82 (m, 2H), 1.66 (m, 4H), 0.75 (m, 3H)

### Example 321

### 2-(2-(3-(trifluoromethyl)benzoylimino)-5,6,7,8-tetrahydro-2H-cyclohepta[d]thiazol-3(4H)-yl)acetic acid

The titled compound as a white solid was prepared in accordance with the same procedures as in Example 200, using 5,6,7,8-tetrahydro-4H-cyclohepta[d][1,3]thiazol-2-amine and 3-(trifluoromethyl)benzoyl chloride, instead of 4,6-difluorobenzo[d]thiazol-2-amine and p-toluoyl chloride, respectively (Yield: 48%).

¹H NMR (DMSO-d₆, 400 MHz) δ 13.38-13.40 (m, 1H), 8.41-8.45 (m, 2H), 7.90 (d, 1H), 7.73 (t, 1H), 5.16 (s, 2H), 2.72 (m, 4H), 1.80 (m, 2H), 1.69 (m, 4H)

### Example 322

### 2-(2-(3-(trifluoromethyl)benzoylimino)-5,6,7,8-tetrahydro-2H-cyclohepta[d]thiazol-3(4H)-yl)propionic acid

The titled compound as a white solid was prepared in accordance with the same procedures as in Example 200, using 5,6,7,8-tetrahydro-4H-cyclohepta[d][1,3]thiazol-2-amine, 3-(trifluoromethyl)benzoyl chloride, and ethyl 2-bromopropionate, instead of 4,6-difluorobenzo[d]thiazol-2-amine, p-toluoyl chloride, and ethyl bromoacetate, respectively (Yield: 42%).

¹H NMR (DMSO-d₆, 400 MHz) δ 13.01-13.05 (m, 1H), 8.41-8.43 (m, 2H), 7.89 (d, 1H), 7.71 (t, 1H), 5.51-5.60 (m, 1H), 2.81-2.82 (m, 2H), 2.71 (m, 2H), 1.82 (m, 2H), 1.70 (m, 4H), 1.62 (d, 3H)

### Example 323

### 2-(2-(3-(trifluoromethyl)benzoylimino)-5,6,7,8-tetrahydro-2H-cyclohepta[d]thiazol-3(4H)-yl)butanoic acid

The titled compound as a white solid was prepared in accordance with the same procedures as in Example 200, using 5,6,7,8-tetrahydro-4H-cyclohepta[d][1,3]thiazol-2-amine, 3-(trifluoromethyl)benzoyl chloride, and methyl 2-bromobutyrate, instead of 4,6-difluorobenzo[d]thiazol-2-amine, p-toluoyl chloride, and ethyl bromoacetate, respectively (Yield: 36%).

¹H NMR (DMSO-d₆, 400 MHz) δ 12.86-13.08 (m, 1H), 8.42 (m, 2H), 7.89 (d, 1H), 7.71 (t, 1H), 5.00-5.59 (m, 1H), 2.72-2.82 (m, 4H), 2.26-2.34 (m, 2H), 1.82 (m, 2H), 1.67 (m, 4H), 0.76 (m, 3H)

### Example 324

### 2-(5,6-dimethyl-2-(4-methylbenzoylimino)benzo[d]thiazol-3(2H)-yl)acetic acid

The titled compound as a white solid was prepared in accordance with the same procedures as in Example 200, using 5,6-dimethylbenzo[d]thiazol-2-amine, instead of 4,6-difluorobenzo[d]thiazol-2-amine (Yield: 22%).

¹H NMR (DMSO-d₆, 400 MHz) δ 13.35 (m, 1H), 8.12 (d, 2H), 7.85 (d, 1H), 7.73 (d, 1H), 7.33 (d, 2H), 5.27 (s, 2H), 2.51 (s, 3H), 2.39 (s, 3H), 2.32 (s, 3H)

### Example 325

### 2-(5-fluoro-6-methyl-2-(4-methylbenzoylimino)benzo[d]thiazol-3(2H)-yl)propionic acid

The titled compound as a white solid was prepared in accordance with the same procedures as in Example 311, using ethyl 2-(5-fluoro-6-methyl-2-(4-methylbenzoylimino)benzo[d]thiazol-3(2H)-yl)propionate, instead of ethyl 2-(5-chloro-2-(3-(trifluoromethyl)benzoylimino)benzo[d]thiazol-3(2H)-yl)acetate (Yield: 65%).

¹H NMR (DMSO-d₆, 400 MHz) δ 13.09 (m, 1H), 8.10 (d, 2H), 7.86 (d, 1H), 7.75 (d, 1H), 7.32 (d, 2H), 5.75 (m, 1H), 2.39 (s, 3H), 2.33 (s, 3H), 1.71 (d, 3H)

### Example 326

### 2-(5-fluoro-6-methyl-2-(3-(trifluoromethyl)benzoylimino)benzo[d]thiazol-3(2H)-yl)acetic acid

The titled compound as a white solid was prepared in accordance with the same procedures as in Example 200, using 5-fluoro-6-methylbenzo[d]thiazol-2-amine and 3-(trifluoromethyl)benzoyl chloride, instead of 4,6-difluorobenzo[d]thiazol-2-amine and p-toluoyl chloride, respectively (Yield: 36%).

¹H NMR (DMSO-d₆, 400 MHz) δ 8.46-8.50 (m, 2H), 7.95 (d, 1H), 7.88 (d, 1H), 7.76 (t, 1H), 7.70 (d, 2H), 5.22 (s, 2H), 2.33 (s, 3H)

### Example 327

### 2-(5-fluoro-6-methyl-2-(3-(trifluoromethyl)benzoylimino)benzo[d]thiazol-3(2H)-yl)propionic acid

The titled compound as a white solid was prepared in accordance with the same procedures as in Example 200, using 5-fluoro-6-methylbenzo[d]thiazol-2-amine, 3-(trifluoromethyl)benzoyl chloride, and ethyl 2-bromopropionate, instead of 4,6-difluorobenzo[d]thiazol-2-amine, p-toluoyl chloride, and ethyl bromoacetate, respectively (Yield: 18%).

¹H NMR (DMSO-d₆, 400 MHz) δ 13.14 (s, 1H), 8.50 (s, 1H), 8.46 (d, 1H), 7.94 (dd, 2H), 7.32-7.85 (m, 2H), 5.78 (m, 1H), 2.34 (s, 3H), 1.73 (d, 3H)

### Example 328

### 2-(2-(3-chlorobenzoylimino)-5-fluoro-6-methylbenzo[d]thiazol-3(2H)-yl)acetic acid

The titled compound as a white solid was prepared in accordance with the same procedures as in Example 200, using 5-fluoro-6-methylbenzo[d]thiazol-2-amine and 3-chlorobenzoyl chloride, instead of 4,6-difluorobenzo[d]thiazol-2-amine and p-toluoyl chloride, respectively (Yield: 16%).

¹H NMR (DMSO-d₆, 400 MHz) δ 8.05-8.07 (m, 2H), 7.77 (d, 1H), 7.55-7.57 (m, 2H), 7.44 (t, 1H), 5.11 (s, 2H), 2.24 (s, 3H)

### Example 329

### 2-(2-(4-methylbenzoylimino)-5-(trifluoromethyl)benzo[d]thiazol-3(2H)-yl)propionic acid

The titled compound as a white solid was prepared in accordance with the same procedures as in Example 266, using 2-fluoro-5-(trifluoromethyl)aniline, p-toluoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 20%).

¹H NMR (DMSO-d₆, 400 MHz) δ 13.05 (m, 1H), 8.20-8.24 (m, 2H), 8.07-8.13 (m, 2H), 7.73 (d, 1H), 7.34 (d, 2H), 5.97 (m, 1H), 2.40 (s, 3H), 1.75 (d, 3H)

### Example 330

### 2-(2-(3-chlorobenzoylimino)-5-ethoxybenzo[d]thiazol-3(2H)-yl)propionic acid

The titled compound as a white solid was prepared in accordance with the same procedures as in Example 200, using 5-ethoxybenzo[d]thiazol-2-amine, 3-chlorobenzoyl chloride, and ethyl 2-bromopropionate, instead of 4,6-difluorobenzo[d]thiazol-2-amine, p-toluoyl chloride, and ethyl bromoacetate, respectively (Yield: 15%).

¹H NMR (DMSO-d₆, 400 MHz) δ 13.09 (s, 1H), 8.20 (s, 1H), 8.14 (d, 1H), 7.85 (d, 1H), 7.66 (d, 1H), 7.55 (t, 1H), 7.45 (s, 1H), 7.02 (d, 1H), 5.85 (m, 1H), 4.15-4.16 (m, 2H), 1.71 (d, 3H), 1.38 (t, 3H)

### Example 331

### 2-(2-(3-chlorobenzoylimino)-5-ethoxybenzo[d]thiazol-3(2H)-yl)butanoic acid

The titled compound as a white solid was prepared in accordance with the same procedures as in Example 200, using 5-ethoxybenzo[d]thiazol-2-amine, 3-chlorobenzoyl chloride, and methyl 2-bromobutyrate, instead of 4,6-difluorobenzo[d]thiazol-2-amine, p-toluoyl chloride, and ethyl bromoacetate, respectively (Yield: 23%).

¹H NMR (DMSO-d₆, 400 MHz) δ 13.09 (m, 1H), 8.18 (s, 1H), 8.12 (d, 1H), 7.85 (d, 1H), 7.66 (d, 1H), 7.55 (t, 1H), 7.48 (m, 1H), 7.02 (d, 1H), 5.74 (m, 1H), 4.14-4.16 (m, 2H), 2.33-2.51 (m, 2H), 1.38 (t, 3H), 0.76 (t, 3H)

### Example 332

### 2-(4,6-difluoro-2-(3-(trifluoromethyl)benzoylimino)benzo[d]thiazol-3(2H)-yl)propionic acid

The titled compound as a white solid was prepared in accordance with the same procedures as in Example 200, using 3-(trifluoromethyl)benzoyl chloride and ethyl 2-bromopropionate, instead of p-toluoyl chloride and ethyl bromoacetate, respectively (Yield: 48%).

¹H NMR (DMSO-d₆, 400 MHz) δ 13.32 (m, 1H), 8.58 (s, 1H), 8.50 (s, 1H), 8.00 (d, 1H), 7.89 (d, 1H), 7.79 (t, 1H), 7.61 (t, 1H), 6.23-6.47 (m, 1H), 1.77 (d, 3H)

### Example 333

### 2-(6,7-difluoro-2-((4-methylbenzoyl)imino)benzo[d]thiazol-3(2H)-yl)acetic acid

### Step 1: 4-methyl-N-((2,3,4-trifluorophenyl)carbamothioyl)benzamide

To a solution of ammonium thiocyanate (5.19 g, 68.18 mmol) in acetone (120 mL) was added dropwise p-toluoyl chloride (8.26 mL, 62.50 mmol) over 5 minutes. The reaction mixture was stirred at 50° C. for 15 minutes and then 2,3,4-trifluoroaniline (8.36 g, 56.82 mmol) was added thereto. The reaction mixture was stirred at 50° C. for 1 hour and then evaporated. Ice water was added to the resulting residue. The residue was filtered, washed with water, and then dried under reduced pressure to give 14.8 g of the titled compound as a yellow solid (Yield: 80%).

### Step 2: N-(6,7-difluorobenzo[d]thiazol-2(3H)-ylidene)-4-methylbenzamide

To a solution of 4-methyl-N-((2,3,4-trifluorophenyl)carbamothioyl)benzamide (3.24 g, 10.00 mmol) prepared in Step 1 in N,N-dimethylformamide (50.0 mL) was added portionwise sodium hydride (60%, 2.00 g, 50.00 mmol) at 0° C. The reaction mixture was stirred at 60° C. for 48 hours. The reaction mixture was cooled in ice water, quenched with water, and then acidified with an aqueous 1N HCl solution. The reaction mixture was diluted with ethyl acetate and then evaporated. The resulting solid was filtered, washed with water and isopropyl ether, and then dried under reduced pressure to give 2.54 g of the titled compound as a yellow solid (Yield: 83%).

### Step 3: ethyl 2-(6,7-difluoro-2-((4-methylbenzoyl)imino)benzo[d]thiazol-3(2H)-yl)acetate

To a solution of N-(6,7-difluorobenzo[d]thiazol-2(3H)-ylidene)-4-methylbenzamide (304 mg, 1.00 mmol) prepared in Step 2 in N,N-dimethylformamide (5.0 mL) were added potassium carbonate (276 mg, 2.00 mmol) and ethyl bromoacetate (166 uL, 1.50 mmol). The reaction mixture was stirred at 50° C. for 5 hours. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=31) to give 301 mg of the titled compound as a white solid (Yield: 77%).

### Step 4: 2-(6,7-difluoro-2-((4-methylbenzoyl)imino)benzo[d]thiazol-3(2H)-yl)acetic acid

To a solution of ethyl 2-(6,7-difluoro-2-((4-methylbenzoyl)imino)benzo[d]thiazol-3(2H)-yl)acetate (250 mg) prepared in Step 3 in tetrahydrofuran (6.0 mL) was added a 1N sodium hydroxide solution (3.0 mL). The reaction mixture was stirred at room temperature overnight. The reaction mixture was acidified with an aqueous 1N HCl solution and then extracted with ethyl acetate. The organic layer was washed with brine, dried over anhydrous magnesium sulfate, filtered, and then evaporated. The residue was dried under reduced pressure to give 180 mg of the titled compound as a white solid (Yield: 78%).

¹H NMR (DMSO-d₆, 400 MHz); 8.14 (d, 2H), 7.65 (m, 2H), 7.35 (d, 2H), 5.33 (s, 2H), 2.40 (s, 3H)

### Example 334

### 2-(6,7-difluoro-2-((4-methylbenzoyl)imino)benzo[d]thiazol-3(2H)-yl)propionic acid

The titled compound as a white solid was prepared in accordance with the same procedures as in Example 333, using ethyl 2-bromopropionate, instead of ethyl bromoacetate (Yield: 32%).

¹H NMR (DMSO-d₆, 400 MHz); 8.12 (d, 2H), 7.68 (m, 2H), 7.35 (d, 2H), 5.88 (brs, 1H), 2.40 (s, 3H), 1.73 (d, 3H)

### Example 335

### 2-(6,7-difluoro-2-((4-methylbenzoyl)imino)benzo[d]thiazol-3(2H)-yl)butanoic acid

The titled compound as a white solid was prepared in accordance with the same procedures as in Example 333, using methyl 2-bromobutyrate, instead of ethyl bromoacetate (Yield: 38%).

¹H NMR (DMSO-d₆, 400 MHz); 8.11 (d, 2H), 7.70 (s, 2H), 7.35 (d, 2H), 5.76 (brs, 1H), 2.50-2.30 (m, 2H), 2.40 (s, 3H), 0.77 (t, 3H)

### Example 336

### 2-(2-((3-chlorobenzoyl)imino)-6,7-difluorobenzo[d]thiazol-3(2H)-yl)acetic acid

The titled compound as a white solid was prepared in accordance with the same procedures as in Example 333, using 3-chlorobenzoyl chloride, instead of p-toluoyl chloride (Yield: 26%).

¹H NMR (DMSO-d₆, 400 MHz); 8.21 (s, 1H), 8.19 (d, 1H), 7.69 (m, 3H), 7.59 (t, 1H), 5.37 (s, 2H)

### Example 337

### 2-(2-((3-chlorobenzoyl)imino)-6,7-difluorobenzo[d]thiazol-3(2H)-yl)propionic acid

The titled compound as a white solid was prepared in accordance with the same procedures as in Example 333, using 3-chlorobenzoyl chloride and ethyl 2-bromopropionate, instead of p-toluoyl chloride and ethyl bromoacetate, respectively (Yield: 21%).

¹H NMR (DMSO-d₆, 400 MHz); 13.28 (s, 1H), 8.19 (s, 1H), 8.15 (d, 1H), 7.70 (m, 3H), 7.57 (t, 1H), 5.92 (s, 1H), 1.74 (d, 3H)

### Example 338

### 2-(2-((3-chlorobenzoyl)imino)-6,7-difluorobenzo[d]thiazol-3(2H)-yl)butanoic acid

The titled compound as a white solid was prepared in accordance with the same procedures as in Example 333, using 3-chlorobenzoyl chloride and methyl 2-bromobutyrate, instead of p-toluoyl chloride and ethyl bromoacetate, respectively (Yield: 31%).

¹H NMR (DMSO-d₆, 400 MHz); 13.28 (s, 1H), 8.18 (s, 1H), 8.15 (d, 1H), 7.70 (m, 3H), 7.58 (t, 1H), 5.80 (brs, 1H), 2.51-2.35 (m, 2H), 0.78 (t, 3H)

### Example 339

### 2-(6,7-difluoro-2-((3-(trifluoromethyl)benzoyl)imino)benzo[d]thiazol-3(2H)-yl)acetic acid

The titled compound as a white solid was prepared in accordance with the same procedures as in Example 333, using 3-(trifluoromethyl)benzoyl chloride, instead of p-toluoyl chloride (Yield: 42%).

¹H NMR (DMSO-d₆, 400 MHz); 13.52 (s, 1H), 8.52 (m, 2H), 8.01 (d, 1H), 7.80 (t, 1H), 7.71 (s, 2H), 5.37 (brs, 1H)

### Example 340

### 2-(6,7-difluoro-2-((3-(trifluoromethyl)benzoyl)imino)benzo[d]thiazol-3(2H)-yl)propionic acid

The titled compound as a white solid was prepared in accordance with the same procedures as in Example 333, using 3-(trifluoromethyl)benzoyl chloride and ethyl 2-bromopropionate, instead of p-toluoyl chloride and ethyl bromoacetate, respectively (Yield: 28%).

¹H NMR (DMSO-d₆, 400 MHz); 13.26 (s, 1H), 8.51 (m, 2H), 8.01 (d, 1H), 7.80 (m, 3H), 5.92 (brs, 1H), 1.75 (d, 3H)

### Example 341

### 2-(6,7-difluoro-2-((3-(trifluoromethyl)benzoyl)imino)benzo[d]thiazol-3(2H)-yl)butanoic acid

The titled compound as a white solid was prepared in accordance with the same procedures as in Example 333, using 3-(trifluoromethyl)benzoyl chloride and methyl 2-bromobutyrate, instead of p-toluoyl chloride and ethyl bromoacetate, respectively (Yield: 24%).

¹H NMR (DMSO-d₆, 400 MHz); 13.21 (s, 1H), 8.43 (m, 2H), 7.94 (d, 1H), 7.75-7.65 (m, 3H), 5.73 (brs, 1H), 2.44-2.29 (m, 2H), 0.73 (t, 3H)

### Example 342

### 2-(5-methoxy-2-((4-methylbenzoyl)imino)benzo[d]thiazol-3(2H)-yl)acetic acid

The titled compound as a white solid was prepared in accordance with the same procedures as in Example 200, using 5-methoxybenzo[d]thiazol-2-amine, instead of 4,6-difluorobenzo[d]thiazol-2-amine (Yield: 35%).

¹H NMR (DMSO-d₆, 400 MHz); 13.31 (s, 1H), 8.13 (d, 2H), 7.82 (d, 1H), 7.38 (s, 1H), 7.33 (d, 2H), 7.00 (d, 1H), 5.31 (s, 2H), 3.86 (s, 3H), 2.39 (s, 3H)

### Example 343

### 2-(5-methoxy-2-((4-methylbenzoyl)imino)benzo[d]thiazol-3(2H)-yl)propionic acid

The titled compound as a white solid was prepared in accordance with the same procedures as in Example 200, using 5-methoxybenzo[d]thiazol-2-amine and ethyl 2-bromopropionate, instead of 4,6-difluorobenzo[d]thiazol-2-amine and ethyl bromoacetate, respectively (Yield: 16%).

¹H NMR (DMSO-d₆, 400 MHz); 13.03 (s, 1H), 8.11 (d, 2H), 7.83 (d, 1H), 7.42 (s, 1H), 7.32 (d, 2H), 7.01 (d, 1H), 5.83 (brs, 1H), 3.87 (s, 3H), 2.39 (s, 3H), 1.73 (d, 3H)

### Example 344

### 2-(7-methyl-2-((4-methylbenzoyl)imino)benzo[d]thiazol-3(2H)-yl)butanoic acid

The titled compound as a white solid was prepared in accordance with the same procedures as in Example 266, using 2-fluoro-3-methylaniline, p-toluoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 12%).

¹H NMR (DMSO-d₆, 400 MHz); 13.17 (s, 1H), 8.12 (d, 2H), 7.70 (s, 1H), 7.62 (d, 1H), 7.35 (m, 3H), 5.78 (brs, 1H), 2.51-2.30 (m, 2H), 2.40 (s, 3H), 0.77 (t, 3H)

### Example 345

### 2-(7-methyl-2-((3-(trifluoromethyl)benzoyl)imino)benzo[d]thiazol-3(2H)-yl)butanoic acid

The titled compound as a white solid was prepared in accordance with the same procedures as in Example 266, using 2-fluoro-3-methylaniline and methyl 2-bromobutyrate, instead of 2-fluoro-3-(trifluoromethyl)aniline and ethyl bromoacetate, respectively (Yield: 27%).

¹H NMR (DMSO-d₆, 400 MHz); 12.91 (s, 1H), 8.30 (s, 1H), 8.27 (d, 1H), 7.77 (d, 1H), 7.58 (t, 1H), 7.52 (s, 1H), 7.30 (t, 1H), 7.07 (d, 1H), 5.55 (brs, 1H), 2.32-2.15 (m, 2H), 2.32 (s, 3H), 0.56 (t, 3H)

### Example 346

### 2-(7-fluoro-2-((3-(trifluoromethyl)benzoyl)imino)benzo[d]thiazol-3(2H)-yl)propionic acid

The titled compound as a white solid was prepared in accordance with the same procedures as in Example 266, using 2,3-difluoroaniline and ethyl 2-bromopropionate, instead of 2-fluoro-3-(trifluoromethyl)aniline and ethyl bromoacetate, respectively (Yield: 18%).

¹H NMR (DMSO-d₆, 400 MHz); 13.21 (s, 1H), 8.52 (s, 1H), 8.50 (d, 1H), 8.01 (d, 1H), 7.78 (m, 2H), 7.66 (d, 1H), 7.35 (t, 1H), 5.94 (brs, 1H), 1.76 (d, 3H)

### Example 347

### 2-(2-((3-chlorobenzoyl)imino)-7-fluorobenzo[d]thiazol-3(2H)-yl)acetic acid

The titled compound as a white solid was prepared in accordance with the same procedures as in Example 266, using 2,3-difluoroaniline and 3-chlorobenzoyl chloride, instead of 2-fluoro-3-(trifluoromethyl)aniline and 3-(trifluoromethyl)benzoyl chloride, respectively (Yield: 8%).

¹H NMR (DMSO-d₆, 400 MHz); 13.49 (s, 1H), 8.22 (s, 1H), 8.20 (d, 1H), 7.69 (d, 2H), 7.59 (m, 2H), 7.32 (t, 1H), 5.39 (s, 2H)

### Example 348

### 2-(2-((4-methylbenzoyl)imino)-5-(trifluoromethyl)benzo[d]thiazol-3(2H)-yl)propionic acid

The titled compound as a white solid was prepared in accordance with the same procedures as in Example 266, using 2-fluoro-5-(trifluoromethyl)aniline, p-toluoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 15%).

¹H NMR (DMSO-d₆, 400 MHz); 13.14 (s, 1H), 8.24 (s, 1H), 8.21 (d, 1H), 8.13 (d, 2H), 7.74 (d, 1H), 7.30 (d, 2H), 5.97 (brs, 1H), 2.40 (s, 3H), 1.75 (d, 3H)

### Example 349

### 2-(5-methyl-2-((3-(trifluoromethyl)benzoyl)imino)benzo[d]thiazol-3(2H)-yl)acetic acid

The titled compound as a white solid was prepared in accordance with the same procedures as in Example 266, using 2-fluoro-5-methylaniline, instead of 2-fluoro-3-(trifluoromethyl)aniline (Yield: 29%).

¹H NMR (DMSO-d₆, 400 MHz); 13.38 (s, 1H), 8.50 (s, 2H), 7.98 (d, 1H), 7.87 (d, 1H), 7.80 (t, 1H), 7.65 (s, 1H), 7.25 (d, 1H), 5.33 (s, 2H), 2.46 (s, 3H)

### Example 350

### 2-(2-((3-chlorobenzoyl)imino)-5-methylbenzo[d]thiazol-3(2H)-yl)butanoic acid

The titled compound as a white solid was prepared in accordance with the same procedures as in Example 266, using 2-fluoro-5-methylaniline, 3-chlorobenzoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 16%).

¹H NMR (DMSO-d₆, 400 MHz); 13.07 (s, 1H), 8.19 (s, 1H), 8.14 (d, 1H), 7.87 (d, 1H), 7.72 (s, 1H), 7.67 (d, 1H), 7.56 (t, 1H), 7.26 (d, 1H), 5.72 (brs, 1H), 2.51-2.30 (m, 2H), 2.47 (s, 3H), 0.77 (t, 3H)

### Example 351

### 2-(2-((3-chlorobenzoyl)imino)-5-ethoxybenzo[d]thiazol-3(2H)-yl)acetic acid

The titled compound as a white solid was prepared in accordance with the same procedures as in Example 200, using 5-ethoxybenzo[d]thiazol-2-amine and 3-chlorobenzoyl chloride, instead of 4,6-difluorobenzo[d]thiazol-2-amine and p-toluoyl chloride, respectively (Yield: 27%).

¹H NMR (DMSO-d₆, 400 MHz); 13.34 (brs, 1H), 8.20 (s, 1H), 8.17 (d, 1H), 7.84 (d, 1H), 7.67 (d, 1H), 7.56 (t, 1H), 7.40 (s, 1H), 7.01 (d, 1H), 5.33 (s, 2H), 4.14 (d, 2H), 1.37 (t, 3H)

### Example 352

### 2-(2-((3-chlorobenzoyl)imino)-7-methylbenzo[d]thiazol-3(2H)-yl)acetic acid

The titled compound as a white solid was prepared in accordance with the same procedures as in Example 266, using 2-fluoro-3-methylaniline and 3-chlorobenzoyl chloride, instead of 2-fluoro-3-(trifluoromethyl)aniline and 3-(trifluoromethyl)benzoyl chloride, respectively (Yield: 8%).

¹H NMR (MeOD, 400 MHz): 8.30-8.15 (m, 2H), 7.55-7.50 (m, 1H), 7.50-7.35 (m, 3H), 7.25-7.15 (m, 1H), 5.30 (s, 2H), 2.52 (s, 3H)

### Example 353

### 2-(2-((3-chlorobenzoyl)imino)-7-methylbenzo[d]thiazol-3(2H)-yl)propionic acid

The titled compound as a white solid was prepared in accordance with the same procedures as in Example 266, using 2-fluoro-3-methylaniline, 3-chlorobenzoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 24%).

¹H NMR (MeOD, 400 MHz): 8.25 (s, 1H), 8.19 (d, 1H), 7.60-7.40 (m, 4H), 7.22 (d, 1H), 5.90-5.70 (m, 1H), 2.54 (s, 3H), 1.83 (d, 3H)

### Example 354

### 2-(2-((3-chlorobenzoyl)imino)-7-methylbenzo[d]thiazol-3(2H)-yl)butanoic acid

The titled compound as a white solid was prepared in accordance with the same procedures as in Example 266, using 2-fluoro-3-methylaniline, 3-chlorobenzoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 11%).

¹H NMR (MeOD, 400 MHz): 8.25 (s, 1H), 8.20 (d, 1H), 7.60-7.40 (m, 4H), 7.23 (d, 1H), 5.80-5.55 (m, 1H), 2.61 (s, 3H), 2.65-2.40 (m, 2H), 0.86 (t, 3H)

### Example 355

### 2-(5-fluoro-2-((3-(trifluoromethyl)benzoyl)imino)benzo[d]thiazol-3(2H)-yl)propionic acid

The titled compound as a white solid was prepared in accordance with the same procedures as in Example 266, using 2,5-difluoroaniline, instead of 2-fluoro-3-(trifluoromethyl)aniline (Yield: 14%).

¹H NMR (MeOD, 400 MHz): 8.55 (s, 1H), 8.52 (d, 1H), 7.90-7.80 (m, 2H), 7.68 (dd, 1H), 7.54 (d, 1H), 7.17 (dd, 1H), 5.85-5.70 (m, 1H), 1.83 (d, 3H)

### Example 356

### 2-(7-fluoro-2-((3-(trifluoromethyl)benzoyl)imino)benzo[d]thiazol-3(2H)-yl)acetic acid

The titled compound as a white solid was prepared in accordance with the same procedures as in Example 266, using 2,3-difluoroaniline, instead of 2-fluoro-3-(trifluoromethyl)aniline (Yield: 29%).

¹H NMR (MeOD, 400 MHz): 8.52 (s, 1H), 8.49 (d, 1H), 7.83 (d, 1H), 7.68 (dd, 1H), 7.55-7.45 (m, 1H), 7.42 (d, 1H), 7.13 (dd, 1H), 5.33 (s, 2H)

### Example 357

### 2-(7-chloro-2-((3-(trifluoromethyl)benzoyl)imino)benzo[d]thiazol-3(2H)-yl)butanoic acid

The titled compound as a white solid was prepared in accordance with the same procedures as in Example 266, using 3-chloro-2-fluoroaniline and methyl 2-bromobutyrate, instead of 2-fluoro-3-(trifluoromethyl)aniline and ethyl bromoacetate, respectively (Yield: 38%).

¹H NMR (DMSO-d₆, 400 MHz) δ 13.09-13.22 (m, 1H), 8.47-8.51 (m, 2H), 8.00 (d, 1H), 7.90 (m, 1H), 7.80 (t, 1H), 7.65 (t, 1H), 7.57 (d, 1H), 5.80-5.82 (m, 1H), 2.37-2.51 (m, 2H), 0.79 (t, 3H)

### Example 358

### 2-(7-chloro-2-((3-chlorobenzoyl)imino)benzo[d]thiazol-3(2H)-yl)acetic acid

The titled compound as a white solid was prepared in accordance with the same procedures as in Example 266, using 3-chloro-2-fluoroaniline and 3-chlorobenzoyl chloride, instead of 2-fluoro-3-(trifluoromethyl)aniline and 3-(trifluoromethyl)benzoyl chloride, respectively (Yield: 42%).

¹H NMR (DMSO-d₆, 400 MHz) δ 8.16-8.19 (m, 2H), 7.71 (d, 1H), 7.66 (d, 1H), 7.50-7.60 (m, 3H), 5.26 (s, 2H)

### Example 359

### 2-(5-chloro-2-((3-(trifluoromethyl)benzoyl)imino)benzo[d]thiazol-3(2H)-yl)propionic acid

The titled compound as a white solid was prepared in accordance with the same procedures as in Example 266, using 5-chloro-2-fluoroaniline and ethyl 2-bromopropionate, instead of 2-fluoro-3-(trifluoromethyl)aniline and ethyl bromoacetate, respectively (Yield: 28%).

¹H NMR (DMSO-d₆, 400 MHz) δ 13.15 (m, 1H), 8.46-8.51 (m, 2H), 7.97-8.08 (m, 3H), 7.79 (t, 1H), 7.48 (d, 1H), 5.85-5.86 (m, 1H), 1.74 (d, 3H)

### Example 360

### 2-(5-chloro-2-((3-chlorobenzoyl)imino)benzo[d]thiazol-3(2H)-yl)propionic acid

The titled compound as a white solid was prepared in accordance with the same procedures as in Example 266, using 5-chloro-2-fluoroaniline, 3-chlorobenzoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 31%).

¹H NMR (DMSO-d₆, 400 MHz) δ 13.17 (s, 1H), 8.20 (s, 1H), 8.14 (d, 1H), 8.02-8.05 (m, 2H), 7.68 (d, 1H), 7.57 (t, 1H), 7.48 (d, 1H), 5.84-5.85 (m, 1H), 1.72 (d, 3H)

### Example 361

### 2-(5-chloro-2-((3-chlorobenzoyl)imino)benzo[d]thiazol-3(2H)-yl)butanoic acid

The titled compound as a white solid was prepared in accordance with the same procedures as in Example 266, using 5-chloro-2-fluoroaniline, 3-chlorobenzoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 21%).

¹H NMR (DMSO-d₆, 400 MHz) δ 13.18 (m, 1H), 8.18 (s, 1H), 8.02-8.14 (m, 3H), 7.68 (d, 1H), 7.57 (t, 1H), 7.48 (d, 1H), 5.74 (m, 1H), 2.33-2.51 (m, 2H), 0.78 (t, 3H)

### Example 362

### 2-(5-fluoro-6-methyl-2-((3-(trifluoromethyl)benzoyl)imino)benzo[d]thiazol-3(2H)-yl)butanoic acid

The titled compound as a white solid was prepared in accordance with the same procedures as in Example 266, using 2,5-difluoro-4-methylaniline and methyl 2-bromobutyrate, instead of 2-fluoro-3-(trifluoromethyl)aniline and ethyl bromoacetate, respectively (Yield: 31%).

¹H NMR (MeOD, 400 MHz): 8.52-8.50 (m, 2H), 7.83 (d, 1H), 7.68 (dd, 2H), 7.33-7.20 (m, 1H), 6.20-5.70 (m, 1H), 2.60-2.40 (m, 2H), 2.35 (s, 3H), 0.83 (t, 3H)

### Example 363

### 2-(5-fluoro-2-((3-(trifluoromethyl)benzoyl)imino)benzo[d]thiazol-3(2H)-yl)butanoic acid

The titled compound as a white solid was prepared in accordance with the same procedures as in Example 266, using 2,5-difluoroaniline and methyl 2-bromobutyrate, instead of 2-fluoro-3-(trifluoromethyl)aniline and ethyl bromoacetate, respectively (Yield: 16%).

¹H NMR (MeOD, 400 MHz): 8.55 (s, 1H), 8.50 (d, 1H), 7.90-7.80 (m, 2H), 7.69 (dd, 1H), 7.59 (d, 1H), 7.19 (dd, 1H), 5.75-5.50 (m, 1H), 2.70-2.40 (m, 2H), 0.88 (t, 3H)

### Example 364

### 2-(2-((3-chlorobenzoyl)imino)-4,6-difluorobenzo[d]thiazol-3(2H)-yl)butanoic acid

The titled compound as a white solid was prepared in accordance with the same procedures as in Example 200, using 3-chlorobenzoyl chloride and methyl 2-bromobutyrate, instead of p-toluoyl chloride and ethyl bromoacetate, respectively (Yield: 22%).

¹H NMR (MeOD, 400 MHz): 8.23 (s, 1H), 8.19 (d, 1H), 7.60-7.52 (m, 2H), 7.48 (dd, 1H), 7.30-7.20 (m, 1H), 6.50-5.65 (brs, 1H), 2.75-2.15 (m, 2H), 0.93 (t, 3H)

### Example 365

### 2-(5-fluoro-2-((3-(trifluoromethyl)benzoyl)imino)benzo[d]thiazol-3(2H)-yl)acetic acid

The titled compound as a white solid was prepared in accordance with the same procedures as in Example 266, using 2,5-difluoroaniline, instead of 2-fluoro-3-(trifluoromethyl)aniline (Yield: 25%).

¹H NMR (DMSO-d₆, 400 MHz); 13.43 (s, 1H), 8.51 (d, 2H), 8.02 (m, 2H), 7.81 (m, 2H), 7.29 (t, 1H), 5.33 (s, 2H)

### Example 366

### 2-(7-fluoro-2-((3-(trifluoromethyl)benzoyl)imino)benzo[d]thiazol-3(2H)-yl)butanoic acid

The titled compound as a white solid was prepared in accordance with the same procedures as in Example 266, using 2,3-difluoroaniline and methyl 2-bromobutyrate, instead of 2-fluoro-3-(trifluoromethyl)aniline and ethyl bromoacetate, respectively (Yield: 18%).

¹H NMR (DMSO-d₆, 400 MHz); 13.24 (brs, 1H), 8.51 (s, 1H), 8.49 (d, 1H), 8.01 (d, 1H), 7.80 (m, 2H), 7.66 (m, 1H), 7.35 (t, 1H), 5.82 (brs, 1H), 2.51-2.37 (m, 2H), 0.79 (t, 3H)

### Example 367

### 2-(2-((4-methylbenzoyl)imino)-5-(trifluoromethyl)benzo[d]thiazol-3(2H)-yl)acetic acid

The titled compound as a white solid was prepared in accordance with the same procedures as in Example 266, using 2-fluoro-5-(trifluoromethyl)aniline and p-toluoyl chloride, instead of 2-fluoro-3-(trifluoromethyl)aniline and 3-(trifluoromethyl)benzoyl chloride, respectively (Yield: 28%).

¹H NMR (DMSO-d₆, 400 MHz); 13.39 (brs, 1H), 8.21 (m, 2H), 8.15 (m, 2H), 7.72 (d, 1H), 7.35 (d, 2H), 5.41 (s, 2H), 2.40 (s, 3H)

### Example 368

### 2-(5-methyl-2-((3-(trifluoromethyl)benzoyl)imino)benzo[d]thiazol-3(2H)-yl)butanoic acid

The titled compound as a white solid was prepared in accordance with the same procedures as in Example 266, using 2-fluoro-5-methylaniline and methyl 2-bromobutyrate, instead of 2-fluoro-3-(trifluoromethyl)aniline and ethyl bromoacetate, respectively (Yield: 14%).

¹H NMR (DMSO-d₆, 400 MHz); 13.09 (brs, 1H), 8.50 (s, 1H), 8.47 (d, 1H), 7.97 (d, 1H), 7.88 (d, 1H), 7.77 (m, 2H), 5.73 (brs, 1H), 2.51-2.36 (m, 2H), 2.47 (s, 3H), 0.78 (t, 3H)

### Example 369

### 2-(2-((3-chlorobenzoyl)imino)-5-methylbenzo[d]thiazol-3(2H)-yl)acetic acid

The titled compound as a white solid was prepared in accordance with the same procedures as in Example 266, using 2-fluoro-5-methylaniline and 3-chlorobenzoyl chloride, instead of 2-fluoro-3-(trifluoromethyl)aniline and 3-(trifluoromethyl)benzoyl chloride, respectively (Yield: 13%).

¹H NMR (DMSO-d₆, 400 MHz); 13.39 (brs, 1H), 8.21 (s, 1H), 8.18 (d, 1H), 7.85 (d, 1H), 7.66 (m, 2H), 7.55 (m, 1H), 7.25 (d, 1H), 5.32 (s, 2H), 2.46 (s, 3H)

### Example 370

### 2-(2-((3-chlorobenzoyl)imino)-5-methylbenzo[d]thiazol-3(2H)-yl)propionic acid

The titled compound as a white solid was prepared in accordance with the same procedures as in Example 266, using 2-fluoro-5-methylaniline, 3-chlorobenzoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 7%).

¹H NMR (DMSO-d₆, 400 MHz); 13.11, (brs, 1H), 8.21 (s, 1H), 8.16 (d, 1H), 7.86 (d, H), 7.67 (m, 2H), 7.56 (m, 1H), 7.26 (d, 1H), 5.84 (brs, 1H), 2.47 (s, 3H), 1.73 (d, 3H)

### Example 371

### 2-(2-((3-chlorobenzoyl)imino)-5-fluoro-6-methylbenzo[d]thiazol-3(2H)-yl)propionic acid

The titled compound as a white solid was prepared in accordance with the same procedures as in Example 200, using 5-fluoro-6-methylbenzo[d]thiazol-2-amine, 3-chlorobenzoyl chloride, and ethyl 2-bromopropionate, instead of 4,6-difluorobenzo[d]thiazol-2-amine, p-toluoyl chloride, and ethyl bromoacetate, respectively (Yield: 41%).

¹H NMR (DMSO-d₆, 400 MHz); 13.16 (brs, 1H), 8.19 (s, 1H), 8.14 (d, 1H), 7.90 (d, 1H), 7.82 (d, 1H), 7.67 (d, 1H), 7.58 (t, 1H), 5.77 (brs, 1H), 2.33 (s, 3H), 1.72 (d, 3H)

### Example 372

### 2-(2-((3-chlorobenzoyl)imino)-5-fluoro-6-methylbenzo[d]thiazol-3(2H)-yl)butanoic acid

The titled compound as a white solid was prepared in accordance with the same procedures as in Example 200, using 5-fluoro-6-methylbenzo[d]thiazol-2-amine, 3-chlorobenzoyl chloride, and methyl 2-bromobutyrate, instead of 4,6-difluorobenzo[d]thiazol-2-amine, p-toluoyl chloride, and ethyl bromoacetate, respectively (Yield: 28%).

¹H NMR (DMSO-d₆, 400 MHz); 13.16 (brs, 1H), 8.17 (s, 1H), 8.13 (d, 1H), 7.91 (d, 1H), 7.85 (s, 1H), 7.67 (d, 1H), 7.56 (t, 1H), 5.65 (brs, 1H), 2.51-2.30 (m, 2H), 2.30 (s, 3H), 0.76 (t, 3H)

### Example 373

### 2-(2-((3-chlorobenzoyl)imino)-7-fluorobenzo[d]thiazol-3(2H)-yl)propionic acid

The titled compound as a white solid was prepared in accordance with the same procedures as in Example 266, using 2,3-difluoroaniline, 3-chlorobenzoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 14%).

¹H NMR (DMSO-d₆, 400 MHz); 13.25 (brs, 1H), 8.21 (s, 1H), 8.17 (d, 1H), 7.73-7.56 (m, 4H), 7.33 (t, 1H), 5.94 (brs, 1H), 1.75 (d, 3H)

### Example 374

### 2-(2-((3-chlorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 3-chlorobenzoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 36%).

¹H NMR (DMSO-d₆, 400 MHz); 13.24 (brs, 1H), 8.20 (s, 1H), 8.16 (d, 1H), 7.75 (s, 1H), 7.70-7.56 (m, 3H), 7.33 (t, 1H), 5.81 (brs, 1H), 2.51-2.36 (m, 2H), 0.78 (t, 3H)

### Example 375

### 2-(2-((3-chlorobenzoyl)imino)-5-fluorobenzo[d]thiazol-3(2H)-yl)acetic acid

The titled compound as a white solid was prepared in accordance with the same procedures as in Example 266, using 2,5-difluoroaniline and 3-chlorobenzoyl chloride, instead of 2-fluoro-3-(trifluoromethyl)aniline and 3-(trifluoromethyl)benzoyl chloride, respectively (Yield: 28%).

¹H NMR (DMSO-d₆, 400 MHz); 13.41 (brs, 1H), 8.25-8.16 (m, 2H), 8.01 (m, 1H), 7.85 (m, 1H), 7.69-7.53 (m, 2H), 7.28 (m, 1H), 5.33 (s, 2H)

### Example 376

### 2-(2-((3-chlorobenzoyl)imino)-5-fluorobenzo[d]thiazol-3(2H)-yl)propionic acid

The titled compound as a white solid was prepared in accordance with the same procedures as in Example 266, using 2,5-difluoroaniline, 3-chlorobenzoyl chloride, and ethyl 2-bromopropionate, instead of 2-fluoro-3-(trifluoromethyl)aniline, 3-(trifluoromethyl)benzoyl chloride, and ethyl bromoacetate, respectively (Yield: 24%).

¹H NMR (DMSO-d₆, 400 MHz); 13.22 (brs, 1H), 8.23-8.14 (m, 2H), 8.02 (s, 1H), 7.86 (d, 1H), 7.68-7.50 (m, 2H), 7.30 (m, 1H), 5.80 (brs, 1H), 1.73 (s, 2H)

### Example 377

### 2-(2-((3-chlorobenzoyl)imino)-5-fluorobenzo[d]thiazol-3(2H)-yl)butanoic acid

The titled compound as a white solid was prepared in accordance with the same procedures as in Example 266, using 2,5-difluoroaniline, 3-chlorobenzoyl chloride, and methyl 2-bromobutyrate, instead of 2-fluoro-3-(trifluoromethyl)aniline, 3-(trifluoromethyl)benzoyl chloride, and ethyl bromoacetate, respectively (Yield: 33%).

¹H NMR (DMSO-d₆, 400 MHz); 13.23 (brs, 1H), 8.18 (m, 2H), 8.14 (s, 1H), 7.85 (d, 1H), 7.68-7.52 (m, 2H), 7.30 (m, 1H), 5.80-5.68 (brs, 1H), 2.51-2.34 (m, 2H), 0.77 (t, 3H)

### Example 378

### 2-(5,6,7-trifluoro-2-((3-(trifluoromethyl)benzoyl)imino)benzo[d]thiazol-3(2H)-yl)acetic acid

The titled compound (9 mg) as a white solid was prepared in accordance with the same procedures as in Example 200, using N-(5,6,7-trifluorobenzo[d]thiazol-2-yl)-3-(trifluoromethyl)benzamide (50 mg, 0.13 mmol) and 3-(trifluoromethyl)benzoyl chloride, instead of N-(4,6-difluorobenzo[d]thiazol-2-yl)-4-methylbenzamide and p-toluoyl chloride, respectively.

¹H NMR (DMSO-d₆, 400 MHz) δ 8.49 (m, 2H), 8.01 (t, 1H), 7.83 (m, 2H), 5.32 (d, 2H)

### Example 379

### 2-(5,6,7-trifluoro-2-((3-(trifluoromethyl)benzoyl)imino)benzo[d]thiazol-3(2H)-yl)butanoic acid

The titled compound (9 mg) as a white solid was prepared in accordance with the same procedures as in Example 200, using N-(5,6,7-trifluorobenzo[d]thiazol-2-yl)-3-(trifluoromethyl)benzamide (50 mg, 0.13 mmol), 3-(trifluoromethyl)benzoyl chloride, and methyl 2-bromobutyrate, instead of N-(4,6-difluorobenzo[d]thiazol-2-yl)-4-methylbenzamide, p-toluoyl chloride, and ethyl bromoacetate, respectively.

¹H NMR (DMSO-d₆, 400 MHz) δ 8.24-8.27 (m, 2H), 7.88-8.00 (m, 1H), 7.80 (d, 1H), 7.59 (t, 1H), 5.45-5.47 (m, 1H), 2.12-2.29 (m, 2H), 0.58 (t, 3H)

### Example 380

### 2-(5,6,7-trifluoro-2-((3-(trifluoromethyl)benzoyl)imino)benzo[d]thiazol-3(2H)-yl)propionic acid

The titled compound (40 mg) as a white solid was prepared in accordance with the same procedures as in Example 200, using N-(5,6,7-trifluorobenzo[d]thiazol-2-yl)-3-(trifluoromethyl)benzamide (100 mg, 0.27 mmol), 3-(trifluoromethyl)benzoyl chloride, and ethyl 2-bromopropionate, instead of N-(4,6-difluorobenzo[d]thiazol-2-yl)-4-methylbenzamide, p-toluoyl chloride, and ethyl bromoacetate, respectively.

¹H NMR (DMSO-d₆, 400 MHz) δ 8.49 (s, 2H), 7.93-8.01 (m, 2H), 7.81 (t, 1H), 5.76-5.78 (m, 1H), 1.70 (d, 3H)

### Example 381

### 2-(6-chloro-7-fluoro-2-((3-(trifluoromethyl)benzoyl)imino)benzo[d]thiazol-3(2H)-yl)acetic acid

To a solution of N-(6-chloro-7-fluorobenzo[d]thiazol-2(3H)-ylidene)-3-(trifluoromethyl)benzamide (150 mg, 0.40 mmol) in N,N-dimethylformamide (4 mL), were added potassium carbonate (111 mg, 0.80 mmol) and ethyl bromoacetate (66 μl, 0.60 mmol). The reaction mixture was stirred at 50° C. overnight. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=10/1-11). Tetrahydrofuran (3 mL) and a 1N sodium hydroxide solution (1 mL) were added to the resulting product. The reaction mixture was stirred at room temperature overnight, washed with dichloromethane, and then acidified to pH 3-4 with an aqueous 1N HCl solution. The resulting solid was filtered, washed with diisopropyl ether, and then dried under reduced pressure to give 38 mg of the titled compound as a white solid.

¹H NMR (MeOD, 400 MHz): 8.53 (s, 1H), 8.51 (d, 1H), 7.86 (d, 1H), 7.69 (dd, 1H), 7.61 (dd, 1H), 7.45 (d, 1H), 5.33 (s, 2H)

### Example 382

### 2-(6-chloro-7-fluoro-2-((3-(trifluoromethyl)benzoyl)imino)benzo[d]thiazol-3(2H)-yl)propionic acid

The titled compound (13 mg) as a white solid was prepared in accordance with the same procedures as in Example 381, using ethyl 2-bromopropionate, instead of ethyl bromoacetate.

¹H NMR (MeOD, 400 MHz): 8.58 (s, 1H), 8.52 (d, 1H), 7.87 (d, 1H), 7.80-7.60 (m, 2H), 7.52 (d, 1H), 5.90-5.70 (m, 1H), 1.85 (d, 3H)

### Example 383

### 2-(6-chloro-7-fluoro-2-((3-(trifluoromethyl)benzoyl)imino)benzo[d]thiazol-3(2H)-yl)butanoic acid

The titled compound (41 mg) as a white solid was prepared in accordance with the same procedures as in Example 381, using methyl 2-bromobutyrate, instead of ethyl bromoacetate.

¹H NMR (MeOD, 400 MHz): 8.55 (s, 1H), 8.51 (d, 1H), 7.87 (d, 1H), 7.74-7.60 (m, 2H), 7.56 (d, 1H), 5.80-5.60 (m, 1H), 2.70-2.40 (m, 2H), 0.89 (t, 3H)

### Example 384

### 2-(6-chloro-2-((3-chlorobenzoyl)imino)-7-fluorobenzo[d]thiazol-3(2H)-yl)acetic acid

The titled compound (40 mg) as a white solid was prepared in accordance with the same procedures as in Example 381, using N-(6-chloro-7-fluorobenzo[d]thiazol-2(3H)-ylidene)-3-chlorobenzamide (150 mg, 0.44 mmol), instead of N-(6-chloro-7-fluorobenzo[d]thiazol-2(3H)-ylidene)-3-(trifluoromethyl)benz amide.

¹H NMR (MeOD, 400 MHz): 8.21 (s, 1H), 8.18 (d, 1H), 7.62-7.50 (m, 2H), 7.45 (dd, 1H), 7.40 (d, 1H), 5.27 (s, 2H)

### Example 385

### 2-(6-chloro-2-((3-chlorobenzoyl)imino)-7-fluorobenzo[d]thiazol-3(2H)-yl)propionic acid

The titled compound (29 mg) as a white solid was prepared in accordance with the same procedures as in Example 381, using N-(6-chloro-7-fluorobenzo[d]thiazol-2(3H)-ylidene)-3-chlorobenzamide (150 mg, 0.44 mmol) and ethyl 2-bromopropionate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.23 (s, 1H), 8.18 (d, 1H), 7.64 (dd, 1H), 7.62-7.42 (m, 3H), 5.90-5.70 (m, 1H), 1.84 (d, 3H)

### Example 386

### 2-(6-chloro-2-((3-chlorobenzoyl)imino)-7-fluorobenzo[d]thiazol-3(2H)-yl)butanoic acid

The titled compound (32 mg) as a white solid was prepared in accordance with the same procedures as in Example 381, using N-(6-chloro-7-fluorobenzo[d]thiazol-2(3H)-ylidene)-3-chlorobenzamide (150 mg, 0.44 mmol) and methyl 2-bromobutyrate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.24 (s, 1H), 8.20 (d, 1H), 7.64 (dd, 1H), 7.62-7.51 (m, 2H), 7.48 (dd, 1H), 5.80-5.60 (m, 1H), 2.70-2.40 (m, 2H), 0.88 (t, 3H)

### Example 387

### 2-(2-((3-chlorobenzoyl)imino)-6-fluoro-5-(trifluoromethyl)benzo[d]thiazol-3(2H)-yl)acetic acid

The titled compound (43 mg) as a white solid was prepared in accordance with the same procedures as in Example 381, using N-(6-fluoro-5-(trifluoromethyl)benzo[d]thiazol-2(3H)-ylidene)-3-chlorobenzamide (150 mg, 0.40 mmol), instead of N-(6-chloro-7-fluorobenzo[d]thiazol-2(3H)-ylidene)-3-(trifluoromethyl)benzamide.

¹H NMR (MeOD, 400 MHz): 8.22 (s, 1H), 8.18 (d, 1H), 7.95-7.85 (m, 2H), 7.55 (d, 1H), 7.46 (dd, 1H), 5.39 (s, 2H)

### Example 388

### 2-(2-((3-chlorobenzoyl)imino)-6-fluoro-5-(trifluoromethyl)benzo[d]thiazol-3(2H)-yl)propionic acid

The titled compound (36 mg) as a white solid was prepared in accordance with the same procedures as in Example 381, using N-(6-fluoro-5-(trifluoromethyl)benzo[d]thiazol-2(3H)-ylidene)-3-chlorobenzamide (150 mg, 0.40 mmol) and ethyl 2-bromopropionate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.25 (s, 1H), 8.19 (d, 1H), 8.02 (d, 1H), 7.92 (d, 1H), 7.57 (d, 1H), 7.47 (dd, 1H), 5.89-5.80 (m, 1H), 1.84 (d, 3H)

### Example 389

### 2-(2-((3-chlorobenzoyl)imino)-6-fluoro-5-(trifluoromethyl)benzo[d]thiazol-3(2H)-yl)butanoic acid

The titled compound (11 mg) as a white solid was prepared in accordance with the same procedures as in Example 381, using N-(6-fluoro-5-(trifluoromethyl)benzo[d]thiazol-2(3H)-ylidene)-3-chlorobenzamide (150 mg, 0.40 mmol) and methyl 2-bromobutyrate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.24 (s, 1H), 8.18 (d, 1H), 8.10-8.00 (m, 1H), 7.94 (d, 1H), 7.60-7.52 (m, 1H), 7.51-7.43 (m, 1H), 5.90-5.65 (m, 1H), 2.70-2.40 (m, 2H), 0.88 (t, 3H)

### Example 390

### 2-(7-methoxy-2-((3-(trifluoromethyl)benzoyl)imino)benzo[d]thiazol-3(2H)-yl)acetic acid

To a solution of N-(7-methoxybenzo[d]thiazol-2(3H)-ylidene)-3-(trifluoromethyl)benzamide (200 mg, 0.57 mmol) in N,N-dimethylformamide (2.0 mL), were added potassium carbonate (236 mg, 1.71 mmol) and ethyl bromoacetate (95 uL, 0.86 mmol). The reaction mixture was stirred at 80° C. for 3 days and quenched with water. The reaction mixture was extracted with ethyl acetate. The organic layer was washed with brine, dried over anhydrous magnesium sulfate, filtered, and then evaporated. The residue was purified with Prep. TLC (n-hexaneethyl acetate=41). Methanol (1.5 mL) and a 10% sodium hydroxide solution (0.5 mL) were added to the resulting liquid product. The reaction mixture was stirred at room temperature overnight, diluted with water, washed with ether three times, and then acidified to pH 3-4 with an aqueous 1N HCl solution. Sodium chloride was added to the aqueous layer, which was then extracted with acetonitrile three times. The organic layer was dried over anhydrous magnesium sulfate, filtered, and then evaporated. The resulting residue was dried under reduced pressure to give 70 mg of the titled compound as a pale yellow solid.

¹H NMR (CDCl₃, 400 MHz) δ 7.65 (s, 1H), 7.55 (m, 2H), 7.34 (t, 1H), 6.92 (t, 1H), 6.83 (dd, 2H), 4.97-4.98 (m, 1H), 4.22 (m, 1H), 3.83 (s, 3H)

### Example 391

### 2-(7-methoxy-2-((3-(trifluoromethyl)benzoyl)imino)benzo[d]thiazol-3(2H)-yl)propionic acid

The titled compound (36 mg) as a colorless oil was prepared in accordance with the same procedures as in Example 390, using ethyl 2-bromopropionate, instead of ethyl bromoacetate.

¹H NMR (CDCl₃, 400 MHz) δ 7.50-7.63 (m, 3H), 7.26-7.31 (m, 1H), 6.85-7.08 (m, 3H), 5.39 (m, 1H), 3.78 (s, 3H), 1.36 (m, 3H)

### Example 392

### 2-(7-methoxy-2-((3-(trifluoromethyl)benzoyl)imino)benzo[d]thiazol-3(2H)-yl)butanoic acid

The titled compound (19 mg) as a colorless oil was prepared in accordance with the same procedures as in Example 390, using methyl 2-bromobutyrate, instead of ethyl bromoacetate.

¹H NMR (CDCl₃, 400 MHz) δ 7.50-7.57 (m, 3H), 7.26-7.31 (m, 1H), 6.90-7.11 (m, 2H), 6.83 (m, 1H), 5.15 (m, 1H), 3.74 (s, 3H), 1.69-1.91 (m, 2H), 1.11 (m, 3H)

### Example 393

### 2-(2-((3-chlorobenzoyl)imino)-7-methoxybenzo[d]thiazol-3(2H)-yl)acetic acid

The titled compound (38 mg) as a white solid was prepared in accordance with the same procedures as in Example 390, using N-(7-methoxybenzo[d]thiazol-2(3H)-ylidene)-3-chlorobenzamide (170 mg, 0.53 mmol), instead of N-(7-methoxybenzo[d]thiazol-2(3H)-ylidene)-3-(trifluoromethyl)benzamide.

¹H NMR (CDCl₃, 400 MHz) δ 7.43 (s, 1H), 7.21 (d, 1H), 7.12 (t, 1H), 6.92 (t, 1H), 6.79-6.86 (m, 2H), 4.87-4.97 (m, 2H), 3.85 (s, 3H)

### Example 394

### 2-(2-((3,4-dichlorobenzoyl)imino)-6,7-difluorobenzo[d]thiazol-3(2H)-yl)butanoic acid

The titled compound as a white solid was prepared in accordance with the same procedures as in Example 266, using 2,3,4-trifluoroaniline, 3,4-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 10%).

¹H NMR (DMSO-d₆, 400 MHz) δ 13.30 (brs, 1H), 8.32 (s, 1H), 8.11 (d, 1H), 7.82 (d, 1H), 7.70 (m, 2H), 5.80 (brs, 1H), 2.51-2.33 (m, 2H), 0.79 (t, 3H)

### Example 395

### 2-(2-((3,5-dichlorobenzoyl)imino)-6,7-difluorobenzo[d]thiazol-3(2H)-yl)butanoic acid

The titled compound as a white solid was prepared in accordance with the same procedures as in Example 266, using 2,3,4-trifluoroaniline, 3,5-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 16%).

¹H NMR (DMSO-d₆, 400 MHz) δ 13.31 (brs, 1H), 8.10 (s, 2H), 7.88 (s, 1H), 7.76 (m, 2H), 5.81 (brs, 1H), 2.50-2.33 (m, 2H), 0.78 (t, 3H)

### Example 396

### 2-(2-((3-chlorobenzoyl)imino)-7-fluoro-6-methoxybenzo[d]thiazol-3(2H)-yl)acetic acid

To a solution of N-(7-fluoro-6-methoxybenzo[d]thiazol-2(3H)-ylidene)-3-chlorobenzamide (167 mg, 0.50 mmol) in N,N-dimethylformamide (3 mL) were added potassium carbonate (140 mg, 1.00 mmol) and ethyl bromoacetate (83 uL, 0.75 mmol). The reaction mixture was stirred at 50° C. overnight. The reaction mixture was quenched with water and then extracted with ethyl acetate. The organic layer was washed with brine, dried over anhydrous magnesium sulfate, filtered, and then evaporated. The residue was purified with silica gel column chromatography (n-hexaneethyl acetate=31). To the resulting solid, were added tetrahydrofuran (6 mL) and a 1N sodium hydroxide solution (3 mL). The reaction mixture was stirred at room temperature overnight, acidified to pH 3-4 with an aqueous 1N HCl solution, and then extracted with ethyl acetate. The organic layer was washed with brine, dried over anhydrous magnesium sulfate, filtered, and then evaporated. The residue was dried under reduced pressure to give 88 mg of the titled compound as a white solid.

¹H NMR (DMSO-d₆, 400 MHz); 13.44 (brs, 1H), 8.19 (s, 1H), 8.16 (d, 1H), 7.68 (d, 1H), 7.58 (t, 2H), 7.45 (m, 1H), 5.32 (s, 2H), 3.92 (s, 3H)

### Example 397

### 2-(2-((3-chlorobenzoyl)imino)-7-fluoro-6-methoxybenzo[d]thiazol-3(2H)-yl)propionic acid

The titled compound (92 mg) as a white solid was prepared in accordance with the same procedures as in Example 396, using ethyl 2-bromopropionate, instead of ethyl bromoacetate.

¹H NMR (DMSO-d₆, 400 MHz); 13.20 (brs, 1H), 8.19 (s, 1H), 8.15 (d, 1H), 7.66 (m, 2H), 7.56 (t, 1H), 7.48 (t, 1H), 5.87 (brs, 1H), 3.93 (s, 3H), 1.71 (d, 3H)

### Example 398

### 2-(2-((3-chlorobenzoyl)imino)-7-fluoro-6-methoxybenzo[d]thiazol-3(2H)-yl)butanoic acid

The titled compound (90 mg) as a white solid was prepared in accordance with the same procedures as in Example 396, using methyl 2-bromobutyrate, instead of ethyl bromoacetate.

¹H NMR (DMSO-d₆, 400 MHz); 13.21 (brs, 1H), 8.18 (s, 1H), 8.14 (d, 1H), 7.68 (d, 2H), 7.56 (t, 1H), 7.47 (t, 1H), 5.76 (brs, 1H), 3.93 (s, 3H), 2.50-2.33 (m, 2H), 0.77 (t, 3H)

### Example 399

### 2-(6-ethoxy-7-fluoro-2-((3-(trifluoromethyl)benzoyl)imino)benzo[d]thiazol-3(2H)-yl)acetic acid

The titled compound (101 mg) as a white solid was prepared in accordance with the same procedures as in Example 396, using N-(6-ethoxy-7-fluorobenzo[d]thiazol-2(3H)-ylidene)-3-(trifluoromethyl)benzamide (192 mg, 0.50 mmol), instead of N-(7-fluoro-6-methoxybenzo[d]thiazol-2(3H)-ylidene)-3-chlorobenzamide.

¹H NMR (DMSO-d₆, 400 MHz); 13.45 (brs, 1H), 8.50 (m, 2H), 7.99 (d, 1H), 7.80 (d, 1H), 7.61 (d, 1H), 7.45 (d, 1H), 5.33 (s, 2H), 4.20 (d, 2H), 1.38 (d, 3H)

### Example 400

### 2-(6-ethoxy-7-fluoro-2-((3-(trifluoromethyl)benzoyl)imino)benzo[d]thiazol-3(2H)-yl)propionic acid

The titled compound (99 mg) as a white solid was prepared in accordance with the same procedures as in Example 396, using N-(6-ethoxy-7-fluorobenzo[d]thiazol-2(3H)-ylidene)-3-(trifluoromethyl)benzamide (192 mg, 0.50 mmol) and ethyl 2-bromopropionate, instead of N-(7-fluoro-6-methoxybenzo[d]thiazol-2(3H)-ylidene)-3-chlorobenzamide and ethyl bromoacetate, respectively.

¹H NMR (DMSO-d₆, 400 MHz); 13.18 (brs, 1H), 8.50 (s, 1H), 8.47 (d, 1H), 7.98 (d, 1H), 7.78 (t, 1H), 7.65 (d, 1H), 7.47 (t, 1H), 5.87 (brs, 1H), 4.20 (d, 2H), 1.74 (d, 3H), 1.37 (t, 3H)

### Example 401

### 2-(6-ethoxy-7-fluoro-2-((3-(trifluoromethyl)benzoyl)imino)benzo[d]thiazol-3(2H)-yl)butanoic acid

The titled compound (90 mg) as a white solid was prepared in accordance with the same procedures as in Example 396, using N-(6-ethoxy-7-fluorobenzo[d]thiazol-2(3H)-ylidene)-3-(trifluoromethyl)benzamide (192 mg, 0.50 mmol) and methyl 2-bromobutyrate, instead of N-(7-fluoro-6-methoxybenzo[d]thiazol-2(3H)-ylidene)-3-chlorobenzamide and ethyl bromoacetate, respectively.

¹H NMR (DMSO-d₆, 400 MHz); 13.19 (brs, 1H), 8.49 (s, 1H), 8.47 (d, 1H), 7.99 (d, 1H), 7.78 (t, 1H), 7.68 (s, 1H), 7.47 (t, 1H), 5.75 (brs, 1H), 4.22 (d, 1H), 2.50-2.34 (m, 2H), 1.38 (t, 3H), 0.78 (t, 3H)

### Example 402

### 2-(7-chloro-5-(trifluoromethyl)-2-((3-(trifluoromethyl)benzoyl)imino)benzo[d]thiazol-3(2H)-yl)acetic acid

The titled compound (104 mg) as a white solid was prepared in accordance with the same procedures as in Example 396, using N-(7-chloro-5-(trifluoromethyl)benzo[d]thiazol-2(3H)-ylidene)-3-(trifluoromethyl)benzamide (212 mg, 0.50 mmol), instead of N-(7-fluoro-6-methoxybenzo[d]thiazol-2(3H)-ylidene)-3-chlorobenzamide.

¹H NMR (DMSO-d₆, 400 MHz); 13.51 (brs, 1H), 8.49 (s, 2H), 8.34 (s, 1H), 8.01 (d, 1H), 7.95 (s, 1H), 7.82 (t, 1H), 5.45 (s, 2H)

### Example 403

### 2-(7-chloro-5-(trifluoromethyl)-2-((3-(trifluoromethyl)benzoyl)imino)benzo[d]thiazol-3(2H)-yl)propionic acid

The titled compound (110 mg) as a white solid was prepared in accordance with the same procedures as in Example 396, using N-(7-chloro-5-(trifluoromethyl)benzo[d]thiazol-2(3H)-ylidene)-3-(trifluoromethyl)benzamide (212 mg, 0.50 mmol) and ethyl 2-bromopropionate, instead of N-(7-fluoro-6-methoxybenzo[d]thiazol-2(3H)-ylidene)-3-chlorobenzamide and ethyl bromoacetate, respectively.

¹H NMR (DMSO-d₆, 400 MHz); 13.28 (brs, 1H), 8.50 (s, 1H), 8.46 (d, 1H), 8.36 (s, 1H), 8.01 (d, 1H), 7.96 (s, 1H), 7.80 (t, 1H), 6.01 (brs, 1H), 1.78 (d, 3H)

### Example 404

### 2-(7-chloro-5-(trifluoromethyl)-2-((3-(trifluoromethyl)benzoyl)imino)benzo[d]thiazol-3(2H)-yl)butanoic acid

The titled compound (98 mg) as a white solid was prepared in accordance with the same procedures as in Example 396, using N-(7-chloro-5-(trifluoromethyl)benzo[d]thiazol-2(3H)-ylidene)-3-(trifluoromethyl)benzamide (212 mg, 0.50 mmol) and methyl 2-bromobutyrate, instead of N-(7-fluoro-6-methoxybenzo[d]thiazol-2(3H)-ylidene)-3-chlorobenzamide and ethyl bromoacetate, respectively.

¹H NMR (DMSO-d₆, 400 MHz); 13.31 (brs, 1H), 8.54-8.41 (m, 3H), 8.12-7.97 (m, 2H), 7.81 (s, 1H), 5.92 (brs, 1H), 2.51-2.38 (m, 2H), 0.82 (t, 3H)

### Example 405

### 2-(7-chloro-2-((3-chlorobenzoyl)imino)-5-(trifluoromethyl)benzo[d]thiazol-3(2H)-yl)acetic acid

The titled compound (90 mg) as a white solid was prepared in accordance with the same procedures as in Example 396, using N-(7-chloro-5-(trifluoromethyl)benzo[d]thiazol-2(3H)-ylidene)-3-chlorobenzamide (196 mg, 0.50 mmol), instead of N-(7-fluoro-6-methoxybenzo[d]thiazol-2(3H)-ylidene)-3-chlorobenzamide.

¹H NMR (DMSO-d₆, 400 MHz); 13.52 (brs, 1H), 8.31 (s, 1H), 8.18 (m, 2H), 7.93 (s, 1H), 7.69 (d, 1H), 7.59 (d, 1H), 5.43 (s, 2H)

### Example 406

### 2-(7-chloro-2-((3-chlorobenzoyl)imino)-5-(trifluoromethyl)benzo[d]thiazol-3(2H)-yl)propionic acid

The titled compound (95 mg) as a white solid was prepared in accordance with the same procedures as in Example 396, using N-(7-chloro-5-(trifluoromethyl)benzo[d]thiazol-2(3H)-ylidene)-3-chlorobenzamide (196 mg, 0.50 mmol) and ethyl 2-bromopropionate, instead of N-(7-fluoro-6-methoxybenzo[d]thiazol-2(3H)-ylidene)-3-chlorobenzamide and ethyl bromoacetate, respectively.

¹H NMR (DMSO-d₆, 400 MHz); 13.29 (brs, 1H), 8.33 (s, 1H), 8.19 (s, 1H), 8.15 (d, 1H), 7.97 (s, 1H), 7.72 (d, 1H), 7.59 (t, 1H), 5.99 (brs, 1H), 1.76 (d, 3H)

### Example 407

### 2-(7-chloro-2-((3-chlorobenzoyl)imino)-5-(trifluoromethyl)benzo[d]thiazol-3(2H)-yl)butanoic acid

The titled compound (88 mg) as a white solid was prepared in accordance with the same procedures as in Example 396, using N-(7-chloro-5-(trifluoromethyl)benzo[d]thiazol-2(3H)-ylidene)-3-chlorobenzamide (196 mg, 0.50 mmol) and methyl 2-bromobutyrate, instead of N-(7-fluoro-6-methoxybenzo[d]thiazol-2(3H)-ylidene)-3-chlorobenzamide and ethyl bromoacetate, respectively.

¹H NMR (DMSO-d₆, 400 MHz); 13.31 (brs, 1H), 8.37 (s, 1H), 8.18 (s, 1H), 8.14 (d, 1H), 7.98 (s, 1H), 7.72 (d, 1H), 7.59 (t, 1H), 5.90 (s, 2H), 2.50-2.36 (m, 2H), 0.80 (t, 3H)

### Example 408

### 2-(7-fluoro-6-methyl-2-((3-(trifluoromethyl)benzoyl)imino)benzo[d]thiazol-3(2H)-yl)acetic acid

The titled compound (60 mg) as a white solid was prepared in accordance with the same procedures as in Example 381, using N-(7-fluoro-6-methylbenzo[d]thiazol-2(3H)-ylidene)-3-(trifluoromethyl)benzamide (150 mg, 0.42 mmol), instead of N-(6-chloro-7-fluorobenzo[d]thiazol-2(3H)-ylidene)-3-(trifluoromethyl)benzamide.

¹H NMR (MeOD, 400 MHz): 8.53 (s, 1H), 8.51 (d, 1H), 7.84 (d, 1H), 7.68 (dd, 1H), 7.39 (dd, 1H), 7.31 (d, 1H), 5.29 (s, 2H), 2.37 (s, 3H)

### Example 409

### 2-(7-fluoro-6-methyl-2-((3-(trifluoromethyl)benzoyl)imino)benzo[d]thiazol-3(2H)-yl)propionic acid

The titled compound (20 mg) as a white solid was prepared in accordance with the same procedures as in Example 381, using N-(7-fluoro-6-methylbenzo[d]thiazol-2(3H)-ylidene)-3-(trifluoromethyl)benzamide (150 mg, 0.42 mmol) and ethyl 2-bromopropionate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.55 (d, 1H), 8.53 (s, 1H), 7.84 (d, 1H), 7.68 (dd, 1H), 7.40-7.28 (m, 2H), 6.10-5.90 (m, 1H), 2.36 (s, 3H), 1.81 (d, 3H)

### Example 410

### 2-(7-fluoro-6-methyl-2-((3-(trifluoromethyl)benzoyl)imino)benzo[d]thiazol-3(2H)-yl)butanoic acid

The titled compound (40 mg) as a white solid was prepared in accordance with the same procedures as in Example 381, using N-(7-fluoro-6-methylbenzo[d]thiazol-2(3H)-ylidene)-3-(trifluoromethyl)benzamide (150 mg, 0.42 mmol) and methyl 2-bromobutyrate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.55 (s, 1H), 8.51 (d, 1H), 7.86 (d, 1H), 7.69 (dd, 1H), 7.46-7.40 (m, 2H), 5.80-5.60 (m, 1H), 2.65-2.45 (m, 2H), 2.39 (s, 3H), 0.87 (t, 3H)

### Example 411

### 2-(2-((3-chlorobenzoyl)imino)-7-fluoro-6-methylbenzo[d]thiazol-3(2H)-yl)acetic acid

The titled compound (11 mg) as a white solid was prepared in accordance with the same procedures as in Example 381, using N-(7-fluoro-6-methylbenzo[d]thiazol-2(3H)-ylidene)-3-chlorobenzamide (150 mg, 0.47 mmol), instead of N-(6-chloro-7-fluorobenzo[d]thiazol-2(3H)-ylidene)-3-(trifluoromethyl)benzamide.

¹H NMR (MeOD, 400 MHz): 8.21 (s, 1H), 8.17 (d, 1H), 7.54 (d, 1H), 7.45 (dd, 1H), 7.40 (dd, 1H), 7.30 (d, 1H), 5.29 (s, 2H), 2.36 (s, 3H)

### Example 412

### 2-(7-fluoro-6-methoxy-2-((3-(trifluoromethyl)benzoyl)imino)benzo[d]thiazol-3(2H)-yl)acetic acid

The titled compound (62 mg) as a white solid was prepared in accordance with the same procedures as in Example 381, using N-(7-fluoro-6-methoxybenzo[d]thiazol-2(3H)-ylidene)-3-(trifluoromethyl)benzamide (150 mg, 0.41 mmol), instead of N-(6-chloro-7-fluorobenzo[d]thiazol-2(3H)-ylidene)-3-(trifluoromethyl)benzamide.

¹H NMR (MeOD, 400 MHz): 8.53 (s, 1H), 8.50 (d, 1H), 7.85 (d, 1H), 7.68 (dd, 1H), 7.40-7.20 (m, 2H), 5.30 (s, 2H), 3.94 (s, 3H)

### Example 413

### 2-(7-fluoro-6-methoxy-2-((3-(trifluoromethyl)benzoyl)imino)benzo[d]thiazol-3(2H)-yl)propionic acid

The titled compound (59 mg) as a white solid was prepared in accordance with the same procedures as in Example 381, using N-(7-fluoro-6-methoxybenzo[d]thiazol-2(3H)-ylidene)-3-(trifluoromethyl)benzamide (150 mg, 0.41 mmol) and ethyl 2-bromopropionate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.55 (s, 1H), 8.51 (d, 1H), 7.85 (d, 1H), 7.69 (dd, 1H), 7.45 (d, 1H), 7.36 (dd, 1H), 5.90-5.65 (m, 1H), 3.96 (s, 3H), 1.84 (d, 3H)

### Example 414

### 2-(7-fluoro-6-methoxy-2-((3-(trifluoromethyl)benzoyl)imino)benzo[d]thiazol-3(2H)-yl)butanoic acid

The titled compound (44 mg) as a white solid was prepared in accordance with the same procedures as in Example 381, using N-(7-fluoro-6-methoxybenzo[d]thiazol-2(3H)-ylidene)-3-(trifluoromethyl)benzamide (150 mg, 0.41 mmol) and methyl 2-bromobutyrate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.54 (s, 1H), 8.50 (d, 1H), 7.85 (d, 1H), 7.69 (dd, 1H), 7.46 (d, 1H), 7.35 (dd, 1H), 5.80-5.55 (m, 1H), 3.96 (s, 3H), 2.70-2.45 (m, 2H), 0.88 (t, 3H)

### Example 415

### 2-(2-((3-chlorobenzoyl)imino)-5,6,7-trifluorobenzo[d]thiazol-3(2H)-yl)acetic acid

To a solution of 3-chloro-N-(5,6,7-trifluorobenzo[d]thiazol-2-yl)benzamide (300 mg, 0.88 mmol) in N,N-dimethylformamide (3.0 mL), were added potassium carbonate (365 mg, 2.64 mmol) and ethyl bromoacetate (146 uL, 1.32 mmol). The reaction mixture was stirred at 80° C. for 2 days and quenched with water. The reaction mixture was extracted with ethyl acetate. The organic layer was washed with brine, dried over anhydrous magnesium sulfate, filtered, and then evaporated. The residue was washed with isopropyl ether and then dried under reduced pressure. To the obtained solid (222 mg), were added methanol (3.0 mL) and a 10% sodium hydroxide solution (1.0 mL). The reaction mixture was stirred at room temperature for 4 days. The reaction mixture was evaporated and diluted with water. The aqueous layer was acidified to pH 3-4 with an aqueous 1N HCl solution. The resulting solid was filtered, washed with water and isopropyl ether, and then dried under reduced pressure to give 162 mg of the titled compound as a pale yellow solid.

¹H NMR (DMSO-d₆, 400 MHz) δ 8.13-8.15 (m, 2H), 7.70 (m, 1H), 7.63 (d, 1H), 7.52 (t, 1H), 4.93 (s, 2H)

### Example 416

### 2-(2-((3-chlorobenzoyl)imino)-5,6,7-trifluorobenzo[d]thiazol-3(2H)-yl)propionic acid

The titled compound (89 mg) as a white solid was prepared in accordance with the same procedures as in Example 415, using ethyl 2-bromopropionate, instead of ethyl bromoacetate.

¹H NMR (DMSO-d₆, 400 MHz) δ 8.16-8.19 (m, 2H), 7.57-7.71 (m, 3H), 5.73 (m, 1H), 1.64 (m, 3H)

### Example 417

### 2-(2-((3-chlorobenzoyl)imino)-5,6,7-trifluorobenzo[d]thiazol-3(2H)-yl)butanoic acid

The titled compound (81 mg) as a white solid was prepared in accordance with the same procedures as in Example 415, using methyl 2-bromobutyrate, instead of ethyl bromoacetate.

¹H NMR (DMSO-d₆, 400 MHz) δ 13.23-13.42 (m, 1H), 8.13-8.17 (m, 2H), 7.85-7.88 (m, 1H), 7.70 (t, 1H), 7.55-7.61 (m, 1H), 5.66 (m, 1H), 2.34-2.51 (m, 2H), 0.77-0.79 (m, 3H)

### Example 418

### 2-(5-acetyl-2-((3-(trifluoromethyl)benzoyl)imino)benzo[d]thiazol-3(2H)-yl)acetic acid

The titled compound (12 mg) as a pale orange color solid was prepared in accordance with the same procedures as in Example 415, using N-(5-acetylbenzo[d]thiazol-2-yl)-3-(trifluoromethyl)benzamide (40 mg, 0.11 mmol), instead of 3-chloro-N-(5,6,7-trifluorobenzo[d]thiazol-2-yl)benzamide.

¹H NMR (DMSO-d₆, 400 MHz) δ 8.52-8.53 (m, 2H), 8.33 (s, 1H), 8.15 (d, 1H), 7.97-7.99 (m, 2H), 7.82 (t, 1H), 5.47 (s, 2H), 2.70 (s, 3H)

### Example 419

### 2-(5-acetyl-2-((3-(trifluoromethyl)benzoyl)imino)benzo[d]thiazol-3(2H)-yl)propionic acid

The titled compound (26 mg) as a pale yellow solid was prepared in accordance with the same procedures as in Example 415, using N-(5-acetylbenzo[d]thiazol-2-yl)-3-(trifluoromethyl)benzamide (40 mg, 0.11 mmol) and ethyl 2-bromopropionate, instead of 3-chloro-N-(5,6,7-trifluorobenzo[d]thiazol-2-yl)benzamide and ethyl bromoacetate, respectively.

¹H NMR (DMSO-d₆, 400 MHz) δ 8.42 (s, 1H), 8.37 (d, 1H), 8.27 (s, 1H), 8.05 (d, 1H), 7.88 (d, 2H), 7.69 (t, 1H), 5.95 (m, 1H), 2.60 (s, 3H), 1.65 (d, 3H)

### Example 420

### 2-(5-acetyl-2-((3-(trifluoromethyl)benzoyl)imino)benzo[d]thiazol-3(2H)-yl)butanoic acid

The titled compound (26 mg) as a pale yellow solid was prepared in accordance with the same procedures as in Example 415, using N-(5-acetylbenzo[d]thiazol-2-yl)-3-(trifluoromethyl)benzamide (40 mg, 0.11 mmol) and methyl 2-bromobutyrate, instead of 3-chloro-N-(5,6,7-trifluorobenzo[d]thiazol-2-yl)benzamide and ethyl bromoacetate, respectively.

¹H NMR (DMSO-d₆, 400 MHz) δ 13.18 (m, 1H), 8.42-8.52 (m, 3H), 8.17 (d, 1H), 7.99 (d, 2H), 7.80 (t, 1H), 5.97 (m, 1H), 2.70 (s, 3H), 2.34-2.51 (m, 2H), 0.78 (t, 3H)

### Example 421

### 2-(5-acetyl-2-((3-chlorobenzoyl)imino)benzo[d]thiazol-3(2H)-yl)acetic acid

The titled compound (6 mg) as a pale yellow solid was prepared in accordance with the same procedures as in Example 415, using N-(5-acetylbenzo[d]thiazol-2-yl)-3-chlorobenzamide (30 mg, 0.09 mmol), instead of 3-chloro-N-(5,6,7-trifluorobenzo[d]thiazol-2-yl)benzamide.

¹H NMR (DMSO-d₆, 400 MHz) δ 13.44 (m, 1H), 8.31 (s, 1H), 8.23 (s, 1H), 8.19 (d, 1H), 8.14 (d, 1H), 7.97 (d, 1H), 7.69 (d, 1H), 7.59 (t, 1H), 5.47 (s, 2H), 2.69 (s, 3H)

### Example 422

### 2-(5-acetyl-2-((3-chlorobenzoyl)imino)benzo[d]thiazol-3(2H)-yl)propionic acid

The titled compound (9 mg) as a pale yellow solid was prepared in accordance with the same procedures as in Example 415, using N-(5-acetylbenzo[d]thiazol-2-yl)-3-chlorobenzamide (30 mg, 0.09 mmol) and ethyl 2-bromopropionate, instead of 3-chloro-N-(5,6,7-trifluorobenzo[d]thiazol-2-yl)benzamide and ethyl bromoacetate, respectively.

¹H NMR (DMSO-d₆, 400 MHz) δ 12.82-13.24 (m, 1H), 8.27 (s, 1H), 8.14 (s, 1H), 8.07 (t, 2H), 7.90 (d, 1H), 7.60 (d, 1H), 7.49 (t, 1H), 5.97 (m, 1H), 2.62 (s, 3H), 1.66 (d, 3H)

### Example 423

### 2-(5-acetyl-2-((3-chlorobenzoyl)imino)benzo[d]thiazol-3(2H)-yl)butanoic acid

The titled compound (15 mg) as a pale yellow solid was prepared in accordance with the same procedures as in Example 415, using N-(5-acetylbenzo[d]thiazol-2-yl)-3-chlorobenzamide (30 mg, 0.09 mmol) and methyl 2-bromobutyrate, instead of 3-chloro-N-(5,6,7-trifluorobenzo[d]thiazol-2-yl)benzamide and ethyl bromoacetate, respectively.

¹H NMR (DMSO-d₆, 400 MHz) δ 13.19-13.29 (m, 1H), 8.38 (m, 1H), 8.21 (s, 1H), 8.15 (d, 2H), 7.99 (d, 1H), 7.69 (d, 1H), 7.58 (t, 1H), 5.97 (m, 1H), 2.70 (s, 3H), 2.36-2.51 (m, 2H), 0.77 (t, 3H)

### Example 424

### 2-(2-((3-chlorobenzoyl)imino)-7-fluoro-6-methylbenzo[d]thiazol-3(2H)-yl)propionic acid

The titled compound (7 mg) as a white solid was prepared in accordance with the same procedures as in Example 381, using 3-chloro-N-(7-fluoro-6-methylbenzo[d]thiazol-2(3H)-ylidene)benzamide (175 mg, 0.55 mmol) and ethyl 2-bromopropionate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.23 (s, 1H), 8.18 (d, 1H), 7.58-7.50 (m, 1H), 7.50-7.35 (m, 3H), 5.85-5.70 (m, 1H), 2.37 (s, 3H), 1.83 (d, 3H)

### Example 425

### 2-(2-((3-chlorobenzoyl)imino)-7-fluoro-6-methylbenzo[d]thiazol-3(2H)-yl)butanoic acid

The titled compound (11 mg) as a white solid was prepared in accordance with the same procedures as in Example 381, using 3-chloro-N-(7-fluoro-6-methylbenzo[d]thiazol-2(3H)-ylidene)benzamide (175 mg, 0.55 mmol) and methyl 2-bromobutyrate, instead of N-(6-chloro-7-fluorobenzo[d]thiazol-2(3H)-ylidene)-3-(trifluoromethyl)benzamide and ethyl bromoacetate, respectively.

¹H NMR (MeOD, 400 MHz): 8.24 (s, 1H), 8.18 (d, 1H), 7.56 (d, 1H), 7.55-7.45 (m, 2H), 5.80-5.55 (m, 1H), 2.65-2.45 (m, 2H), 2.39 (s, 3H), 0.86 (t, 3H)

### Example 426

### 2-(2-((3-chlorobenzoyl)imino)-6-ethoxy-7-fluorobenzo[d]thiazol-3(2H)-yl)propionic acid

The titled compound as a white solid was prepared in accordance with the same procedures as in Example 266, using 4-ethoxy-2,3-difluoroaniline, 3-chlorobenzoyl chloride, and ethyl 2-bromopropionate, instead of 2-fluoro-3-(trifluoromethyl)aniline, 3-(trifluoromethyl)benzoyl chloride and ethyl bromoacetate, respectively (Yield: 22%).

¹H NMR (CD₃OD, 400 MHz) δ 8.23 (s, 1H), 8.19 (d, 1H), 7.56 (d, 1H), 7.46 (t, 1H), 7.41 (d, 1H), 7.32 (t, 1H), 5.75 (d, 1H), 4.19 (q, 2H), 1.83 (t, 3H), 1.43 (t, 3H)

### Example 427

### 2-(2-((3-chlorobenzoyl)imino)-6-ethoxy-7-fluorobenzo[d]thiazol-3(2H)-yl)butanoic acid

The titled compound as a white solid was prepared in accordance with the same procedures as in Example 266, using 4-ethoxy-2,3-difluoroaniline, 3-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 33%).

¹H NMR (CD₃OD, 400 MHz) δ 8.22 (s, 1H), 8.18 (d, 1H), 7.56 (d, 1H), 7.48 (d, 1H), 7.44 (m, 1H), 7.31 (t, 1H), 5.65 (brs, 1H), 4.20 (q, 2H), 2.51 (m, 2H), 1.43 (t, 3H), 0.87 (t, 3H)

### Example 428

### 2-(2-((3-chlorobenzoyl)imino)-7-fluoro-6-(trifluoromethyl)benzo[d]thiazol-3(2H)-yl)acetic acid

The titled compound as a white solid was prepared in accordance with the same procedures as in Example 266, using 2,3-difluoro-4-(trifluoromethyl)aniline and 3-chlorobenzoyl chloride, instead of 2-fluoro-3-(trifluoromethyl)aniline and 3-(trifluoromethyl)benzoyl chloride, respectively (Yield: 26%).

¹H NMR (CD₃OD, 400 MHz) δ 8.19 (s, 1H), 8.17 (d, 1H), 7.78 (t, 1H), 7.53 (m, 2H), 7.44 (t, 1H), 5.34 (s, 2H)

### Example 429

### 2-(2-((3-chlorobenzoyl)imino)-7-fluoro-6-(trifluoromethyl)benzo[d]thiazol-3(2H)-yl)propionic acid

The titled compound as a white solid was prepared in accordance with the same procedures as in Example 266, using 2,3-difluoro-4-(trifluoromethyl)aniline, 3-chlorobenzoyl chloride, and ethyl 2-bromopropionate, instead of 2-fluoro-3-(trifluoromethyl)aniline, 3-(trifluoromethyl)benzoyl chloride and ethyl bromoacetate, respectively (Yield: 28%).

¹H NMR (CD₃OD, 400 MHz) δ d 8.23 (s, 1H), 8.19 (d, 1H), 7.83 (t, 1H), 7.65 (d, 1H), 7.57 (d, 1H), 7.46 (t, 1H), 5.85 (d, 1H), 1.87 (d, 3H)

### Example 430

### 2-(2-((3-chlorobenzoyl)imino)-7-fluoro-6-(trifluoromethyl)benzo[d]thiazol-3(2H)-yl)butanoic acid

The titled compound as a white solid was prepared in accordance with the same procedures as in Example 266, using 2,3-difluoro-4-(trifluoromethyl)aniline, 3-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 18%).

¹H NMR (CD₃OD, 400 MHz) 6 d 8.25 (s, 1H), 8.23 (d, 1H), 7.83 (t, 1H), 7.69 (m, 1H), 7.57 (m, 1H), 7.48 (m, 1H), 5.75 (brs, 1H), 2.55 (m, 2H), 0.89 (t, 3H)

### Example 431

### 2-(7-fluoro-6-(trifluoromethyl)-2-((3-(trifluoromethyl)benzoyl)imino)benzo[d]thiazol-3(2H)-yl)acetic acid

The titled compound as a white solid was prepared in accordance with the same procedures as in Example 266, using 2,3-difluoro-4-(trifluoromethyl)aniline, instead of 2-fluoro-3-(trifluoromethyl)aniline (Yield: 17%).

¹H NMR (CD₃OD, 400 MHz) δ 8.54 (s, 1H), 8.51 (d, 1H), 7.87 (d, 1H), 7.81 (t, 1H), 7.70 (t, 1H), 7.59 (d, 1H), 5.38 (s, 2H)

### Example 432

### 2-(7-fluoro-6-(trifluoromethyl)-2-((3-(trifluoromethyl)benzoyl)imino)benzo[d]thiazol-3(2H)-yl)propionic acid

The titled compound as a white solid was prepared in accordance with the same procedures as in Example 266, using 2,3-difluoro-4-(trifluoromethyl)aniline and ethyl 2-bromopropionate, instead of 2-fluoro-3-(trifluoromethyl)aniline and ethyl bromoacetate, respectively (Yield: 15%).

¹H NMR (CD₃OD, 400 MHz) δ 8.60 (s, 1H), 8.51 (d, 1H), 7.86 (m, 2H), 7.70 (m, 2H), 5.85 (d, 1H), 1.88 (d, 3H)

### Example 433

### 2-(7-fluoro-6-(trifluoromethyl)-2-((3-(trifluoromethyl)benzoyl)imino)benzo[d]thiazol-3(2H)-yl)butanoic acid

The titled compound as a white solid was prepared in accordance with the same procedures as in Example 266, using 2,3-difluoro-4-(trifluoromethyl)aniline and methyl 2-bromobutyrate, instead of 2-fluoro-3-(trifluoromethyl)aniline and ethyl bromoacetate, respectively (Yield: 26%).

¹H NMR (CD₃OD, 400 MHz) δ 8.30 (s, 1H), 8.25 (d, 1H), 7.99 (m, 2H), 7.78 (m, 2H), 5.65 (brs, 1H), 2.50-2.26 (m, 2H), 0.78 (t, 3H)

### Example 434

### 2-(2-((2-chlorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 2-chlorobenzoyl chloride, and methyl 2-bromobutyrate, instead of 6-methoxybenzo[d]thiazol-2-amine, 3-chlorobenzoyl chloride and ethyl bromoacetate, respectively (Yield: 21%).

¹H NMR (DMSO-d₆, 400 MHz) δ 13.20 (brs, 1H), 8.22 (t, 1H), 8.11 (s, 1H), 8.03 (d, 1H), 7.54 (m, 2H), 7.44 (m, 1H), 5.68 (brs, 1H), 2.50-2.26 (m, 2H), 0.76 (t, 3H)

### Example 435

### 2-(2-((4-chlorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 4-chlorobenzoyl chloride, and methyl 2-bromobutyrate, instead of 6-methoxybenzo[d]thiazol-2-amine, 3-chlorobenzoyl chloride and ethyl bromoacetate, respectively (Yield: 35%).

¹H NMR (DMSO-d₆, 400 MHz) δ 13.19 (brs, 1H), 8.21-8.16 (m, 4H), 7.60 (d, 2H), 5.65 (brs, 1H), 2.50-2.28 (m, 2H), 0.77 (t, 3H)

### Example 436

### 2-(5,6-difluoro-2-((2-(trifluoromethyl)benzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 2-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 35%).

¹H NMR (DMSO-d₆, 400 MHz) δ 13.24 (brs, 1H), 8.22 (t, 1H), 8.09 (s, 1H), 8.01 (d, 1H), 7.86 (d, 1H), 7.79-7.70 (m, 2H), 5.67 (brs, 1H), 2.50-2.25 (m, 2H), 0.76 (t, 3H)

### Example 437

### 2-(5,6-difluoro-2-((4-(trifluoromethyl)benzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 4-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 41%).

¹H NMR (DMSO-d₆, 400 MHz) δ 13.22 (brs, 1H), 8.37 (d, 2H), 8.24 (m, 1H), 8.16 (s, 1H), 7.91 (d, 2H), 5.68 (brs, 1H), 2.50-2.31 (m, 2H), 0.80 (t, 3H)

### Example 438

### 2-(5,6-difluoro-2-((2-methoxybenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 2-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 15%).

¹H NMR (DMSO-d₆, 400 MHz) δ 13.05 (brs, 1H), 8.17 (t, 1H), 8.07 (s, 1H), 7.92 (d, 1H), 7.49 (t, 1H), 7.13 (d, 1H), 7.00 (t, 1H), 5.59 (brs, 1H), 3.82 (s, 3H), 2.50-2.23 (m, 2H), 0.76 (t, 3H)

### Example 439

### 2-(5,6-difluoro-2-((3-methoxybenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 3-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 25%).

¹H NMR (DMSO-d₆, 400 MHz) δ 13.09 (brs, 1H), 8.21-8.16 (m, 2H), 7.76 (d, 2H), 7.43 (m, 1H), 7.15 (d, 1H), 5.60 (brs, 1H), 3.85 (s, 3H), 2.50-2.30 (m, 2H), 0.77 (t, 3H)

### Example 440

### 2-(5,6-difluoro-2-((4-methoxybenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 4-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (DMSO-d₆, 400 MHz) δ 13.12 (brs, 1H), 8.17-8.10 (m, 4H), 7.05 (d, 2H), 5.59 (brs, 1H), 3.90 (s, 3H), 2.50-2.28 (m, 2H), 0.77 (t, 3H)

### Example 441

### 2-(5,6-difluoro-2-((2-fluorobenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 2-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 15%).

¹H NMR (DMSO-d₆, 400 MHz) δ 13.18 (brs, 1H), 8.22 (m, 1H), 8.12 (m, 1H), 7.62 (m, 1H), 7.33-7.28 (m, 2H), 5.64 (brs, 1H), 2.50-2.26 (m, 2H), 0.77 (t, 3H)

### Example 442

### 2-(5,6-difluoro-2-((3-fluorobenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 3-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 21%).

¹H NMR (DMSO-d₆, 400 MHz) δ 13.20 (brs, 1H), 8.23-8.18 (m, 2H), 8.02 (d, 1H), 7.91 (d, 1H), 7.58 (q, 1H), 7.46 (t, 1H), 5.65 (brs, 1H), 2.50-2.29 (m, 2H), 0.77 (t, 3H)

### Example 443

### 2-(5,6-difluoro-2-((4-fluorobenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 20%).

¹H NMR (DMSO-d₆, 400 MHz) δ 13.18 (brs, 1H), 8.25-8.13 (m, 4H), 7.34 (t, 2H), 5.63 (brs, 1H), 2.50-2.30 (m, 2H), 0.77 (t, 3H)

### Example 444

### 2-(2-((2,4-difluorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 2,4-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 15%).

¹H NMR (DMSO-d₆, 400 MHz) δ 13.19 (brs, 1H), 8.23-8.12 (m, 3H), 7.37 (q, 1H), 7.22 (t, 1H), 5.64 (brs, 1H), 2.50-2.80 (m, 2H), 0.77 (t, 3H)

### Example 445

### 2-(5,6-difluoro-2-((5-fluoro-2-(trifluoromethyl)benzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 5-fluoro-2-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 16%).

¹H NMR (DMSO-d₆, 400 MHz) δ 13.28 (brs, 1H), 8.23 (t, 1H), 8.12 (s, 1H), 7.94 (m, 1H), 7.82 (d, 1H), 7.58 (t, 1H), 5.70 (brs, 1H), 2.50-2.25 (m, 2H), 0.76 (t, 3H)

### Example 446

### 2-(2-((2-bromobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 2-bromobenzoyl chloride, and methyl 2-bromobutyrate, instead of 6-methoxybenzo[d]thiazol-2-amine, 3-chlorobenzoyl chloride and ethyl bromoacetate, respectively (Yield: 28%).

¹H NMR (DMSO-d₆, 400 MHz) δ 13.21 (brs, 1H), 8.22 (t, 1H), 8.18 (s, 1H), 8.00 (d, 1H), 7.72 (d, 1H), 7.51-7.41 (m, 2H), 5.66 (brs, 1H), 2.50-2.25 (m, 2H), 0.76 (t, 3H)

### Example 447

### 2-(2-((4-bromobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 4-bromobenzoyl chloride, and methyl 2-bromobutyrate, instead of 6-methoxybenzo[d]thiazol-2-amine, 3-chlorobenzoyl chloride and ethyl bromoacetate, respectively (Yield: 24%).

¹H NMR (DMSO-d₆, 400 MHz) δ 13.19 (brs, 1H), 8.22 (t, 1H), 8.10 (m, 2H), 7.72 (d, 2H), 5.65 (brs, 1H), 2.50-2.28 (m, 2H), 0.77 (t, 3H)

### Example 448

### 2-(2-((2,6-difluorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 2,6-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 22%).

¹H NMR (DMSO-d₆, 400 MHz) δ 13.16 (brs, 1H), 8.25 (t, 1H), 8.11 (s, 1H), 7.54 (m, 1H), 7.18 (m, 2H), 5.64 (brs, 1H), 2.50-2.19 (m, 2H), 0.74 (t, 3H)

### Example 449

### 2-(2-((3,5-difluorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 3,5-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (DMSO-d₆, 400 MHz) δ 13.24 (brs, 1H), 8.24 (t, 1H), 8.20 (s, 1H), 7.78 (d, 2H), 7.53 (t, 1H), 5.67 (brs, 1H), 2.50-2.28 (m, 2H), 0.77 (t, 3H)

### Example 450

### 2-(5,6-difluoro-2-((2,3,6-trifluorobenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 2,3,6-trifluorobenzoyl chloride, and methyl 2-bromobutyrate, instead of 6-methoxybenzo[d]thiazol-2-amine, 3-chlorobenzoyl chloride and ethyl bromoacetate, respectively (Yield: 8%).

¹H NMR (DMSO-d₆, 400 MHz) δ 13.18 (brs, 1H), 8.27 (t, 1H), 8.20 (s, 1H), 7.65 (m, 1H), 7.24 (t, 1H), 5.68 (brs, 1H), 2.50-2.30 (m, 2H), 0.74 (t, 3H)

### Example 451

### 2-(5,6-difluoro-2-((2,4,5-trifluorobenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 2,4,5-trifluorobenzoyl chloride, and methyl 2-bromobutyrate, instead of 6-methoxybenzo[d]thiazol-2-amine, 3-chlorobenzoyl chloride and ethyl bromoacetate, respectively (Yield: 31%).

¹H NMR (DMSO-d₆, 400 MHz) δ 13.27 (brs, 1H), 8.24 (t, 1H), 8.09 (m, 2H), 7.68 (m, 1H), 5.67 (brs, 1H), 2.50-2.26 (m, 2H), 0.76 (t, 3H)

### Example 452

### 2-(5,6-difluoro-2-((5-fluoro-2-methylbenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 5-fluoro-2-methylbenzoyl chloride, and methyl 2-bromobutyrate, instead of 6-methoxybenzo[d]thiazol-2-amine, 3-chlorobenzoyl chloride and ethyl bromoacetate, respectively (Yield: 23%).

¹H NMR (DMSO-d₆, 400 MHz) δ 13.20 (brs, 1H), 8.20 (t, 1H), 8.17 (s, 1H), 7.93 (d, 1H), 7.36-7.27 (m, 2H), 5.62 (brs, 1H), 2.61 (s, 3H), 2.50-2.26 (m, 2H), 0.76 (t, 3H)

### Example 453

### 2-(5,6-difluoro-2-((4-(trifluoromethoxy)benzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 4-(trifluoromethoxy)benzoyl chloride, and methyl 2-bromobutyrate, instead of 6-methoxybenzo[d]thiazol-2-amine, 3-chlorobenzoyl chloride and ethyl bromoacetate, respectively (Yield: 24%).

¹H NMR (DMSO-d₆, 400 MHz) δ 13.20 (brs, 1H), 8.30 (d, 2H), 8.20 (t, 1H), 8.15 (s, 1H), 7.52 (d, 2H), 5.65 (brs, 1H), 2.50-2.31 (m, 2H), 0.78 (t, 3H)

### Example 454

### 2-(5,7-difluoro-2-((4-methylbenzoyl)imino)benzo[d]thiazol-3(2H)-yl)acetic acid

The titled compound as a white solid was prepared in accordance with the same procedures as in Example 266, using 22,3,5-trifluoroaniline and p-toluoyl chloride, instead of 2-fluoro-3-(trifluoromethyl)aniline and 3-(trifluoromethyl)benzoyl chloride, respectively (Yield: 27%).

¹H NMR (DMSO-d₆, 400 MHz) δ 13.39 (brs, 1H), 8.14 (d, 2H), 7.75 (d, 1H), 7.50-7.30 (m, 3H), 5.30 (s, 2H), 2.40 (s, 3H)

### Example 455

### 2-(5,7-difluoro-2-((4-methylbenzoyl)imino)benzo[d]thiazol-3(2H)-yl)propionic acid

The titled compound as a white solid was prepared in accordance with the same procedures as in Example 266, using 2,3,5-trifluoroaniline, p-toluoyl chloride, and ethyl 2-bromopropionate, instead of 2-fluoro-3-(trifluoromethyl)aniline, 3-(trifluoromethyl)benzoyl chloride and ethyl bromoacetate, respectively (Yield: 15%).

¹H NMR (CD₃OD, 400 MHz) δ 8.16 (d, 2H), 7.42 (d, 1H), 7.30 (d, 2H), 7.06 (dd, 1H), 5.80-5.65 (m, 1H), 2.42 (s, 3H), 1.83 (d, 3H)

### Example 456

### 2-(5,7-difluoro-2-((4-methylbenzoyl)imino)benzo[d]thiazol-3(2H)-yl)butanoic acid

The titled compound as a white solid was prepared in accordance with the same procedures as in Example 266, using 2,3,5-trifluoroaniline, p-toluoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 19%).

¹H NMR (CD₃OD, 400 MHz) δ 8.15 (d, 2H), 7.43 (d, 1H), 7.30 (d, 2H), 7.06 (dd, 1H), 5.61 (brs, 1H), 2.70-2.40 (m, 2H), 2.42 (s, 3H), 0.88 (t, 3H)

### Example 457

### 2-(5,7-difluoro-2-((3-(trifluoromethyl)benzoyl)imino)benzo[d]thiazol-3(2H)-yl)acetic acid

The titled compound as a white solid was prepared in accordance with the same procedures as in Example 266, using 2,3,5-trifluoroaniline instead of 2-fluoro-3-(trifluoromethyl)aniline (Yield: 21%).

¹H NMR (CD₃OD, 400 MHz) δ 8.60-8.45 (m, 2H), 7.86 (d, 1H), 7.70 (dd, 1H), 7.40 (d, 1H), 7.07 (dd, 1H), 5.32 (s, 2H)

### Example 458

### 2-(5,7-difluoro-2-((3-(trifluoromethyl)benzoyl)imino)benzo[d]thiazol-3(2H)-yl)propionic acid

The titled compound as a white solid was prepared in accordance with the same procedures as in Example 266, using 2,3,5-trifluoroaniline and ethyl 2-bromopropionate, instead of 2-fluoro-3-(trifluoromethyl)aniline and ethyl bromoacetate, respectively (Yield: 11%).

¹H NMR (CD₃OD, 400 MHz) δ 8.60-8.45 (m, 2H), 7.87 (d, 1H), 7.70 (dd, 1H), 7.45 (d, 1H), 7.08 (dd, 1H), 5.85-5.70 (m, 1H), 1.83 (d, 3H)

### Example 459

### 2-(5,7-difluoro-2-((3-(trifluoromethyl)benzoyl)imino)benzo[d]thiazol-3(2H)-yl)butanoic acid

The titled compound as a white solid was prepared in accordance with the same procedures as in Example 266, using 2,3,5-trifluoroaniline and methyl 2-bromobutyrate, instead of 2-fluoro-3-(trifluoromethyl)aniline and ethyl bromoacetate, respectively (Yield: 16%).

¹H NMR (CD₃OD, 400 MHz) δ 8.54 (s, 1H), 8.50 (d, 1H), 7.87 (d, 2H), 7.70 (dd, 1H), 7.51 (d, 1H), 7.10 (dd, 1H), 5.61 (brs, 1H), 2.70-2.45 (m, 2H), 0.90 (t, 3H)

### Example 460

### 2-(2-((3-chlorobenzoyl)imino)-5,7-difluorobenzo[d]thiazol-3(2H)-yl)acetic acid

The titled compound as a white solid was prepared in accordance with the same procedures as in Example 266, using 2,3,5-trifluoroaniline and 3-chlorobenzoyl chloride, instead of 2-fluoro-3-(trifluoromethyl)aniline and 3-(trifluoromethyl)benzoyl chloride, respectively (Yield: 27%).

¹H NMR (DMSO-d₆, 400 MHz) δ 13.51 (brs, 1H), 8.20 (s, 1H), 8.16 (d, 1H), 7.79 (d, 1H), 7.69 (d, 1H), 7.58 (dd, 1H), 7.42 (dd, 1H), 5.33 (s, 2H)

### Example 461

### 2-(2-((3-chlorobenzoyl)imino)-5,7-difluorobenzo[d]thiazol-3(2H)-yl)propionic acid

The titled compound as a white solid was prepared in accordance with the same procedures as in Example 266, using 2,3,5-trifluoroaniline, 3-chlorobenzoyl chloride, and ethyl 2-bromopropionate, instead of 2-fluoro-3-(trifluoromethyl)aniline, 3-(trifluoromethyl)benzoyl chloride and ethyl bromoacetate, respectively (Yield: 30%).

¹H NMR (CD₃OD, 400 MHz) δ 8.23 (s, 1H), 8.18 (d, 1H), 7.56 (d, 1H), 7.55-7.40 (m, 2H), 7.07 (dd, 1H), 5.80-5.65 (m, 1H), 1.79 (d, 3H)

### Example 462

### 2-(2-((3-chlorobenzoyl)imino)-5,7-difluorobenzo[d]thiazol-3(2H)-yl)butanoic acid

The titled compound as a white solid was prepared in accordance with the same procedures as in Example 266, using 2,3,5-trifluoroaniline, 3-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 15%).

¹H NMR (CD₃OD, 400 MHz) δ 8.23 (s, 1H), 8.18 (d, 1H), 7.57 (d, 1H), 7.55-7.40 (m, 2H), 7.09 (dd, 1H), 5.62 (brs, 1H), 2.65-2.40 (m, 2H), 0.89 (t, 3H)

### Example 463

### 2-(2-((3,4-difluorobenzoyl)imino)-5,6-difluorobenzo[d]thiazol-3(2H)-yl)propionic acid

The titled compound as a white solid was prepared in accordance with the same procedures as in Example 52, using 5,6-difluorobenzo[d]thiazol-2-amine, 3,4-difluorobenzoyl chloride, and ethyl 2-bromopropionate, instead of 6-methoxybenzo[d]thiazol-2-amine, 3-chlorobenzoyl chloride and ethyl bromoacetate, respectively (Yield: 27%).

¹H NMR (DMSO-d₆, 400 MHz) δ 13.22 (brs, 1H), 8.19 (t, 1H), 8.14 (m, 2H), 8.04 (m, 1H), 7.61 (q, 1H), 5.78 (d, 1H), 1.71 (t, 3H)

### Example 464

### 2-(2-((3,4-difluorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 3,4-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 22%).

¹H NMR (DMSO-d₆, 400 MHz) δ 13.23 (brs, 1H), 8.23-8.07 (m, 3H), 8.02 (s, 1H), 7.61 (q, 1H), 5.65 (brs, 1H), 2.50-2.29 (m, 2H), 0.77 (t, 3H)

### Example 465

### 2-(2-((3,5-bis(trifluoromethyl)benzoyl)imino)-5,6-difluorobenzo[d]thiazol-3(2H)-yl)propionic acid

The titled compound as a white solid was prepared in accordance with the same procedures as in Example 52, using 5,6-difluorobenzo[d]thiazol-2-amine, 3,5-bis(trifluoromethyl)benzoyl chloride, and ethyl 2-bromopropionate, instead of 6-methoxybenzo[d]thiazol-2-amine, 3-chlorobenzoyl chloride and ethyl bromoacetate, respectively (Yield: 25%).

¹H NMR (DMSO-d₆, 400 MHz) δ 13.20 (brs, 1H), 8.72 (s, 2H), 8.38 (s, 1H), 8.21 (m, 2H), 5.79 (d, 1H), 1.74 (d, 3H)

### Example 466

### 2-(2-((3-chloro-4-fluorobenzoyl)imino)-5,6-difluorobenzo[d]thiazol-3(2H)-yl)propionic acid

The titled compound as a white solid was prepared in accordance with the same procedures as in Example 52, using 5,6-difluorobenzo[d]thiazol-2-amine, 3-chloro-4-fluorobenzoyl chloride, and ethyl 2-bromopropionate, instead of 6-methoxybenzo[d]thiazol-2-amine, 3-chlorobenzoyl chloride and ethyl bromoacetate, respectively (Yield: 19%).

¹H NMR (DMSO-d₆, 400 MHz) δ 13.22 (brs, 1H), 8.33 (d, 1H), 8.21-8.10 (m, 3H), 7.58 (t, 1H), 5.77 (d, 1H), 1.71 (d, 3H)

### Example 467

### 2-(2-((3-chloro-4-fluorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 3-chloro-4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (DMSO-d₆, 400 MHz) δ 13.15 (brs, 1H), 8.31 (d, 1H), 8.23-8.14 (m, 3H), 7.58 (t, 1H), 5.65 (brs, 1H), 2.50-2.30 (m, 2H), 0.77 (t, 3H)

### Example 468

### 2-(2-((3,5-dichlorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 3,5-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (DMSO-d₆, 400 MHz) δ 13.41 (brs, 1H), 8.35-8.05 (m, 3H), 7.88 (s, 1H), 5.80-5.50 (m, 1H), 2.50-2.25 (m, 2H), 0.90-0.65 (m, 3H)

### Example 469

### 2-(5,6-difluoro-2-((3-fluoro-5-(trifluoromethyl)benzoyl)iminobenzo[d]thiazol-3(2H)-yl)butanoic acid

The titled compound as a white solid was prepared in accordance with the same procedures as in Example 52, using 5,6-difluorobenzo[d]thiazol-2-amine, 3-fluoro-5-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 26%).

¹H NMR (DMSO-d₆, 400 MHz) δ 13.36 (brs, 1H), 8.40-8.05 (m, 3H), 7.98 (s, 1H), 5.69 (brs, 1H), 2.50-2.20 (m, 2H), 0.78 (d, 3H)

### Example 470

### 2-(5,6-difluoro-2-((3-fluoro-4-methylbenzoyl)imino)benzo[d]thiazol-3(2H)-yl)butanoic acid

The titled compound as a white solid was prepared in accordance with the same procedures as in Example 52, using 5,6-difluorobenzo[d]thiazol-2-amine, 3-fluoro-4-methylbenzoyl chloride, and methyl 2-bromobutyrate, instead of 6-methoxybenzo[d]thiazol-2-amine, 3-chlorobenzoyl chloride and ethyl bromoacetate, respectively (Yield: 15%).

¹H NMR (CD₃OD, 400 MHz) δ 8.05-7.95 (m, 1H), 7.95-7.80 (m, 2H), 7.80-7.70 (m, 1H), 7.45-7.25 (m, 1H), 5.61 (brs, 1H), 2.70-2.40 (m, 2H), 2.38 (s, 3H), 0.89 (t, 3H)

### Example 471

### 2-(5,6-difluoro-2-((4-fluoro-3-(trifluoromethyl)benzoyl)iminobenzo[d]thiazol-3(2H)-yl)butanoic acid

The titled compound as a white solid was prepared in accordance with the same procedures as in Example 52, using 5,6-difluorobenzo[d]thiazol-2-amine, 4-fluoro-3-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 14%).

¹H NMR (CD₃OD, 400 MHz) δ 8.65-8.50 (m, 2H), 7.95-7.70 (m, 2H), 7.55-7.40 (m, 1H), 5.60 (brs, 1H), 2.70-2.40 (m, 2H), 0.90 (t, 3H)

### Example 472

### 2-(2-((3,4-dichlorobenzoyl)imino)-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 3,4-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 10%).

¹H NMR (CD₃OD, 400 MHz) δ 8.35 (s, 1H), 8.13 (d, 1H), 7.90-7.75 (m, 2H), 7.64 (d, 1H), 5.61 (brs, 1H), 2.65-2.40 (m, 2H), 0.87 (t, 3H)

### Example 473

### 2-(2-((3,5-dimethoxybenzoyl)imino-5,6-difluorobenzo[d]thiazol-3(2H)-yl)butanoic acid

The titled compound as a white solid was prepared in accordance with the same procedures as in Example 52, using 5,6-difluorobenzo[d]thiazol-2-amine, 3,5-dimethoxybenzoyl chloride, and methyl 2-bromobutyrate, instead of 6-methoxybenzo[d]thiazol-2-amine, 3-chlorobenzoyl chloride and ethyl bromoacetate, respectively (Yield: 15%).

¹H NMR (CD₃OD, 400 MHz) δ 7.90-7.70 (m, 2H), 7.45 (s, 2H), 6.65 (s, 1H), 5.48 (brs, 1H), 3.86 (s, 6H), 2.70-2.35 (m, 2H), 0.88 (t, 3H)

### Example 474

### 2-(5,6-difluoro-2-((3-(trifluoromethoxy)benzoyl)iminobenzo[d]thiazol-3(2H)-yl)butanoic acid

The titled compound as a white solid was prepared in accordance with the same procedures as in Example 52, using 5,6-difluorobenzo[d]thiazol-2-amine, 3-(trifluoromethoxy)benzoyl chloride, and methyl 2-bromobutyrate, instead of 6-methoxybenzo[d]thiazol-2-amine, 3-chlorobenzoyl chloride and ethyl bromoacetate, respectively (Yield: 9%).

¹H NMR (CD₃OD, 400 MHz) δ 8.24 (d, 1H), 8.12 (s, 1H), 7.95-7.70 (m, 2H), 7.58 (dd, 1H), 7.47 (d, 1H), 5.60 (brs, 1H), 2.65-2.40 (m, 2H), 0.88 (t, 3H)

### Example 475

### 2-(6-ethoxy-2-((3-fluorobenzoyl)imino)benzo[d]thiazol-3(2H)-yl)butanoic acid

The titled compound as a white solid was prepared in accordance with the same procedures as in Example 52, using 6-ethoxybenzo[d]thiazol-2-amine, 3-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (CD₃OD, 400 MHz) δ 8.10 (d, 1H), 7.92 (d, 1H), 7.57 (d, 1H), 7.47 (m, 1H), 7.40 (s, 1H), 7.28 (t, 1H), 7.09 (d, 1H), 5.66 (brs, 1H), 4.10 (q, 2H), 2.57-2.45 (m, 2H), 1.41 (t, 3H), 0.85 (t, 3H)

### Example 476

### 2-(6-ethoxy-2-((4-fluorobenzoyl)imino)benzo[d]thiazol-3(2H)-yl)butanoic acid

The titled compound as a white solid was prepared in accordance with the same procedures as in Example 52, using 6-ethoxybenzo[d]thiazol-2-amine, 4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 35%).

¹H NMR (CD₃OD, 400 MHz) δ 8.30 (m, 2H), 7.55 (d, 1H), 7.39 (s, 1H), 7.17 (t, 2H), 7.08 (d, 1H), 5.66 (brs, 1H), 4.10 (q, 2H), 2.55-2.44 (m, 2H), 1.41 (t, 3H), 0.85 (t, 3H)

### Example 477

### 2-(6-ethoxy-2-((3-methoxybenzoyl)iminobenzo[d]thiazol-3(2H)-yl)butanoic acid

The titled compound as a white solid was prepared in accordance with the same procedures as in Example 52, using 6-ethoxybenzo[d]thiazol-2-amine, 3-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 32%).

¹H NMR (CD₃OD, 400 MHz) δ 7.84 (s, 1H), 7.82 (d, 1H), 7.56 (d, 1H), 7.39 (s, 1H), 7.35 (m, 1H), 7.11-7.07 (m, 2H), 5.59 (brs, 1H), 4.10 (q, 2H), 3.88 (s, 3H), 2.62-2.43 (m, 2H), 1.41 (t, 3H), 0.86 (t, 3H)

### Example 478

### 2-(6-ethoxy-2-((4-methoxybenzoyl)iminobenzo[d]thiazol-3(2H)-yl)butanoic acid

The titled compound as a white solid was prepared in accordance with the same procedures as in Example 52, using 6-ethoxybenzo[d]thiazol-2-amine, 4-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 26%).

¹H NMR (CD₃OD, 400 MHz) δ 8.22 (d, 2H), 7.52 (d, 1H), 7.38 (s, 1H), 7.09 (m, 1H), 6.99 (m, 2H), 5.64 (brs, 1H), 4.10 (q, 2H), 3.86 (s, 3H), 2.55-2.45 (m, 2H), 1.41 (t, 3H), 0.85 (t, 3H)

### Example 479

### 2-(2-((4-chlorobenzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 4-chlorobenzoyl chloride, and methyl 2-bromobutyrate, instead of 6-methoxybenzo[d]thiazol-2-amine, 3-chlorobenzoyl chloride and ethyl bromoacetate, respectively (Yield: 29%).

¹H NMR (CD₃OD, 400 MHz) δ 8.23 (d, 2H), 7.55 (d, 1H), 7.47 (d, 2H), 7.40 (s, 1H), 7.11 (m, 1H), 5.67 (brs, 1H), 4.11 (q, 2H), 2.55-2.44 (m, 2H), 1.41 (t, 3H), 0.85 (t, 3H)

### Example 480

### 2-(6-ethoxy-2-((3-(trifluoromethoxy)benzoyl)iminobenzo[d]thiazol-3(2H)-yl)butanoic acid

The titled compound as a white solid was prepared in accordance with the same procedures as in Example 52, using 6-ethoxybenzo[d]thiazol-2-amine, 3-(trifluoromethoxy)benzoyl chloride, and methyl 2-bromobutyrate, instead of 6-methoxybenzo[d]thiazol-2-amine, 3-chlorobenzoyl chloride and ethyl bromoacetate, respectively (Yield: 24%).

¹H NMR (DMSO-d₆, 400 MHz) δ 13.08 (brs, 1H), 8.21 (d, 1H), 8.08 (s, 1H), 7.74 (s, 1H), 7.68-7.57 (m, 3H), 7.16 (q, 1H), 5.73 (brs, 1H), 4.11 (q, 2H), 2.50-2.29 (m, 2H), 1.36 (t, 3H), 0.75 (t, 3H)

### Example 481

### 2-(2-((3,5-dimethoxybenzoyl)imino-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3,5-dimethoxybenzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (DMSO-d₆, 400 MHz) δ 12.98 (brs, 1H), 7.75 (d, 1H), 7.58 (d, 1H), 7.38 (d, 2H), 7.13 (d, 1H), 6.68 (s, 1H), 5.62 (brs, 1H), 4.10 (q, 2H), 3.82 (s, 6H), 2.50-2.29 (m, 2H), 1.36 (t, 3H), 0.74 (t, 3H)

### Example 482

### 2-(2-((3,4-difluorobenzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3,4-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 14%).

¹H NMR (CD₃OD, 400 MHz) δ 8.09-8.05 (m, 2H), 7.57 (d, 1H), 7.38 (s, 1H), 7.32 (m, 1H), 7.10 (m, 1H), 5.64 (brs, 1H), 4.09 (q, 2H), 2.54-2.44 (m, 2H), 1.40 (t, 3H), 0.85 (t, 3H)

### Example 483

### 2-(2-((3,5-difluorobenzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3,5-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 16%).

¹H NMR (CD₃OD, 400 MHz) δ 7.79 (d, 2H), 7.60 (d, 1H), 7.42 (s, 1H), 7.14-7.11 (m, 2H), 5.64 (brs, 1H), 4.12 (q, 2H), 2.53-2.47 (m, 2H), 1.42 (t, 3H), 0.85 (t, 3H)

### Example 484

### 2-(6-ethoxy-2-((3-fluoro-5-(trifluoromethyl)benzoyl)iminobenzo[d]thiazol-3(2H)-yl)butanoic acid

The titled compound as a white solid was prepared in accordance with the same procedures as in Example 52, using 6-ethoxybenzo[d]thiazol-2-amine, 3-fluoro-5-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 24%).

¹H NMR (DMSO-d₆, 400 MHz) δ 13.12 (brs, 1H), 8.30 (s, 1H), 8.20 (m, 1H), 7.95 (d, 1H), 7.79 (s, 1H), 7.64 (m, 1H), 7.18 (d, 1H), 5.74 (brs, 1H), 4.10 (q, 2H), 2.50-2.32 (m, 2H), 1.36 (t, 3H), 0.76 (t, 3H)

### Example 485

### 2-(2-((3,4-dichlorobenzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3,4-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 16%).

¹H NMR (DMSO-d₆, 400 MHz) δ 13.12 (brs, 1H), 8.38 (s, 1H), 8.09 (d, 1H), 7.83 (m, 2H), 7.61 (s, 1H), 7.16 (d, 1H), 5.73 (brs, 1H), 4.11 (q, 2H), 2.50-2.28 (m, 2H), 1.36 (t, 3H), 1.07 (t, 3H)

### Example 486

### 2-(2-((3,5-dichlorobenzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3,5-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 36%).

¹H NMR (DMSO-d₆, 400 MHz) δ 13.14 (brs, 1H), 8.14 (s, 2H), 7.92 (s, 2H), 7.83 (s, 1H), 7.61 (s, 1H), 7.17 (d, 1H), 5.73 (brs, 1H), 4.11 (q, 2H), 2.50-2.28 (m, 2H), 1.36 (t, 3H), 0.75 (t, 3H)

### Example 487

### 2-(2-((3-chloro-4-fluorobenzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3-chloro-4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 28%).

¹H NMR (DMSO-d₆, 400 MHz) δ 13.12 (brs, 1H), 8.32 (d, 1H), 8.18 (d, 1H), 7.73 (s, 1H), 7.60 (m, 2H), 7.16 (m, 1H), 5.73 (brs, 1H), 4.09 (q, 2H), 2.50-2.28 (m, 2H), 1.36 (t, 3H), 0.75 (t, 3H)

### Example 488

### 2-(2-((3,5-bis(trifluoromethyl)benzoyl)imino)-6-ethoxybenzo[d]thiazol-3(2H)-yl)butanoic acid

The titled compound as a white solid was prepared in accordance with the same procedures as in Example 52, using 6-ethoxybenzo[d]thiazol-2-amine, 3,5-bis(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 23%).

¹H NMR (CD₃OD, 400 MHz) δ 8.77 (s, 2H), 8.12 (s, 1H), 7.64 (d, 1H), 7.42 (s, 1H), 7.14 (d, 1H), 5.62 (brs, 1H), 4.10 (q, 2H), 2.56-2.48 (m, 2H), 1.41 (t, 3H), 0.88 (t, 3H)

### Example 489

### 2-(6-ethoxy-2-((3-fluoro-4-methylbenzoyl)iminobenzo[d]thiazol-3(2H)-yl)butanoic acid

The titled compound as a white solid was prepared in accordance with the same procedures as in Example 52, using 6-ethoxybenzo[d]thiazol-2-amine, 3-fluoro-4-methylbenzoyl chloride, and methyl 2-bromobutyrate, instead of 6-methoxybenzo[d]thiazol-2-amine, 3-chlorobenzoyl chloride and ethyl bromoacetate, respectively (Yield: 14%).

¹H NMR (DMSO-d₆, 400 MHz) δ 13.07 (brs, 1H), 7.92 (d, 1H), 7.87 (d, 1H), 7.71 (s, 1H), 7.59 (s, 1H), 7.41 (t, 1H), 7.14 (d, 1H), 5.70 (brs, 1H), 4.10 (q, 2H), 2.50-2.28 (m, 2H), 2.41 (s, 3H), 1.36 (t, 3H), 1.04 (t, 3H)

### Example 490

### 2-(6-ethoxy-2-((4-fluoro-3-(trifluoromethyl)benzoyl)iminobenzo[d]thiazol-3(2H)-yl)butanoic acid

The titled compound as a white solid was prepared in accordance with the same procedures as in Example 52, using 6-ethoxybenzo[d]thiazol-2-amine, 4-fluoro-3-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (DMSO-d₆, 400 MHz) δ 13.08 (brs, 1H), 8.53 (m, 2H), 7.95 (m, 1H), 7.64 (m, 2H), 7.16 (d, 1H), 5.70 (brs, 1H), 4.11 (q, 2H), 2.50-2.30 (m, 2H), 1.36 (t, 3H), 1.15 (t, 3H)

### Example 491

### 2-(2-((3-fluorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 11%).

¹H NMR (CD₃OD, 400 MHz) δ 8.23 (s, 1H), 8.09 (d, 1H), 7.95 (d, 1H), 7.90-7.75 (m, 2H), 7.51 (dd, 1H), 7.31 (dd, 1H), 5.77 (brs, 1H), 2.70-2.45 (m, 2H), 0.88 (t, 3H)

### Example 492

### 2-(2-((4-fluorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (CD₃OD, 400 MHz) δ 8.50-8.25 (m, 2H), 8.21 (s, 1H), 7.90-7.70 (m, 2H), 7.30-7.10 (m, 2H), 5.77 (brs, 1H), 2.70-2.40 (m, 2H), 0.88 (t, 3H)

### Example 493

### 2-(2-((3-methoxybenzoyl)imino-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 6%).

¹H NMR (CD₃OD, 400 MHz) δ 8.21 (s, 1H), 7.90-7.75 (m, 4H), 7.38 (dd, 1H), 7.12 (d, 1H), 5.70 (brs, 1H), 2.70-2.45 (m, 2H), 0.88 (t, 3H)

### Example 494

### 2-(2-((4-methoxybenzoyl)imino-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 4-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 28%).

¹H NMR (CD₃OD, 400 MHz) δ 8.35-8.15 (m, 3H), 7.78 (s, 1H), 7.00 (d, 2H), 5.75 (brs, 1H), 3.87 (s, 3H), 2.70-2.40 (m, 2H), 0.88 (t, 3H)

### Example 495

### 2-(2-((4-chlorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 4-chlorobenzoyl chloride, and methyl 2-bromobutyrate, instead of 6-methoxybenzo[d]thiazol-2-amine, 3-chlorobenzoyl chloride and ethyl bromoacetate, respectively (Yield: 25%).

¹H NMR (CD₃OD, 400 MHz) δ 8.35-8.15 (m, 3H), 7.90-7.75 (m, 2H), 7.48 (d, 2H), 5.78 (brs, 1H), 2.70-2.45 (m, 2H), 0.88 (t, 3H)

### Example 496

### 2-(2-((3-(trifluoromethoxy)benzoyl)imino-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3-(trifluoromethoxy)benzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (CD₃OD, 400 MHz) δ 8.27 (d, 1H), 8.24 (s, 1H), 8.14 (s, 1H), 7.95-7.75 (m, 2H), 7.59 (dd, 1H), 7.47 (d, 1H), 5.95 (brs, 1H), 2.75-2.45 (m, 2H), 0.89 (t, 3H)

### Example 497

### 2-(2-((3,5-dimethoxybenzoyl)imino-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3,5-dimethoxybenzoyl chloride, and methyl 2-bromobutyrate, instead of 6-methoxybenzo[d]thiazol-2-amine, 3-chlorobenzoyl chloride and ethyl bromoacetate, respectively (Yield: 21%).

¹H NMR (CD₃OD, 400 MHz) δ 8.21 (s, 1H), 7.95-7.80 (m, 2H), 7.48 (s, 2H), 6.66 (s, 1H), 5.75 (brs, 1H), 3.81 (s, 6H), 2.75-2.45 (m, 2H), 0.88 (t, 3H)

### Example 498

### 2-(2-((3,4-difluorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3,4-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (CD₃OD, 400 MHz) δ 8.22 (s, 1H), 8.20-8.05 (m, 2H), 7.95-7.75 (m, 2H), 7.37 (dd, 1H), 5.76 (brs, 1H), 2.65-2.45 (m, 2H), 0.88 (t, 3H)

### Example 499

### 2-(2-((3,5-difluorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3,5-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 29%).

¹H NMR (CD₃OD, 400 MHz) δ 8.24 (s, 1H), 7.95-7.75 (m, 4H), 7.16 (dd, 1H), 5.76 (brs, 1H), 2.65-2.45 (m, 2H), 0.88 (t, 3H)

### Example 500

### 2-(2-((3-fluoro-5-(trifluoromethyl)benzoyl)imino-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3-fluoro-5-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 16%).

¹H NMR (CD₃OD, 400 MHz) δ 8.38 (s, 1H), 8.25 (s, 1H), 8.21 (d, 1H), 7.95-7.80 (m, 2H), 7.66 (d, 1H), 5.90 (brs, 1H), 2.70-2.45 (m, 2H), 0.89 (t, 3H)

### Example 501

### 2-(2-((3,4-dichlorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3,4-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (CD₃OD, 400 MHz) δ 8.37 (s, 1H), 8.23 (s, 1H), 8.16 (d, 1H), 7.95-7.80 (m, 2H), 7.64 (d, 1H), 5.95 (brs, 1H), 2.65-2.45 (m, 2H), 0.88 (t, 3H)

### Example 502

### 2-(2-((3,5-dichlorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3,5-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 25%).

¹H NMR (CD₃OD, 400 MHz) δ 8.23 (s, 1H), 8.16 (s, 2H), 7.95-7.80 (m, 2H), 7.62 (s, 1H), 5.90 (brs, 1H), 2.70-2.45 (m, 2H), 0.88 (t, 3H)

### Example 503

### 2-(2-((3-chloro-4-fluorobenzoyl)imino)-6-(trifluoromethyl)benzo[d]thiazol-3(2H)-yl)butanoic acid

The titled compound as a white solid was prepared in accordance with the same procedures as in Example 52, using 6-(trifluoromethyl)benzo[d]thiazol-2-amine, 3-chloro-4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 22%).

¹H NMR (CD₃OD, 400 MHz) δ 8.35 (d, 1H), 8.30-8.15 (m, 2H), 7.95-7.75 (m, 2H), 7.34 (dd, 1H), 5.95 (brs, 1H), 2.65-2.45 (m, 2H), 0.88 (t, 3H)

### Example 504

### 2-(2-((3-fluorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 18%).

¹H NMR (CD₃OD, 400 MHz) δ 8.09 (d, 1H), 7.96 (d, 1H), 7.89 (s, 1H), 7.78 (d, 1H), 7.55-7.45 (m, 2H), 7.35-7.25 (m, 1H), 5.74 (brs, 1H), 2.70-2.45 (m, 2H), 0.90 (t, 3H)

### Example 505

### 2-(2-((4-fluorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 13%).

¹H NMR (CD₃OD, 400 MHz) δ 8.45-8.25 (m, 2H), 7.87 (s, 1H), 7.76 (d, 1H), 7.47 (d, 1H), 7.22 (dd, 2H), 5.75 (brs, 1H), 2.70-2.40 (m, 2H), 0.90 (t, 3H)

### Example 506

### 2-(2-((3-methoxybenzoyl)imino-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 13%).

¹H NMR (CD₃OD, 400 MHz) δ 7.95-7.80 (m, 3H), 7.77 (d, 1H), 7.48 (d, 1H), 7.40 (dd, 1H), 7.25-7.10 (m, 1H), 5.68 (brs, 1H), 3.91 (s, 3H), 2.75-2.45 (m, 2H), 0.90 (t, 3H)

### Example 507

### 2-(2-((4-methoxybenzoyl)imino-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 4-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 26%).

¹H NMR (CD₃OD, 400 MHz) δ 8.24 (d, 2H), 7.85 (s, 1H), 7.72 (d, 1H), 7.46 (d, 1H), 7.02 (d, 2H), 5.72 (brs, 1H), 3.89 (s, 3H), 2.70-2.45 (m, 2H), 0.89 (t, 3H)

### Example 508

### 2-(2-((4-chlorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 4-chlorobenzoyl chloride, and methyl 2-bromobutyrate, instead of 6-methoxybenzo[d]thiazol-2-amine, 3-chlorobenzoyl chloride and ethyl bromoacetate, respectively (Yield: 22%).

¹H NMR (CD₃OD, 400 MHz) δ 8.25 (d, 2H), 7.88 (s, 1H), 7.76 (d, 1H), 7.60-7.45 (m, 3H), 5.75 (brs, 1H), 2.70-2.40 (m, 2H), 0.89 (t, 3H)

### Example 509

### 2-(6-(trifluoromethoxy)-2-((3-(trifluoromethoxy)benzoyl)iminobenzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3-(trifluoromethoxy)benzoyl chloride, and methyl 2-bromobutyrate, instead of 6-methoxybenzo[d]thiazol-2-amine, 3-chlorobenzoyl chloride and ethyl bromoacetate, respectively (Yield: 25%).

¹H NMR (CD₃OD, 400 MHz) δ 8.26 (d, 1H), 8.14 (s, 1H), 7.89 (s, 1H), 7.77 (d, 1H), 7.59 (t, 1H), 7.47 (m, 2H), 5.73 (brs, 1H), 2.70-2.45 (m, 2H), 0.89 (t, 3H)

### Example 510

### 2-(2-((3,5-dimethoxybenzoyl)imino-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3,5-dimethoxybenzoyl chloride, and methyl 2-bromobutyrate, instead of 6-methoxybenzo[d]thiazol-2-amine, 3-chlorobenzoyl chloride and ethyl bromoacetate, respectively (Yield: 22%).

¹H NMR (CD₃OD, 400 MHz) δ 7.86 (s, 1H), 7.81 (d, 1H), 7.47 (s, 2H), 7.16 (s, 1H), 6.65 (s, 1H), 5.70 (brs, 1H), 3.86 (s, 6H), 2.70-2.45 (m, 2H), 0.88 (t, 3H)

### Example 511

### 2-(2-((3,4-difluorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3,4-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 18%).

¹H NMR (CD₃OD, 400 MHz) δ 8.25-8.05 (m, 2H), 7.88 (s, 1H), 7.78 (d, 1H), 7.49 (d, 1H), 7.39 (dd, 1H), 5.73 (brs, 1H), 2.70-2.45 (m, 2H), 0.89 (t, 3H)

### Example 512

### 2-(2-((3,5-difluorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3,5-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 19%).

¹H NMR (CD₃OD, 400 MHz) δ 7.88 (s, 1H), 7.90-7.75 (m, 3H), 7.48 (d, 1H), 7.30-7.05 (m, 1H), 5.71 (brs, 1H), 2.65-2.40 (m, 2H), 0.88 (t, 3H)

### Example 513

### 2-(2-((3-fluoro-5-(trifluoromethyl)benzoyl)imino-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3-fluoro-5-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 18%).

¹H NMR (CD₃OD, 400 MHz) δ 8.38 (s, 1H), 8.21 (d, 1H), 7.90 (s, 1H), 7.81 (d, 1H), 7.65 (d, 1H), 7.49 (d, 1H), 5.80 (brs, 1H), 2.70-2.45 (m, 2H), 0.89 (t, 3H)

### Example 514

### 2-(2-((3,4-dichlorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3,4-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 12%).

¹H NMR (CD₃OD, 400 MHz) δ 8.36 (s, 1H), 8.26 (d, 1H), 7.87 (s, 1H), 7.77 (d, 1H), 7.63 (d, 1H), 7.47 (d, 1H), 5.85 (brs, 1H), 2.65-2.45 (m, 2H), 0.88 (t, 3H)

### Example 515

### 2-(2-((3,5-dichlorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3,5-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 14%).

¹H NMR (CD₃OD, 400 MHz) δ 8.16 (s, 2H), 7.89 (s, 1H), 7.79 (d, 1H), 7.62 (s, 1H), 7.48 (d, 1H), 5.75 (brs, 1H), 2.70-2.40 (m, 2H), 0.88 (t. 3H)

### Example 516

### 2-(2-((3-chloro-4-fluorobenzoyl)imino)-6-(trifluoromethoxy)benzo[d]thiazol-3(2H)-yl)butanoic acid

The titled compound as a white solid was prepared in accordance with the same procedures as in Example 52, using 6-(trifluoromethoxy)benzo[d]thiazol-2-amine, 3-chloro-4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 27%).

¹H NMR (CD₃OD, 400 MHz) δ 8.35 (d, 1H), 8.30-8.15 (m, 1H), 7.87 (s, 1H), 7.76 (d, 1H), 7.47 (d, 1H), 7.34 (dd, 1H), 5.90 (brs, 1H), 2.65-2.45 (m, 2H), 0.88 (t, 3H)

### Example 517

### 2-(5,6,7-trifluoro-2-((3-fluorobenzoyl)imino)benzo[d]thiazol-3(2H)-yl)butanoic acid

The titled compound as a white solid was prepared in accordance with the same procedures as in Example 52, using 5,6,7-trifluorobenzo[d]thiazol-2-amine, 3-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 19%).

¹H NMR (CD₃OD, 400 MHz) δ 8.07 (d, 1H), 7.92 (d, 1H), 7.75-7.60 (m, 1H), 7.60-7.45 (m, 1H), 7.40-7.25 (m, 1H), 5.70 (brs, 1H), 2.70-2.40 (m, 2H), 0.89 (t, 3H)

### Example 518

### 2-(5,6,7-trifluoro-2-((4-fluorobenzoyl)imino)benzo[d]thiazol-3(2H)-yl)butanoic acid

The titled compound as a white solid was prepared in accordance with the same procedures as in Example 52, using 5,6,7-trifluorobenzo[d]thiazol-2-amine, 4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 15%).

¹H NMR (CD₃OD, 400 MHz) δ 8.35-8.25 (m, 2H), 7.75-7.60 (m, 1H), 7.30-7.15 (m, 2H), 5.70 (brs, 1H), 2.65-2.40 (m, 2H), 0.89 (t, 3H)

### Example 519

### 2-(5,6,7-trifluoro-2-((3-methoxybenzoyl)iminobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (CD₃OD, 400 MHz) δ 7.90-7.80 (m, 2H), 7.75-7.60 (m, 1H), 7.37 (dd, 1H), 7.15-7.05 (m, 1H), 5.65 (brs, 1H), 3.89 (s, 3H), 2.70-2.40 (m, 2H), 0.90 (t, 3H)

### Example 520

### 2-(5,6,7-trifluoro-2-((4-methoxybenzoyl)iminobenzo[d]thiazol-3(2H)-yl)butanoic acid

The titled compound as a white solid was prepared in accordance with the same procedures as in Example 52, using 5,6,7-trifluorobenzo[d]thiazol-2-amine, 4-methoxybenzoyl chloride, and methyl 2-bromobutyrate, instead of 6-methoxybenzo[d]thiazol-2-amine, 3-chlorobenzoyl chloride and ethyl bromoacetate, respectively (Yield: 17%).

¹H NMR (CD₃OD, 400 MHz) δ 8.20 (d, 2H), 7.75-7.55 (m, 1H), 6.99 (d, 2H), 5.70 (brs, 1H), 3.87 (s, 3H), 2.70-2.40 (m, 2H), 0.89 (t, 3H)

### Example 521

### 2-(2-((4-chlorobenzoyl)imino)-5,6,7-trifluorobenzo[d]thiazol-3(2H)-yl)butanoic acid

The titled compound as a white solid was prepared in accordance with the same procedures as in Example 52, using 5,6,7-trifluorobenzo[d]thiazol-2-amine, 4-chlorobenzoyl chloride, and methyl 2-bromobutyrate, instead of 6-methoxybenzo[d]thiazol-2-amine, 3-chlorobenzoyl chloride and ethyl bromoacetate, respectively (Yield: 19%).

¹H NMR (CD₃OD, 400 MHz) δ 8.21 (d, 2H), 7.75-7.60 (m, 1H), 7.48 (d, 2H), 5.80 (brs, 1H), 2.70-2.40 (m, 2H), 0.89 (t, 3H)

### Example 522

### 2-(5,6,7-trifluoro-2-((3-fluoro-4-methylbenzoyl)iminobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3-(trifluoromethoxy)benzoyl chloride, and methyl 2-bromobutyrate, instead of 6-methoxybenzo[d]thiazol-2-amine, 3-chlorobenzoyl chloride and ethyl bromoacetate, respectively (Yield: 10%).

¹H NMR (CD₃OD, 400 MHz) δ 7.96 (d, 1H), 7.86 (d, 1H), 7.75-7.60 (m, 1H), 7.45-7.30 (m, 1H), 5.70 (brs, 1H), 2.65-2.40 (m, 2H), 2.34 (s, 3H), 0.88 (t, 3H)

### Example 523

### 2-(2-((3-chloro-4-fluorobenzoyl)imino)-6,7-difluorobenzo[d]thiazol-3(2H)-yl)butanoic acid

The titled compound as a white solid was prepared in accordance with the same procedures as in Example 266, using 2,3,4-trifluoroaniline, 3-chloro-4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 14%).

¹H NMR (DMSO-d₆, 400 MHz) δ 13.26 (brs, 1H), 8.33-8.07 (m, 2H), 7.72 (m, 2H), 7.62-7.07 (m, 1H), 5.77 (brs, 1H), 2.50-2.33 (m, 2H), 1.05 (t, 3H)

### Example 524

### 2-(2-((3,4-difluorobenzoyl)imino)-5,6,7-trifluorobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3,4-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 20%).

¹H NMR (CD₃OD, 400 MHz) δ 8.15-8.00 (m, 2H), 7.80-7.65 (m, 1H), 7.45-7.30 (m, 1H), 5.70 (brs, 1H), 2.65-2.35 (m, 2H), 0.90 (t, 3H)

### Example 525

### 2-(2-((3,5-difluorobenzoyl)imino)-5,6,7-trifluorobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3,5-difluorobenzoyl chloride, and methyl 2-bromobutyrate, instead of 6-methoxybenzo[d]thiazol-2-amine, 3-chlorobenzoyl chloride and ethyl bromoacetate, respectively (Yield: 25%).

¹H NMR (CD₃OD, 400 MHz) δ 7.78 (d, 2H), 7.75-7.65 (m, 1H), 7.25-7.10 (m, 1H), 5.70 (brs, 1H), 2.65-2.40 (m, 2H), 0.89 (t, 3H)

### Example 526

### 2-(5,6,7-trifluoro-2-((3-fluoro-5-(trifluoromethyl)benzoyl)iminobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3-fluoro-5-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 14%).

¹H NMR (CD₃OD, 400 MHz) δ 8.35 (s, 1H), 8.18 (d, 1H), 7.85-7.70 (m, 1H), 7.67 (d, 1H), 5.75 (brs, 1H), 2.65-2.40 (m, 2H), 0.91 (t, 3H)

### Example 527

### 2-(5,6,7-trifluoro-2-((4-fluoro-3-(trifluoromethyl)benzoyl)imino)benzo[d]thiazol-3(2H)-yl)butanoic acid

The titled compound as a white solid was prepared in accordance with the same procedures as in Example 52, using 5,6,7-trifluorobenzo[d]thiazol-2-amine, 4-fluoro-3-(trifluoromethyl)benzoyl chloride, and methyl 2-bromobutyrate, instead of 6-methoxybenzo[d]thiazol-2-amine, 3-chlorobenzoyl chloride and ethyl bromoacetate, respectively (Yield: 16%).

¹H NMR (CD₃OD, 400 MHz) δ 8.70-8.50 (m, 2H), 7.80-7.60 (m, 1H), 7.46 (dd, 1H), 5.75 (brs, 1H), 2.65-2.45 (m, 2H), 0.90 (t, 3H)

### Example 528

### 2-(2-((3,5-dichlorobenzoyl)imino)-5,6,7-trifluorobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3,5-dichlorobenzoyl chloride, and methyl 2-bromobutyrate, instead of 6-methoxybenzo[d]thiazol-2-amine, 3-chlorobenzoyl chloride and ethyl bromoacetate, respectively (Yield: 9%).

¹H NMR (CD₃OD, 400 MHz) δ 8.15 (s, 2H), 7.80-7.64 (m, 1H), 7.64 (s, 1H), 5.70 (m, 1H), 2.65-2.35 (m, 2H), 0.89 (t, 3H)

### Example 529

### 2-(2-((3-chloro-4-fluorobenzoyl)imino)-5,6,7-trifluorobenzo[d]thiazol-3(2H)-yl)butanoic acid

The titled compound as a white solid was prepared in accordance with the same procedures as in Example 52, using 5,6,7-trifluorobenzo[d]thiazol-2-amine, 3-chloro-4-fluorobenzoyl chloride, and methyl 2-bromobutyrate, instead of 6-methoxybenzo[d]thiazol-2-amine, 3-chlorobenzoyl chloride and ethyl bromoacetate, respectively (Yield: 7%).

¹H NMR (CD₃OD, 400 MHz) δ 8.40-8.30 (m, 1H), 8.25-8.15 (m, 1H), 7.70-7.50 (m, 1H), 7.34 (dd, 1H), 5.80 (brs, 1H), 2.60-2.40 (m, 2H), 0.88 (t, 3H)

### Example 530

### 2-(7-fluoro-2-((3-fluorobenzoyl)imino)benzo[d]thiazol-3(2H)-yl)butanoic acid

The titled compound as a white solid was prepared in accordance with the same procedures as in Example 266, using 2,3-difluoroaniline, 3-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 15%).

¹H NMR (CD₃OD, 400 MHz) δ 8.10 (d, 1H), 7.93 (d, 1H), 7.58-7.47 (m, 3H), 7.31 (m, 1H), 7.16 (t, 1H), 5.70 (brs, 1H), 2.60-2.46 (m, 2H), 0.87 (t, 3H)

### Example 531

### 2-(7-fluoro-2-((4-fluorobenzoyl)imino)benzo[d]thiazol-3(2H)-yl)butanoic acid

The titled compound as a white solid was prepared in accordance with the same procedures as in Example 266, using 2,3-difluoroaniline, 4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 18%).

¹H NMR (CD₃OD, 400 MHz) δ 8.32 (q, 2H), 7.53 (m, 2H), 7.21-7.13 (m, 3H), 5.70 (brs, 1H), 2.58-2.47 (m, 2H), 0.87 (t, 3H)

### Example 532

### 2-(7-fluoro-2-((3-methoxybenzoyl)iminobenzo[d]thiazol-3(2H)-yl)butanoic acid

The titled compound as a white solid was prepared in accordance with the same procedures as in Example 266, using 2,3-difluoroaniline, 3-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 15%).

¹H NMR (CD₃OD, 400 MHz) δ 7.86 (s, 1H), 7.84 (d, 1H), 7.55 (m, 2H), 7.37 (t, 1H), 7.17-7.09 (m, 2H), 5.63 (brs, 1H), 3.89 (s, 3H), 2.63-2.48 (m, 2H), 0.87 (t, 3H)

### Example 533

### 2-(7-fluoro-2-((4-methoxybenzoyl)iminobenzo[d]thiazol-3(2H)-yl)butanoic acid

The titled compound as a white solid was prepared in accordance with the same procedures as in Example 266, using 2,3-difluoroaniline, 4-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 28%).

¹H NMR (CD₃OD, 400 MHz) δ 8.24 (d, 2H), 7.55-7.46 (m, 2H), 7.12 (t, 1H), 7.01 (d, 2H), 5.68 (brs, 1H), 3.87 (s, 3H), 2.59-2.44 (m, 2H), 0.87 (t, 3H)

### Example 534

### 2-(2-((4-chlorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 4-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 26%).

¹H NMR (DMSO-d₆, 400 MHz) δ 13.22 (brs, 1H), 8.22 (d, 2H), 7.71 (s, 1H), 7.63 (m, 3H), 7.32 (t, 1H), 5.81 (brs, 1H), 2.50-2.32 (m, 2H), 0.78 (t, 3H)

### Example 535

### 2-(7-fluoro-2-((3-(trifluoromethoxy)benzoyl)iminobenzo[d]thiazol-3(2H)-yl)butanoic acid

The titled compound as a white solid was prepared in accordance with the same procedures as in Example 266, using 2,3-difluoroaniline, 3-(trifluoromethoxy)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 18%).

¹H NMR (DMSO-d₆, 400 MHz) δ 13.21 (brs, 1H), 8.23 (d, 1H), 8.10 (s, 1H), 7.75-7.64 (m, 4H), 7.33 (t, 1H), 5.82 (brs, 1H), 2.50-2.34 (m, 2H), 0.78 (t, 3H)

### Example 536

### 2-(2-((3,5-dimethoxybenzoyl)imino-7-fluorobenzo[d]thiazol-3(2H)-yl)butanoic acid

The titled compound as a white solid was prepared in accordance with the same procedures as in Example 266, using 2,3-difluoroaniline, 3,5-dimethoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 16%).

¹H NMR (DMSO-d₆, 400 MHz) δ 13.12 (brs, 1H), 7.77 (s, 1H), 7.62 (m, 1H), 7.39 (d, 2H), 7.33 (t, 1H), 6.71 (s, 1H), 5.73 (brs, 1H), 3.83 (s, 6H), 2.50-2.33 (m, 2H), 0.77 (t, 3H)

### Example 537

### 2-(2-((3,4-difluorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 3,4-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 24%).

¹H NMR (DMSO-d₆, 400 MHz) δ 13.23 (brs, 1H), 8.17-7.97 (m, 2H), 7.73-7.58 (m, 3H), 7.33 (m, 1H), 5.81 (brs, 1H), 2.50-2.35 (m, 2H), 0.95 (t, 3H)

### Example 538

### 2-(2-((3,5-difluorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 3,5-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 22%).

¹H NMR (DMSO-d₆, 400 MHz) δ 13.25 (brs, 1H), 7.80 (m, 3H), 7.64 (m, 1H), 7.54 (t, 1H), 7.34 (t, 1H), 5.84 (brs, 1H), 2.50-2.32 (m, 2H), 0.79 (t, 3H)

### Example 539

### 2-(7-fluoro-2-((3-fluoro-5-(trifluoromethyl)benzoyl)iminobenzo[d]thiazol-3(2H)-yl)butanoic acid

The titled compound as a white solid was prepared in accordance with the same procedures as in Example 266, using 2,3-difluoroaniline, 3-fluoro-5-(trifluoromethyl)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 20%).

¹H NMR (DMSO-d₆, 400 MHz) δ 13.26 (brs, 1H), 8.32 (s, 1H), 8.22 (d, 1H), 7.99 (d, 1H), 7.79 (s, 1H), 7.67 (m, 1H), 7.35 (t, 1H), 5.84 (brs, 1H), 2.50-2.34 (m, 2H), 0.78 (t, 3H)

### Example 540

### 2-(2-((3,4-dichlorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 3,4-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 15%).

¹H NMR (DMSO-d₆, 400 MHz) δ 13.27 (brs, 1H), 8.35 (s, 1H), 8.13 (d, 1H), 7.82 (d, 1H), 7.75 (s, 1H), 7.65 (m, 1H), 7.33 (t, 1H), 5.82 (brs, 1H), 2.50-2.32 (m, 2H), 0.78 (t, 3H)

### Example 541

### 2-(2-((3,5-dichlorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 3,5-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 18%).

¹H NMR (DMSO-d₆, 400 MHz) δ 13.28 (brs, 1H), 8.19 (s, 2H), 7.94 (s, 1H), 7.88 (s, 1H), 7.66 (m, 1H), 7.35 (t, 1H), 5.83 (brs, 1H), 2.50-2.33 (m, 2H), 0.77 (t, 3H)

### Example 542

### 2-(2-((3-chloro-4-fluorobenzoyl)imino)-7-fluorobenzo[d]thiazol-3(2H)-yl)butanoic acid

The titled compound as a white solid was prepared in accordance with the same procedures as in Example 266, using 2,3-difluoroaniline, 3-chloro-4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (DMSO-d₆, 400 MHz) δ 13.23 (brs, 1H), 8.34-8.08 (m, 3H), 7.73 (s, 1H), 7.60 (m, 1H), 7.33 (m, 1H), 5.77 (brs, 1H), 2.50-2.35 (m, 2H), 1.07 (t, 3H)

### Example 543

### 2-(7-chloro-2-((3-fluorobenzoyl)imino)benzo[d]thiazol-3(2H)-yl)butanoic acid

The titled compound as a white solid was prepared in accordance with the same procedures as in Example 266, using 3-chloro-2-fluoroaniline, 3-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 29%).

¹H NMR (CD₃OD, 400 MHz) δ 8.15 (d, 1H), 7.94 (d, 1H), 7.65 (d, 1H), 7.52 (m, 2H), 7.43 (d, 1H), 7.28 (m, 1H), 5.69 (brs, 1H), 2.60-2.48 (m, 2H), 0.87 (t, 3H)

### Example 544

### 2-(7-chloro-2-((4-fluorobenzoyl)imino)benzo[d]thiazol-3(2H)-yl)butanoic acid

The titled compound as a white solid was prepared in accordance with the same procedures as in Example 266, using 3-chloro-2-fluoroaniline, 4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 16%).

¹H NMR (CD₃OD, 400 MHz) δ 8.33 (m, 2H), 7.63 (d, 1H), 7.52 (t, 1H), 7.41 (d, 1H), 7.19 (t, 2H), 5.69 (brs, 1H), 2.58-2.47 (m, 2H), 0.87 (t, 3H)

### Example 545

### 2-(7-chloro-2-((3-methoxybenzoyl)iminobenzo[d]thiazol-3(2H)-yl)butanoic acid

The titled compound as a white solid was prepared in accordance with the same procedures as in Example 266, using 3-chloro-2-fluoroaniline, 3-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 28%).

¹H NMR (CD₃OD, 400 MHz) δ 7.86 (s, 1H), 7.84 (d, 1H), 7.64 (d, 1H), 7.52 (t, 1H), 7.39 (m, 2H), 7.11 (m, 1H), 5.61 (brs, 1H), 3.89 (s, 3H), 2.63-2.46 (m, 2H), 0.87 (t, 3H)

### Example 546

### 2-(7-chloro-2-((4-methoxybenzoyl)iminobenzo[d]thiazol-3(2H)-yl)butanoic acid

The titled compound as a white solid was prepared in accordance with the same procedures as in Example 266, using 3-chloro-2-fluoroaniline, 4-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 22%).

¹H NMR (CD₃OD, 400 MHz) δ 8.24 (d, 2H), 7.59 (d, 1H), 7.50 (t, 1H), 7.39 (d, 1H), 7.01 (d, 2H), 5.67 (brs, 1H), 3.87 (s, 3H), 2.59-2.44 (m, 2H), 0.87 (t, 3H)

### Example 547

### 2-(7-chloro-2-((4-chlorobenzoyl)imino)benzo[d]thiazol-3(2H)-yl)butanoic acid

The titled compound as a white solid was prepared in accordance with the same procedures as in Example 266, using 3-chloro-2-fluoroaniline, 4-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 12%).

¹H NMR (DMSO-d₆, 400 MHz) δ 13.23 (brs, 1H), 8.22 (d, 2H), 7.84 (s, 1H), 7.61 (m, 3H), 7.54 (d, 1H), 5.80 (brs, 1H), 2.50-2.32 (m, 2H), 0.78 (t, 3H)

### Example 548

### 2-(7-chloro-2-((3-(trifluoromethoxy)benzoyl)iminobenzo[d]thiazol-3(2H)-yl)butanoic acid

The titled compound as a white solid was prepared in accordance with the same procedures as in Example 266, using 3-chloro-2-fluoroaniline, 3-(trifluoromethoxy)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (DMSO-d₆, 400 MHz) δ 13.21 (brs, 1H), 8.23 (d, 1H), 8.10 (s, 1H), 7.71 (s, 1H), 7.67 (m, 3H), 7.54 (d, 1H), 5.80 (brs, 1H), 2.50-2.33 (m, 2H), 0.78 (t, 3H)

### Example 549

### 2-(7-chloro-2-((3,5-dimethoxybenzoyl)iminobenzo[d]thiazol-3(2H)-yl)butanoic acid

The titled compound as a white solid was prepared in accordance with the same procedures as in Example 266, using 3-chloro-2-fluoroaniline, 3,5-dimethoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 28%).

¹H NMR (DMSO-d₆, 400 MHz) δ 13.11 (brs, 1H), 7.88 (s, 1H), 7.61 (t, 1H), 7.53 (d, 1H), 7.39 (s, 2H), 6.72 (s, 1H), 5.70 (brs, 1H), 3.83 (s, 6H), 2.50-2.32 (m, 2H), 0.77 (t, 3H)

### Example 550

### 2-(7-chloro-2-((3,4-difluorobenzoyl)imino)benzo[d]thiazol-3(2H)-yl)butanoic acid

The titled compound as a white solid was prepared in accordance with the same procedures as in Example 266, using 3-chloro-2-fluoroaniline, 3,4-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (DMSO-d₆, 400 MHz) δ 13.20 (brs, 1H), 8.16-7.84 (m, 3H), 7.60 (m, 2H), 7.52 (m, 1H), 5.77 (brs, 1H), 2.50-2.35 (m, 2H), 1.05 (t, 3H)

### Example 551

### 2-(7-chloro-2-((3,5-difluorobenzoyl)imino)benzo[d]thiazol-3(2H)-yl)butanoic acid

The titled compound as a white solid was prepared in accordance with the same procedures as in Example 266, using 3-chloro-2-fluoroaniline, 3,5-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 6%).

¹H NMR (DMSO-d₆, 400 MHz) δ 13.27 (brs, 1H), 7.87 (s, 1H), 7.81 (d, 2H), 7.64 (t, 1H), 7.55 (m, 2H), 5.82 (brs, 1H), 2.50-2.33 (m, 2H), 0.77 (t, 3H)

### Example 552

### 2-(7-chloro-2-((3-fluoro-5-(trifluoromethyl)benzoyl)iminobenzo[d]thiazol-3(2H)-yl)butanoic acid

The titled compound as a white solid was prepared in accordance with the same procedures as in Example 266, using 3-chloro-2-fluoroaniline, 3-fluoro-5-(trifluoromethyl)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 30%).

¹H NMR (DMSO-d₆, 400 MHz) δ 13.26 (brs, 1H), 8.32 (s, 1H), 8.22 (d, 1H), 7.99 (d, 1H), 7.91 (s, 2H), 7.67 (t, 1H), 7.58 (d, 1H), 5.83 (brs, 1H), 2.50-2.33 (m, 2H), 0.79 (t, 3H)

### Example 553

### 2-(7-chloro-2-((3,4-dichlorobenzoyl)imino)benzo[d]thiazol-3(2H)-yl)butanoic acid

The titled compound as a white solid was prepared in accordance with the same procedures as in Example 266, using 3-chloro-2-fluoroaniline, 3,4-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 6%).

¹H NMR (DMSO-d₆, 400 MHz) δ 13.25 (brs, 1H), 8.34 (s, 1H), 8.15 (m, 1H), 7.87 (s, 1H), 7.83 (d, 1H), 7.66 (t, 1H), 7.56 (d, 1H), 5.81 (brs, 1H), 2.50-2.34 (m, 2H), 0.78 (t, 3H)

### Example 554

### 2-(7-chloro-2-((3,5-dichlorobenzoyl)imino)benzo[d]thiazol-3(2H)-yl)butanoic acid

The titled compound as a white solid was prepared in accordance with the same procedures as in Example 266, using 3-chloro-2-fluoroaniline, 3,5-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 11%).

¹H NMR (DMSO-d₆, 400 MHz) δ 13.27 (brs, 1H), 8.12 (s, 2H), 7.88 (s, 2H), 7.65 (t, 1H), 7.57 (d, 1H), 5.81 (brs, 1H), 2.50-2.33 (m, 2H), 0.77 (t, 3H)

### Example 555

### 2-(7-chloro-2-((3-chloro-4-fluorobenzoyl)imino)benzo[d]thiazol-3(2H)-yl)butanoic acid

The titled compound as a white solid was prepared in accordance with the same procedures as in Example 266, using 3-chloro-2-fluoroaniline, 3-chloro-4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 12%).

¹H NMR (DMSO-d₆, 400 MHz) δ 13.20 (brs, 1H), 8.34-8.20 (m, 2H), 8.09 (m, 1H), 7.65-7.51 (m, 2H), 7.09 (m, 1H), 5.77 (brs, 1H), 2.50-2.36 (m, 2H), 0.77 (t, 3H)

### Example 556

### 2-(2-((3-fluorobenzoyl)imino)-7-(trifluoromethyl)benzo[d]thiazol-3(2H)-yl)butanoic acid

The titled compound as a white solid was prepared in accordance with the same procedures as in Example 266, using 3-fluorobenzoyl chloride and methyl 2-bromobutyrate, instead of 3-(trifluoromethyl)benzoyl chloride and ethyl bromoacetate, respectively (Yield: 15%).

¹H NMR (CD₃OD, 400 MHz) δ 8.11 (d, 1H), 7.97 (m, 2H), 7.73 (d, 2H), 7.51 (q, 1H), 7.30 (m, 1H), 5.79 (brs, 1H), 2.61-2.51 (m, 2H), 0.88 (t, 3H)

### Example 557

### 2-(2-((4-fluorobenzoyl)imino)-7-(trifluoromethyl)benzo[d]thiazol-3(2H)-yl)butanoic acid

The titled compound as a white solid was prepared in accordance with the same procedures as in Example 266, using 4-fluorobenzoyl chloride and methyl 2-bromobutyrate, instead of 3-(trifluoromethyl)benzoyl chloride and ethyl bromoacetate, respectively (Yield: 25%).

¹H NMR (CD₃OD, 400 MHz) δ 8.33 (m, 2H), 7.95 (s, 1H), 7.71 (d, 2H), 7.20 (t, 2H), 5.79 (brs, 1H), 2.60-2.49 (m, 2H), 0.88 (t, 3H)

### Example 558

### 2-(2-((3-methoxybenzoyl)imino-7-(trifluoromethyl)benzo[d]thiazol-3(2H)-yl)butanoic acid

The titled compound as a white solid was prepared in accordance with the same procedures as in Example 266, using 3-methoxybenzoyl chloride and methyl 2-bromobutyrate, instead of 3-(trifluoromethyl)benzoyl chloride and ethyl bromoacetate, respectively (Yield: 16%).

¹H NMR (CD₃OD, 400 MHz) δ 7.96 (s, 1H), 7.87 (s, 1H), 7.85 (d, 1H), 7.72 (m, 2H), 7.38 (t, 1H), 7.11 (d, 1H), 5.72 (brs, 1H), 3.89 (s, 3H), 2.64-2.49 (m, 2H), 0.88 (t, 3H)

### Example 559

### 2-(2-((4-methoxybenzoyl)imino-7-(trifluoromethyl)benzo[d]thiazol-3(2H)-yl)butanoic acid

The titled compound as a white solid was prepared in accordance with the same procedures as in Example 266, using 4-methoxybenzoyl chloride and methyl 2-bromobutyrate, instead of 3-(trifluoromethyl)benzoyl chloride and ethyl bromoacetate, respectively (Yield: 12%).

¹H NMR (CD₃OD, 400 MHz) δ 8.25 (d, 2H), 7.90 (s, 1H), 7.68 (d, 2H), 7.01 (d, 2H), 5.76 (brs, 1H), 3.87 (s, 3H), 2.59-2.49 (m, 2H), 0.88 (t, 3H)

### Example 560

### 2-(2-((4-chlorobenzoyl)imino)-7-(trifluoromethyl)benzo[d]thiazol-3(2H)-yl)butanoic acid

The titled compound as a white solid was prepared in accordance with the same procedures as in Example 266, using 4-chlorobenzoyl chloride and methyl 2-bromobutyrate, instead of 3-(trifluoromethyl)benzoyl chloride and ethyl bromoacetate, respectively (Yield: 27%).

¹H NMR (DMSO-d₆, 400 MHz) δ 13.28 (brs, 1H), 8.22 (m, 3H), 7.80 (m, 2H), 7.62 (d, 2H), 5.89 (brs, 1H), 2.50-2.34 (m, 2H), 0.79 (t, 3H)

### Example 561

### 2-(2-((3-(trifluoromethoxy)benzoyl)imino-7-(trifluoromethyl)benzo[d]thiazol-3(2H)-yl)butanoic acid

The titled compound as a white solid was prepared in accordance with the same procedures as in Example 266, using 3-(trifluoromethoxy)benzoyl chloride and methyl 2-bromobutyrate, instead of 3-(trifluoromethyl)benzoyl chloride and ethyl bromoacetate, respectively (Yield: 22%).

¹H NMR (DMSO-d₆, 400 MHz) δ 13.26 (brs, 1H), 8.21 (m, 2H), 8.11 (s, 1H), 7.83 (s, 2H), 7.68 (m, 2H), 5.88 (brs, 1H), 2.50-2.40 (m, 2H), 0.79 (t, 3H)

### Example 562

### 2-(2-((3,5-dimethoxybenzoyl)imino-7-(trifluoromethyl)benzo[d]thiazol-3(2H)-yl)butanoic acid

The titled compound as a white solid was prepared in accordance with the same procedures as in Example 266, using 3,5-dimethoxybenzoyl chloride and methyl 2-bromobutyrate, instead of 3-(trifluoromethyl)benzoyl chloride and ethyl bromoacetate, respectively (Yield: 16%).

¹H NMR (DMSO-d₆, 400 MHz) δ 13.16 (brs, 1H), 8.21 (s, 1H), 7.81 (d, 2H), 7.40 (d, 2H), 6.73 (s, 1H), 5.81 (brs, 1H), 3.83 (s, 6H), 2.50-2.37 (m, 2H), 0.79 (t, 3H)

### Example 563

### 2-(2-((3,4-difluorobenzoyl)imino)-7-(trifluoromethyl)benzo[d]thiazol-3(2H)-yl)butanoic acid

The titled compound as a white solid was prepared in accordance with the same procedures as in Example 266, using 3,4-difluorobenzoyl chloride and methyl 2-bromobutyrate, instead of 3-(trifluoromethyl)benzoyl chloride and ethyl bromoacetate, respectively (Yield: 18%).

¹H NMR (DMSO-d₆, 400 MHz) δ 13.24 (brs, 1H), 8.17-8.05 (m, 2H), 7.95 (m, 2H), 7.81 (m, 2H), 5.90 (brs, 1H), 2.50-2.33 (m, 2H), 0.78 (t, 3H)

### Example 564

### 2-(2-((3,5-difluorobenzoyl)imino)-7-(trifluoromethyl)benzo[d]thiazol-3(2H)-yl)butanoic acid

The titled compound as a white solid was prepared in accordance with the same procedures as in Example 266, using 3,5-difluorobenzoyl chloride and methyl 2-bromobutyrate, instead of 3-(trifluoromethyl)benzoyl chloride and ethyl bromoacetate, respectively (Yield: 19%).

¹H NMR (DMSO-d₆, 400 MHz) δ 13.30 (brs, 1H), 8.21 (s, 1H), 7.82 (m, 4H), 7.55 (t, 1H), 5.92 (brs, 1H), 2.50-2.35 (m, 2H), 0.78 (t, 3H)

### Example 565

### 2-(2-((3-fluoro-5-(trifluoromethyl)benzoyl)imino-7-(trifluoromethyl)benzo[d]thiazol-3(2H)-yl)butanoic acid

The titled compound as a white solid was prepared in accordance with the same procedures as in Example 266, using 3-fluoro-5-(trifluoromethyl)benzoyl chloride and methyl 2-bromobutyrate, instead of 3-(trifluoromethyl)benzoyl chloride and ethyl bromoacetate, respectively (Yield: 23%).

¹H NMR (DMSO-d₆, 400 MHz) δ 13.30 (brs, 1H), 8.33 (s, 1H), 8.22 (m, 2H), 8.01 (d, 1H), 7.84 (m, 2H), 5.93 (brs, 1H), 2.50-2.36 (m, 2H), 0.79 (t, 3H)

### Example 566

### 2-(2-((3,4-dichlorobenzoyl)imino)-7-(trifluoromethyl)benzo[d]thiazol-3(2H)-yl)butanoic acid

The titled compound as a white solid was prepared in accordance with the same procedures as in Example 266, using 3,4-dichlorobenzoyl chloride and methyl 2-bromobutyrate, instead of 3-(trifluoromethyl)benzoyl chloride and ethyl bromoacetate, respectively (Yield: 22%).

¹H NMR (DMSO-d₆, 400 MHz) δ 13.31 (brs, 1H), 8.35 (s, 1H), 8.20 (s, 1H), 8.13 (m, 1H), 7.84 (d, 3H), 5.91 (brs, 1H), 2.51-2.35 (m, 2H), 0.79 (t, 3H)

### Example 567

### 2-(2-((3,5-dichlorobenzoyl)imino)-7-(trifluoromethyl)benzo[d]thiazol-3(2H)-yl)butanoic acid

The titled compound as a white solid was prepared in accordance with the same procedures as in Example 266, using 3,5-dichlorobenzoyl chloride and methyl 2-bromobutyrate, instead of 3-(trifluoromethyl)benzoyl chloride and ethyl bromoacetate, respectively (Yield: 28%).

¹H NMR (DMSO-d₆, 400 MHz) δ 13.32 (brs, 1H), 8.23 (s, 1H), 8.13 (s, 1H), 7.89 (s, 1H), 7.83 (s, 2H), 5.91 (brs, 1H), 2.50-2.36 (m, 2H), 0.79 (t, 3H)

### Example 568

### 2-(2-((3-chloro-4-fluorobenzoyl)imino)-7-(trifluoromethyl)benzo[d]thiazol-3(2H)-yl)butanoic acid

The titled compound as a white solid was prepared in accordance with the same procedures as in Example 266, using 3-chloro-4-fluorobenzoyl chloride and methyl 2-bromobutyrate, instead of 3-(trifluoromethyl)benzoyl chloride and ethyl bromoacetate, respectively (Yield: 27%).

¹H NMR (DMSO-d₆, 400 MHz) δ 13.26 (brs, 1H), 8.35-8.08 (m, 3H), 7.83 (m, 2H), 7.77-7.07 (m, 1H), 5.86 (brs, 1H), 2.50-2.37 (m, 2H), 0.78 (t, 3H)

### Example 569

### 2-(5-chloro-2-((3-fluorobenzoyl)imino)benzo[d]thiazol-3(2H)-yl)butanoic acid

The titled compound as a white solid was prepared in accordance with the same procedures as in Example 266, using 5-chloro-2-fluoroaniline, 3-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 25%).

¹H NMR (DMSO-d₆, 400 MHz) δ 13.16 (brs, 1H), 8.08-8.01 (m, 3H), 7.90 (m, 1H), 7.58 (m, 1H), 7.46 (m, 2H), 5.74 (brs, 1H), 2.50-2.31 (m, 2H), 0.77 (t, 3H)

### Example 570

### 2-(5-chloro-2-((4-fluorobenzoyl)imino)benzo[d]thiazol-3(2H)-yl)butanoic acid

The titled compound as a white solid was prepared in accordance with the same procedures as in Example 266, using 5-chloro-2-fluoroaniline, 4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 15%).

¹H NMR (DMSO-d₆, 400 MHz) δ 13.20 (brs, 1H), 8.06-8.03 (m, 3H), 7.88 (m, 1H), 7.56 (m, 1H), 7.42 (m, 2H), 5.67 (brs, 1H), 2.50-2.29 (m, 2H), 0.78 (t, 3H)

### Example 571

### 2-(5-chloro-2-((3-methoxybenzoyl)iminobenzo[d]thiazol-3(2H)-yl)butanoic acid

The titled compound as a white solid was prepared in accordance with the same procedures as in Example 266, using 5-chloro-2-fluoroaniline, 3-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 14%).

¹H NMR (CD₃OD, 400 MHz) δ 7.85-7.78 (m, 4H), 7.36 (m, 2H), 7.11 (d, 1H), 5.61 (brs, 1H), 3.89 (s, 3H), 2.62-2.46 (m, 2H), 0.87 (t, 3H)

### Example 572

### 2-(5-chloro-2-((4-methoxybenzoyl)iminobenzo[d]thiazol-3(2H)-yl)butanoic acid

The titled compound as a white solid was prepared in accordance with the same procedures as in Example 266, using 5-chloro-2-fluoroaniline, 4-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 7%).

¹H NMR (CD₃OD, 400 MHz) δ 8.23 (d, 2H), 7.78 (s, 1H), 7.73 (s, 1H), 7.36 (d, 1H), 7.00 (d, 2H), 5.65 (brs, 1H), 3.87 (s, 3H), 2.59-2.43 (m, 2H), 0.87 (t, 3H)

### Example 573

### 2-(5-chloro-2-((4-chlorobenzoyl)imino)benzo[d]thiazol-3(2H)-yl)butanoic acid

The titled compound as a white solid was prepared in accordance with the same procedures as in Example 266, using 5-chloro-2-fluoroaniline, 4-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 4%).

¹H NMR (DMSO-d₆, 400 MHz) δ 13.16 (brs, 1H), 8.19 (d, 2H), 8.06 (m, 2H), 7.62 (d, 2H), 7.45 (d, 1H), 5.74 (brs, 1H), 2.50-2.29 (m, 2H), 0.78 (t, 3H)

### Example 574

### 2-(5-chloro-2-((3-(trifluoromethoxy)benzoyl)iminobenzo[d]thiazol-3(2H)-yl)butanoic acid

The titled compound as a white solid was prepared in accordance with the same procedures as in Example 266, using 5-chloro-2-fluoroaniline, 3-(trifluoromethoxy)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (DMSO-d₆, 400 MHz) δ 13.13 (brs, 1H), 8.21 (m, 1H), 8.09-8.02 (m, 3H), 7.68-7.62 (m, 2H), 7.48 (d, 1H), 5.75 (brs, 1H), 2.50-2.34 (m, 2H), 0.78 (t, 3H)

### Example 575

### 2-(5-chloro-2-((3,5-dimethoxybenzoyl)iminobenzo[d]thiazol-3(2H)-yl)butanoic acid

The titled compound as a white solid was prepared in accordance with the same procedures as in Example 266, using 5-chloro-2-fluoroaniline, 3,5-dimethoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 23%).

¹H NMR (DMSO-d₆, 400 MHz) δ 13.07 (brs, 1H), 8.09 (s, 1H), 8.01 (d, 1H), 7.45 (d, 1H), 7.38 (s, 2H), 6.71 (s, 1H) 5.68 (brs, 1H), 3.83 (s, 6H), 2.50-2.32 (m, 2H), 0.77 (t, 3H)

### Example 576

### 2-(5-chloro-2-((3,4-difluorobenzoyl)imino)benzo[d]thiazol-3(2H)-yl)butanoic acid

The titled compound as a white solid was prepared in accordance with the same procedures as in Example 266, using 5-chloro-2-fluoroaniline, 3,4-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 22%).

¹H NMR (DMSO-d₆, 400 MHz) δ 13.14 (brs, 1H), 8.27-7.91 (m, 4H), 7.68-7.57 (m, 1H), 7.48-7.38 (m, 1H), 5.71 (brs, 1H), 2.45-2.33 (m, 2H), 0.85 (t, 3H)

### Example 577

### 2-(5-chloro-2-((3,5-difluorobenzoyl)imino)benzo[d]thiazol-3(2H)-yl)butanoic acid

The titled compound as a white solid was prepared in accordance with the same procedures as in Example 266, using 5-chloro-2-fluoroaniline, 3,5-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 31%).

¹H NMR (DMSO-d₆, 400 MHz) δ 13.20 (brs, 1H), 8.10 (s, 1H), 8.05 (d, 1H), 7.79 (d, 2H), 7.55-7.47 (m, 2H), 5.76 (brs, 1H), 2.50-2.29 (m, 2H), 0.77 (t, 3H)

### Example 578

### 2-(5-chloro-2-((3-fluoro-5-(trifluoromethyl)benzoyl)iminobenzo[d]thiazol-3(2H)-yl)butanoic acid

The titled compound as a white solid was prepared in accordance with the same procedures as in Example 266, using 5-chloro-2-fluoroaniline, 3-fluoro-5-(trifluoromethyl)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (DMSO-d₆, 400 MHz) δ 13.26 (brs, 1H), 8.31-7.96 (m, 5H), 7.51 (d, 1H), 5.77 (brs, 1H), 2.50-2.33 (m, 2H), 0.77 (t, 3H)

### Example 579

### 2-(5-chloro-2-((3,4-dichlorobenzoyl)imino)benzo[d]thiazol-3(2H)-yl)butanoic acid

The titled compound as a white solid was prepared in accordance with the same procedures as in Example 266, using 5-chloro-2-fluoroaniline, 3,4-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (DMSO-d₆, 400 MHz) δ 13.21 (brs, 1H), 8.33 (s, 1H), 8.11-7.97 (m, 3H), 7.82 (t, 1H), 7.49 (d, 1H), 5.75 (brs, 1H), 2.50-2.29 (m, 2H), 0.77 (t, 3H)

### Example 580

### 2-(5-chloro-2-((3,5-dichlorobenzoyl)imino)benzo[d]thiazol-3(2H)-yl)butanoic acid

The titled compound as a white solid was prepared in accordance with the same procedures as in Example 266, using 5-chloro-2-fluoroaniline, 3,5-dichlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 22%).

¹H NMR (DMSO-d₆, 400 MHz) δ 13.20 (brs, 1H), 8.27-8.01 (m, 4H), 7.87 (s, 1H), 7.48 (m, 1H), 5.75 (brs, 1H), 2.50-2.30 (m, 2H), 0.76 (t, 3H)

### Example 581

### 2-(5-chloro-2-((3-chloro-4-fluorobenzoyl)imino)benzo[d]thiazol-3(2H)-yl)butanoic acid

The titled compound as a white solid was prepared in accordance with the same procedures as in Example 266, using 5-chloro-2-fluoroaniline, 3-chloro-4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 26%).

¹H NMR (DMSO-d₆, 400 MHz) δ 13.16 (brs, 1H), 8.32-7.98 (m, 4H), 7.61-7.05 (m, 2H), 5.71 (brs, 1H), 2.50-2.33 (m, 2H), 0.78 (t, 3H)

### Example 582

### 2-(6,7-difluoro-2-((3-fluorobenzoyl)imino)benzo[d]thiazol-3(2H)-yl)butanoic acid

The titled compound as a white solid was prepared in accordance with the same procedures as in Example 266, using 2,3,4-trifluoroaniline, 3-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 24%).

¹H NMR (CD₃OD, 400 MHz) δ 8.09 (s, 1H), 7.95 (s, 1H), 7.51 (m, 3H), 7.30 (m, 1H), 5.66 (brs, 1H), 2.58-2.47 (m, 2H), 0.87 (t, 3H)

### Example 583

### 2-(6,7-difluoro-2-((4-fluorobenzoyl)imino)benzo[d]thiazol-3(2H)-yl)butanoic acid

The titled compound as a white solid was prepared in accordance with the same procedures as in Example 266, using 2,3,4-trifluoroaniline, 4-fluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 17%).

¹H NMR (CD₃OD, 400 MHz) δ 8.31 (m, 2H), 7.48 (m, 2H), 7.19 (t, 2H), 5.67 (brs, 1H), 2.59-2.44 (m, 2H), 0.88 (t, 3H)

### Example 584

### 2-(6,7-difluoro-2-((3-methoxybenzoyl)iminobenzo[d]thiazol-3(2H)-yl)butanoic acid

The titled compound as a white solid was prepared in accordance with the same procedures as in Example 266, using 2,3,4-trifluoroaniline, 3-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 14%).

¹H NMR (CD₃OD, 400 MHz) δ 7.84 (s, 1H), 7.83 (d, 1H), 7.49 (m, 2H), 7.37 (t, 1H), 7.10 (m, 1H), 5.60 (brs, 1H), 3.89 (s, 3H), 2.61-2.45 (m, 2H), 0.88 (t, 3H)

### Example 585

### 2-(6,7-difluoro-2-((4-methoxybenzoyl)iminobenzo[d]thiazol-3(2H)-yl)butanoic acid

The titled compound as a white solid was prepared in accordance with the same procedures as in Example 266, using 2,3,4-trifluoroaniline, 4-methoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 15%).

¹H NMR (CD₃OD, 400 MHz) δ 8.23 (d, 2H), 7.45 (m, 2H), 7.00 (d, 2H), 5.66 (brs, 1H), 3.87 (s, 3H), 2.58-2.45 (m, 2H), 0.87 (t, 3H)

### Example 586

### 2-(2-((4-chlorobenzoyl)imino)-6,7-difluorobenzo[d]thiazol-3(2H)-yl)butanoic acid

The titled compound as a white solid was prepared in accordance with the same procedures as in Example 266, using 2,3,4-trifluoroaniline, 4-chlorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 10%).

¹H NMR (DMSO-d₆, 400 MHz) δ 13.25 (brs, 1H), 8.20 (d, 2H), 7.72 (s, 2H), 7.61 (d, 2H), 5.79 (brs, 1H), 2.50-2.32 (m, 2H), 0.78 (t, 3H)

### Example 587

### 2-(6,7-difluoro-2-((3-(trifluoromethoxy)benzoyl)iminobenzo[d]thiazol-3(2H)-yl)butanoic acid

The titled compound as a white solid was prepared in accordance with the same procedures as in Example 266, using 2,3,4-trifluoroaniline, 3-(trifluoromethoxy)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 29%).

¹H NMR (DMSO-d₆, 400 MHz) δ 13.24 (brs, 1H), 8.22 (d, 1H), 8.09 (s, 1H), 7.76-7.62 (m, 4H), 5.80 (brs, 1H), 2.50-2.33 (m, 2H), 0.78 (t, 3H)

### Example 588

### 2-(2-((3,5-dimethoxybenzoyl)imino-6,7-difluorobenzo[d]thiazol-3(2H)-yl)butanoic acid

The titled compound as a white solid was prepared in accordance with the same procedures as in Example 266, using 2,3,4-trifluoroaniline, 3,5-dimethoxybenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 29%).

¹H NMR (DMSO-d₆, 400 MHz) δ 13.15 (brs, 1H), 7.72 (m, 2H), 7.38 (d, 2H), 6.73 (s, 1H), 5.71 (brs, 1H), 3.83 (s, 6H), 2.50-2.31 (m, 2H), 0.77 (t, 3H)

### Example 589

### 2-(2-((3,4-difluorobenzoyl)imino)-6,7-difluorobenzo[d]thiazol-3(2H)-yl)butanoic acid

The titled compound as a white solid was prepared in accordance with the same procedures as in Example 266, using 2,3,4-trifluoroaniline, 3,4-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 25%).

¹H NMR (DMSO-d₆, 400 MHz) δ 13.25 (brs, 1H), 8.13 (t, 1H), 8.05 (s, 1H), 7.97-7.72 (m, 3H), 7.60 (m, 1H), 5.81 (brs, 1H), 2.51-2.34 (m, 2H), 1.05 (t, 3H)

### Example 590

### 2-(2-((3,5-difluorobenzoyl)imino)-6,7-difluorobenzo[d]thiazol-3(2H)-yl)butanoic acid

The titled compound as a white solid was prepared in accordance with the same procedures as in Example 266, using 2,3,4-trifluoroaniline, 3,5-difluorobenzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 22%).

¹H NMR (DMSO-d₆, 400 MHz) δ 13.29 (brs, 1H), 7.80-7.71 (m, 4H), 7.55 (t, 1H), 5.83 (brs, 1H), 2.43-2.32 (m, 2H), 0.78 (t, 3H)

### Example 591

### 2-(6,7-difluoro-2-((3-fluoro-5-(trifluoromethyl)benzoyl)iminobenzo[d]thiazol-3(2H)-yl)butanoic acid

The titled compound as a white solid was prepared in accordance with the same procedures as in Example 266, using 2,3,4-trifluoroaniline, 3-fluoro-5-(trifluoromethyl)benzoyl chloride, and methyl 2-bromobutyrate, instead of 2-fluoro-3-(trifluoromethyl)aniline, 3-(trifluoromethyl)benzoyl chloride and ethyl bromoacetate, respectively (Yield: 11%).

¹H NMR (DMSO-d₆, 400 MHz) δ 13.29 (brs, 1H), 8.31 (s, 1H), 8.20 (d, 1H), 8.00 (d, 1H), 7.77 (m, 2H), 5.83 (brs, 1H), 2.50-2.33 (m, 2H), 0.79 (t, 3H)

### Test Example 1

### Evaluation of Inhibitory Activity Against the Interaction Between the KRS Protein and the Laminin Receptor (LR)

The inhibitory activity against the interaction between the KRS protein and the laminin receptor (LR) was evaluated according to the Yeast Two Hybrid method described in WO2011/056021. According to the disclosures of WO2011/056021, we prepared a vector obtained by cloning the KRS gene in the LexA vector (Clontech) and a vector obtained by cloning the LR gene in the B42 vector (Clontech). The yeast cells, i.e., EGY/SH cells were co-transformed with the LexA-KRS vector and the B42-LR vector. The co-transformation was carried out using the Yeastmaker yeast transformation system2 kit (Clontech), according to the manufacturer's instruction. The resulting transformed yeast cells were cultured in a 40% galactose SD medium which does not have uracil (Ura), histidine (His), tryptophan (Trp) and leucine (Leu), at 30° C. for 16 hours. The cultured yeast cells were diluted with the said galactose medium, until the absorbance at 540 nm attains to about 0.15 to 0.2. The diluted culture (200 μL), containing the yeast cells, was added to each well of a 96-well plate. Each test compound was dissolved in dimethyl sulfoxide at a concentration of 20 mM and then the resulting solutions (1 μL) were added to each well. The plate was cultured at 30° C. for 6 days and then the absorbance of the each well was measured at 540 nm.

And also, specificity of the inhibitory activity was evaluated, using the yeast cells cotransformed with the LexA-KRS vector and the B42-AIMP2 vector; and the yeast cells co-transformed with the LexA-KRS vector and the B42-AIMP3 vector, which were respectively prepared according to WO2011/056021. The co-transformation was carried out using the Yeastmaker yeast transformation system2 kit (Clontech), according to the manufacturer's instruction. The resulting transformed yeast cells were cultured in a 40% galactose SD medium which does not have uracil (Ura), histidine (His), tryptophan (Trp) and leucine (Leu). The cultured yeast cells were diluted with the said galactose medium, until the absorbance at 540 nm attains to about 0.15 to 0.2. The diluted culture (200 μL), containing the yeast cells, was added to each well of a 96-well plate. Each test compound was dissolved in dimethyl sulfoxide at a concentration of 20 mM and then the resulting solutions (1 μL) were added to each well. The plate was cultured at 30° C. for 6 days and then the absorbance of each well was measured at 540 nm. The respective yeast cell proliferation rates (%) in the treated samples were calculated in comparison with those of the non-treated control (100%) and the results thereof are shown in Table 1 below.

TABLE 1
Yeast cell proliferation rates

| Test | | KRS- | KRS- |
|---|---|---|---|
| compound | KRS-LR | AIMP2 | AIMP3 |
| (Example No.) | (%) | (%) | (%) |
| 7 | 36.9 | 106.4 | 100.1 |
| 8 | 17.4 | 84.6 | 84.1 |
| 55 | 41.4 | 106.0 | 105.3 |
| 56 | 31.2 | 103.3 | 104.7 |
| 59 | 31.7 | 97.7 | 80.8 |
| 64 | 28.8 | 99.2 | 104.1 |
| 91 | 27.8 | 116.6 | 98.5 |
| 93 | 33.2 | 88.5 | 102.7 |
| 109 | 30.2 | 102.0 | 98.2 |
| 133 | 33.2 | 82.1 | 92.8 |
| 139 | 31.5 | 79.4 | 102.1 |
| 142 | 39.4 | 95.0 | 106.5 |
| 155 | 29.7 | 102.4 | 100.0 |
| 172 | 36.3 | 90.0 | 104.1 |
| 237 | 34.1 | 99.0 | 114.8 |
| 290 | 35.1 | 87.0 | 99.0 |
| 299 | 13.8 | 92.0 | 90.4 |
| 309 | 14.5 | 95.8 | 101.4 |
| 310 | 15.9 | 100.2 | 105.4 |
| 312 | 14.6 | 89.6 | 99.0 |
| 335 | 10.8 | 101.6 | 105.3 |
| 337 | 12.3 | 92.5 | 100.3 |
| 338 | 12.6 | 104.0 | 107.2 |
| 344 | 11.7 | 99.0 | 102.8 |
| 353 | 28.2 | 89.4 | 99.5 |
| 354 | 30.2 | 103.0 | 106.4 |
| 355 | 22.1 | 97.2 | 104.4 |
| 359 | 21.9 | 101.6 | 107.0 |
| 361 | 34.6 | 104.3 | 108.4 |
| 385 | 11.6 | 108.2 | 99.1 |
| 386 | 11.2 | 103.9 | 106.3 |
| 416 | 24.7 | 99.6 | 99.3 |
| 424 | 15.6 | 99.9 | 104.6 |
| 458 | 30.8 | 96.4 | 100.0 |
| 463 | 34.3 | 96.7 | 93.2 |
| 476 | 29.6 | 91.0 | 86.1 |
| 507 | 45.9 | 99.3 | 99.0 |
| 532 | 43.7 | 98.6 | 97.3 |
| 533 | 39.0 | 94.8 | 98.3 |
| 543 | 36.1 | 97.1 | 90.3 |
| 582 | 49.3 | 99.0 | 93.0 |
| 585 | 46.3 | 95.8 | 84.0 |

When KRS is bound to LR, the trans-activation domain is come close to the DNA-binding domain and the genes for synthesizing essential amino acids are expressed, which leads to normal growth of the transformed yeasts, thereby increasing the absorbance. However, if the binding between KRS and LR is inhibited, the gene expression is also inhibited; and therefore the transformed yeasts cannot grow normally. As shown in Table 1, the compounds of the present invention potently inhibit the binding between KRS and LR. And also, as shown in Table 1, the inhibitory activity against the binding between KRS and LR was significantly higher, than the inhibitory activity against the binding between KRS and AIMP2 or the inhibitory activity against the binding between KRS and AIMP3. Therefore, it can be seen that the compounds of the present invention selectively inhibit the binding between KRS and LR.

### Test Example 2

### Cell Migration Assay

Cell migration was measured with a 24-well transwell having a polycarbonate membrane (8.0 μm pore size, Costar) at the bottom of the upper chamber. Each lower well was treated with laminin (10 μg/mL) and test compounds (0.39˜100 μM) along with a serum-free RPMI1640 medium. A549 cells (Korean Cell Line Bank) were suspended in a serum-free RPMI1640 medium at a concentration of 1×10⁶ cells/mL and then the test compounds were added thereto at the indicated concentrations. Each resulting solution (100 μL) was added to the upper chamber. The cells were cultured at 37° C. for 6 hours in a CO₂ incubator. The upper chamber was taken out and then the cells were fixed with PBS containing 70% methyl alcohol for 10 minutes, followed by washing with PBS twice. The cells were stained with hematoxylin (Sigma) for 10 minutes and then washed with distilled water. The non-migrated cells were removed from the upper portion of the polycarbonate membrane with a cotton swab. The membranes were excised and separated from the chamber, and then mounted to a slide using a gel mount reagent (Biomeda, USA). The migrated cells (attached to the lower face of the membrane) were counted at three randomly selected sites under a microscope (×20). The cell counting was performed with the Image J program. From the counted cell numbers, each IC₅₀ value was calculated by non-linear regression curve fit using the “GraphPad Prism” program. The results are shown in Table 2 and Table 3 below.

TABLE 2

| Test compounds | |
|---|---|
| (Example No.) | IC₅₀ (μM) |
| 6 | 26.5 |
| 14 | 21.5 |
| 16 | 11.8 |
| 23 | 23.7 |
| 53 | 59.0 |
| 55 | 22.0 |
| 56 | 2.7 |
| 59 | 29.6 |
| 60 | 26.9 |
| 64 | 20.1 |
| 78 | 15.9 |
| 83 | 12.3 |
| 90 | 31.6 |
| 93 | 17.6 |
| 98 | 5.2 |
| 100 | 43.0 |
| 103 | 28.0 |
| 105 | 19.0 |
| 109 | 57.0 |
| 123 | 40.1 |
| 125 | 8.4 |
| 133 | 11.4 |
| 139 | 18.9 |
| 142 | 14.7 |
| 147 | 5.7 |
| 152 | 22.7 |
| 153 | 31.1 |
| 155 | 16.7 |
| 172 | 37.9 |
| 173 | 16.5 |
| 176 | 5.8 |
| 186 | 101.0 |
| 189 | 87.0 |
| 192 | 33.0 |
| 195 | 87.0 |
| 212 | 33.0 |

TABLE 3

| Test compounds | |
|---|---|
| (Example No.) | IC₅₀ (μM) |
| 240 | 16.0 |
| 246 | 12.0 |
| 270 | 1.0 |
| 282 | 25.0 |
| 283 | 10.0 |
| 289 | 9.8 |
| 290 | 3.2 |
| 293 | 1.0 |
| 295 | 60.0 |
| 299 | 7.9 |
| 308 | 6.6 |
| 309 | 12.9 |
| 310 | 6.5 |
| 312 | 36.0 |
| 314 | 46.0 |
| 338 | 81.3 |
| 346 | 124.5 |
| 347 | 109.4 |
| 356 | 40.9 |
| 358 | 29.4 |
| 361 | 142.4 |
| 373 | 92.4 |
| 380 | 36.2 |
| 385 | 25.4 |
| 386 | 8.3 |
| 388 | 37.4 |
| 396 | 43 |
| 417 | 22 |
| 424 | 78 |
| 427 | 18 |
| 442 | 80 |
| 455 | 80 |
| 458 | 61 |
| 462 | 43 |
| 466 | 49 |
| 467 | 19 |
| 468 | 41 |
| 469 | 66 |
| 471 | 32 |

The cells are pulled by laminin residing in the lower chamber and then passed through the polycarbonate membrane, so as to attach to the lower face of the membrane. As shown in Table 2 and Table 3, the compounds of the present invention efficiently inhibit the cell migration, thereby showing excellent activity against a disease associated with cancer cell metastasis.

### Test Example 3

### Cytotoxicity Study

A549 cells (Korean Cell Line Bank) was added to a 96-well plate in a concentration of 2×10⁴ cells per well and then a RPMI1640 medium supplemented with 10% fetal bovine serum was added to each well. After culturing 24 hours, the medium was changed with a serum-free RPMI1640 medium and then test compounds (0.39˜100 μM) were treated. After culturing at 37° C. for 5 hours in a CO₂ incubator, 10 μL of a WST reagent (Roche) was added to each well, which was then cultured for additional 2 hours. A microplate reader was used to measure the absorbance at 450 nm. From the absorbance values, each IC₅₀ value was calculated by non-linear regression curve fit using the ‘GraphPad Prism” program. The results are shown in Table 4 below.

TABLE 4

| Test compounds | |
|---|---|
| (Example No.) | IC50 (μM) |
| 56 | 95.5 |
| 78 | 636.1 |
| 81 | 827.5 |
| 98 | 74.9 |
| 125 | 7490.0 |
| 136 | 281.0 |
| 176 | 247.1 |
| 246 | 245.0 |
| 283 | 108.0 |
| 289 | 877.0 |
| 290 | 172.0 |
| 293 | 144.0 |
| 299 | 344.0 |
| 308 | 267.0 |
| 309 | 134.0 |
| 356 | 1187.0 |
| 361 | 581 |
| 388 | 368 |
| 442 | 361 |

The cytotoxicity study was performed, using the reduction of WST by the NADH produced by the electron transport system of mitochondria. As shown in Table 4, the IC₅₀ values showing cytotoxicity is remarkably higher than the IC₅₀ values for inhibiting cell migration. Therefore, the compounds of the present invention have therapeutic activity against a disease associated with cancer cell metastasis through (i) selective inhibition against the binding between KRS and LR and (ii) inhibition of cell migration, rather than non-specific cytotoxicity.

1. A compound of Formula 1 or its pharmaceutically acceptable salt: 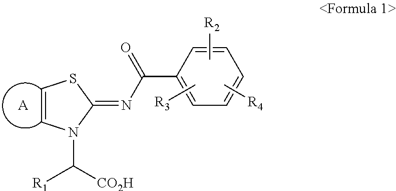
wherein,
R₁ is hydrogen; a C₁˜C₆ alkyl group optionally substituted with hydroxy; a C₂˜C₆ alkenyl group; a halogen group; a trifluoromethyl group; a phenyl group; or a benzyl group,
R₂, R₃, and R₄ are, independently each other, hydrogen; a C₁˜C₆ alkyl group optionally substituted with one or more substituents selected from the group consisting of C₁˜C₃ alkoxy and halogen; a C₁˜C₃ alkoxy group optionally one or more substituted with halogen; a halogen group; a nitro group; a cyano group; a phenoxy group; a benzyloxy group; an amino group; an amino group mono- or di-substituted with C₁˜C₆ alkyl; a phenyl group; or a hydroxycarbonyl group, or
the Ring A is a benzene ring of the following formula A, a cyclohexene ring of the following formula B, or a cycloheptene ring of the following formula C, 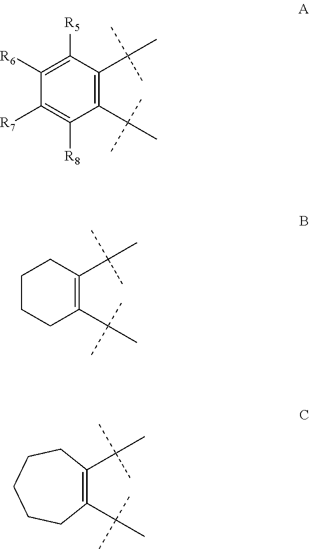
R₅, R₆, R₇, and R₈ are, independently each other, hydrogen; a halogen group; a trifluoromethyl group; a trifluoromethoxy group; a hydroxy group; a hydroxymethyl group; a C₁˜C₆ alkyl group; a C₁˜C₆ alkoxy group optionally substituted with morpholine; C₃˜C₆ cycloalkyloxy group; a phenyl group optionally substituted with halogen; a phenoxy group; a benzyloxy group; a nitro group; a thiophenyl group; an amino group; a dimethylamino group; a R₉—NH group; a R₁₀—C(═O)—NH group; a R₁₀—SO₂—NH group; a R₁₁-carbonyl group; or a R₁₂—NH-carbonyl group, or
R₆ and R₇ may be cyclized each other to form a tricyclic ring of the following formula D, 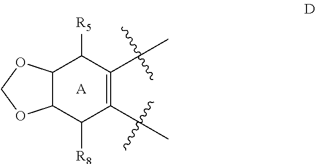
R₉ is C₁˜C₃ alkyl; phenyl optionally substituted with halogen; or pyrimidyl,
R₁₀ is C₁˜C₅ alkyl; C₃˜C₆ cycloalkyl; phenyl; phenylamino; or benzyl (the phenyl group, the phenylamino group, and the benzyl group may be optionally substituted with halogen or trifluoromethyl, respectively),
R₁₁ is hydroxy; C₁˜C₆ alkyl; azetidinyl; pyrrolidinyl; piperidinyl; or morpholinyl, and
R₁₂ is C₁˜C₆ alkyl; phenyl; or phenyl-C₁˜C₃ alkyl.

2. The compound or its pharmaceutically acceptable salt of claim 1, wherein the ring A is the benzene ring of the formula A, and R₅, R₆, R₇, and R₈ are not hydrogen at the same time.

3. The compound or its pharmaceutically acceptable salt of claim 2, wherein
R₁ is hydrogen; or a C₁˜C₆ alkyl group,
R₂, R₃, and R₄ are, independently each other, hydrogen; a C₁˜C₆ alkyl group; a C₁˜C₃ alkoxy group optionally substituted with halogen; a trifluoromethyl group; or a halogen group, and
R₅, R₆, R₇, and R₈ are, independently each other, hydrogen; a halogen group; a trifluoromethyl group; a trifluoromethoxy group; a C₁˜C₆ alkyl group; a C₁˜C₆ alkoxy group; or a nitro group, or
R₆ and R₇ may be cyclized each other to form a tricyclic ring of the following formula D.

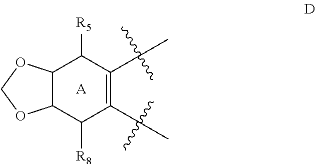

4. The compound or its pharmaceutically acceptable salt of claim 1, wherein
the ring A is the benzene ring of the formula A,
R₅, R₆, R₇, and R₈ are hydrogen,
R₁ is hydrogen; or a C₁˜C₆ alkyl group, and
R₂, R₃, and R₄ are, independently each other, hydrogen; a C₁˜C₃ alkoxy group; or a halogen group, with the provisos that R₂, R₃, and R₄ can not be hydrogen at the same time.

5. The compound or its pharmaceutically acceptable salt of claim 1, wherein
the ring A is the cyclohexene ring of the formula B,
R₁ is a C₁˜C₆ alkyl group, and
R₂, R₃, and R₄ are, independently each other, hydrogen; a trifluoromethyl group; or halogen.

6. The compound or its pharmaceutically acceptable salt of claim 1, which is selected from the group consisting of:
2-[5-bromo-2-(4-methylbenzoylimino)benzo[d]thiazol-3(2H)-yl]butanoic acid;
2-[6-(2,3-difluorophenyl)-2-(4-methylbenzoylimino)benzo[d]thiazol-3(2H)-yl]butanoic acid;
2-[2-(4-methylbenzoylimino)-6-(thiophen-3-yl)benzo[d]thiazol-3(2H)-yl]butanoic acid;
2-[6-(4-fluorophenyl)-2-(4-methylbenzoylimino)benzo[d]thiazol-3(2H)-yl]butanoic acid;
2-{6-methyl-2-[(4-methylbenzoyl)imino]benzo[d]thiazol-3(2H)-yl}butanoic acid;
2-{5,6-dimethyl-2-[(4-methylbenzoyl)imino]benzo[d]thiazol-3(2H)-yl}butanoic acid;
2-{6-ethoxy-2-[(4-methylbenzoyl)imino]benzo[d]thiazol-3(2H)-yl}butanoic acid;
2-{4-methoxy-2-[(4-methylbenzoyl)imino]benzo[d]thiazol-3(2H)-yl}butanoic acid;
2-{6-bromo-2-[(4-methylbenzoyl)imino]benzo[d]thiazol-3(2H)-yl}butanoic acid;
2-{6-chloro-2-[(4-methylbenzoyl)imino]benzo[d]thiazol-3(2H)-yl}butanoic acid;
2-{6-methoxy-2-[(4-methylbenzoyl)imino]benzo[d]thiazol-3(2H)-yl}butanoic acid;
2-{2-[(4-methylbenzoyl)imino]-6-phenoxybenzo[d]thiazol-3(2H)-yl}butanoic acid;
2-{5,6-difluoro-2-[(4-methylbenzoyl)imino]benzo[d]thiazol-3(2H)-yl}butanoic acid;
2-{2-[(4-methylbenzoyl)imino]-6-(trifluoromethoxy)benzo[d]thiazol-3(2H)-yl}butanoic acid;
2-{2-[(4-methylbenzoyl)imino]-6-phenylbenzo[d]thiazol-3(2H)-yl}butanoic acid;
2-{5-fluoro-6-methyl-2-[(4-methylbenzoyl)imino]benzo[d]thiazol-3(2H)-yl}butanoic acid;
2-{6-butyl-2-[(4-methylbenzoyl)imino]benzo[d]thiazol-3(2H)-yl}butanoic acid;
2-{6-(tert-butyl)-2-[(4-methylbenzoyl)imino]benzo[d]thiazol-3(2H)-yl}butanoic acid;
2-{2-[(4-methylbenzoyl)imino]-6-propylbenzo[d]thiazol-3(2H)-yl}butanoic acid;
2-{6-isopropyl-2-[(4-methylbenzoyl)imino]benzo[d]thiazol-3(2H)-yl}butanoic acid;
2-{5-methoxy-2-[(4-methylbenzoyl)imino]benzo[d]thiazol-3(2H)-yl}butanoic acid;
2-{5-(benzyloxy)-2-[(4-methylbenzoyl)imino]benzo[d]thiazol-3(2H)-yl}butanoic acid;
2-{2-[(4-methylbenzoyl)imino]-6-nitrobenzo[d]thiazol-3(2H)-yl}butanoic acid;
2-{6-acetamido-2-[(4-methylbenzoyl)imino]benzo[d]thiazol-3(2H)-yl}butanoic acid;
2-{2-[(4-methylbenzoyl)imino]-6-propionamidobenzo[d]thiazol-3(2H)-yl}butanoic acid;
2-{6-isobutyramido-2-[(4-methylbenzoyl)imino]benzo[d]thiazol-3(2H)-yl}butanoic acid;
2-{2-[(4-methylbenzoyl)imino]-6-pivalamidobenzo[d]thiazol-3(2H)-yl}butanoic acid;
2-{6-benzamido-2-[(4-methylbenzoyl)imino]benzo[d]thiazol-3(2H)-yl}butanoic acid;
2-{2-[(4-methylbenzoyl)imino]-6-(2-phenylacetamino)benzo[d]thiazol-3(2H)-yl)butanoic acid;
2-{2-[(4-methylbenzoyl)imino]-6-(phenylsulfonamido)benzo[d]thiazol-3(2H)-yl}butanoic acid;
2-{6-(cyclopentanecarboxamido)-2-[(4-methylbenzoyl)imino]benzo[d]thiazol-3(2H)-yl}butanoic acid;
2-{6-(2-fluorobenzamido)-2-[(4-methylbenzoyl)imino]benzo[d]thiazol-3(2H)-yl}butanoic acid;
2-{6-[3-(4-fluorophenyl)ureido]-2-[(4-methylbenzoyl)imino]benzo[d]thiazol-3(2H)-yl}butanoic acid;
2-{6-[3-(3-chlorophenyl)ureido]-2-[(4-methylbenzoyl)imino]benzo[d]thiazol-3(2H)-yl}butanoic acid;
2-{6-(4-fluorophenylsulfonamido)-2-[(4-methylbenzoyl)imino]benzo[d]thiazol-3(2H)-yl}butanoic acid;
2-{2-[(4-methylbenzoyl)imino]-6-[4-(trifluoromethyl)phenylsulfonamido]benzo[d]thiazol-3(2H)-yl}butanoic acid;
2-{2-[(4-methylbenzoyl)imino]-5-nitrobenzo[d]thiazol-3(2H)-yl}butanoic acid;
2-{5-acetamido-2-[(4-methylbenzoyl)imino]benzo[d]thiazol-3(2H)-yl}butanoic acid;
2-{2-[(4-methylbenzoyl)imino]-5-propionamidobenzo[d]thiazol-3(2H)-yl}butanoic acid;
2-{5-isobutyramido-2-[(4-methylbenzoyl)imino]benzo[d]thiazol-3(2H)-yl}butanoic acid;
2-{2-[(4-methylbenzoyl)imino]-5-pivalamidobenzo[d]thiazol-3(2H)-yl}butanoic acid;
2-{5-benzamido-2-[(4-methylbenzoyl)imino]benzo[d]thiazol-3(2H)-yl}butanoic acid;
2-{2-[(4-methylbenzoyl)imino]-5-(2-phenylacetamido)benzo[d]thiazol-3(2H)-yl}butanoic acid;
2-{2-[(4-methylbenzoyl)imino]-5-(phenylsulfonamido)benzo[d]thiazol-3(2H)-yl}butanoic acid;
2-{5-(cyclopentanecarboxamido)-2-[(4-methylbenzoyl)imino]benzo[d]thiazol-3(2H)-yl}butanoic acid;
2-{5-(2-fluorobenzamido)-2-[(4-methylbenzoyl)imino]benzo[d]thiazol-3(2H)-yl}butanoic acid;
2-{5-[3-(4-fluorophenyl)ureido]-2-[(4-methylbenzoyl)imino]benzo[d]thiazol-3(2H)-yl}butanoic acid;
2-{5-[3-(3-chlorophenyl)ureido]-2-[(4-methylbenzoyl)imino]benzo[d]thiazol-3(2H)-yl}butanoic acid;
2-{5-(4-fluorophenylsulfonamido)-2-[(4-methylbenzoyl)imino]benzo[d]thiazol-3(2H)-yl}butanoic acid;
2-{2-[(4-methylbenzoyl)imino]-5-[4-(trifluoromethyl)phenylsulfonamido]benzo[d]thiazol-3(2H)-yl}butanoic acid;
2-{2-[(4-methylbenzoyl)imino]-7-nitrobenzo[d]thiazol-3(2H)-yl}butanoic acid;
2-{2-[(3-chlorobenzoyl)imino]-6-methoxybenzo[d]thiazol-3(2H)-yl}acetic acid;
2-{2-[(3-chlorobenzoyl)imino]-6-methoxybenzo[d]thiazol-3(2H)-yl}propionic acid;
2-{2-[(3-chlorobenzoyl)imino]-6-methoxybenzo[d]thiazol-3(2H)-yl}butanoic acid;
2-{2-[(3-chlorobenzoyl)imino]-6-ethoxybenzo[d]thiazol-3(2H)-yl}propionic acid;
2-{2-[(3-chlorobenzoyl)imino]-6-ethoxybenzo[d]thiazol-3(2H)-yl}butanoic acid;
2-{2-[(3-chlorobenzoyl)imino]-6-methylbenzo[d]thiazol-3(2H)-yl}acetic acid;
2-{2-[(3-chlorobenzoyl)imino]-6-methylbenzo[d]thiazol-3(2H)-yl}propionic acid;
2-{2-[(3-chlorobenzoyl)imino]-6-methylbenzo[d]thiazol-3(2H)-yl}butanoic acid;
2-[2-(4-methoxybenzoylimino)benzo[d]thiazol-3(2H)-yl]butanoic acid;
2-[2-(3-methoxybenzoylimino)benzo[d]thiazol-3(2H)-yl]butanoic acid;
2-[2-(3-fluorobenzoylimino)benzo[d]thiazol-3(2H)-yl]butanoic acid;
2-[2-(2-methylbenzoylimino)benzo[d]thiazol-3(2H)-yl]butanoic acid;
2-{2-[4-(trifluoromethyl)benzoylimino]benzo[d]thiazol-3(2H)-yl}butanoic acid;
2-[2-(2,6-dichlorobenzoylimino)benzo[d]thiazol-3(2H)-yl]butanoic acid;
2-[2-(2-methoxybenzoylimino)benzo[d]thiazol-3(2H)-yl]butanoic acid;
2-{2-[3-(benzyloxy)benzoylimino]benzo[d]thiazol-3(2H)-yl}butanoic acid;
2-[2-(2-fluorobenzoylimino)benzo[d]thiazol-3(2H)-yl]butanoic acid;
2-[2-(5-fluoro-2-methylbenzoylimino)benzo[d]thiazol-3(2H)-yl]butanoic acid;
2-[2-(4-(dimethylamino)benzoylimino)benzo[d]thiazol-3(2H)-yl]butanoic acid;
3-{[3-(1-carboxypropyl)benzo[d]thiazol-2(3H)-ylidene]carbamoyl}benzoic acid;
2-{2-[2-(trifluoromethyl)benzoylimino]benzo[d]thiazol-3(2H)-yl}butanoic acid;
2-{[3-(1-carboxypropyl)benzo[d]thiazol-2(3H)-ylidene]carbamoyl}benzoic acid;
2-{2-[(1,1′-biphenyl)-3-carbonylimino]benzo[d]thiazol-3(2H)-yl}butanoic acid;
2-(5,6-difluoro-2-((3-fluoro-4-(trifluoromethyl)benzoyl)imino)benzo[d]thiazol-3(2H)-yl)butanoic acid;
2-[2-(2,4-dichloro-5-fluorobenzoylimino)benzo[d]thiazol-3(2H)-yl]butanoic acid;
2-{2-[5-fluoro-2-(trifluoromethyl)benzoylimino]benzo[d]thiazol-3(2H)-yl}butanoic acid;
2-[2-(3,5-difluorobenzoylimino)benzo[d]thiazol-3(2H)-yl]butanoic acid;
2-[2-(4-tert-butylbenzoylimino)benzo[d]thiazol-3(2H)-yl]butanoic acid;
2-[2-(4-(methoxymethyl)benzoylimino)benzo[d]thiazol-3(2H)-yl]butanoic acid;
2-[2-(4-nitrobenzoylimino)benzo[d]thiazol-3(2H)-yl]butanoic acid;
2-[2-(3-nitrobenzoylimino)benzo[d]thiazol-3(2H)-yl]butanoic acid;
2-[2-(3-bromobenzoylimino)benzo[d]thiazol-3(2H)-yl]butanoic acid;
2-[2-(4-cyanobenzoylimino)benzo[d]thiazol-3(2H)-yl]butanoic acid;
4-{[3-(1-carboxypropyl)benzo[d]thiazol-2(3H)-ylidene]carbamoyl}benzoic acid;
2-[2-(4-phenoxybenzoylimino)benzo[d]thiazol-3(2H)-yl]butanoic acid;
2-{2-[(1,1′-biphenyl)-2-carbonylimino]benzo[d]thiazol-3(2H)-yl}butanoic acid;
2-[2-(4-aminobenzoylimino)benzo[d]thiazol-3(2H)-yl]butanoic acid;
2-[2-(3-aminobenzoylimino)benzo[d]thiazol-3(2H)-yl]butanoic acid;
2-[5-fluoro-2-(4-methylbenzoylimino)benzo[d]thiazol-3(2H)-yl]butanoic acid;
2-[5-fluoro-2-(4-methylbenzoylimino)benzo[d]thiazol-3(2H)-yl]propionic acid;
2-[5-fluoro-2-(4-methylbenzoylimino)benzo[d]thiazol-3(2H)-yl]acetic acid;
2-[7-fluoro-2-(4-methylbenzoylimino)benzo[d]thiazol-3(2H)-yl]butanoic acid;
2-[7-fluoro-2-(4-methylbenzoylimino)benzo[d]thiazol-3(2H)-yl]propionic acid;
2-[7-fluoro-2-(4-methylbenzoylimino)benzo[d]thiazol-3(2H)-yl]acetic acid;
2-[2-(4-methylbenzoylimino)-5-(trifluoromethyl)benzo[d]thiazol-3(2H)-yl]butanoic acid;
2-[2-(4-methylbenzoylimino)-7-(trifluoromethyl)benzo[d]thiazol-3(2H)-yl]butanoic acid;
2-[2-(4-methylbenzoylimino)-7-(trifluoromethyl)benzo[d]thiazol-3(2H)-yl]propionic acid;
2-[2-(4-methylbenzoylimino)-7-(trifluoromethyl)benzo[d]thiazol-3(2H)-yl]acetic acid;
2-[5-methyl-2-(4-methylbenzoylimino)benzo[d]thiazol-3(2H)-yl]butanoic acid;
2-[5-methyl-2-(4-methylbenzoylimino)benzo[d]thiazol-3(2H)-yl]propionic acid;
2-[5-methyl-2-(4-methylbenzoylimino)benzo[d]thiazol-3(2H)-yl)acetic acid;
2-[7-methyl-2-(4-methylbenzoylimino)benzo[d]thiazol-3(2H)-yl]propionic acid;
2-[7-methyl-2-(4-methylbenzoylimino)benzo[d]thiazol-3(2H)-yl]acetic acid;
2-[5-chloro-2-(4-methylbenzoylimino)benzo[d]thiazol-3(2H)-yl]butanoic acid;
2-[5-chloro-2-(4-methylbenzoylimino)benzo[d]thiazol-3(2H)-yl]propionic acid;
2-[5-chloro-2-(4-methylbenzoylimino)benzo[d]thiazol-3(2H)-yl]acetic acid;
2-[7-chloro-2-(4-methylbenzoylimino)benzo[d]thiazol-3(2H)-yl]butanoic acid;
2-[7-chloro-2-(4-methylbenzoylimino)benzo[d]thiazol-3(2H)-yl]propionic acid;
2-[7-chloro-2-(4-methylbenzoylimino)benzo[d]thiazol-3(2H)-yl]acetic acid;
2-[2-(4-methylbenzoylimino)-5-propoxybenzo[d]thiazol-3(2H)-yl]butanoic acid;
2-[5-butoxy-2-(4-methylbenzoylimino)benzo[d]thiazol-3(2H)-yl]butanoic acid;
2-[5-isopropoxy-2-(4-methylbenzoylimino)benzo[d]thiazol-3(2H)-yl]butanoic acid;
2-[5-(cyclohexyloxy)-2-(4-methylbenzoylimino)benzo[d]thiazol-3(2H)-yl]butanoic acid;
2-[5-hydroxy-2-(4-methylbenzoylimino)benzo[d]thiazol-3(2H)-yl]butanoic acid;
2-[6-(4-fluorophenylamino)-2-(4-methylbenzoylimino)benzo[d]thiazol-3(2H)-yl]butanoic acid;
2-[2-(4-methylbenzoylimino)-6-(pyrimidin-2-ylamino)benzo[d]thiazol-3(2H)-yl]butanoic acid;
2-[7-bromo-2-(4-methylbenzoylimino)benzo[d]thiazol-3(2H)-yl]butanoic acid;
2-[2-(benzoylimino)benzo[d]thiazol-3(2H)-yl]acetic acid;
2-[2-(4-methylbenzoylimino)benzo[d]thiazol-3(2H)-yl]acetic acid;
2-[2-(3-methylbenzoylimino)benzo[d]thiazol-3(2H)-yl]acetic acid;
2-[2-(4-chlorobenzoylimino)benzo[d]thiazol-3(2H)-yl]acetic acid;
2-[2-(3-chlorobenzoylimino)benzo[d]thiazol-3(2H)-yl]acetic acid;
2-[2-(2-chlorobenzoylimino)benzo[d]thiazol-3(2H)-yl]acetic acid;
2-[2-(3-(trifluoromethyl)benzoylimino)benzo[d]thiazol-3(2H)-yl]acetic acid;
2-[2-(4-(trifluoromethoxy)benzoylimino)benzo[d]thiazol-3(2H)-yl]acetic acid;
2-[2-(2,6-dimethoxybenzoylimino)benzo[d]thiazol-3(2H)-yl]acetic acid;
2-[2-(4-methylbenzoylimino)benzo[d]thiazol-3(2H)-yl]propionic acid;
2-[2-(3-methylbenzoylimino)benzo[d]thiazol-3(2H)-yl]propionic acid;
2-[2-(4-chlorobenzoylimino)benzo[d]thiazol-3(2H)-yl]propionic acid;
2-[2-(3-chlorobenzoylimino)benzo[d]thiazol-3(2H)-yl]propionic acid;
2-[2-(2-chlorobenzoylimino)benzo[d]thiazol-3(2H)-yl]propionic acid;
2-{2-[3-(trifluoromethyl)benzoylimino]benzo[d]thiazol-3(2H)-yl}propionic acid;
2-{2-[4-(trifluoromethoxy)benzoylimino]benzo[d]thiazol-3(2H)-yl}propionic acid;
2-[2-(2,6-dimethoxybenzoylimino)benzo[d]thiazol-3(2H)-yl]propionic acid;
2-[2-(benzoylimino)benzo[d]thiazol-3(2H)-yl]butanoic acid;
2-(2-((3,5-bis(trifluoromethyl)benzoyl)imino)-5,6-difluorobenzo[d]thiazol-3(2H)-yl)butanoic acid;
2-[2-(4-chlorobenzoylimino)benzo[d]thiazol-3(2H)-yl]butanoic acid;
2-[2-(3-chlorobenzoylimino)benzo[d]thiazol-3(2H)-yl]butanoic acid;
2-[2-(2-chlorobenzoylimino)benzo[d]thiazol-3(2H)-yl]butanoic acid;
2-[2-(2,6-difluorobenzoylimino)benzo[d]thiazol-3(2H)-yl]butanoic acid;
2-[2-(3-(trifluoromethyl)benzoylimino)benzo[d]thiazol-3(2H)-yl]butanoic acid;
2-[2-(4-(trifluoromethoxy)benzoylimino)benzo[d]thiazol-3(2H)-yl]butanoic acid;
2-[2-(2,6-dimethoxybenzoylimino)benzo[d]thiazol-3(2H)-yl]butanoic acid;
2-[2-(4-methylbenzoylimino)benzo[d]thiazol-3(2H)-yl]pentanoic acid;
2-[2-(4-methylbenzoylimino)benzo[d]thiazol-3(2H)-yl]hexanoic acid;
2-[2-(4-methylbenzoylimino)benzo[d]thiazol-3(2H)-yl]octanoic acid;
3-methyl-2-[2-(4-methylbenzoylimino)benzo[d]thiazol-3(2H)-yl]butanoic acid;
2-[2-(4-methylbenzoylimino)benzo[d]thiazol-3(2H)-yl]-3-butenoic acid;
2-fluoro-2-[2-(4-methylbenzoylimino)benzo[d]thiazol-3(2H)-yl]acetic acid;
3,3,3-trifluoro-2-[2-(4-methylbenzoylimino)benzo[d]thiazol-3(2H)-yl]propionic acid;
2-[(2-(4-methylbenzoylimino)benzo[d]thiazol-3(2H)-yl]-2-phenylacetic acid;
2-[6-fluoro-2-(4-methylbenzoylimino)benzo[d]thiazol-3(2H)-yl]butanoic acid;
2-[4,6-difluoro-2-(4-methylbenzoylimino)benzo[d]thiazol-3(2H)-yl]butanoic acid;
2-[2-(4-methylbenzoylimino)-6-(trifluoromethyl)benzo[d]thiazol-3(2H)-yl]butanoic acid;
3-(1-carboxypropyl)-2-(4-methylbenzoylimino)-2,3-dihydrobenzo[d]thiazol-6-carboxylic acid;
2-[2-(4-methylbenzoylimino)-6-(phenylcarbamoyl)benzo[d]thiazol-3(2H)-yl]butanoic acid;
2-[6-(benzylcarbamoyl)-2-(4-methylbenzoylimino)benzo[d]thiazol-3(2H)-yl]butanoic acid;
2-[2-(4-methylbenzoylimino)-6-(phenethylcarbamoyl)benzo[d]thiazol-3(2H)-yl]butanoic acid;
2-[2-(4-methylbenzoylimino)-4,5,6,7-tetrahydrobenzo[d]thiazol-3(2H)-yl]butanoic acid;
2-(5,6-dimethyl-2-((4-methylbenzoyl)imino)benzo[d]thiazol-3(2H)-yl)propionic acid;
2-(6-ethoxy-2-((4-methylbenzoyl)imino)benzo[d]thiazol-3(2H)-yl)propionic acid;
2-(4-methoxy-2-((4-methylbenzoyl)imino)benzo[d]thiazol-3(2H)-yl)propionic acid;
2-(6-methyl-2-((4-methylbenzoyl)imino)benzo[d]thiazol-3(2H)-yl)propionic acid;
2-(6-(hydroxymethyl)-2-((4-methylbenzoyl)imino)benzo[d]thiazol-3(2H)-yl)butanoic acid;
2-(6-(benzyloxy)-2-((4-methylbenzoyl)imino)benzo[d]thiazol-3(2H)-yl)butanoic acid;
2-(2((4-methylbenzoyl)imino)-6-(2-morpholinoethoxy)benzo[d]thiazol-3(2H)-yl)butanoic acid;
2-(6-hydroxy-2-((4-methylbenzoyl)imino)benzo[d]thiazol-3(2H)-yl)butanoic acid;
2-[2-(4-methylbenzoylimino)-4-methoxybenzo[d]thiazol-3(2H)-yl]acetic acid;
2-(6-(azetidin-1-carbonyl)-2-(4-methylbenzoylimino)benzo[d]thiazol-3(2H)-yl)butanoic acid;
2-(2-((3-chlorobenzoyl)imino)-6-(trifluoromethyl)benzo[d]thiazol-3(2H)-yl)acetic acid;
2-(2-((3-chlorobenzoyl)imino)-6-(trifluoromethyl)benzo[d]thiazol-3(2H)-yl)propionic acid;
2-(2-((3-chlorobenzoyl)imino)-6-(trifluoromethyl)benzo[d]thiazol-3(2H)-yl)butanoic acid;
2-(2-((3-chlorobenzoyl)imino)-6-(trifluoromethoxy)benzo[d]thiazol-3(2H)-yl)acetic acid;
2-(2-((3-chlorobenzoyl)imino)-6-(trifluoromethoxy)benzo[d]thiazol-3(2H)-yl)propionic acid;
2-(2-((3-chlorobenzoyl)imino)-6-(trifluoromethoxy)benzo[d]thiazol-3(2H)-yl)butanoic acid;
2-(6-chloro-2-((3-chlorobenzoyl)imino)benzo[d]thiazol-3(2H)-yl)acetic acid;
2-(6-chloro-2-((3-chlorobenzoyl)imino)benzo[d]thiazol-3(2H)-yl)propionic acid;
2-(6-chloro-2-((3-chlorobenzoyl)imino)benzo[d]thiazol-3(2H)-yl)butanoic acid;
2-(2-((3-bromobenzoyl)imino)benzo[d]thiazol-3(2H)-yl)acetic acid;
2-(2-((3-bromobenzoyl)imino)benzo[d]thiazol-3(2H)-yl)propionic acid;
2-(2-(4-methylbenzoylimino)-6-(methylcarbamoyl)benzo[d]thiazol-3(2H)-yl)butanoic acid;
2-(2-(4-methylbenzoylimino)-6-(propylcarbamoyl)benzo[d]thiazol-3(2H)-yl)butanoic acid;
2-(2-(4-(trifluoromethyl)benzoylimino)benzo[d]thiazol-3(2H)-yl)acetic acid;
2-(2-(4-methylbenzoylimino)-4,5,6,7-tetrahydrobenzo[d]thiazol-3(2H)-yl)acetic acid;
2-(2-(4-(trifluoromethyl)benzoylimino)benzo[d]thiazol-3(2H)-yl)propionic acid;
2-(2-(4-methylbenzoylimino)-4,5,6,7-tetrahydrobenzo[d]thiazol-3(2H)-yl)propionic acid;
2-(6-methoxy-2-((3-(trifluoromethyl)benzoyl)imino)benzo[d]thiazol-3(2H)-yl)acetic acid;
2-(6-methoxy-2-((3-(trifluoromethyl)benzoyl)imino)benzo[d]thiazol-3(2H)-yl)propionic acid;
2-(6-methoxy-2-((3-(trifluoromethyl)benzoyl)imino)benzo[d]thiazol-3(2H)-yl)butanoic acid;
2-(6-ethoxy-2-((3-(trifluoromethyl)benzoyl)imino)benzo[d]thiazol-3(2H)-yl)acetic acid;
2-(6-ethoxy-2-((3-(trifluoromethyl)benzoyl)imino)benzo[d]thiazol-3(2H)-yl)propionic acid;
2-(6-ethoxy-2-((3-(trifluoromethyl)benzoyl)imino)benzo[d]thiazol-3(2H)-yl)butanoic acid;
2-(6-methyl-2-((3-(trifluoromethyl)benzoyl)imino)benzo[d]thiazol-3(2H)-yl)acetic acid;
2-(6-methyl-2-((3-(trifluoromethyl)benzoyl)imino)benzo[d]thiazol-3(2H)-yl)propionic acid;
2-(6-methyl-2-((3-(trifluoromethyl)benzoyl)imino)benzo[d]thiazol-3(2H)-yl)butanoic acid;
2-(6-(trifluoromethoxy)-2-((3-(trifluoromethyl)benzoyl)imino)benzo[d]thiazol-3(2H)-yl)acetic acid;
2-(6-(trifluoromethoxy)-2-((3-(trifluoromethyl)benzoyl)imino)benzo[d]thiazol-3(2H)-yl)propionic acid;
2-(6-(trifluoromethoxy)-2-((3-(trifluoromethyl)benzoyl)imino)benzo[d]thiazol-3(2H)-yl)butanoic acid;
2-(4,6-difluoro-2-(4-methylbenzoylimino)benzo[d]thiazol-3(2H)-yl)acetic acid;
2-(4,6-difluoro-2-(4-methylbenzoylimino)benzo[d]thiazol-3(2H)-yl)propionic acid;
2-(2-(4-methylbenzoylimino)-6-(pyrrolidin-1-carbonyl)benzo[d]thiazol-3(2H)-yl)butanoic acid;
2-(2-(4-methylbenzoylimino)-6-(piperidin-1-carbonyl)benzo[d]thiazol-3(2H)-yl)butanoic acid;
2-(2-(4-methylbenzoylimino)-6-(morpholine-4-carbonyl)benzo[d]thiazol-3(2H)-yl)butanoic acid;
2-(6-chloro-2-((3-(trifluoromethyl)benzoyl)imino)benzo[d]thiazol-3(2H)-yl)acetic acid;
2-(6-chloro-2-((3-(trifluoromethyl)benzoyl)imino)benzo[d]thiazol-3(2H)-yl)propionic acid;
2-(6-chloro-2-((3-(trifluoromethyl)benzoyl)imino)benzo[d]thiazol-3(2H)-yl)butanoic acid;
2-(2-((3-chlorobenzoyl)imino)-6-fluorobenzo[d]thiazol-3(2H)-yl)acetic acid;
2-(2-((3-chlorobenzoyl)imino)-6-fluorobenzo[d]thiazol-3(2H)-yl)propionic acid;
2-(2-((3-chlorobenzoyl)imino)-6-fluorobenzo[d]thiazol-3(2H)-yl)butanoic acid;
2-(2-((3-chlorobenzoyl)imino)-5,6-dimethylbenzo[d]thiazol-3(2H)-yl)acetic acid;
2-(2-((3-chlorobenzoyl)imino)-5,6-dimethylbenzo[d]thiazol-3(2H)-yl)propionic acid;
2-(2-((3-chlorobenzoyl)imino)-5,6-dimethylbenzo[d]thiazol-3(2H)-yl)butanoic acid;
2-(6-(trifluoromethyl)-2-((3-(trifluoromethyl)benzoyl)imino)benzo[d]thiazol-3(2H)-yl)acetic acid;
2-(6-(trifluoromethyl)-2-((3-(trifluoromethyl)benzoyl)imino)benzo[d]thiazol-3(2H)-yl)propionic acid;
2-(6-(trifluoromethyl)-2-((3-(trifluoromethyl)benzoyl)imino)benzo[d]thiazol-3(2H)-yl)butanoic acid;
2-(5,6-dimethyl-2-((3-(trifluoromethyl)benzoyl)imino)benzo[d]thiazol-3(2H)-yl)acetic acid;
2-(5,6-dimethyl-2-((3-(trifluoromethyl)benzoyl)imino)benzo[d]thiazol-3(2H)-yl)propionic acid;
2-(5,6-dimethyl-2-((3-(trifluoromethyl)benzoyl)imino)benzo[d]thiazol-3(2H)-yl)butanoic acid;
2-(6-fluoro-2-((3-(trifluoromethyl)benzoyl)imino)benzo[d]thiazol-3(2H)-yl)acetic acid;
2-(6-fluoro-2-((3-(trifluoromethyl)benzoyl)imino)benzo[d]thiazol-3(2H)-yl)propionic acid;
2-(6-fluoro-2-((3-(trifluoromethyl)benzoyl)imino)benzo[d]thiazol-3(2H)-yl)butanoic acid;
2-(2-(3,5-difluorobenzoylimino)benzo[d]thiazol-3(2H)-yl)propionic acid;
2-(2-(3,5-difluorobenzoylimino)benzo[d]thiazol-3(2H)-yl)acetic acid;
2-(5,6-difluoro-2-((3-methyl-5-(trifluoromethoxy)benzoyl)imino)benzo[d]thiazol-3(2H)-yl)butanoic acid;
2-(6-ethyl-2-((4-methylbenzoyl)imino)benzo[d]thiazol-3(2H)-yl)acetic acid;
2-(6-ethyl-2-((4-methylbenzoyl)imino)benzo[d]thiazol-3(2H)-yl)propionic acid;
2-(6-ethyl-2-((4-methylbenzoyl)imino)benzo[d]thiazol-3(2H)-yl)butanoic acid;
2-(2-((3-chlorobenzoyl)imino)-6-ethylbenzo[d]thiazol-3(2H)-yl)acetic acid;
2-(2-((3-chlorobenzoyl)imino)-6-ethylbenzo[d]thiazol-3(2H)-yl)propionic acid;
2-(2-((3-chlorobenzoyl)imino)-6-ethylbenzo[d]thiazol-3(2H)-yl)butanoic acid;
2-(6-ethyl-2-((3-(trifluoromethyl)benzoyl)imino)benzo[d]thiazol-3(2H)-yl)acetic acid;
2-(6-ethyl-2-((3-(trifluoromethyl)benzoyl)imino)benzo[d]thiazol-3(2H)-yl)propionic acid;
2-(6-ethyl-2-((3-(trifluoromethyl)benzoyl)imino)benzo[d]thiazol-3(2H)-yl)butanoic acid;
2-(6-((4-methylbenzoyl)imino)-[1,3]dioxolo[4′,5′:4,5]benzo[1,2-d]thiazol-7(6H)-yl)acetic acid;
2-(6-((4-methylbenzoyl)imino)-[1,3]dioxolo[4′,5′:4,5]benzo[1,2-d]thiazol-7(6H)-yl)propionic acid;
2-(6-((4-methylbenzoyl)imino)-[1,3]dioxolo[4′,5′:4,5]benzo[1,2-d]thiazol-7(6H)-yl)butanoic acid;
2-(6-((3-chlorobenzoyl)imino)-[1,3]dioxolo[4′,5′:4,5]benzo[1,2-d]thiazol-7(6H)-yl)acetic acid;
2-(6-((3-chlorobenzoyl)imino)-[1,3]dioxolo[4′,5′:4,5]benzo[1,2-d]thiazol-7(6H)-yl)propionic acid;
2-(6-((3-chlorobenzoyl)imino)-[1,3]dioxolo[4′,5′:4,5]benzo[1,2-d]thiazol-7(6H)-yl)butanoic acid;
2-(6-((3-(trifluoromethyl)benzoyl)imino)-[1,3]dioxolo[4′,5′:4,5]benzo[1,2-d]thiazol-7(6H)-yl)acetic acid;
2-(6-((3-(trifluoromethyl)benzoyl)imino)-[1,3]dioxolo[4′,5′:4,5]benzo[1,2-d]thiazol-7(6H)-yl)propionic acid;
2-(6-((3-(trifluoromethyl)benzoyl)imino)-[1,3]dioxolo[4′,5′:4,5]benzo[1,2-d]thiazol-7(6H)-yl)butanoic acid;
2-(6-fluoro-2-((4-methylbenzoyl)imino)benzo[d]thiazol-3(2H)-yl)acetic acid;
2-(6-fluoro-2-((4-methylbenzoyl)imino)benzo[d]thiazol-3(2H)-yl)propionic acid;
2-(2-((4-methylbenzoyl)imino)-6-(trifluoromethyl)benzo[d]thiazol-3(2H)-yl)acetic acid;
2-(2-((4-methylbenzoyl)imino)-6-(trifluoromethyl)benzo[d]thiazol-3(2H)-yl)propionic acid;
2-(2-((4-methylbenzoyl)imino)-6-(trifluoromethoxy)benzo[d]thiazol-3(2H)-yl)acetic acid;
2-(2-((4-methylbenzoyl)imino)-6-(trifluoromethoxy)benzo[d]thiazol-3(2H)-yl)propionic acid;
2-(6-ethoxy-2-((4-methylbenzoyl)imino)benzo[d]thiazol-3(2H)-yl)acetic acid;
2-(5,6-dimethyl-2-((4-methylbenzoyl)imino)benzo[d]thiazol-3(2H)-yl)acetic acid;
2-(5,6-difluoro-2-((4-methylbenzoyl)imino)benzo[d]thiazol-3(2H)-yl)acetic acid;
2-(5,6-difluoro-2-((4-methylbenzoyl)imino)benzo[d]thiazol-3(2H)-yl)propionic acid;
3-hydroxy-2-(2-(4-methylbenzoylimino)benzo[d]thiazol-3(2H)-yl)propionic acid;
3-hydroxy-2-(2-(3-(trifluoromethyl)benzoylimino)benzo[d]thiazol-3(2H)-yl)propionic acid;
2-(2-(3-chlorobenzoylimino)benzo[d]thiazol-3(2H)-yl)-3-hydroxypropionic acid;
2-(2-(4-methylbenzoylimino)benzo[d]thiazol-3(2H)-yl)-3-phenylpropionic acid;
3-phenyl-2-(2-(3-(trifluoromethyl)benzoylimino)benzo[d]thiazol-3(2H)-yl)propionic acid;
2-(2-(3-chlorobenzoylimino)benzo[d]thiazol-3(2H)-yl)-3-phenylpropionic acid;
2-(2-(3-chlorobenzoylimino)benzo[d]thiazol-3(2H)-yl)octanoic acid;
2-(2-(3-chlorobenzoylimino)benzo[d]thiazol-3(2H)-yl)-3-methylbutanoic acid;
2-(2-(3-(trifluoromethyl)benzoylimino)benzo[d]thiazol-3(2H)-yl)octanoic acid;
3-methyl-2-(2-(3-(trifluoromethyl)benzoylimino)benzo[d]thiazol-3(2H)-yl)butanoic acid;
2-(2-(3-chlorobenzoylimino)benzo[d]thiazol-3(2H)-yl)-2-phenylacetic acid;
2-phenyl-2-(2-(3-(trifluoromethyl)benzoylimino)benzo[d]thiazol-3(2H)-yl)acetic acid;
2-(7-(trifluoromethyl)-2-(3-(trifluoromethyl)benzoyl imino)benzo[d]thiazol-3(2H)-yl)acetic acid;
2-(7-(trifluoromethyl)-2-(3-(trifluoromethyl)benzoyl imino)benzo[d]thiazol-3(2H)-yl)propionic acid;
2-(7-(trifluoromethyl)-2-(3-(trifluoromethyl)benzoylimino)benzo[d]thiazol-3(2H)-yl)butanoic acid;
2-(2-(3-chlorobenzoylimino)-7-(trifluoromethyl)benzo[d]thiazol-3(2H)-yl)acetic acid;
2-(2-(3-chlorobenzoylimino)-7-(trifluoromethyl)benzo[d]thiazol-3(2H)-yl)propionic acid;
2-(2-(3-chlorobenzoylimino)-7-(trifluoromethyl)benzo[d]thiazol-3(2H)-yl)butanoic acid;
2-(2-(2-amino-4-fluorobenzoylimino)benzo[d]thiazol-3(2H)-yl)butanoic acid;
2-(2-(4-(benzyloxy)benzoylimino)benzo[d]thiazol-3(2H)-yl)butanoic acid;
2-(2-(2-amino-4-fluorobenzoylimino)benzo[d]thiazol-3(2H)-yl)propionic acid;
2-(2-(4-(benzyloxy)benzoylimino)benzo[d]thiazol-3(2H)-yl)propionic acid;
2-(2-(3-fluorobenzoylimino)benzo[d]thiazol-3(2H)-yl)propionic acid;
2-(2-(4-butylbenzoylimino)benzo[d]thiazol-3(2H)-yl)butanoic acid;
2-(2-(3-fluorobenzoylimino)benzo[d]thiazol-3(2H)-yl)acetic acid;
2-(2-(4-butylbenzoylimino)benzo[d]thiazol-3(2H)-yl)propionic acid;
2-(2-(3-chlorobenzoylimino)-4-methoxybenzo[d]thiazol-3(2H)-yl)acetic acid;
2-(4-methoxy-2-(3-(trifluoromethyl)benzoylimino)benzo[d]thiazol-3(2H)-yl)acetic acid;
2-(2-(3-chlorobenzoylimino)-4-methoxybenzo[d]thiazol-3(2H)-yl)propionic acid;
2-(4-methoxy-2-(3-(trifluoromethyl)benzoylimino)benzo[d]thiazol-3(2H)-yl)propionic acid;
2-(2-(3-chlorobenzoylimino)-4-methoxybenzo[d]thiazol-3(2H)-yl)butanoic acid;
2-(4-methoxy-2-(3-(trifluoromethyl)benzoylimino)benzo[d]thiazol-3(2H)-yl)butanoic acid;
2-(5,6-difluoro-2-((4-methylbenzoyl)imino)benzo[d]thiazol-3(2H)-yl)acetic acid;
2-(5,6-difluoro-2-((4-methylbenzoyl)imino)benzo[d]thiazol-3(2H)-yl)propionic acid;
2-(2-((3-chlorobenzoyl)imino)-5,6-difluorobenzo[d]thiazol-3(2H)-yl)acetic acid;
2-(2-((3-chlorobenzoyl)imino)-5,6-difluorobenzo[d]thiazol-3(2H)-yl)propionic acid;
2-(2-((3-chlorobenzoyl)imino)-5,6-difluorobenzo[d]thiazol-3(2H)-yl)butanoic acid;
2-(5,6-difluoro-2-((3-(trifluoromethyl)benzoyl)imino)benzo[d]thiazol-3(2H)-yl)acetic acid;
2-(5,6-difluoro-2-((3-(trifluoromethyl)benzoyl)imino)benzo[d]thiazol-3(2H)-yl)propionic acid;
2-(5,6-difluoro-2-((3-(trifluoromethyl)benzoyl)imino)benzo[d]thiazol-3(2H)-yl)butanoic acid;
2-(2-(3-(trifluoromethyl)benzoylimino)-4,5,6,7-tetrahydrobenzo[d]thiazol-3(2H)-yl)acetic acid;
2-(2-(3-(trifluoromethyl)benzoylimino)-4,5,6,7-tetrahydrobenzo[d]thiazol-3(2H)-yl)propionic acid;
2-(2-(3-(trifluoromethyl)benzoylimino)-4,5,6,7-tetrahydrobenzo[d]thiazol-3(2H)-yl)butanoic acid;
2-(2-(3-chlorobenzoylimino)-4,5,6,7-tetrahydrobenzo[d]thiazol-3(2H)-yl)acetic acid;
2-(2-(3-chlorobenzoylimino)-4,5,6,7-tetrahydrobenzo[d]thiazol-3(2H)-yl)propionic acid;
2-(2-(3-chlorobenzoylimino)-4,5,6,7-tetrahydrobenzo[d]thiazol-3(2H)-yl)butanoic acid;
2-(6-methoxy-2-(4-methylbenzoylimino)benzo[d]thiazol-3(2H)-yl)acetic acid;
2-(6-methoxy-2-(4-methylbenzoylimino)benzo[d]thiazol-3(2H)-yl)propionic acid;
2-(6-chloro-2-(4-methylbenzoylimino)benzo[d]thiazol-3(2H)-yl)acetic acid;
2-(6-chloro-2-(4-methylbenzoylimino)benzo[d]thiazol-3(2H)-yl)propionic acid;
2-(4,6-difluoro-2-((3-(trifluoromethyl)benzoyl)imino)benzo[d]thiazol-3(2H)-yl)acetic acid;
2-(2-((3-chlorobenzoyl)imino)-4,6-difluorobenzo[d]thiazol-3(2H)-yl)acetic acid;
2-(2-((3-chlorobenzoyl)imino)-4,6-difluorobenzo[d]thiazol-3(2H)-yl)propionic acid;
2-(7-chloro-2-((3-(trifluoromethyl)benzoyl)imino)benzo[d]thiazol-3(2H)-yl)acetic acid;
2-(7-chloro-2-((3-(trifluoromethyl)benzoyl)imino)benzo[d]thiazol-3(2H)-yl)propionic acid;
2-(7-chloro-2-((3-chlorobenzoyl)imino)benzo[d]thiazol-3(2H)-yl)propionic acid;
2-(7-chloro-2-((3-chlorobenzoyl)imino)benzo[d]thiazol-3(2H)-yl)butanoic acid;
2-(5-chloro-2-(3-(trifluoromethyl)benzoylimino)benzo[d]thiazol-3(2H)-yl)acetic acid;
2-(5-chloro-2-(3-chlorobenzoylimino)benzo[d]thiazol-3(2H)-yl)acetic acid;
2-(7-methyl-2-(3-(trifluoromethyl)benzoylimino)benzo[d]thiazol-3(2H)-yl)acetic acid;
2-(7-methyl-2-(3-(trifluoromethyl)benzoylimino)benzo[d]thiazol-3(2H)-yl)propionic acid;
2-(2-(4-methylbenzoylimino)-5,6,7,8-tetrahydro-2H-cyclohepta[d]thiazol-3(4H)-yl)acetic acid;
2-(2-(4-methylbenzoylimino)-5,6,7,8-tetrahydro-2H-cyclohepta[d]thiazol-3(4H)-yl)propionic acid;
2-(2-(4-methylbenzoylimino)-5,6,7,8-tetrahydro-2H-cyclohepta[d]thiazol-3(4H)-yl)butanoic acid;
2-(2-(3-chlorobenzoylimino)-5,6,7,8-tetrahydro-2H-cyclohepta[d]thiazol-3(4H)-yl)acetic acid;
2-(2-(3-chlorobenzoylimino)-5,6,7,8-tetrahydro-2H-cyclohepta[d]thiazol-3(4H)-yl)propionic acid;
2-(2-(3-chlorobenzoylimino)-5,6,7,8-tetrahydro-2H-cyclohepta[d]thiazol-3(4H)-yl)butanoic acid;
2-(2-(3-(trifluoromethyl)benzoylimino)-5,6,7,8-tetrahydro-2H-cyclohepta[d]thiazol-3(4H)-yl)acetic acid;
2-(2-(3-(trifluoromethyl)benzoylimino)-5,6,7,8-tetrahydro-2H-cyclohepta[d]thiazol-3(4H)-yl)propionic acid;
2-(2-(3-(trifluoromethyl)benzoylimino)-5,6,7,8-tetrahydro-2H-cyclohepta[d]thiazol-3(4H)-yl)butanoic acid;
2-(5,6-dimethyl-2-(4-methylbenzoylimino)benzo[d]thiazol-3(2H)-yl)acetic acid;
2-(5-fluoro-6-methyl-2-(4-methylbenzoylimino)benzo[d]thiazol-3(2H)-yl)propionic acid;
2-(5-fluoro-6-methyl-2-(3-(trifluoromethyl)benzoyl imino)benzo[d]thiazol-3(2H)-yl)acetic acid;
2-(5-fluoro-6-methyl-2-(3-(trifluoromethyl)benzoyl imino)benzo[d]thiazol-3(2H)-yl)propionic acid;
2-(2-(3-chlorobenzoylimino)-5-fluoro-6-methylbenzo[d]thiazol-3(2H)-yl)acetic acid;
2-(2-(4-methylbenzoylimino)-5-(trifluoromethyl)benzo[d]thiazol-3(2H)-yl)propionic acid;
2-(2-(3-chlorobenzoylimino)-5-ethoxybenzo[d]thiazol-3(2H)-yl)propionic acid;
2-(2-(3-chlorobenzoylimino)-5-ethoxybenzo[d]thiazol-3(2H)-yl)butanoic acid;
2-(4,6-difluoro-2-(3-(trifluoromethyl)benzoylimino)benzo[d]thiazol-3(2H)-yl)propionic acid;
2-(6,7-difluoro-2-((4-methylbenzoyl)imino)benzo[d]thiazol-3(2H)-yl)acetic acid;
2-(6,7-difluoro-2-((4-methylbenzoyl)imino)benzo[d]thiazol-3(2H)-yl)propionic acid;
2-(6,7-difluoro-2-((4-methylbenzoyl)imino)benzo[d]thiazol-3(2H)-yl)butanoic acid;
2-(2-((3-chlorobenzoyl)imino)-6,7-difluorobenzo[d]thiazol-3(2H)-yl)acetic acid;
2-(2-((3-chlorobenzoyl)imino)-6,7-difluorobenzo[d]thiazol-3(2H)-yl)propionic acid;
2-(2-((3-chlorobenzoyl)imino)-6,7-difluorobenzo[d]thiazol-3(2H)-yl)butanoic acid;
2-(6,7-difluoro-2-((3-(trifluoromethyl)benzoyl)imino)benzo[d]thiazol-3(2H)-yl)acetic acid;
2-(6,7-difluoro-2-((3-(trifluoromethyl)benzoyl)imino)benzo[d]thiazol-3(2H)-yl)propionic acid;
2-(6,7-difluoro-2-((3-(trifluoromethyl)benzoyl)imino)benzo[d]thiazol-3(2H)-yl)butanoic acid;
2-(5-methoxy-2-((4-methylbenzoyl)imino)benzo[d]thiazol-3(2H)-yl)acetic acid;
2-(5-methoxy-2-((4-methylbenzoyl)imino)benzo[d]thiazol-3(2H)-yl)propionic acid;
2-(7-methyl-2-((4-methylbenzoyl)imino)benzo[d]thiazol-3(2H)-yl)butanoic acid;
2-(7-methyl-2-((3-(trifluoromethyl)benzoyl)imino)benzo[d]thiazol-3(2H)-yl)butanoic acid;
2-(7-fluoro-2-((3-(trifluoromethyl)benzoyl)imino)benzo[d]thiazol-3(2H)-yl)propionic acid;
2-(2-((3-chlorobenzoyl)imino)-7-fluorobenzo[d]thiazol-3(2H)-yl)acetic acid;
2-(2-((4-methylbenzoyl)imino)-5-(trifluoromethyl)benzo[d]thiazol-3(2H)-yl)propionic acid;
2-(5-methyl-2-((3-(trifluoromethyl)benzoyl)imino)benzo[d]thiazol-3(2H)-yl)acetic acid;
2-(2-((3-chlorobenzoyl)imino)-5-methylbenzo[d]thiazol-3(2H)-yl)butanoic acid;
2-(2-((3-chlorobenzoyl)imino)-5-ethoxybenzo[d]thiazol-3(2H)-yl)acetic acid;
2-(2-((3-chlorobenzoyl)imino)-7-methylbenzo[d]thiazol-3(2H)-yl)acetic acid;
2-(2-((3-chlorobenzoyl)imino)-7-methylbenzo[d]thiazol-3(2H)-yl)propionic acid;
2-(2-((3-chlorobenzoyl)imino)-7-methylbenzo[d]thiazol-3(2H)-yl)butanoic acid;
2-(5-fluoro-2-((3-(trifluoromethyl)benzoyl)imino)benzo[d]thiazol-3(2H)-yl)propionic acid;
2-(7-fluoro-2-((3-(trifluoromethyl)benzoyl)imino)benzo[d]thiazol-3(2H)-yl)acetic acid;
2-(7-chloro-2-((3-(trifluoromethyl)benzoyl)imino)benzo[d]thiazol-3(2H)-yl)butanoic acid;
2-(7-chloro-2-((3-chlorobenzoyl)imino)benzo[d]thiazol-3(2H)-yl)acetic acid;
2-(5-chloro-2-((3-(trifluoromethyl)benzoyl)imino)benzo[d]thiazol-3(2H)-yl)propionic acid;
2-(5-chloro-2-((3-chlorobenzoyl)imino)benzo[d]thiazol-3(2H)-yl)propionic acid;
2-(5-chloro-2-((3-chlorobenzoyl)imino)benzo[d]thiazol-3(2H)-yl)butanoic acid;
2-(5-fluoro-6-methyl-2-((3-(trifluoromethyl)benzoyl)imino)benzo[d]thiazol-3(2H)-yl)butanoic acid;
2-(5-fluoro-2-((3-(trifluoromethyl)benzoyl)imino)benzo[d]thiazol-3(2H)-yl)butanoic acid;
2-(2-((3-chlorobenzoyl)imino)-4,6-difluorobenzo[d]thiazol-3(2H)-yl)butanoic acid;
2-(5-fluoro-2-((3-(trifluoromethyl)benzoyl)imino)benzo[d]thiazol-3(2H)-yl)acetic acid;
2-(7-fluoro-2-((3-(trifluoromethyl)benzoyl)imino)benzo[d]thiazol-3(2H)-yl)butanoic acid;
2-(2-((4-methylbenzoyl)imino)-5-(trifluoromethyl)benzo[d]thiazol-3(2H)-yl)acetic acid;
2-(5-methyl-2-((3-(trifluoromethyl)benzoyl)imino)benzo[d]thiazol-3(2H)-yl)butanoic acid;
2-(2-((3-chlorobenzoyl)imino)-5-methylbenzo[d]thiazol-3(2H)-yl)acetic acid;
2-(2-((3-chlorobenzoyl)imino)-5-methylbenzo[d]thiazol-3(2H)-yl)propionic acid;
2-(2-((3-chlorobenzoyl)imino)-5-fluoro-6-methylbenzo[d]thiazol-3(2H)-yl)propionic acid;
2-(2-((3-chlorobenzoyl)imino)-5-fluoro-6-methylbenzo[d]thiazol-3(2H)-yl)butanoic acid;
2-(2-((3-chlorobenzoyl)imino)-7-fluorobenzo[d]thiazol-3(2H)-yl)propionic acid;
2-(2-((3-chlorobenzoyl)imino)-7-fluorobenzo[d]thiazol-3(2H)-yl)butanoic acid;
2-(2-((3-chlorobenzoyl)imino)-5-fluorobenzo[d]thiazol-3(2H)-yl)acetic acid;
2-(2-((3-chlorobenzoyl)imino)-5-fluorobenzo[d]thiazol-3(2H)-yl)propionic acid;
2-(2-((3-chlorobenzoyl)imino)-5-fluorobenzo[d]thiazol-3(2H)-yl)butanoic acid;
2-(5,6,7-trifluoro-2-((3-(trifluoromethyl)benzoyl)imino)benzo[d]thiazol-3(2H)-yl)acetic acid;
2-(5,6,7-trifluoro-2-((3-(trifluoromethyl)benzoyl)imino)benzo[d]thiazol-3(2H)-yl)butanoic acid;
2-(5,6,7-trifluoro-2-((3-(trifluoromethyl)benzoyl)imino)benzo[d]thiazol-3(2H)-yl)propionic acid;
2-(6-chloro-7-fluoro-2-((3-(trifluoromethyl)benzoyl)imino)benzo[d]thiazol-3(2H)-yl)acetic acid;
2-(6-chloro-7-fluoro-2-((3-(trifluoromethyl)benzoyl)imino)benzo[d]thiazol-3(2H)-yl)propionic acid;
2-(6-chloro-7-fluoro-2-((3-(trifluoromethyl)benzoyl)imino)benzo[d]thiazol-3(2H)-yl)butanoic acid;
2-(6-chloro-2-((3-chlorobenzoyl)imino)-7-fluorobenzo[d]thiazol-3(2H)-yl)acetic acid;
2-(6-chloro-2-((3-chlorobenzoyl)imino)-7-fluorobenzo[d]thiazol-3(2H)-yl)propionic acid;
2-(6-chloro-2-((3-chlorobenzoyl)imino)-7-fluorobenzo[d]thiazol-3(2H)-yl)butanoic acid;
2-(2-((3-chlorobenzoyl)imino)-6-fluoro-5-(trifluoromethyl)benzo[d]thiazol-3(2H)-yl)acetic acid;
2-(2-((3-chlorobenzoyl)imino)-6-fluoro-5-(trifluoromethyl)benzo[d]thiazol-3(2H)-yl)propionic acid;
2-(2-((3-chlorobenzoyl)imino)-6-fluoro-5-(trifluoromethyl)benzo[d]thiazol-3(2H)-yl)butanoic acid;
2-(7-methoxy-2-((3-(trifluoromethyl)benzoyl)imino)benzo[d]thiazol-3(2H)-yl)acetic acid;
2-(7-methoxy-2-((3-(trifluoromethyl)benzoyl)imino)benzo[d]thiazol-3(2H)-yl)propionic acid;
2-(7-methoxy-2-((3-(trifluoromethyl)benzoyl)imino)benzo[d]thiazol-3(2H)-yl)butanoic acid;
2-(2-((3-chlorobenzoyl)imino)-7-methoxybenzo[d]thiazol-3(2H)-yl)acetic acid;
2-(2-((3,4-dichlorobenzoyl)imino)-6,7-difluorobenzo[d]thiazol-3(2H)-yl)butanoic acid;
2-(24(3,5-dichlorobenzoyl)imino)-6,7-difluorobenzo[d]thiazol-3(2H)-yl)butanoic acid.
2-(2-((3-chlorobenzoyl)imino)-7-fluoro-6-methoxybenzo[d]thiazol-3(2H)-yl)acetic acid;
2-(2-((3-chlorobenzoyl)imino)-7-fluoro-6-methoxybenzo[d]thiazol-3(2H)-yl)propionic acid;
2-(2-((3-chlorobenzoyl)imino)-7-fluoro-6-methoxybenzo[d]thiazol-3(2H)-yl)butanoic acid;
2-(6-ethoxy-7-fluoro-2-((3-(trifluoromethyl)benzoyl)imino)benzo[d]thiazol-3(2H)-yl)acetic acid;
2-(6-ethoxy-7-fluoro-2-((3-(trifluoromethyl)benzoyl)imino)benzo[d]thiazol-3(2H)-yl)propionic acid;
2-(6-ethoxy-7-fluoro-2-((3-(trifluoromethyl)benzoyl)imino)benzo[d]thiazol-3(2H)-yl)butanoic acid;
2-(7-chloro-5-(trifluoromethyl)-2-((3-(trifluoromethyl)benzoyl)imino)benzo[d]thiazol-3(2H)-yl)acetic acid;
2-(7-chloro-5-(trifluoromethyl)-2-((3-(trifluoromethyl)benzoyl)imino)benzo[d]thiazol-3(2H)-yl)propionic acid;
2-(7-chloro-5-(trifluoromethyl)-2-((3-(trifluoromethyl)benzoyl)imino)benzo[d]thiazol-3(2H)-yl)butanoic acid;
2-(7-chloro-2-((3-chlorobenzoyl)imino)-5-(trifluoromethyl)benzo[d]thiazol-3(2H)-yl)acetic acid;
2-(7-chloro-2-((3-chlorobenzoyl)imino)-5-(trifluoromethyl)benzo[d]thiazol-3(2H)-yl)propionic acid;
2-(7-chloro-2-((3-chlorobenzoyl)imino)-5-(trifluoromethyl)benzo[d]thiazol-3(2H)-yl)butanoic acid;
2-(7-fluoro-6-methyl-2-((3-(trifluoromethyl)benzoyl)imino)benzo[d]thiazol-3(2H)-yl)acetic acid;
2-(7-fluoro-6-methyl-2-((3-(trifluoromethyl)benzoyl)imino)benzo[d]thiazol-3(2H)-yl)propionic acid;
2-(7-fluoro-6-methyl-2-((3-(trifluoromethyl)benzoyl)imino)benzo[d]thiazol-3(2H)-yl)butanoic acid;
2-(2-((3-chlorobenzoyl)imino)-7-fluoro-6-methylbenzo[d]thiazol-3(2H)-yl)acetic acid;
2-(7-fluoro-6-methoxy-2-((3-(trifluoromethyl)benzoyl)imino)benzo[d]thiazol-3(2H)-yl)acetic acid;
2-(7-fluoro-6-methoxy-2-((3-(trifluoromethyl)benzoyl)imino)benzo[d]thiazol-3(2H)-yl)propionic acid;
2-(7-fluoro-6-methoxy-2-((3-(trifluoromethyl)benzoyl)imino)benzo[d]thiazol-3(2H)-yl)butanoic acid;
2-(2-((3-chlorobenzoyl)imino)-5,6,7-trifluorobenzo[d]thiazol-3(2H)-yl)acetic acid;
2-(2-((3-chlorobenzoyl)imino)-5,6,7-trifluorobenzo[d]thiazol-3(2H)-yl)propionic acid;
2-(2-((3-chlorobenzoyl)imino)-5,6,7-trifluorobenzo[d]thiazol-3(2H)-yl)butanoic acid;
2-(5-acetyl-2-((3-(trifluoromethyl)benzoyl)imino)benzo[d]thiazol-3(2H)-yl)acetic acid;
2-(5-acetyl-2-((3-(trifluoromethyl)benzoyl)imino)benzo[d]thiazol-3(2H)-yl)propionic acid;
2-(5-acetyl-2-((3-(trifluoromethyl)benzoyl)imino)benzo[d]thiazol-3(2H)-yl)butanoic acid;
2-(5-acetyl-2-((3-chlorobenzoyl)imino)benzo[d]thiazol-3(2H)-yl)acetic acid;
2-(5-acetyl-2-((3-chlorobenzoyl)imino)benzo[d]thiazol-3(2H)-yl)propionic acid;
2-(5-acetyl-2-((3-chlorobenzoyl)imino)benzo[d]thiazol-3(2H)-yl)butanoic acid;
2-(2-((3-chlorobenzoyl)imino)-7-fluoro-6-methylbenzo[d]thiazol-3(2H)-yl)propionic acid;
2-(2-((3-chlorobenzoyl)imino)-7-fluoro-6-methylbenzo[d]thiazol-3(2H)-yl)butanoic acid;
2-(2-((3-chlorobenzoyl)imino)-6-ethoxy-7-fluorobenzo[d]thiazol-3(2H)-yl)propionic acid;
2-(2-((3-chlorobenzoyl)imino)-6-ethoxy-7-fluorobenzo[d]thiazol-3(2H)-yl)butanoic acid;
2-(2-((3-chlorobenzoyl)imino)-7-fluoro-6-(trifluoromethyl)benzo[d]thiazol-3(2H)-yl)acetic acid;
2-(2-((3-chlorobenzoyl)imino)-7-fluoro-6-(trifluoromethyl)benzo[d]thiazol-3(2H)-yl)propionic acid;
2-(2-((3-chlorobenzoyl)imino)-7-fluoro-6-(trifluoromethyl)benzo[d]thiazol-3(2H)-yl)butanoic acid;
2-(7-fluoro-6-(trifluoromethyl)-2-((3-(trifluoromethyl)benzoyl)imino)benzo[d]thiazol-3(2H)-yl)acetic acid;
2-(7-fluoro-6-(trifluoromethyl)-2-((3-(trifluoromethyl)benzoyl)imino)benzo[d]thiazol-3(2H)-yl)propionic acid;
2-(7-fluoro-6-(trifluoromethyl)-2-((3-(trifluoromethyl)benzoyl)imino)benzo[d]thiazol-3(2H)-yl)butanoic acid;
2-(2-((2-chlorobenzoyl)imino)-5,6-difluorobenzo[d]thiazol-3(2H)-yl)butanoic acid;
2-(2-((4-chlorobenzoyl)imino)-5,6-difluorobenzo[d]thiazol-3(2H)-yl)butanoic acid;
2-(5,6-difluoro-2-((2-(trifluoromethyl)benzoyl)imino)benzo[d]thiazol-3(2H)-yl)butanoic acid;
2-(5,6-difluoro-2-((4-(trifluoromethyl)benzoyl)imino)benzo[d]thiazol-3(2H)-yl)butanoic acid;
2-(5,6-difluoro-2-((2-methoxybenzoyl)imino)benzo[d]thiazol-3(2H)-yl)butanoic acid;
2-(5,6-difluoro-2-((3-methoxybenzoyl)imino)benzo[d]thiazol-3(2H)-yl)butanoic acid;
2-(5,6-difluoro-2-((4-methoxybenzoyl)imino)benzo[d]thiazol-3(2H)-yl)butanoic acid;
2-(5,6-difluoro-2-((2-fluorobenzoyl)imino)benzo[d]thiazol-3(2H)-yl)butanoic acid;
2-(5,6-difluoro-2-((3-fluorobenzoyl)imino)benzo[d]thiazol-3(2H)-yl)butanoic acid;
2-(5,6-difluoro-2-((4-fluorobenzoyl)imino)benzo[d]thiazol-3(2H)-yl)butanoic acid;
2-(2-((2,4-difluorobenzoyl)imino)-5,6-difluorobenzo[d]thiazol-3(2H)-yl)butanoic acid;
2-(5,6-difluoro-2-((5-fluoro-2-(trifluoromethyl)benzoyl)imino)benzo[d]thiazol-3(2H)-yl)butanoic acid;
2-(2-((2-bromobenzoyl)imino)-5,6-difluorobenzo[d]thiazol-3(2H)-yl)butanoic acid;
2-(2-((4-bromobenzoyl)imino)-5,6-difluorobenzo[d]thiazol-3(2H)-yl)butanoic acid;
2-(2-((2,6-difluorobenzoyl)imino)-5,6-difluorobenzo[d]thiazol-3(2H)-yl)butanoic acid;
2-(2-((3,5-difluorobenzoyl)imino)-5,6-difluorobenzo[d]thiazol-3(2H)-yl)butanoic acid;
2-(5,6-difluoro-2-((2,3,6-trifluorobenzoyl)imino)benzo[d]thiazol-3(2H)-yl)butanoic acid;
2-(5,6-difluoro-2-((2,4,5-trifluorobenzoyl)imino)benzo[d]thiazol-3(2H)-yl)butanoic acid;
2-(5,6-difluoro-2-((5-fluoro-2-methylbenzoyl)imino)benzo[d]thiazol-3(2H)-yl)butanoic acid;
2-(5,6-difluoro-2-((4-(trifluoromethoxy)benzoyl)imino)benzo[d]thiazol-3(2H)-yl)butanoic acid;
2-(5,7-difluoro-2-((4-methylbenzoyl)imino)benzo[d]thiazol-3(2H)-yl)acetic acid;
2-(5,7-difluoro-2-((4-methylbenzoyl)imino)benzo[d]thiazol-3(2H)-yl)propionic acid;
2-(5,7-difluoro-2-((4-methylbenzoyl)imino)benzo[d]thiazol-3(2H)-yl)butanoic acid;
2-(5,7-difluoro-2-((3-(trifluoromethyl)benzoyl)imino)benzo[d]thiazol-3(2H)-yl)acetic acid;
2-(5,7-difluoro-2-((3-(trifluoromethyl)benzoyl)imino)benzo[d]thiazol-3(2H)-yl)propionic acid;
2-(5,7-difluoro-2-((3-(trifluoromethyl)benzoyl)imino)benzo[d]thiazol-3(2H)-yl)butanoic acid;
2-(2-((3-chlorobenzoyl)imino)-5,7-difluorobenzo[d]thiazol-3(2H)-yl)acetic acid;
2-(2-((3-chlorobenzoyl)imino)-5,7-difluorobenzo[d]thiazol-3(2H)-yl)propionic acid;
2-(2-((3-chlorobenzoyl)imino)-5,7-difluorobenzo[d]thiazol-3(2H)-yl)butanoic acid;
2-(2-((3,4-difluorobenzoyl)imino)-5,6-difluorobenzo[d]thiazol-3(2H)-yl)propionic acid;
2-(2-((3,4-difluorobenzoyl)imino)-5,6-difluorobenzo[d]thiazol-3(2H)-yl)butanoic acid;
2-(2-((3,5-bis(trifluoromethyl)benzoyl)imino)-5,6-difluorobenzo[d]thiazol-3(2H)-yl)propionic acid;
2-(2-((3-chloro-4-fluorobenzoyl)imino)-5,6-difluorobenzo[d]thiazol-3(2H)-yl)propionic acid;
2-(2-((3-chloro-4-fluorobenzoyl)imino)-5,6-difluorobenzo[d]thiazol-3(2H)-yl)butanoic acid;
2-(2-((3,5-dichlorobenzoyl)imino)-5,6-difluorobenzo[d]thiazol-3(2H)-yl)butanoic acid;
2-(5,6-difluoro-2-((3-fluoro-5-(trifluoromethyl)benzoyl)iminobenzo[d]thiazol-3(2H)-yl)butanoic acid;
2-(5,6-difluoro-2-((3-fluoro-4-methylbenzoyl)imino)benzo[d]thiazol-3(2H)-yl)butanoic acid;
2-(5,6-difluoro-2-((4-fluoro-3-(trifluoromethyl)benzoyl)iminobenzo[d]thiazol-3(2H)-yl)butanoic acid;
2-(2-((3,4-dichlorobenzoyl)imino)-5,6-difluorobenzo[d]thiazol-3(2H)-yl)butanoic acid;
2-(2-((3,5-dimethoxybenzoyl)imino-5,6-difluorobenzo[d]thiazol-3(2H)-yl)butanoic acid;
2-(5,6-difluoro-2-((3-(trifluoromethoxy)benzoyl)iminobenzo[d]thiazol-3(2H)-yl)butanoic acid;
2-(6-ethoxy-2-((3-fluorobenzoyl)imino)benzo[d]thiazol-3(2H)-yl)butanoic acid;
2-(6-ethoxy-2-((4-fluorobenzoyl)imino)benzo[d]thiazol-3(2H)-yl)butanoic acid;
2-(6-ethoxy-2-((3-methoxybenzoyl)iminobenzo[d]thiazol-3(2H)-yl)butanoic acid;
2-(6-ethoxy-2-((4-methoxybenzoyl)iminobenzo[d]thiazol-3(2H)-yl)butanoic acid;
2-(2-((4-chlorobenzoyl)imino)-6-ethoxybenzo[d]thiazol-3(2H)-yl)butanoic acid;
2-(6-ethoxy-2-((3-(trifluoromethoxy)benzoyl)iminobenzo[d]thiazol-3(2H)-yl)butanoic acid;
2-(2-((3,5-dimethoxybenzoyl)imino-6-ethoxybenzo[d]thiazol-3(2H)-yl)butanoic acid;
2-(2-((3,4-difluorobenzoyl)imino)-6-ethoxybenzo[d]thiazol-3(2H)-yl)butanoic acid;
2-(2-((3,5-difluorobenzoyl)imino)-6-ethoxybenzo[d]thiazol-3(2H)-yl)butanoic acid;
2-(6-ethoxy-2-((3-fluoro-5-(trifluoromethyl)benzoyl)iminobenzo[d]thiazol-3(2H)-yl)butanoic acid;
2-(2-((3,4-dichlorobenzoyl)imino)-6-ethoxybenzo[d]thiazol-3(2H)-yl)butanoic acid;
2-(2-((3,5-dichlorobenzoyl)imino)-6-ethoxybenzo[d]thiazol-3(2H)-yl)butanoic acid;
2-(2-((3-chloro-4-fluorobenzoyl)imino)-6-ethoxybenzo[d]thiazol-3(2H)-yl)butanoic acid;
2-(2-((3,5-bis(trifluoromethyl)benzoyl)imino)-6-ethoxybenzo[d]thiazol-3(2H)-yl)butanoic acid;
2-(6-ethoxy-2-((3-fluoro-4-methylbenzoyl)iminobenzo[d]thiazol-3(2H)-yl)butanoic acid;
2-(6-ethoxy-2-((4-fluoro-3-(trifluoromethyl)benzoyl)iminobenzo[d]thiazol-3(2H)-yl)butanoic acid;
2-(2-((3-fluorobenzoyl)imino)-6-(trifluoromethyl)benzo[d]thiazol-3(2H)-yl)butanoic acid;
2-(2-((4-fluorobenzoyl)imino)-6-(trifluoromethyl)benzo[d]thiazol-3(2H)-yl)butanoic acid;
2-(2-((3-methoxybenzoyl)imino-6-(trifluoromethyl)benzo[d]thiazol-3(2H)-yl)butanoic acid;
2-(2-((4-methoxybenzoyl)imino-6-(trifluoromethyl)benzo[d]thiazol-3(2H)-yl)butanoic acid;
2-(2-((4-chlorobenzoyl)imino)-6-(trifluoromethyl)benzo[d]thiazol-3(2H)-yl)butanoic acid;
2-(2-((3-(trifluoromethoxy)benzoyl)imino-6-(trifluoromethyl)benzo[d]thiazol-3(2H)-yl)butanoic acid;
2-(2-((3,5-dimethoxybenzoyl)imino-6-(trifluoromethyl)benzo[d]thiazol-3(2H)-yl)butanoic acid;
2-(2-((3,4-difluorobenzoyl)imino)-6-(trifluoromethyl)benzo[d]thiazol-3(2H)-yl)butanoic acid;
2-(2-((3,5-difluorobenzoyl)imino)-6-(trifluoromethyl)benzo[d]thiazol-3(2H)-yl)butanoic acid;
2-(2-((3-fluoro-5-(trifluoromethyl)benzoyl)imino-6-(trifluoromethyl)benzo[d]thiazol-3(2H)-yl)butanoic acid;
2-(2-((3,4-dichlorobenzoyl)imino)-6-(trifluoromethyl)benzo[d]thiazol-3(2H)-yl)butanoic acid;
2-(2-((3,5-dichlorobenzoyl)imino)-6-(trifluoromethyl)benzo[d]thiazol-3(2H)-yl)butanoic acid;
2-(2-((3-chloro-4-fluorobenzoyl)imino)-6-(trifluoromethyl)benzo[d]thiazol-3(2H)-yl)butanoic acid;
2-(2-((3-fluorobenzoyl)imino)-6-(trifluoromethoxy)benzo[d]thiazol-3(2H)-yl)butanoic acid;
2-(2-((4-fluorobenzoyl)imino)-6-(trifluoromethoxy)benzo[d]thiazol-3(2H)-yl)butanoic acid;
2-(2-((3-methoxybenzoyl)imino-6-(trifluoromethoxy)benzo[d]thiazol-3(2H)-yl)butanoic acid;
2-(2-((4-methoxybenzoyl)imino-6-(trifluoromethoxy)benzo[d]thiazol-3(2H)-yl)butanoic acid;
2-(2-((4-chlorobenzoyl)imino)-6-(trifluoromethoxy)benzo[d]thiazol-3(2H)-yl)butanoic acid;
2-(6-(trifluoromethoxy)-2-((3-(trifluoromethoxy)benzoyl)iminobenzo[d]thiazol-3(2H)-yl)butanoic acid;
2-(2-((3,5-dimethoxybenzoyl)imino-6-(trifluoromethoxy)benzo[d]thiazol-3(2H)-yl)butanoic acid;
2-(2-((3,4-difluorobenzoyl)imino)-6-(trifluoromethoxy)benzo[d]thiazol-3(2H)-yl)butanoic acid;
2-(2-((3,5-difluorobenzoyl)imino)-6-(trifluoromethoxy)benzo[d]thiazol-3(2H)-yl)butanoic acid;
2-(2-((3-fluoro-5-(trifluoromethyl)benzoyl)imino-6-(trifluoromethoxy)benzo[d]thiazol-3(2H)-yl)butanoic acid;
2-(2-((3,4-dichlorobenzoyl)imino)-6-(trifluoromethoxy)benzo[d]thiazol-3(2H)-yl)butanoic acid;
2-(2-((3,5-dichlorobenzoyl)imino)-6-(trifluoromethoxy)benzo[d]thiazol-3(2H)-yl)butanoic acid;
2-(2-((3-chloro-4-fluorobenzoyl)imino)-6-(trifluoromethoxy)benzo[d]thiazol-3(2H)-yl)butanoic acid;
2-(5,6,7-trifluoro-2-((3-fluorobenzoyl)imino)benzo[d]thiazol-3(2H)-yl)butanoic acid;
2-(5,6,7-trifluoro-2-((4-fluorobenzoyl)imino)benzo[d]thiazol-3(2H)-yl)butanoic acid;
2-(5,6,7-trifluoro-2-((3-methoxybenzoyl)iminobenzo[d]thiazol-3(2H)-yl)butanoic acid;
2-(5,6,7-trifluoro-2-((4-methoxybenzoyl)iminobenzo[d]thiazol-3(2H)-yl)butanoic acid;
2-(2-((4-chlorobenzoyl)imino)-5,6,7-trifluorobenzo[d]thiazol-3(2H)-yl)butanoic acid;
2-(5,6,7-trifluoro-2-((3-fluoro-4-methylbenzoyl)iminobenzo[d]thiazol-3(2H)-yl)butanoic acid;
2-(2-((3-chloro-4-fluorobenzoyl)imino)-6,7-difluorobenzo[d]thiazol-3(2H)-yl)butanoic acid;
2-(2-((3,4-difluorobenzoyl)imino)-5,6,7-trifluorobenzo[d]thiazol-3(2H)-yl)butanoic acid;
2-(2-((3,5-difluorobenzoyl)imino)-5,6,7-trifluorobenzo[d]thiazol-3(2H)-yl)butanoic acid;
2-(5,6,7-trifluoro-2-((3-fluoro-5-(trifluoromethyl)benzoyl)iminobenzo[d]thiazol-3(2H)-yl)butanoic acid;
2-(5,6,7-trifluoro-2-((4-fluoro-3-(trifluoromethyl)benzoyl)imino)-benzo[d]thiazol-3(2H)-yl)butanoic acid;
2-(2-((3,5-dichlorobenzoyl)imino)-5,6,7-trifluorobenzo[d]thiazol-3(2H)-yl)butanoic acid;
2-(2-((3-chloro-4-fluorobenzoyl)imino)-5,6,7-trifluorobenzo[d]thiazol-3(2H)-yl)butanoic acid;
2-(7-fluoro-2-((3-fluorobenzoyl)imino)benzo[d]thiazol-3(2H)-yl)butanoic acid;
2-(7-fluoro-2-((4-fluorobenzoyl)imino)benzo[d]thiazol-3(2H)-yl)butanoic acid;
2-(7-fluoro-2-((3-methoxybenzoyl)iminobenzo[d]thiazol-3(2H)-yl)butanoic acid;
2-(7-fluoro-2-((4-methoxybenzoyl)iminobenzo[d]thiazol-3(2H)-yl)butanoic acid;
2-(2-((4-chlorobenzoyl)imino)-7-fluorobenzo[d]thiazol-3(2H)-yl)butanoic acid;
2-(7-fluoro-2-((3-(trifluoromethoxy)benzoyl)iminobenzo[d]thiazol-3(2H)-yl)butanoic acid;
2-(2-((3,5-dimethoxybenzoyl)imino-7-fluorobenzo[d]thiazol-3(2H)-yl)butanoic acid;
2-(2-((3,4-difluorobenzoyl)imino)-7-fluorobenzo[d]thiazol-3(2H)-yl)butanoic acid;
2-(2-((3,5-difluorobenzoyl)imino)-7-fluorobenzo[d]thiazol-3(2H)-yl)butanoic acid;
2-(7-fluoro-2-((3-fluoro-5-(trifluoromethyl)benzoyl)iminobenzo[d]thiazol-3(2H)-yl)butanoic acid;
2-(2-((3,4-dichlorobenzoyl)imino)-7-fluorobenzo[d]thiazol-3(2H)-yl)butanoic acid;
2-(2-((3,5-dichlorobenzoyl)imino)-7-fluorobenzo[d]thiazol-3(2H)-yl)butanoic acid;
2-(2-((3-chloro-4-fluorobenzoyl)imino)-7-fluorobenzo[d]thiazol-3(2H)-yl)butanoic acid;
2-(7-chloro-2-((3-fluorobenzoyl)imino)benzo[d]thiazol-3(2H)-yl)butanoic acid;
2-(7-chloro-2-((4-fluorobenzoyl)imino)benzo[d]thiazol-3(2H)-yl)butanoic acid;
2-(7-chloro-2-((3-methoxybenzoyl)iminobenzo[d]thiazol-3(2H)-yl)butanoic acid;
2-(7-chloro-2-((4-methoxybenzoyl)iminobenzo[d]thiazol-3(2H)-yl)butanoic acid;
2-(7-chloro-2-((4-chlorobenzoyl)imino)benzo[d]thiazol-3(2H)-yl)butanoic acid;
2-(7-chloro-2-((3-(trifluoromethoxy)benzoyl)iminobenzo[d]thiazol-3(2H)-yl)butanoic acid;
2-(7-chloro-2-((3,5-dimethoxybenzoyl)iminobenzo[d]thiazol-3(2H)-yl)butanoic acid;
2-(7-chloro-2-((3,4-difluorobenzoyl)imino)benzo[d]thiazol-3(2H)-yl)butanoic acid;
2-(7-chloro-2-((3,5-difluorobenzoyl)imino)benzo[d]thiazol-3(2H)-yl)butanoic acid;
2-(7-chloro-2-((3-fluoro-5-(trifluoromethyl)benzoyl)iminobenzo[d]thiazol-3(2H)-yl)butanoic acid;
2-(7-chloro-2-((3,4-dichlorobenzoyl)imino)benzo[d]thiazol-3(2H)-yl)butanoic acid;
2-(7-chloro-2-((3,5-dichlorobenzoyl)imino)benzo[d]thiazol-3(2H)-yl)butanoic acid;
2-(7-chloro-2-((3-chloro-4-fluorobenzoyl)imino)benzo[d]thiazol-3(2H)-yl)butanoic acid;
2-(2-((3-fluorobenzoyl)imino)-7-(trifluoromethyl)benzo[d]thiazol-3(2H)-yl)butanoic acid;
2-(2-((4-fluorobenzoyl)imino)-7-(trifluoromethyl)benzo[d]thiazol-3(2H)-yl)butanoic acid;
2-(2-((3-methoxybenzoyl)imino-7-(trifluoromethyl)benzo[d]thiazol-3(2H)-yl)butanoic acid;
2-(2-((4-methoxybenzoyl)imino-7-(trifluoromethyl)benzo[d]thiazol-3(2H)-yl)butanoic acid;
2-(2-((4-chlorobenzoyl)imino)-7-(trifluoromethyl)benzo[d]thiazol-3(2H)-yl)butanoic acid;
2-(2-((3-(trifluoromethoxy)benzoyl)imino-7-(trifluoromethyl)benzo[d]thiazol-3(2H)-yl)butanoic acid;
2-(2-((3,5-dimethoxybenzoyl)imino-7-(trifluoromethyl)benzo[d]thiazol-3(2H)-yl)butanoic acid;
2-(2-((3,4-difluorobenzoyl)imino)-7-(trifluoromethyl)benzo[d]thiazol-3(2H)-yl)butanoic acid;
2-(2-((3,5-difluorobenzoyl)imino)-7-(trifluoromethyl)benzo[d]thiazol-3(2H)-yl)butanoic acid;
2-(2-((3-fluoro-5-(trifluoromethyl)benzoyl)imino-7-(trifluoromethyl)benzo[d]thiazol-3(2H)-yl)butanoic acid;
2-(2-((3,4-dichlorobenzoyl)imino)-7-(trifluoromethyl)benzo[d]thiazol-3(2H)-yl)butanoic acid;
2-(2-((3,5-dichlorobenzoyl)imino)-7-(trifluoromethyl)benzo[d]thiazol-3(2H)-yl)butanoic acid;
2-(2-((3-chloro-4-fluorobenzoyl)imino)-7-(trifluoromethyl)benzo[d]thiazol-3(2H)-yl)butanoic acid;
2-(5-chloro-2-((3-fluorobenzoyl)imino)benzo[d]thiazol-3(2H)-yl)butanoic acid;
2-(5-chloro-2-((4-fluorobenzoyl)imino)benzo[d]thiazol-3(2H)-yl)butanoic acid;
2-(5-chloro-2-((3-methoxybenzoyl)iminobenzo[d]thiazol-3(2H)-yl)butanoic acid;
2-(5-chloro-2-((4-methoxybenzoyl)iminobenzo[d]thiazol-3(2H)-yl)butanoic acid;
2-(5-chloro-2-((4-chlorobenzoyl)imino)benzo[d]thiazol-3(2H)-yl)butanoic acid;
2-(5-chloro-2-((3-(trifluoromethoxy)benzoyl)iminobenzo[d]thiazol-3(2H)-yl)butanoic acid;
2-(5-chloro-2-((3,5-dimethoxybenzoyl)iminobenzo[d]thiazol-3(2H)-yl)butanoic acid;
2-(5-chloro-2-((3,4-difluorobenzoyl)imino)benzo[d]thiazol-3(2H)-yl)butanoic acid;
2-(5-chloro-2-((3,5-difluorobenzoyl)imino)benzo[d]thiazol-3(2H)-yl)butanoic acid;
2-(5-chloro-2-((3-fluoro-5-(trifluoromethyl)benzoyl)iminobenzo[d]thiazol-3(2H)-yl)butanoic acid;
2-(5-chloro-2-((3,4-dichlorobenzoyl)imino)benzo[d]thiazol-3(2H)-yl)butanoic acid;
2-(5-chloro-2-((3,5-dichlorobenzoyl)imino)benzo[d]thiazol-3(2H)-yl)butanoic acid;
2-(5-chloro-2-((3-chloro-4-fluorobenzoyl)imino)benzo[d]thiazol-3(2H)-yl)butanoic acid;
2-(6,7-difluoro-2-((3-fluorobenzoyl)imino)benzo[d]thiazol-3(2H)-yl)butanoic acid;
2-(6,7-difluoro-2-((4-fluorobenzoyl)imino)benzo[d]thiazol-3(2H)-yl)butanoic acid;
2-(6,7-difluoro-2-((3-methoxybenzoyl)iminobenzo[d]thiazol-3(2H)-yl)butanoic acid;
2-(6,7-difluoro-2-((4-methoxybenzoyl)iminobenzo[d]thiazol-3(2H)-yl)butanoic acid;
2-(2-((4-chlorobenzoyl)imino)-6,7-difluorobenzo[d]thiazol-3(2H)-yl)butanoic acid;
2-(6,7-difluoro-2-((3-(trifluoromethoxy)benzoyl)iminobenzo[d]thiazol-3(2H)-yl)butanoic acid;
2-(2-((3,5-dimethoxybenzoyl)imino-6,7-difluorobenzo[d]thiazol-3(2H)-yl)butanoic acid;
2-(2-((3,4-difluorobenzoyl)imino)-6,7-difluorobenzo[d]thiazol-3(2H)-yl)butanoic acid;
2-(2-((3,5-difluorobenzoyl)imino)-6,7-difluorobenzo[d]thiazol-3(2H)-yl)butanoic acid; and
2-(6,7-difluoro-2-((3-fluoro-5-(trifluoromethyl)benzoyl)iminobenzo[d]thiazol-3(2H)-yl)butanoic acid.

7. The compound or its pharmaceutically acceptable salt of claim 3, which is selected from the group consisting of:
2-{5,6-dimethyl-2-[(4-methylbenzoyl)imino]benzo[d]thiazol-3(2H)-yl}butanoic acid;
2-{2-[(4-methylbenzoyl)imino]-6-(trifluoromethoxy)benzo[d]thiazol-3(2H)-yl]butanoic acid;
2-{5-fluoro-6-methyl-2-[(4-methylbenzoyl)imino]benzo[d]thiazol-3(2H)-yl}butanoic acid;
2-{2-[(4-methylbenzoyl)imino]-6-nitrobenzo[d]thiazol-3(2H)-yl}butanoic acid;
2-{2-[(3-chlorobenzoyl)imino]-6-methoxybenzo[d]thiazol-3(2H)-yl}propionic acid;
2-{2-[(3-chlorobenzoyl)imino]-6-ethoxybenzo[d]thiazol-3(2H)-yl}propionic acid;
2-{2-[(3-chlorobenzoyl)imino]-6-ethoxybenzo[d]thiazol-3(2H)-yl}butanoic acid;
2-{2-[(3-chlorobenzoyl)imino]-6-methylbenzo[d]thiazol-3(2H)-yl}butanoic acid;
2-[5-fluoro-2-(4-methylbenzoylimino)benzo[d]thiazol-3(2H)-yl]butanoic acid;
2-[7-fluoro-2-(4-methylbenzoylimino)benzo[d]thiazol-3(2H)-yl]butanoic acid;
2-[2-(4-methylbenzoylimino)-7-(trifluoromethyl)benzo[d]thiazol-3(2H)-yl]propionic acid;
2-[5-methyl-2-(4-methylbenzoylimino)benzo[d]thiazol-3(2H)-yl]butanoic acid;
2-[7-methyl-2-(4-methylbenzoylimino)benzo[d]thiazol-3(2H)-yl]propionic acid;
2-[5-chloro-2-(4-methylbenzoylimino)benzo[d]thiazol-3(2H)-yl]butanoic acid;
2-[7-chloro-2-(4-methylbenzoylimino)benzo[d]thiazol-3(2H)-yl]propionic acid;
2-[6-fluoro-2-(4-methylbenzoylimino)benzo[d]thiazol-3(2H)-yl]butanoic acid;
2-[2-(4-methylbenzoylimino)-6-(trifluoromethyl)benzo[d]thiazol-3(2H)-yl]butanoic acid;
2-(2-((3-chlorobenzoyl)imino)-6-(trifluoromethyl)benzo[d]thiazol-3(2H)-yl)propionic acid;
2-(2-((3-chlorobenzoyl)imino)-6-(trifluoromethyl)benzo[d]thiazol-3(2H)-yl)butanoic acid;
2-(2-((3-chlorobenzoyl)imino)-6-(trifluoromethoxy)benzo[d]thiazol-3(2H)-yl)butanoic acid;
2-(6-methoxy-2-((3-(trifluoromethyl)benzoyl)imino)benzo[d]thiazol-3(2H)-yl)propionic acid;
2-(6-ethoxy-2-((3-(trifluoromethyl)benzoyl)imino)benzo[d]thiazol-3(2H)-yl)propionic acid;
2-(6-methyl-2-((3-(trifluoromethyl)benzoyl)imino)benzo[d]thiazol-3(2H)-yl)propionic acid;
2-(2-((3-chlorobenzoyl)imino)-5,6-dimethylbenzo[d]thiazol-3(2H)-yl)propionic acid;
2-(6-((3-chlorobenzoyl)imino)-[1,3]dioxolo[4′,5′:4,5]benzo[1,2-d]thiazol-7(6H)-yl)butanoic acid;
2-(2-((4-methylbenzoyl)imino)-6-(trifluoromethyl)benzo[d]thiazol-3(2H)-yl)acetic acid;
2-(2-(3-chlorobenzoylimino)-7-(trifluoromethyl)benzo[d]thiazol-3(2H)-yl)propionic acid;
2-(2-(3-chlorobenzoylimino)-4-methoxybenzo[d]thiazol-3(2H)-yl)propionic acid;
2-(4-methoxy-2-(3-(trifluoromethyl)benzoylimino)benzo[d]thiazol-3(2H)-yl)propionic acid;
2-(2-((3-chlorobenzoyl)imino)-5,6-difluorobenzo[d]thiazol-3(2H)-yl)propionic acid;
2-(2-((3-chlorobenzoyl)imino)-5,6-difluorobenzo[d]thiazol-3(2H)-yl)butanoic acid;
2-(5,6-difluoro-2-((3-(trifluoromethyl)benzoyl)imino)benzo[d]thiazol-3(2H)-yl)butanoic acid;
2-(7-chloro-2-((3-(trifluoromethyl)benzoyl)imino)benzo[d]thiazol-3(2H)-yl)propionic acid;
2-(7-chloro-2-((3-chlorobenzoyl)imino)benzo[d]thiazol-3(2H)-yl)propionic acid;
2-(7-chloro-2-((3-chlorobenzoyl)imino)benzo[d]thiazol-3(2H)-yl)butanoic acid;
2-(5-chloro-2-(3-chlorobenzoylimino)benzo[d]thiazol-3(2H)-yl)acetic acid;
2-(7-methyl-2-(3-(trifluoromethyl)benzoylimino)benzo[d]thiazol-3(2H)-yl)propionic acid;
2-(2-((3-chlorobenzoyl)imino)-6,7-difluorobenzo[d]thiazol-3(2H)-yl)butanoic acid;
2-(7-fluoro-2-((3-(trifluoromethyl)benzoyl)imino)benzo[d]thiazol-3(2H)-yl)propionic acid;
2-(2-((3-chlorobenzoyl)imino)-7-fluorobenzo[d]thiazol-3(2H)-yl)acetic acid;
2-(7-fluoro-2-((3-(trifluoromethyl)benzoyl)imino)benzo[d]thiazol-3(2H)-yl)acetic acid;
2-(7-chloro-2-((3-chlorobenzoyl)imino)benzo[d]thiazol-3(2H)-yl)acetic acid;
2-(5-chloro-2-((3-chlorobenzoyl)imino)benzo[d]thiazol-3(2H)-yl)butanoic acid;
2-(2-((3-chlorobenzoyl)imino)-7-fluorobenzo[d]thiazol-3(2H)-yl)propionic acid;
2-(5,6,7-trifluoro-2-((3-(trifluoromethyl)benzoyl)imino)benzo[d]thiazol-3(2H)-yl)propionic acid;
2-(6-chloro-2-((3-chlorobenzoyl)imino)-7-fluorobenzo[d]thiazol-3(2H)-yl)propionic acid;
2-(6-chloro-2-((3-chlorobenzoyl)imino)-7-fluorobenzo[d]thiazol-3(2H)-yl)butanoic acid;
2-(2-((3-chlorobenzoyl)imino)-6-fluoro-5-(trifluoromethyl)benzo[d]thiazol-3(2H)-yl)propionic acid;
2-(2-((3-chlorobenzoyl)imino)-7-fluoro-6-methoxybenzo[d]thiazol-3(2H)-yl)acetic acid;
2-(2-((3-chlorobenzoyl)imino)-5,6,7-trifluorobenzo[d]thiazol-3(2H)-yl)butanoic acid;
2-(2-((3-chlorobenzoyl)imino)-7-fluoro-6-methylbenzo[d]thiazol-3(2H)-yl)propionic acid;
2-(2-((3-chlorobenzoyl)imino)-6-ethoxy-7-fluorobenzo[d]thiazol-3(2H)-yl)butanoic acid;
2-(5,6-difluoro-2-((3-fluorobenzoyl)imino)benzo[d]thiazol-3(2H)-yl)butanoic acid;
2-(5,7-difluoro-2-((4-methylbenzoyl)imino)benzo[d]thiazol-3(2H)-yl)propionic acid;
2-(5,7-difluoro-2-((3-(trifluoromethyl)benzoyl)imino)benzo[d]thiazol-3(2H)-yl)propionic acid;
2-(2-((3-chlorobenzoyl)imino)-5,7-difluorobenzo[d]thiazol-3(2H)-yl)butanoic acid;
2-(2-((3-chloro-4-fluorobenzoyl)imino)-5,6-difluorobenzo[d]thiazol-3(2H)-yl)propionic acid;
2-(2-((3-chloro-4-fluorobenzoyl)imino)-5,6-difluorobenzo[d]thiazol-3(2H)-yl)butanoic acid;
2-(2-((3,5-dichlorobenzoyl)imino)-5,6-difluorobenzo[d]thiazol-3(2H)-yl)butanoic acid;
2-(5,6-difluoro-2-((3-fluoro-5-(trifluoromethyl)benzoyl)iminobenzo[d]thiazol-3(2H)-yl)butanoic acid;
2-(5,6-difluoro-2-((4-fluoro-3-(trifluoromethyl)benzoyl)iminobenzo[d]thiazol-3(2H)-yl)butanoic acid;
2-{6-ethoxy-2-[(4-methylbenzoyl)imino]benzo[d]thiazol-3(2H)-yl}butanoic acid;
2-{4-methoxy-2-[(4-methylbenzoyl)imino]benzo[d]thiazol-3(2H)-yl}butanoic acid;
2-[5-fluoro-2-(4-methylbenzoylimino)benzo[d]thiazol-3(2H)-yl]propionic acid;
2-(6-((4-methylbenzoyl)imino)-[1,3]dioxolo[4′,5′:4,5]benzo[1,2-d]thiazol-7(6H)-yl)butanoic acid;
2-(6,7-difluoro-2-((4-methylbenzoyl)imino)benzo[d]thiazol-3(2H)-yl)butanoic acid;
2-(2-((3-chlorobenzoyl)imino)-6,7-difluorobenzo[d]thiazol-3(2H)-yl)propionic acid;
2-(7-methyl-2-((4-methylbenzoyl)imino)benzo[d]thiazol-3(2H)-yl)butanoic acid;
2-(2-((3-chlorobenzoyl)imino)-7-methylbenzo[d]thiazol-3(2H)-yl)propionic acid;
2-(2-((3-chlorobenzoyl)imino)-7-methylbenzo[d]thiazol-3(2H)-yl)butanoic acid;
2-(5-fluoro-2-((3-(trifluoromethyl)benzoyl)imino)benzo[d]thiazol-3(2H)-yl)propionic acid;
2-(5-chloro-2-((3-(trifluoromethyl)benzoyl)imino)benzo[d]thiazol-3(2H)-yl)propionic acid; and
2-(2-((3-chlorobenzoyl)imino)-5,6,7-trifluorobenzo[d]thiazol-3(2H)-yl)propionic acid.

8. The compound or its pharmaceutically acceptable salt of claim 3, which is selected from the group consisting of:
2-{2-[(3-chlorobenzoyl)imino]-6-ethoxybenzo[d]thiazol-3(2H)-yl}butanoic acid;
2-[7-fluoro-2-(4-methylbenzoylimino)benzo[d]thiazol-3(2H)-yl]butanoic acid;
2-[2-(4-methylbenzoylimino)-7-(trifluoromethyl)benzo[d]thiazol-3(2H)-yl]propionic acid;
2-(2-((3-chlorobenzoyl)imino)-6-(trifluoromethyl)benzo[d]thiazol-3(2H)-yl)butanoic acid;
2-(2-((3-chlorobenzoyl)imino)-6-(trifluoromethoxy)benzo[d]thiazol-3(2H)-yl)butanoic acid;
2-(4-methoxy-2-(3-(trifluoromethyl)benzoylimino)benzo[d]thiazol-3(2H)-yl)propionic acid;
2-(2-((3-chlorobenzoyl)imino)-5,6-difluorobenzo[d]thiazol-3(2H)-yl)propionic acid;
2-(2-((3-chlorobenzoyl)imino)-5,6-difluorobenzo[d]thiazol-3(2H)-yl)butanoic acid;
2-(5,6-difluoro-2-((3-(trifluoromethyl)benzoyl)imino)benzo[d]thiazol-3(2H)-yl)butanoic acid;
2-(7-chloro-2-((3-(trifluoromethyl)benzoyl)imino)benzo[d]thiazol-3(2H)-yl)propionic acid;
2-(7-chloro-2-((3-chlorobenzoyl)imino)benzo[d]thiazol-3(2H)-yl)butanoic acid;
2-(5-chloro-2-((3-chlorobenzoyl)imino)benzo[d]thiazol-3(2H)-yl)butanoic acid;
2-(5,6,7-trifluoro-2-((3-(trifluoromethyl)benzoyl)imino)benzo[d]thiazol-3(2H)-yl)propionic acid;
2-(6-chloro-2-((3-chlorobenzoyl)imino)-7-fluorobenzo[d]thiazol-3(2H)-yl)propionic acid;
2-(6-chloro-2-((3-chlorobenzoyl)imino)-7-fluorobenzo[d]thiazol-3(2H)-yl)butanoic acid; and
2-(2-((3-chlorobenzoyl)imino)-6-fluoro-5-(trifluoromethyl)benzo[d]thiazol-3(2H)-yl)propionic acid.

9. The compound or its pharmaceutically acceptable salt of claim 4, which is selected from the group consisting of:
2-[2-(4-methoxybenzoylimino)benzo[d]thiazol-3(2H)-yl]butanoic acid;
2-[2-(3,5-difluorobenzoylimino)benzo[d]thiazol-3(2H)-yl]butanoic acid;
2-[2-(3-bromobenzoylimino)benzo[d]thiazol-3(2H)-yl]butanoic acid;
2-[2-(3-chlorobenzoylimino)benzo[d]thiazol-3(2H)-yl]acetic acid; and
2-[2-(3-chlorobenzoylimino)benzo[d]thiazol-3(2H)-yl]butanoic acid.

10. The compound or its pharmaceutically acceptable salt of claim 5, which is selected from the group consisting of:
2-(2-(3-(trifluoromethyl)benzoylimino)-4,5,6,7-tetrahydrobenzo[d]thiazol-3(2H)-yl)propionic acid; and
2-(2-(3-chlorobenzoylimino)-4,5,6,7-tetrahydrobenzo[d]thiazol-3(2H)-yl)butanoic acid.

11. 2-(2-(3-Chlorobenzoylimino)-4,5,6,7-tetrahydrobenzo[d]thiazol-3(2H)-yl)butanoic acid or its pharmaceutically acceptable salt.

12. A pharmaceutical composition for preventing or treating a disease associated with cancer cell metastasis, comprising a therapeutically effective amount of the compound or its pharmaceutically acceptable salt according to claim 1; and a pharmaceutically acceptable carrier.

13. The pharmaceutical composition of claim 12, wherein the disease associated with cancer cell metastasis is selected from the group consisting of colon cancer, lung cancer, hepatic cancer, gastric cancer, esophageal cancer, pancreatic cancer, gallbladder cancer, kidney cancer, bladder cancer, prostate cancer, testicular cancer, cervical cancer, endometrial cancer, choriocarcinoma, ovarian cancer, breast cancer, thyroid cancer, brain tumor, head and neck cancer, malignant melanoma, lymphoma, and aplastic anemia.

14. A pharmaceutical composition for inhibiting cancer cell metastasis, comprising a therapeutically effective amount of the compound or its pharmaceutically acceptable salt according to claim 1; and a pharmaceutically acceptable carrier.
